(19) United States
(12) Patent Application Publication
(10) Pub. No.: US 2022/0186322 A1
(43) Pub. Date: Jun. 16, 2022

(54) MARKER FOR HOMOLOGOUS RECOMBINATION DEFICIENCY, METHOD AND SYSTEM FOR DETECTION THEREOF

(71) Applicant: NANJING SHIHE GENE BIOTECHNOLOGY CO.,LTD., Jiangsu (CN)

(72) Inventors: Chenglong NA, Jiangsu (CN); Yang SHAO, Jiangsu (CN); Hua BAO, Jiangsu (CN); Rui LIU, Jiangsu (CN); Shuyu WU, Jiangsu (CN); Yi SHEN, Jiangsu (CN); Xiaonan WANG, Jiangsu (CN); Xue WU, Jiangsu (CN)

(73) Assignee: NANJING SHIHE GENE BIOTECHNOLOGY CO.,LTD., Jiangsu (CN)

(21) Appl. No.: 17/686,428

(22) Filed: Mar. 4, 2022

Related U.S. Application Data
(63) Continuation of application No. PCT/CN2021/072605, filed on Jan. 19, 2021.

(30) Foreign Application Priority Data
Nov. 3, 2020 (CN) 202011208296.8

(51) Int. Cl. C12Q 1/6886

(52) U.S. Cl. CPC C12Q 1/6886; C12Q 2600/156

(57) ABSTRACT

The present invention relates to a field of medical molecular biology, and in particular, a marker for homologous recombination deficiency (HRD), a method and a system for detection thereof. In the present invention, the genome-wide single nucleotide polymorphisms (SNPs) with a high heterozygous rate suitable for the Chinese population are provided by screening SNP sites on the gnomAD records and over 300 Chinese whole-genome sequencing (WGS) data, thereby establishing a HRD scoring method. The HRD markers provided by the present invention allow HRD to be effectively scored, having a calculation result in high consistent with the WGS result.

### CROSS-REFERENCE TO RELATED APPLICATION

The presented application is a continuation of international PCT application serial No. PCT/CN2021/072605 filed on Jan. 19, 2021, which claims the priority of the Chinese Patent Application No. 202011208296.8, filed on Nov. 3, 2020 and entitled “MARKER FOR HOMOLOGOUS RECOMBINATION DEFICIENCY, METHOD AND SYSTEM FOR DETECTION THEREOF”. The entirety of each of the above mentioned patent applications is hereby incorporated by reference herein and made a part of this specification.

### BACKGROUND

### Technical Field

The present invention relates to a field of medical molecular biology, and in particular, a marker for homologous recombination deficiency (HRD), a method and a system for detection thereof.

### Description of Related Art

Homologous recombination repair (HRR) for DNA is an important way to repair DNA double-strand damage. HRR, an extremely precise pathway, is essential to maintain the stability and integrity of a genome. Approximately 50% of ovarian cancer patients have HRD.

At present, three PARP inhibitors (niraparib, olaparib, and rucaparib) have been approved for maintenance therapy of platinum-sensitive recurrent ovarian cancer. Wild-type patients with changes in the HRR pathway and molecular characteristics of BRCA mutant tumors (the so-called “BRCAness” phenotype) may also benefit from the therapy with the PARP inhibitors. The main clinical challenge is how to accurately identify such patients.

At present, there are various methods to identify BRCA wild-type tumor patients with HRD. The specific methods are as follows: (1) Record a large amount of genomic abnormalities, that is, the so-called “genomic scars”; (2) Mutation signal analysis; and (3) Two methods for identifying and detecting point mutations in HRR genes by using DNA sequencing panels:

One is the Myriad “myChoice HRD” assay method to calculate scores based on values of three Genomic Scars:

loss of heterozygosity (LOH),

telomeric allelic imbalance (TAI), and

large-scale state transition (LST).

HRD score≥42 or the BRCA1/2 mutant indicates HRD positive; and

HRD score<42 and the BRCA1/2 wild type indicate HRD negative.

The other is the “FoundationFocus CDx BRCA LOH” detection method including two main factors:

BRCA mutation status of tumors, and

genomic LOH level.

High LOH: proportion≥16%; and low LOH: proportion<16%.

The definition of HRD negative: BRCA wild type with low LOH.

However, in the foregoing detection methods, evaluation of the HRD score generally requires the data copy number of the whole genome, and requires information about a large amount of SNPs evenly distributed on the genome. In commercial high-density SNP microsequencing, such as Myriad, a large amount of heterozygous SNP sites have been specially designed on the genome to detect the status of specific regions of chromosomes, but it is low in sequencing depth so that the less abundant somatic mutations are not allowed to be detected. In large customized multi-gene panels, such as FoundationMedicine, its sequencing depth allows the somatic mutation to be detected, but it has a small number of heterozygous SNPs, allowing detection for LOH in specific regions, while insufficiency for assessment of the status of chromosomes of the whole genome.

### SUMMARY

The present invention provides a marker for evaluating HRD of the Chinese population, having a determining result in high consistent with the scoring of the whole exome sequencing result.

A homologous recombination deficiency (HRD) marker is provided, including 8000-9000 SNP sites.

In an implementation, the information about the SNP sites is shown in Table 1.

A method for detecting homologous recombination deficiency (HRD) is provided, including the following steps:

sequencing a sample by the NGS method to obtain the foregoing information about the SNP sites;

obtaining information about loss of heterozygosity (LOH), telomeric allelic imbalance (TAI), or large-scale state transition (LST) at the SNP sites; and

calculating a scoring result of HRD of the sample according to a formula HRD=LOH+TAI+LST.

Use of a reagent for detecting the foregoing marker in the preparation of a reagent for evaluating homologous recombination deficiency (HRD) is provided.

In an implementation, the use further includes a step of scoring HRD, the scoring being calculated according to the following formula:

HRD=LOH+TAI+LST,

wherein,

LOH refers to the number of heterozygous loss regions that cannot span the entire chromosome and is at least 15 Mb;

TAI refers to the number of allelic imbalances in front of telomere ends of chromosomes; and

LST refers to the number of a chromosomal break region not greater than 3 Mb and a fragment of at least 10 Mb.

A device for evaluating homologous recombination deficiency (HRD) is provided, including:

a sequencer, configured to detect SNP sites shown in the table in a sample;

an LOH counting module, configured to determine and count the number of loss of heterozygosity (LOH) at the SNP sites;

a TAI counting module, configured to determine and count the number of telomeric allelic imbalances (TAIs) at the SNP sites;

an LST counting module, configured to determine and count the number of large-scale state transitions (LSTs) at the SNP sites;

an HRD counting module, configured to calculate an HRD score according to HRD=LOH+TAI+LST; and

a classification module, configured to evaluate whether the sample has HRD according to the HRD score.

### Advantageous Effect

The HRD marker provided by the present invention allows HRD to be effectively scored, having a calculation result in high consistent with the whole-genome sequencing (WGS) result.

### BRIEF DESCRIPTION OF THE DRAWINGS

FIG. 1 shows comparison of the consistency of HRD results in selected regions.

FIG. 2 shows location distribution of SNP sites on the chromosome.

FIG. 3 shows comparison of the consistency of HRD scores.

FIG. 4 shows comparison of HRD scoring results of an actual sample.

FIG. 5 shows comparison of HRD scoring results of an actual sample.

FIG. 6 shows comparison of the consistency of scoring results between gnomAD and SNP sites in the present invention for the Chinese population.

### DESCRIPTION OF THE EMBODIMENTS

LOH is often found in cancer, and the chromosomal region with heterozygous loss has varied B-allele frequency (BAF). As shown in the figure, BAF=50% in this region in healthy samples, and BAF=100% in tumor tissues due to LOH. Therefore, to fully detect HRD, a large amount of heterozygous SNP sites are designed in a panel.

In the present invention, the most common polymorphic sites based on Asians are obtained through screening, and in the present invention, the heterozygous SNP sites are uniformly provided on the entire human genome by NGS sequencing by using the foregoing panel composed of a plurality of SNP site regions spanning the entire genome. gnomAD v2.1 contains SNV and InDel records of 15708 WGS samples, including 780 East Asian samples. A gnomAD site heterozygosity rate is calculated based on an allele frequency of the East Asian population (EAS_AF) of gnomAD (gnomAD heterozygous rate=2*EAS_AF*(1−EAS_AF)). Sites with the highest heterozygous SNP in each region are selected with an interval of ˜300 bp. After the SNP sites are obtained by the foregoing method, the influence of the number of the SNP sites on the final classification performance is further investigated, and the HRD score is calculated by using heterozygous SNP capture regions.

In the present invention, the HRD score is calculated as follows:

The formula for calculation of HRD: HRD=LOH+TAI+LST, wherein:

1. LOH refers to the number of heterozygous loss regions that cannot span the entire chromosome and is at least 15 Mb.

2. TAI refers to the number of allelic imbalances in front of telomere ends of chromosomes.

3. LST refers to the number of a chromosomal break region not greater than 3 Mb and a fragment of at least 10 Mb.

The foregoing information can be obtained through the analysis of SNP site detection results by using existing bio-info software.

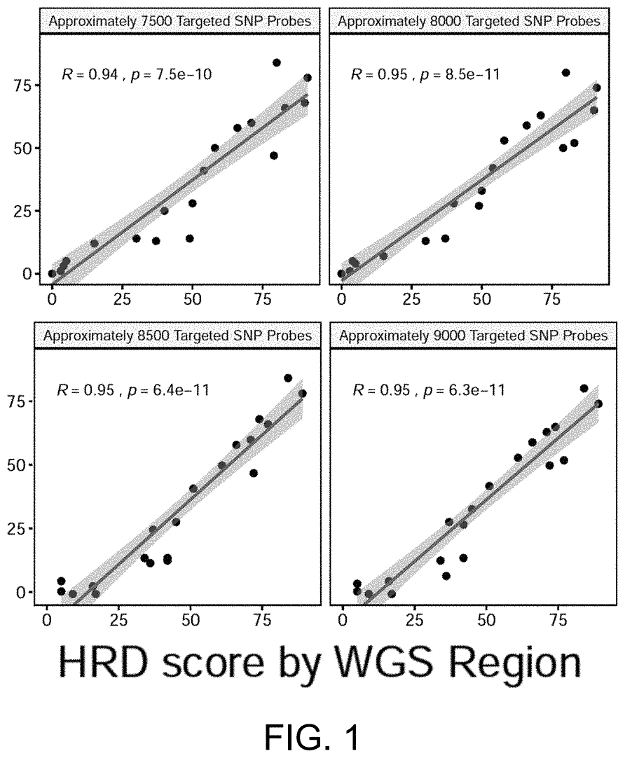

To determine proper number of heterozygous SNPs, 20 WGS samples were used for selective simulation, and the consistency of HRD results in selected regions was shown in FIG. 1.

First, calculation of the HRD values were performed on 4000, 4500, . . . , and 9000 SNP sites having a high heterozygous rate, which are randomly selected from the SNP sites having a gnomAD heterozygous rate≥9.5% with an interval of 200 kb to 400 kb according to a ratio of the length of each chromosome in the human reference genome, and the minimal SNP sites that enabled optimal classification performance were investigated, with results shown in FIG. 1.

It was found that when heterozygous SNP was between 8000 and 9000, Pearson R can reach 95%.

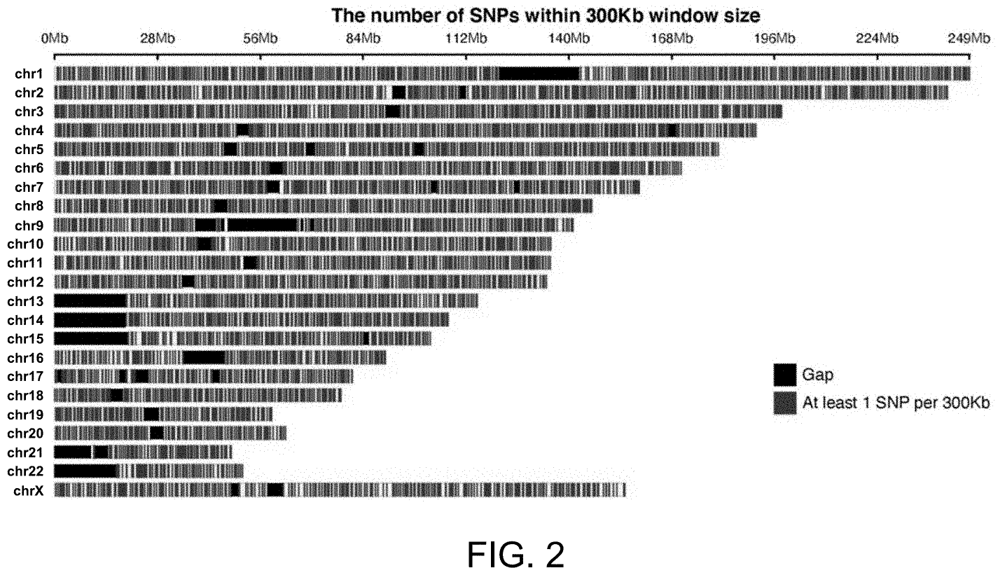

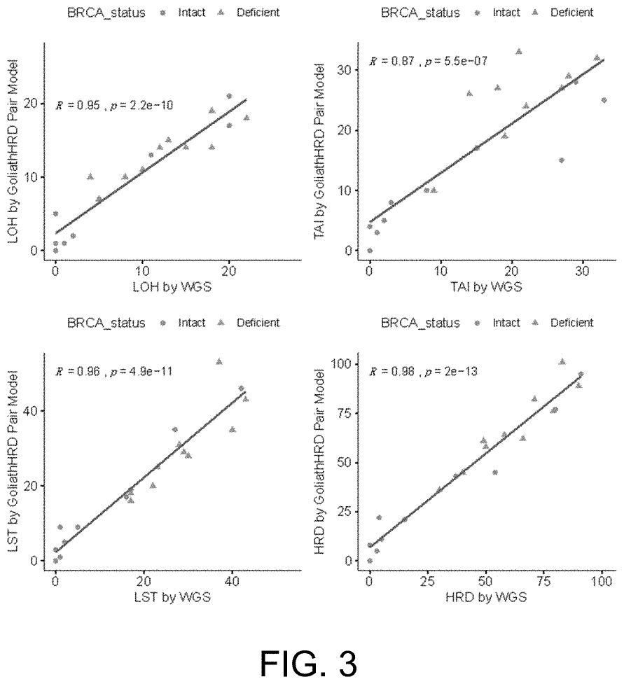

Next, the SNP sites with a high heterozygous rate suitable for the Chinese population were provided by screening SNP sites on these high heterozygous rate gnomAD records and 355 Chinese WGS Data. The actual SNP heterozygous rate (actual SNP heterozygous rate=number of people with heterozygosity/total number of people, WGS normal N=355) of each record was calculated. About 8300 SNP sites with a high heterozygous rate of the Chinese population were selected according to a heterozygous SNP gradient selection result (specific information about the SNP sites was shown in Table 1). FIG. 2 shows distribution of the selected SNP sites on the chromosome.

In the present invention, deficiency of the BRCA set is determined on a basis of double inactivation, that is, pathogenic mutations or deficiency occurs on two chains of chromosome corresponding to each gene, causing complete loss of function for the gene.

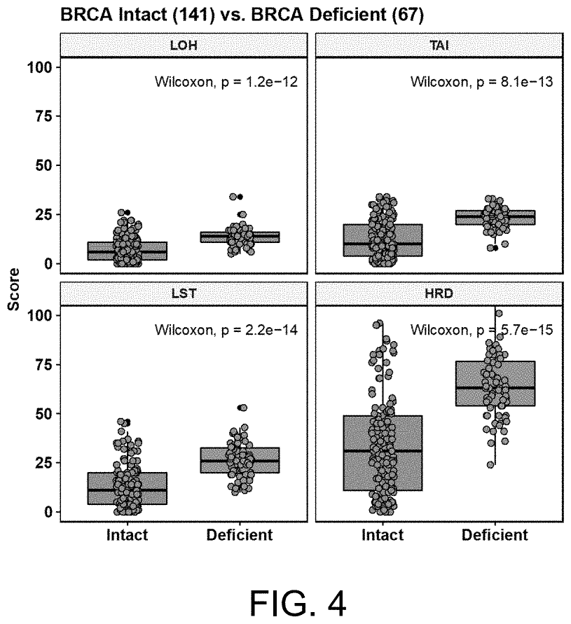

WGS and GoliathHRD sequencing were performed respectively on 20 samples from breast and ovarian cancer in the Chinese population (10 for BRCA Deficient, and 10 for BRCA Intact). The consistency of HRD scores was analyzed using the HRD score result of WGS as a gold standard, with results shown in FIG. 4. It can be seen from the figure that the result from determining by the HRD score is the most significant, and is better than other indicators.

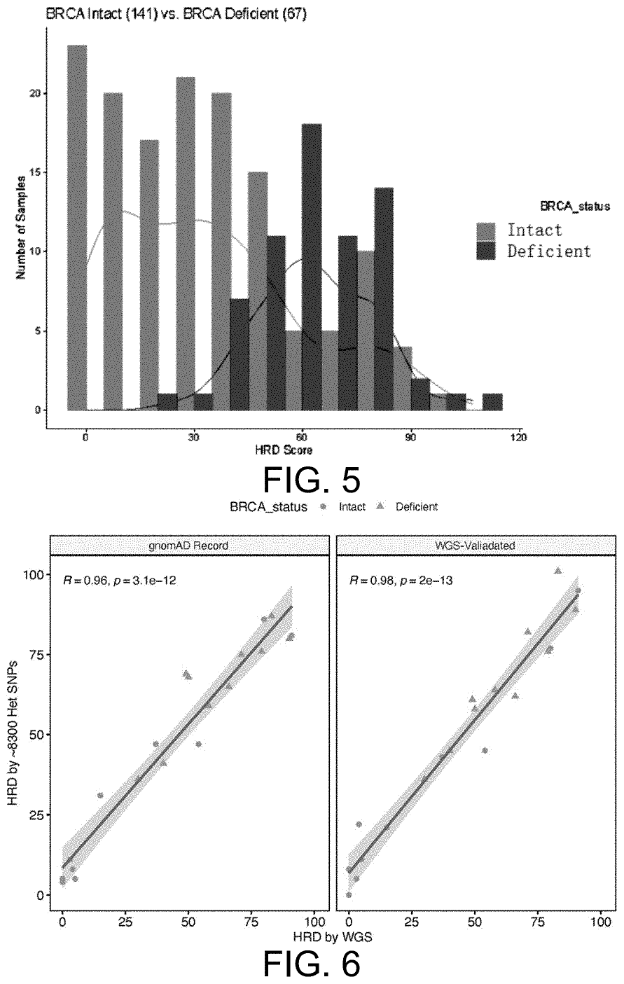

In addition, GoliathHRD sequencing was performed on 208 samples from the Chinese population (141 for BRCA Intact, and 67 for BRCA Deficient), and the difference in the HRD score between the two groups was the most significant. The differentiation result was shown in FIG. 5. It can also be seen from the figure that the determining method of this patent also shows the best accuracy for classification, with p value significantly smaller than other determining methods having p value of 5.7e−15.

According to the same interval length, the same number of SNP sites with the highest gnomAD EAS heterozygous rate in each interval were selected to calculate the HRD value, and it was compared with the SNP result verified by WGS from the true Chinese population which is more consistent with the WGS result (see FIG. 6). It can be seen that the scores recorded by gnomeAD result in R of about 0.96 with p value of about 3.1e−12, while the SNP site combination in the present invention provides R value of 0.98 with p value of about 2e−13. Therefore, the site combination in the present invention is more suitable for scoring HRD for the Chinese population.

TABLE 1
SNP sites

| Chromo- | | Refer- | Varia- | Chromo- | | Refer- | Varia- | Chromo- | | Refer- | Varia- |
|---|---|---|---|---|---|---|---|---|---|---|---|
| some | Location | ence | tion | some | Location | ence | tion | some | Location | ence | tion |
| 1 | 105279 | G | C | 1 | 251663 | C | G | 1 | 649113 | C | T |
| 1 | 649192 | A | T | 1 | 743072 | C | A | 1 | 924528 | C | A |
| 1 | 1127507 | G | A | 1 | 1127523 | C | T | 1 | 1128429 | G | A |
| 1 | 1128521 | C | T | 1 | 1650410 | T | C | 1 | 2488930 | G | A |
| 1 | 2631804 | G | A | 1 | 2693121 | A | T | 1 | 3097439 | C | T |
| 1 | 3097459 | C | T | 1 | 3097464 | A | G | 1 | 3097498 | A | T |
| 1 | 3097509 | C | T | 1 | 3097522 | T | C | 1 | 3097532 | T | G |
| 1 | 3097534 | G | A | 1 | 3098219 | C | A | 1 | 3098360 | A | G |
| 1 | 3098386 | G | T | 1 | 4196428 | G | A | 1 | 4967739 | G | C |
| 1 | 4967744 | C | A | 1 | 5393906 | G | A | 1 | 5876714 | T | A |
| 1 | 5876956 | A | G | 1 | 5876977 | C | A | 1 | 5876988 | A | G |
| 1 | 5877088 | C | G | 1 | 5877112 | C | T | 1 | 5877121 | C | A |
| 1 | 5877138 | G | A | 1 | 5877145 | C | A | 1 | 5877169 | C | A |
| 1 | 5877171 | T | C | 1 | 5877327 | A | C | 1 | 5877329 | A | G |
| 1 | 5877337 | G | A | 1 | 5877367 | G | A | 1 | 5877384 | C | T |
| 1 | 6481623 | G | A | 1 | 9493601 | C | T | 1 | 9494062 | A | G |
| 1 | 9494142 | T | C | 1 | 10357406 | A | G | 1 | 11167760 | C | T |
| 1 | 11167829 | C | T | 1 | 11168529 | A | G | 1 | 11174067 | A | T |
| 1 | 11175176 | T | C | 1 | 11181630 | G | A | 1 | 11189191 | G | A |
| 1 | 11193401 | A | C | 1 | 11193406 | C | T | 1 | 11193408 | C | T |
| 1 | 11209899 | T | C | 1 | 11227224 | C | T | 1 | 11269796 | T | C |
| 1 | 11292753 | A | G | 1 | 11293200 | C | T | 1 | 11293216 | A | G |
| 1 | 11293792 | A | C | 1 | 11303610 | T | C | 1 | 11307412 | G | C |
| 1 | 11315853 | G | A | 1 | 11318763 | C | G | 1 | 11318983 | T | C |
| 1 | 12188028 | C | A | 1 | 12854713 | G | T | 1 | 12854716 | A | G |
| 1 | 12886052 | C | T | 1 | 12886098 | T | C | 1 | 12886436 | C | A |
| 1 | 12920067 | C | G | 1 | 12920132 | A | G | 1 | 12920191 | T | C |
| 1 | 12920200 | G | A | 1 | 12920622 | C | T | 1 | 12920708 | C | A |
| 1 | 12940618 | A | G | 1 | 12940822 | A | T | 1 | 12941059 | C | A |
| 1 | 12941086 | C | A | 1 | 13143375 | G | T | 1 | 13143405 | A | G |
| 1 | 13143441 | C | T | 1 | 13143453 | C | T | 1 | 13367310 | C | G |
| 1 | 13367310 | C | T | 1 | 14228227 | C | G | 1 | 14228236 | A | G |
| 1 | 14228340 | G | C | 1 | 14228356 | T | C | 1 | 15829457 | C | G |
| 1 | 16451413 | C | T | 1 | 16460541 | T | C | 1 | 17345113 | G | C |
| 1 | 17348847 | G | C | 1 | 17350243 | T | C | 1 | 17353988 | T | G |
| 1 | 17370970 | C | T | 1 | 17687161 | G | C | 1 | 18476868 | T | C |
| 1 | 18476876 | A | G | 1 | 18477394 | A | C | 1 | 18477450 | G | A |
| 1 | 20058523 | A | G | 1 | 20915418 | C | G | 1 | 20916080 | C | G |
| 1 | 20931774 | A | G | 1 | 20944721 | G | A | 1 | 22234424 | G | C |
| 1 | 22301165 | C | T | 1 | 22301180 | G | A | 1 | 22301199 | C | T |
| 1 | 22301755 | A | G | 1 | 22301769 | G | A | 1 | 22301770 | C | T |
| 1 | 22301824 | T | C | 1 | 22326109 | T | C | 1 | 22326455 | T | C |
| 1 | 22326488 | T | C | 1 | 22326548 | A | G | 1 | 22326587 | G | A |
| 1 | 23909324 | T | A | 1 | 24192453 | T | C | 1 | 26228409 | A | G |
| 1 | 28439964 | C | T | 1 | 28563391 | C | T | 1 | 29232578 | C | G |
| 1 | 31581492 | C | T | 1 | 31857847 | G | A | 1 | 31857869 | G | A |
| 1 | 31857916 | C | T | 1 | 33819830 | A | C | 1 | 34989237 | G | A |
| 1 | 37049763 | C | T | 1 | 37568312 | C | G | 1 | 38010651 | G | A |
| 1 | 38010751 | C | T | 1 | 39944488 | T | G | 1 | 40367607 | G | C |
| 1 | 40367625 | G | T | 1 | 40799427 | G | A | 1 | 41940980 | C | T |
| 1 | 42042317 | C | T | 1 | 42042915 | C | T | 1 | 43803222 | G | A |
| 1 | 44606254 | C | T | 1 | 44606565 | A | G | 1 | 45880669 | C | T |
| 1 | 45880751 | C | T | 1 | 46727304 | C | T | 1 | 46737961 | C | T |
| 1 | 47009757 | G | A | 1 | 47009799 | A | G | 1 | 47009829 | G | T |
| 1 | 47541531 | C | A | 1 | 48119989 | C | T | 1 | 48120087 | T | C |
| 1 | 50591705 | C | G | 1 | 50591743 | C | T | 1 | 53116251 | A | G |
| 1 | 55390669 | T | C | 1 | 55834642 | G | A | 1 | 55835253 | G | A |
| 1 | 57548321 | C | G | 1 | 59621601 | C | T | 1 | 59621636 | A | T |
| 1 | 59622219 | G | A | 1 | 59622225 | T | G | 1 | 61465647 | C | G |
| 1 | 62148611 | A | G | 1 | 63521709 | T | C | 1 | 63521755 | T | C |
| 1 | 64117913 | A | G | 1 | 65301317 | G | A | 1 | 65301490 | G | A |
| 1 | 65301611 | T | G | 1 | 65303338 | A | G | 1 | 65303390 | G | A |
| 1 | 65312583 | C | T | 1 | 65312949 | A | G | 1 | 65335343 | C | G |
| 1 | 65338745 | C | T | 1 | 65338775 | C | T | 1 | 67822377 | A | G |
| 1 | 68628372 | G | A | 1 | 71012395 | C | A | 1 | 72571656 | C | A |
| 1 | 73095148 | G | T | 1 | 77259478 | G | A | 1 | 77754620 | A | G |
| 1 | 80363334 | G | C | 1 | 82467045 | T | C | 1 | 82843604 | G | T |
| 1 | 83054685 | T | C | 1 | 83054799 | C | T | 1 | 83055055 | T | C |
| 1 | 83055137 | G | T | 1 | 83055238 | G | T | 1 | 84295861 | A | G |
| 1 | 85229792 | G | C | 1 | 86050077 | C | G | 1 | 86873847 | C | T |
| 1 | 87106420 | T | C | 1 | 89849268 | T | C | 1 | 90271586 | T | C |
| 1 | 90673701 | C | T | 1 | 90673754 | G | A | 1 | 91658629 | G | A |
| 1 | 91659249 | G | A | 1 | 91659289 | A | T | 1 | 92082962 | C | T |
| 1 | 96479066 | T | A | 1 | 96993374 | C | A | 1 | 96993449 | T | C |
| 1 | 97838854 | C | T | 1 | 97839413 | C | G | 1 | 97981706 | C | T |
| 1 | 99692027 | A | G | 1 | 100270805 | G | C | 1 | 102251801 | A | G |
| 1 | 102427608 | C | T | 1 | 103047194 | C | T | 1 | 105484566 | G | A |
| 1 | 106740553 | T | G | 1 | 107182505 | A | T | 1 | 110231592 | T | C |
| 1 | 110231777 | G | A | 1 | 110232087 | C | T | 1 | 110233138 | G | C |
| 1 | 110233147 | C | T | 1 | 110235607 | A | G | 1 | 110255174 | T | G |
| 1 | 110256473 | C | G | 1 | 111857685 | G | C | 1 | 111858291 | T | C |
| 1 | 112033285 | G | C | 1 | 113234456 | G | A | 1 | 117786992 | G | A |
| 1 | 117787502 | C | A | 1 | 118136824 | T | C | 1 | 118136861 | G | A |
| 1 | 118136912 | G | A | 1 | 119762037 | T | C | 1 | 119992928 | T | C |
| 1 | 119992995 | C | A | 1 | 119992999 | A | G | 1 | 120462468 | C | T |
| 1 | 120463230 | C | T | 1 | 120483921 | G | C | 1 | 120495934 | G | C |
| 1 | 120498086 | G | C | 1 | 120508803 | T | C | 1 | 120509783 | A | G |
| 1 | 120547695 | A | G | 1 | 144838618 | A | C | 1 | 145931852 | A | C |
| 1 | 146465784 | C | G | 1 | 146555193 | T | C | 1 | 147534441 | T | A |
| 1 | 148004440 | C | A | 1 | 148004899 | T | A | 1 | 148756363 | A | G |
| 1 | 148756397 | C | T | 1 | 148756432 | C | A | 1 | 148756499 | A | G |
| 1 | 148756515 | A | C | 1 | 148854651 | A | C | 1 | 148855855 | C | T |
| 1 | 149067299 | G | A | 1 | 149089962 | A | C | 1 | 149521316 | A | G |
| 1 | 149521391 | A | T | 1 | 149606839 | C | G | 1 | 149607021 | A | G |
| 1 | 149607027 | G | A | 1 | 152277345 | C | T | 1 | 152277396 | T | C |
| 1 | 152278689 | C | A | 1 | 152279406 | T | G | 1 | 152280032 | T | C |
| 1 | 152280170 | C | G | 1 | 152281007 | A | G | 1 | 152281008 | A | G |
| 1 | 152281039 | G | A | 1 | 152760075 | A | G | 1 | 152760093 | T | C |
| 1 | 152760096 | A | T | 1 | 152760628 | T | C | 1 | 152760642 | C | T |
| 1 | 152760644 | C | T | 1 | 152760647 | C | A | 1 | 152760651 | C | T |
| 1 | 152770521 | G | A | 1 | 152770533 | G | A | 1 | 152770589 | A | G |
| 1 | 152770828 | A | G | 1 | 153277768 | C | T | 1 | 154351073 | T | C |
| 1 | 155583937 | A | G | 1 | 156785356 | T | C | 1 | 158165578 | G | A |
| 1 | 158165601 | T | C | 1 | 158165661 | T | C | 1 | 158165722 | G | A |
| 1 | 158165742 | A | T | 1 | 158165938 | A | C | 1 | 158165984 | C | T |
| 1 | 158220870 | T | C | 1 | 158736215 | G | A | 1 | 159086711 | A | G |
| 1 | 160093648 | G | A | 1 | 160236470 | T | A | 1 | 161293193 | C | T |
| 1 | 161293215 | A | G | 1 | 161293646 | T | C | 1 | 161293708 | G | A |
| 1 | 161297993 | T | C | 1 | 161310719 | C | T | 1 | 161326201 | C | T |
| 1 | 161326274 | A | G | 1 | 161326293 | T | C | 1 | 161558725 | C | T |
| 1 | 161559082 | C | T | 1 | 162470503 | G | A | 1 | 163033342 | A | C |
| 1 | 166245169 | A | G | 1 | 166245170 | G | C | 1 | 166541921 | T | C |
| 1 | 167638203 | T | C | 1 | 167638251 | T | G | 1 | 168365929 | C | G |
| 1 | 168513243 | G | A | 1 | 168513692 | C | T | 1 | 168545758 | C | T |
| 1 | 177940341 | C | T | 1 | 177940348 | C | T | 1 | 178922201 | A | T |
| 1 | 179335158 | C | T | 1 | 179335162 | G | C | 1 | 180162293 | G | C |
| 1 | 180162364 | C | T | 1 | 181394677 | G | C | 1 | 181828178 | T | G |
| 1 | 186256808 | A | G | 1 | 188674740 | C | A | 1 | 189555573 | G | C |
| 1 | 191151643 | G | T | 1 | 191152225 | C | T | 1 | 191719275 | A | T |
| 1 | 192017093 | C | G | 1 | 194952546 | G | C | 1 | 195371632 | G | A |
| 1 | 196788601 | C | T | 1 | 196912415 | C | T | 1 | 198340898 | T | C |
| 1 | 199449355 | C | T | 1 | 199548706 | C | G | 1 | 201178470 | G | A |
| 1 | 201489285 | G | C | 1 | 201489286 | C | G | 1 | 201489302 | C | T |
| 1 | 201489565 | C | T | 1 | 201978049 | C | T | 1 | 201978068 | G | A |
| 1 | 202096562 | T | C | 1 | 203274536 | C | A | 1 | 203842900 | C | T |
| 1 | 203843484 | T | C | 1 | 204315839 | T | G | 1 | 204494436 | C | T |
| 1 | 204495253 | G | A | 1 | 204500257 | G | A | 1 | 204511704 | T | C |
| 1 | 204513402 | G | A | 1 | 205816893 | T | C | 1 | 205816923 | G | A |
| 1 | 206646274 | G | T | 1 | 206646311 | C | T | 1 | 206647387 | T | G |
| 1 | 206647450 | A | G | 1 | 206669136 | C | A | 1 | 207807908 | C | T |
| 1 | 207807921 | G | C | 1 | 207807928 | G | A | 1 | 207808385 | C | G |
| 1 | 208164511 | C | T | 1 | 208780934 | A | C | 1 | 210371949 | A | G |
| 1 | 210372455 | T | C | 1 | 210827321 | T | C | 1 | 210827322 | G | A |
| 1 | 210827379 | C | T | 1 | 211126407 | G | A | 1 | 211855564 | G | A |
| 1 | 211855593 | T | A | 1 | 212224761 | A | G | 1 | 212224946 | G | T |
| 1 | 212240533 | A | G | 1 | 212245792 | A | G | 1 | 214386346 | T | G |
| 1 | 214386351 | C | T | 1 | 214386365 | A | T | 1 | 214571527 | C | G |
| 1 | 215128241 | C | T | 1 | 216840332 | G | C | 1 | 216840371 | C | T |
| 1 | 216840798 | C | T | 1 | 218238495 | T | C | 1 | 218572004 | G | A |
| 1 | 219969405 | T | C | 1 | 222477115 | C | A | 1 | 222642478 | C | A |
| 1 | 222642700 | G | A | 1 | 222677791 | C | G | 1 | 223089124 | G | A |
| 1 | 223718462 | G | A | 1 | 223718518 | C | A | 1 | 224164610 | G | A |
| 1 | 224220492 | A | G | 1 | 224220734 | G | A | 1 | 224644312 | C | T |
| 1 | 226549498 | G | A | 1 | 226553934 | A | C | 1 | 226557878 | T | C |
| 1 | 226580187 | T | C | 1 | 227680600 | G | A | 1 | 227680783 | T | C |
| 1 | 227680868 | T | C | 1 | 227681213 | T | C | 1 | 227681563 | A | G |
| 1 | 227681567 | T | C | 1 | 227695408 | T | G | 1 | 227695429 | C | G |
| 1 | 227695486 | G | A | 1 | 227695818 | C | T | 1 | 227695830 | C | T |
| 1 | 227695868 | G | A | 1 | 227696002 | A | G | 1 | 227696033 | T | C |
| 1 | 227696110 | G | A | 1 | 228156074 | C | T | 1 | 228163615 | A | C |
| 1 | 228163691 | A | G | 1 | 228164114 | G | C | 1 | 228164190 | G | A |
| 1 | 228164584 | A | G | 1 | 228165010 | A | G | 1 | 228457036 | T | A |
| 1 | 228457543 | G | A | 1 | 228716894 | C | T | 1 | 229824770 | T | C |
| 1 | 231146762 | G | A | 1 | 231146770 | G | C | 1 | 232491583 | T | G |
| 1 | 233280999 | T | A | 1 | 234126736 | G | A | 1 | 234127282 | C | G |
| 1 | 234127319 | C | T | 1 | 234128435 | A | G | 1 | 234128441 | A | T |
| 1 | 234128967 | C | T | 1 | 234948160 | T | C | 1 | 235049923 | A | C |
| 1 | 235702712 | C | T | 1 | 236919092 | T | G | 1 | 236919146 | G | A |
| 1 | 237410497 | C | G | 1 | 237410504 | G | C | 1 | 237445099 | T | C |
| 1 | 238113949 | T | A | 1 | 239800851 | C | T | 1 | 240451555 | T | C |
| 1 | 240451568 | C | T | 1 | 241665510 | A | G | 1 | 241671638 | C | T |
| 1 | 242453104 | C | T | 1 | 242819974 | T | G | 1 | 243204262 | G | T |
| 1 | 243245426 | C | T | 1 | 243662773 | T | C | 1 | 243801190 | T | C |
| 1 | 243828306 | T | A | 1 | 244891367 | A | G | 1 | 244891415 | G | A |
| 1 | 245856763 | G | A | 1 | 246362962 | T | C | 1 | 246363094 | G | C |
| 1 | 246641904 | T | C | 1 | 246642046 | A | G | 1 | 246642078 | T | C |
| 1 | 246642155 | A | G | 1 | 247337911 | A | C | 1 | 247338427 | A | G |
| 1 | 247591141 | G | A | 1 | 248084854 | A | G | 1 | 248084909 | T | G |
| 1 | 248085341 | T | A | 1 | 248436611 | G | A | 1 | 248436616 | A | G |
| 1 | 248907914 | G | T | 1 | 248907917 | C | T | 1 | 248907919 | C | T |
| 2 | 197601 | A | G | 2 | 197712 | T | C | 2 | 1366783 | G | A |
| 2 | 1366859 | G | A | 2 | 1833356 | T | C | 2 | 1833781 | G | A |
| 2 | 2413270 | T | G | 2 | 2413783 | C | T | 2 | 2940325 | G | A |
| 2 | 2940329 | A | G | 2 | 3332735 | A | G | 2 | 3333243 | T | G |
| 2 | 3877140 | C | T | 2 | 3877747 | G | A | 2 | 4561867 | T | C |
| 2 | 5389739 | A | G | 2 | 5768841 | C | G | 2 | 6426700 | T | C |
| 2 | 6426703 | C | T | 2 | 6943743 | C | A | 2 | 7068710 | C | G |
| 2 | 7999597 | G | A | 2 | 9446167 | C | A | 2 | 9446205 | G | A |
| 2 | 10381717 | C | T | 2 | 10676227 | C | G | 2 | 10676309 | C | T |
| 2 | 10676315 | A | G | 2 | 10676317 | T | G | 2 | 10676320 | G | A |
| 2 | 10676368 | T | C | 2 | 10676498 | T | C | 2 | 10676610 | G | A |
| 2 | 11093401 | T | C | 2 | 11093430 | T | C | 2 | 11242610 | C | T |
| 2 | 11994573 | A | C | 2 | 11994575 | G | A | 2 | 11994645 | A | G |
| 2 | 11994813 | A | G | 2 | 11994822 | C | G | 2 | 11994829 | T | A |
| 2 | 11994840 | T | A | 2 | 11994849 | T | C | 2 | 11994863 | A | C |
| 2 | 11994865 | G | A | 2 | 11994907 | T | C | 2 | 13981557 | C | T |
| 2 | 14082251 | G | T | 2 | 15832606 | G | A | 2 | 16086469 | C | T |
| 2 | 19524172 | T | G | 2 | 21634473 | T | C | 2 | 24167417 | T | G |
| 2 | 24439048 | A | G | 2 | 25458379 | G | A | 2 | 25472784 | C | T |
| 2 | 25505793 | T | C | 2 | 25522795 | G | A | 2 | 26367863 | T | C |
| 2 | 26835397 | C | A | 2 | 26835419 | G | A | 2 | 27615389 | C | T |
| 2 | 28350922 | G | A | 2 | 29416946 | T | C | 2 | 29432982 | C | T |
| 2 | 29436633 | G | T | 2 | 29437182 | T | C | 2 | 29451511 | C | G |
| 2 | 29940820 | C | T | 2 | 30142547 | G | C | 2 | 31552688 | G | C |
| 2 | 31553158 | C | G | 2 | 33896721 | G | A | 2 | 35130065 | T | G |
| 2 | 36768719 | C | G | 2 | 37050374 | A | G | 2 | 37996061 | C | T |
| 2 | 38002857 | T | G | 2 | 40783500 | C | G | 2 | 40783501 | T | C |
| 2 | 41273446 | A | C | 2 | 41273450 | T | C | 2 | 43779665 | T | C |
| 2 | 43783241 | C | A | 2 | 43783273 | G | A | 2 | 43787758 | G | C |
| 2 | 43798616 | C | G | 2 | 43802347 | T | A | 2 | 43802380 | C | G |
| 2 | 43803897 | A | C | 2 | 44274227 | G | C | 2 | 44274729 | A | G |
| 2 | 44292977 | C | T | 2 | 44985166 | C | G | 2 | 45576533 | C | A |
| 2 | 45577081 | A | G | 2 | 46064330 | A | G | 2 | 46525391 | G | C |
| 2 | 46588488 | G | T | 2 | 46597296 | T | C | 2 | 46603260 | C | T |
| 2 | 46603438 | A | G | 2 | 46604857 | T | C | 2 | 46605659 | A | G |
| 2 | 46611471 | G | A | 2 | 47597029 | C | A | 2 | 47602241 | T | C |
| 2 | 47606683 | T | C | 2 | 47612156 | A | G | 2 | 47639871 | T | G |
| 2 | 47643191 | C | G | 2 | 47672475 | T | A | 2 | 47698384 | G | A |
| 2 | 47703241 | A | G | 2 | 47703984 | T | G | 2 | 47709704 | T | C |
| 2 | 48022900 | A | G | 2 | 48914615 | C | G | 2 | 48941428 | C | T |
| 2 | 48952574 | C | T | 2 | 48956475 | C | T | 2 | 48956512 | A | G |
| 2 | 48958595 | A | G | 2 | 48958626 | A | G | 2 | 48958647 | C | T |
| 2 | 48982358 | G | A | 2 | 49070593 | G | C | 2 | 49071094 | G | C |
| 2 | 49660621 | A | T | 2 | 50231869 | G | A | 2 | 50232465 | C | T |
| 2 | 50763570 | A | G | 2 | 51450722 | A | G | 2 | 52153976 | T | C |
| 2 | 53792407 | T | C | 2 | 55066617 | G | C | 2 | 56338148 | G | T |
| 2 | 58453585 | T | C | 2 | 58453620 | C | G | 2 | 59260307 | G | A |
| 2 | 61183901 | A | G | 2 | 62373915 | T | C | 2 | 64720254 | G | A |
| 2 | 64892858 | G | T | 2 | 65863984 | T | G | 2 | 65864061 | C | G |
| 2 | 66063089 | A | G | 2 | 66955742 | C | T | 2 | 68999640 | G | A |
| 2 | 71256898 | G | T | 2 | 71256905 | C | A | 2 | 71256965 | G | A |
| 2 | 71283017 | G | T | 2 | 71283128 | G | A | 2 | 71283247 | G | A |
| 2 | 71283425 | T | G | 2 | 73295583 | G | A | 2 | 73869120 | G | A |
| 2 | 73869558 | T | C | 2 | 75089569 | A | G | 2 | 75090155 | G | A |
| 2 | 76124531 | C | T | 2 | 76124535 | C | T | 2 | 76890353 | T | A |
| 2 | 77637227 | A | T | 2 | 79445642 | A | T | 2 | 80101025 | G | A |
| 2 | 80101040 | A | G | 2 | 81423752 | T | C | 2 | 83419234 | G | C |
| 2 | 84073752 | A | T | 2 | 84074312 | A | G | 2 | 85141323 | A | G |
| 2 | 85685734 | A | G | 2 | 87405001 | T | C | 2 | 87405397 | G | A |
| 2 | 87444116 | A | G | 2 | 87444177 | A | G | 2 | 87444438 | C | T |
| 2 | 87615504 | A | G | 2 | 88433305 | T | C | 2 | 88739133 | T | A |
| 2 | 88739134 | C | G | 2 | 88739141 | C | A | 2 | 88739644 | C | G |
| 2 | 89080379 | C | T | 2 | 89080394 | C | A | 2 | 89080854 | C | G |
| 2 | 90447633 | T | C | 2 | 90448948 | A | G | 2 | 90448956 | C | T |
| 2 | 90449010 | G | T | 2 | 91671081 | C | T | 2 | 91671100 | T | C |
| 2 | 91671122 | C | G | 2 | 91715572 | T | C | 2 | 92151397 | G | A |
| 2 | 95849653 | T | C | 2 | 96596710 | G | C | 2 | 96809331 | T | C |
| 2 | 96920993 | C | T | 2 | 97366088 | T | C | 2 | 97366599 | A | G |
| 2 | 98223750 | A | G | 2 | 99537295 | A | C | 2 | 101350715 | G | A |
| 2 | 102254291 | T | G | 2 | 102254307 | G | A | 2 | 102254364 | C | G |
| 2 | 102616745 | G | T | 2 | 102617204 | T | C | 2 | 104432433 | T | C |
| 2 | 104432494 | A | C | 2 | 104599371 | T | G | 2 | 105671951 | C | T |
| 2 | 105672240 | T | G | 2 | 106543154 | A | T | 2 | 107019359 | T | C |
| 2 | 107019383 | G | T | 2 | 107019457 | T | C | 2 | 107019874 | T | C |
| 2 | 107424428 | C | T | 2 | 108481932 | C | G | 2 | 109928180 | C | T |
| 2 | 109928207 | G | T | 2 | 110800798 | C | A | 2 | 110800846 | G | A |
| 2 | 112289514 | C | A | 2 | 112289579 | T | C | 2 | 112506112 | C | T |
| 2 | 112506288 | T | C | 2 | 112544651 | G | A | 2 | 113691060 | T | C |
| 2 | 113691288 | T | C | 2 | 113869803 | C | T | 2 | 113869843 | C | A |
| 2 | 114256098 | G | A | 2 | 114256343 | A | G | 2 | 114259673 | C | A |
| 2 | 114288352 | G | T | 2 | 119480974 | C | T | 2 | 120130317 | C | T |
| 2 | 120130337 | C | T | 2 | 120130347 | G | T | 2 | 121321830 | C | T |
| 2 | 122995587 | G | T | 2 | 122996267 | C | T | 2 | 123153811 | A | G |
| 2 | 123154175 | A | G | 2 | 123921841 | C | A | 2 | 124206212 | G | T |
| 2 | 124206727 | G | C | 2 | 124206743 | G | A | 2 | 125189819 | A | T |
| 2 | 127420931 | A | G | 2 | 127811810 | C | T | 2 | 127812457 | G | A |
| 2 | 127812468 | A | G | 2 | 128018522 | T | C | 2 | 129473008 | C | T |
| 2 | 130126515 | A | G | 2 | 130801047 | T | C | 2 | 130805820 | T | C |
| 2 | 131233331 | C | T | 2 | 131945811 | C | A | 2 | 131947586 | C | T |
| 2 | 131955247 | G | C | 2 | 132045098 | C | G | 2 | 132046355 | C | T |
| 2 | 132107348 | A | C | 2 | 132181110 | C | T | 2 | 132533478 | T | C |
| 2 | 132534075 | G | T | 2 | 132579856 | G | A | 2 | 132579930 | A | G |
| 2 | 132580131 | A | C | 2 | 132796086 | G | A | 2 | 132796335 | A | G |
| 2 | 132796339 | A | G | 2 | 132797478 | G | A | 2 | 132809855 | A | G |
| 2 | 132809875 | G | A | 2 | 132809905 | G | T | 2 | 132872466 | C | A |
| 2 | 132872691 | A | G | 2 | 132872847 | G | T | 2 | 132905500 | C | T |
| 2 | 132905513 | C | A | 2 | 132905976 | G | A | 2 | 133644992 | T | A |
| 2 | 133645369 | G | A | 2 | 133645476 | G | A | 2 | 134945535 | A | G |
| 2 | 134946060 | T | C | 2 | 137181641 | C | A | 2 | 137181641 | C | G |
| 2 | 137181661 | C | T | 2 | 137900678 | A | C | 2 | 140000027 | A | G |
| 2 | 140991087 | A | C | 2 | 140992063 | G | C | 2 | 141028164 | C | T |
| 2 | 141055080 | T | G | 2 | 141072930 | C | T | 2 | 141114235 | C | G |
| 2 | 141128001 | C | G | 2 | 141129072 | T | C | 2 | 141136007 | T | C |
| 2 | 141202412 | C | T | 2 | 141202499 | T | A | 2 | 141245486 | G | A |
| 2 | 141271959 | T | G | 2 | 141272545 | G | A | 2 | 141283130 | T | C |
| 2 | 141284090 | C | G | 2 | 141293912 | G | T | 2 | 141299730 | T | C |
| 2 | 141299775 | C | T | 2 | 141355954 | C | T | 2 | 141457603 | G | C |
| 2 | 141460317 | T | A | 2 | 141526501 | T | C | 2 | 141570937 | G | T |
| 2 | 141571590 | C | T | 2 | 141624902 | T | C | 2 | 141641859 | C | A |
| 2 | 141644076 | T | A | 2 | 141660215 | T | C | 2 | 141661007 | A | G |
| 2 | 141707558 | T | C | 2 | 141707610 | A | G | 2 | 141777212 | C | G |
| 2 | 141812434 | C | T | 2 | 141819422 | G | A | 2 | 142004512 | A | C |
| 2 | 142238325 | A | T | 2 | 143225895 | G | A | 2 | 143226379 | A | G |
| 2 | 143226439 | G | T | 2 | 143748793 | A | G | 2 | 144559694 | G | T |
| 2 | 147187335 | T | C | 2 | 148300099 | G | A | 2 | 148300132 | G | A |
| 2 | 149284724 | G | A | 2 | 150920975 | T | C | 2 | 152025225 | A | G |
| 2 | 152943265 | T | C | 2 | 154500721 | T | A | 2 | 155316251 | T | C |
| 2 | 155316904 | C | T | 2 | 156332987 | G | C | 2 | 157262577 | T | G |
| 2 | 160895559 | T | C | 2 | 160895602 | C | G | 2 | 161487015 | T | A |
| 2 | 161822563 | G | A | 2 | 162101078 | A | G | 2 | 166025675 | A | G |
| 2 | 167857455 | G | C | 2 | 170143494 | A | C | 2 | 170624543 | A | G |
| 2 | 171678379 | C | T | 2 | 172034684 | A | G | 2 | 172035195 | G | T |
| 2 | 173664873 | T | C | 2 | 174482931 | G | A | 2 | 175584721 | G | C |
| 2 | 176675940 | A | G | 2 | 177107092 | G | C | 2 | 178098502 | G | T |
| 2 | 178494520 | A | G | 2 | 178528351 | T | C | 2 | 178528360 | A | G |
| 2 | 178528874 | T | C | 2 | 178545736 | T | C | 2 | 178576681 | G | A |
| 2 | 178681294 | T | C | 2 | 178682329 | T | A | 2 | 178682901 | C | T |
| 2 | 178684720 | G | C | 2 | 178704709 | A | G | 2 | 178740334 | T | G |
| 2 | 178740393 | C | T | 2 | 178740398 | C | G | 2 | 178969434 | G | C |
| 2 | 179296821 | G | A | 2 | 179729081 | C | T | 2 | 180308028 | C | T |
| 2 | 180734989 | A | C | 2 | 181490053 | C | T | 2 | 181490633 | G | C |
| 2 | 182433865 | G | A | 2 | 182433873 | G | T | 2 | 182434468 | G | A |
| 2 | 182434507 | A | G | 2 | 183726713 | C | T | 2 | 184128825 | A | G |
| 2 | 184912216 | T | C | 2 | 184912648 | T | C | 2 | 187169224 | G | A |
| 2 | 187866471 | C | T | 2 | 187867057 | G | A | 2 | 190176234 | G | T |
| 2 | 190176235 | C | A | 2 | 190708982 | C | A | 2 | 190741733 | G | A |
| 2 | 190742374 | A | G | 2 | 192422875 | G | T | 2 | 193726153 | C | T |
| 2 | 193726212 | T | C | 2 | 193726268 | A | G | 2 | 194468850 | T | C |
| 2 | 195079687 | G | C | 2 | 196335442 | T | G | 2 | 197037730 | G | A |
| 2 | 198273591 | C | T | 2 | 198274929 | G | A | 2 | 198285554 | C | G |
| 2 | 198905073 | T | A | 2 | 201487990 | C | T | 2 | 212252955 | C | T |
| 2 | 212292929 | G | C | 2 | 212530466 | C | T | 2 | 212543510 | T | C |
| 2 | 212544193 | T | C | 2 | 212566477 | G | A | 2 | 212569828 | C | T |
| 2 | 213296501 | T | G | 2 | 214019110 | T | C | 2 | 215595440 | T | A |
| 2 | 215595445 | C | A | 2 | 215610777 | T | C | 2 | 215617500 | T | C |
| 2 | 215631987 | A | G | 2 | 215632629 | G | T | 2 | 215645030 | T | C |
| 2 | 215645046 | C | T | 2 | 215672347 | C | T | 2 | 215672827 | C | T |
| 2 | 215672833 | A | G | 2 | 216203296 | A | G | 2 | 216860550 | A | T |
| 2 | 217021055 | A | G | 2 | 217453875 | T | C | 2 | 217453939 | C | A |
| 2 | 220136059 | C | A | 2 | 220634227 | C | A | 2 | 220634291 | A | G |
| 2 | 222231318 | G | A | 2 | 222231319 | A | G | 2 | 222231322 | C | T |
| 2 | 222231334 | A | G | 2 | 222232311 | A | G | 2 | 222233697 | T | C |
| 2 | 222721065 | C | T | 2 | 223900314 | C | T | 2 | 224277502 | T | C |
| 2 | 225346377 | A | G | 2 | 225347043 | T | A | 2 | 225360975 | T | C |
| 2 | 225360980 | T | C | 2 | 225360985 | T | C | 2 | 225362798 | A | G |
| 2 | 225367995 | C | T | 2 | 225367996 | C | G | 2 | 225368094 | G | T |
| 2 | 225371246 | A | C | 2 | 225434256 | C | G | 2 | 225658104 | G | A |
| 2 | 228264500 | C | T | 2 | 228725134 | C | T | 2 | 229039040 | C | T |
| 2 | 230470702 | A | G | 2 | 230471160 | T | G | 2 | 233311776 | G | T |
| 2 | 233311824 | A | T | 2 | 234071665 | A | C | 2 | 234676118 | A | G |
| 2 | 234680679 | A | C | 2 | 234681416 | T | C | 2 | 235386951 | T | C |
| 2 | 236618069 | T | C | 2 | 236618494 | A | T | 2 | 236618499 | A | G |
| 2 | 237032523 | A | G | 2 | 237032837 | G | A | 2 | 237820187 | C | T |
| 2 | 238512001 | G | A | 2 | 239675968 | C | T | 2 | 239675977 | T | G |
| 2 | 240872090 | G | A | 2 | 241063584 | G | A | 2 | 241588811 | T | A |
| 2 | 242793849 | G | A | 2 | 243009529 | A | G | 2 | 243059290 | A | T |
| 2 | 243059293 | C | A | 2 | 243071262 | G | T | 2 | 243088710 | G | C |
| 3 | 142069 | A | C | 3 | 1947348 | A | G | 3 | 1947477 | T | C |
| 3 | 2409278 | A | G | 3 | 2409817 | T | C | 3 | 3142901 | T | C |
| 3 | 3142943 | T | C | 3 | 4025464 | A | G | 3 | 4025573 | T | C |
| 3 | 4665123 | A | G | 3 | 5131185 | C | G | 3 | 7888983 | T | C |
| 3 | 8730717 | G | A | 3 | 8730781 | A | C | 3 | 9877716 | G | A |
| 3 | 9877783 | G | A | 3 | 10030236 | T | C | 3 | 10030521 | T | C |
| 3 | 10034925 | G | C | 3 | 10035601 | C | T | 3 | 10075983 | T | C |
| 3 | 10075991 | A | G | 3 | 10078247 | A | G | 3 | 10078289 | G | C |
| 3 | 10080722 | T | C | 3 | 10083169 | C | T | 3 | 10085816 | G | T |
| 3 | 10094418 | T | C | 3 | 10102377 | T | A | 3 | 10104186 | A | T |
| 3 | 10106924 | G | A | 3 | 10108745 | G | C | 3 | 10119603 | C | A |
| 3 | 10122575 | T | C | 3 | 10131831 | C | T | 3 | 10133710 | G | A |
| 3 | 10134766 | C | T | 3 | 10140115 | G | A | 3 | 10183274 | C | T |
| 3 | 10183337 | G | A | 3 | 10191219 | C | G | 3 | 10191943 | G | A |
| 3 | 11041606 | G | A | 3 | 11042207 | T | C | 3 | 11767152 | A | G |
| 3 | 11902162 | G | A | 3 | 11911822 | C | A | 3 | 11912512 | A | C |
| 3 | 11912534 | T | C | 3 | 11912557 | G | A | 3 | 11912622 | G | A |
| 3 | 11912635 | C | T | 3 | 11913124 | A | G | 3 | 11913188 | A | G |
| 3 | 11915481 | C | T | 3 | 11924678 | C | G | 3 | 11925226 | G | A |
| 3 | 11925254 | T | C | 3 | 11926243 | C | G | 3 | 11926269 | G | A |
| 3 | 11926423 | A | G | 3 | 11926467 | A | G | 3 | 11926508 | A | G |
| 3 | 11926604 | A | G | 3 | 11926734 | T | G | 3 | 11928126 | T | G |
| 3 | 11928127 | A | T | 3 | 11928469 | G | T | 3 | 11932455 | T | G |
| 3 | 11933223 | T | C | 3 | 12299313 | A | G | 3 | 12632652 | G | C |
| 3 | 12633552 | T | C | 3 | 12829030 | A | G | 3 | 12829036 | C | T |
| 3 | 12829040 | G | T | 3 | 12829095 | A | C | 3 | 12829156 | A | G |
| 3 | 13152051 | T | C | 3 | 13152457 | C | T | 3 | 14189147 | A | G |
| 3 | 14212258 | C | A | 3 | 15516820 | C | T | 3 | 16408371 | C | T |
| 3 | 16408389 | G | T | 3 | 16408401 | G | C | 3 | 16408402 | G | A |
| 3 | 16408413 | G | A | 3 | 16408424 | C | T | 3 | 16408445 | G | A |
| 3 | 16408451 | C | T | 3 | 16408457 | C | T | 3 | 16408487 | C | G |
| 3 | 16408494 | T | C | 3 | 16408499 | C | T | 3 | 16408500 | A | G |
| 3 | 16408503 | G | T | 3 | 16408519 | G | A | 3 | 16408533 | C | T |
| 3 | 16408534 | A | G | 3 | 16408536 | G | A | 3 | 16408793 | T | G |
| 3 | 16408949 | T | C | 3 | 16408990 | G | T | 3 | 16740865 | A | G |
| 3 | 18412689 | G | T | 3 | 19405951 | T | A | 3 | 21805928 | G | A |
| 3 | 22485461 | T | C | 3 | 22998179 | C | T | 3 | 22998724 | G | T |
| 3 | 22998753 | C | T | 3 | 22998818 | T | C | 3 | 22998824 | A | G |
| 3 | 23128544 | T | C | 3 | 24355183 | A | T | 3 | 24355275 | G | A |
| 3 | 25619630 | G | C | 3 | 25620233 | T | A | 3 | 26445467 | T | C |
| 3 | 27432779 | C | T | 3 | 27432871 | G | A | 3 | 27756251 | T | C |
| 3 | 30686627 | T | C | 3 | 30686647 | T | C | 3 | 30715977 | C | T |
| 3 | 30729636 | G | A | 3 | 32071969 | G | A | 3 | 32072004 | T | C |
| 3 | 32195427 | T | C | 3 | 32232782 | G | T | 3 | 32301422 | C | A |
| 3 | 32301426 | C | T | 3 | 33086893 | T | C | 3 | 33841065 | A | G |
| 3 | 36148273 | C | T | 3 | 36148306 | T | G | 3 | 36148330 | C | T |
| 3 | 36148763 | G | C | 3 | 37067050 | A | G | 3 | 37627598 | T | C |
| 3 | 37790112 | C | T | 3 | 38737643 | T | C | 3 | 40439614 | G | A |
| 3 | 40440116 | G | A | 3 | 40537215 | C | A | 3 | 41269152 | A | T |
| 3 | 45251978 | G | A | 3 | 46511969 | G | A | 3 | 47083782 | C | G |
| 3 | 47103376 | C | T | 3 | 47108330 | C | T | 3 | 48486773 | G | C |
| 3 | 48487338 | G | A | 3 | 48487353 | A | C | 3 | 51102839 | G | A |
| 3 | 51755441 | G | A | 3 | 52597664 | G | A | 3 | 52621839 | C | T |
| 3 | 52659124 | T | C | 3 | 52678494 | G | A | 3 | 53884074 | G | C |
| 3 | 53999979 | A | G | 3 | 55738985 | T | G | 3 | 56235603 | C | A |
| 3 | 56737197 | C | T | 3 | 58036651 | T | C | 3 | 58594418 | C | A |
| 3 | 59701045 | C | T | 3 | 60439446 | T | A | 3 | 60545661 | T | A |
| 3 | 61557787 | G | A | 3 | 63442932 | A | T | 3 | 63443008 | A | C |
| 3 | 64184676 | G | A | 3 | 65289882 | C | T | 3 | 65734943 | T | C |
| 3 | 66077019 | T | C | 3 | 66077076 | A | T | 3 | 66077167 | G | A |
| 3 | 66077485 | G | A | 3 | 66746914 | T | C | 3 | 67145056 | T | C |
| 3 | 69392244 | G | T | 3 | 69392263 | T | C | 3 | 69392331 | C | A |
| 3 | 69392754 | G | A | 3 | 69392762 | G | A | 3 | 69392793 | T | C |
| 3 | 70013349 | A | G | 3 | 71847123 | A | G | 3 | 72978434 | T | C |
| 3 | 73433369 | G | A | 3 | 74513397 | A | G | 3 | 74514007 | T | C |
| 3 | 75365484 | T | C | 3 | 75366035 | C | T | 3 | 75397290 | A | G |
| 3 | 75475805 | A | G | 3 | 75572090 | T | C | 3 | 75572133 | C | G |
| 3 | 75572165 | G | C | 3 | 75572199 | C | T | 3 | 75572202 | T | C |
| 3 | 75572238 | G | A | 3 | 75572329 | G | C | 3 | 75572368 | A | C |
| 3 | 75572374 | G | C | 3 | 75875639 | A | C | 3 | 75875740 | G | A |
| 3 | 75875973 | G | A | 3 | 75876015 | T | G | 3 | 75876023 | A | G |
| 3 | 75876115 | G | A | 3 | 76779549 | C | G | 3 | 76779577 | T | G |
| 3 | 76780491 | A | G | 3 | 77338588 | A | C | 3 | 77338630 | G | T |
| 3 | 77338634 | T | C | 3 | 77338635 | A | C | 3 | 77338636 | A | C |
| 3 | 77338640 | T | C | 3 | 77338835 | G | C | 3 | 77338920 | T | C |
| 3 | 77339156 | G | C | 3 | 77339675 | T | G | 3 | 77340008 | T | C |
| 3 | 77345372 | T | G | 3 | 77345549 | G | A | 3 | 77345598 | G | A |
| 3 | 77459994 | T | C | 3 | 80457094 | G | A | 3 | 81833577 | T | C |
| 3 | 81833966 | G | A | 3 | 81834060 | G | A | 3 | 81834065 | T | A |
| 3 | 83497272 | A | G | 3 | 84924659 | T | C | 3 | 85870199 | A | T |
| 3 | 86702627 | G | T | 3 | 87386674 | T | C | 3 | 87695509 | C | T |
| 3 | 89176645 | A | G | 3 | 89444743 | G | T | 3 | 89448936 | T | A |
| 3 | 89498096 | C | A | 3 | 89521965 | T | G | 3 | 89528313 | T | G |
| 3 | 93903910 | G | A | 3 | 95886072 | A | G | 3 | 97940846 | C | T |
| 3 | 97957539 | C | T | 3 | 97957580 | G | C | 3 | 97957609 | C | T |
| 3 | 97957611 | G | A | 3 | 97983257 | C | G | 3 | 97983265 | C | A |
| 3 | 97983711 | T | C | 3 | 98840224 | C | T | 3 | 101620615 | G | A |
| 3 | 101620682 | A | G | 3 | 102650845 | G | C | 3 | 103705800 | A | T |
| 3 | 104106527 | G | A | 3 | 104106528 | G | A | 3 | 104106562 | C | T |
| 3 | 104639822 | T | C | 3 | 105377515 | C | T | 3 | 105400846 | A | G |
| 3 | 105420602 | C | T | 3 | 105422536 | T | C | 3 | 105439311 | T | A |
| 3 | 105452593 | A | G | 3 | 105459717 | A | G | 3 | 105571964 | C | T |
| 3 | 105586714 | T | C | 3 | 107805553 | C | G | 3 | 108034167 | A | C |
| 3 | 109128553 | G | T | 3 | 109128692 | C | T | 3 | 109775371 | G | A |
| 3 | 109775869 | G | A | 3 | 110496808 | T | C | 3 | 112243112 | A | C |
| 3 | 112352718 | A | G | 3 | 112933625 | A | G | 3 | 112933635 | T | C |
| 3 | 117156240 | G | A | 3 | 117156255 | C | T | 3 | 117156294 | C | A |
| 3 | 117156738 | T | C | 3 | 119272235 | T | C | 3 | 119419376 | G | A |
| 3 | 120402581 | G | T | 3 | 122380722 | C | A | 3 | 123957278 | C | T |
| 3 | 123957294 | C | G | 3 | 124410346 | A | G | 3 | 124840312 | T | A |
| 3 | 125431505 | A | C | 3 | 125431673 | A | T | 3 | 125453782 | G | T |
| 3 | 125453797 | C | T | 3 | 125453908 | G | T | 3 | 125453977 | A | T |
| 3 | 125454162 | C | G | 3 | 125454180 | A | C | 3 | 125482012 | T | G |
| 3 | 125482430 | C | T | 3 | 125484006 | G | C | 3 | 125484009 | C | T |
| 3 | 125494802 | T | C | 3 | 125494864 | T | C | 3 | 125495356 | A | G |
| 3 | 125495487 | T | C | 3 | 125495558 | A | G | 3 | 125639885 | C | A |
| 3 | 125639911 | C | T | 3 | 125643329 | G | A | 3 | 125643332 | A | G |
| 3 | 125643719 | A | G | 3 | 125643772 | G | A | 3 | 126254692 | G | A |
| 3 | 128201013 | A | C | 3 | 129309455 | G | A | 3 | 129722895 | G | A |
| 3 | 129724802 | C | T | 3 | 129804885 | C | G | 3 | 129805060 | C | A |
| 3 | 129805077 | C | T | 3 | 129805236 | A | G | 3 | 129821816 | C | G |
| 3 | 129821817 | C | T | 3 | 129822217 | C | T | 3 | 129822218 | A | G |
| 3 | 129822230 | T | C | 3 | 129825954 | T | G | 3 | 129825955 | A | G |
| 3 | 129826002 | G | T | 3 | 129826513 | T | C | 3 | 129828037 | C | A |
| 3 | 129828078 | T | G | 3 | 129919713 | C | T | 3 | 129920258 | C | T |
| 3 | 130745968 | C | T | 3 | 130745981 | A | G | 3 | 131138571 | C | T |
| 3 | 131138589 | A | G | 3 | 131138658 | C | T | 3 | 133124049 | C | T |
| 3 | 141485046 | T | C | 3 | 142232743 | A | T | 3 | 142233990 | T | C |
| 3 | 142234534 | G | A | 3 | 142234589 | C | A | 3 | 142271797 | A | C |
| 3 | 142273032 | T | C | 3 | 142274534 | C | T | 3 | 142279796 | T | C |
| 3 | 142659361 | A | G | 3 | 143426876 | C | T | 3 | 143427340 | C | G |
| 3 | 144276648 | A | G | 3 | 145139463 | T | C | 3 | 145139946 | A | T |
| 3 | 147955974 | G | T | 3 | 148649659 | C | A | 3 | 148649678 | C | T |
| 3 | 148650252 | G | A | 3 | 149181661 | C | T | 3 | 149271087 | T | C |
| 3 | 149657658 | C | T | 3 | 151053633 | T | C | 3 | 152685955 | T | C |
| 3 | 152686473 | A | G | 3 | 152686523 | G | A | 3 | 152686545 | C | T |
| 3 | 153099380 | T | C | 3 | 155829938 | A | C | 3 | 155830631 | C | T |
| 3 | 156174496 | G | A | 3 | 156880573 | T | C | 3 | 156880587 | A | G |
| 3 | 156880688 | A | G | 3 | 157559512 | A | G | 3 | 158780937 | A | G |
| 3 | 159490097 | C | T | 3 | 162693420 | C | T | 3 | 162693961 | T | A |
| 3 | 164940782 | A | G | 3 | 164941295 | G | A | 3 | 165796202 | A | G |
| 3 | 165796228 | A | T | 3 | 166033539 | G | A | 3 | 166580001 | C | T |
| 3 | 166749739 | T | C | 3 | 167800290 | T | C | 3 | 169200968 | C | A |
| 3 | 169201226 | G | A | 3 | 169427817 | A | G | 3 | 169977589 | T | C |
| 3 | 169978059 | A | G | 3 | 169981058 | G | A | 3 | 169985539 | C | A |
| 3 | 169986068 | T | G | 3 | 169988658 | G | A | 3 | 169998506 | A | G |
| 3 | 170009952 | G | A | 3 | 170021160 | G | A | 3 | 171071949 | T | G |
| 3 | 171684965 | G | A | 3 | 172126162 | C | T | 3 | 173114305 | A | G |
| 3 | 173874385 | G | A | 3 | 173874469 | G | T | 3 | 174056020 | C | G |
| 3 | 174562570 | G | T | 3 | 174562655 | G | T | 3 | 174562942 | A | T |
| 3 | 174563022 | G | A | 3 | 175937783 | T | C | 3 | 176405586 | C | T |
| 3 | 177744450 | G | A | 3 | 178927111 | C | A | 3 | 178927149 | T | C |
| 3 | 178935799 | T | C | 3 | 178938554 | A | T | 3 | 178948385 | A | T |
| 3 | 180424060 | C | T | 3 | 181994593 | C | T | 3 | 181994594 | G | A |
| 3 | 182332176 | T | A | 3 | 182735211 | T | G | 3 | 183204239 | A | G |
| 3 | 184220524 | G | T | 3 | 184220528 | T | C | 3 | 184220531 | G | A |
| 3 | 184220534 | T | C | 3 | 184220549 | A | G | 3 | 184220588 | C | T |
| 3 | 184220608 | G | C | 3 | 184220819 | G | A | 3 | 184220848 | G | C |
| 3 | 184220849 | G | A | 3 | 184220864 | C | T | 3 | 184220865 | A | G |
| 3 | 184220878 | G | C | 3 | 184220879 | G | A | 3 | 184220880 | T | C |
| 3 | 184220890 | T | C | 3 | 184220894 | T | C | 3 | 184220902 | A | C |
| 3 | 184220907 | A | T | 3 | 184220910 | T | C | 3 | 184220915 | A | G |
| 3 | 184220920 | T | C | 3 | 184220924 | C | T | 3 | 184220940 | C | T |
| 3 | 184220945 | G | A | 3 | 184220948 | T | A | 3 | 184220950 | T | C |
| 3 | 184928239 | T | C | 3 | 186508574 | G | C | 3 | 188293043 | T | C |
| 3 | 189382904 | T | C | 3 | 189383430 | C | T | 3 | 190093573 | G | C |
| 3 | 190093689 | G | T | 3 | 190093915 | T | C | 3 | 190094003 | T | C |
| 3 | 190094024 | G | A | 3 | 191093053 | T | C | 3 | 191093080 | A | G |
| 3 | 191741924 | C | T | 3 | 191742322 | G | A | 3 | 194426037 | C | A |
| 3 | 194712317 | C | A | 3 | 194723092 | G | T | 3 | 195384760 | G | C |
| 3 | 195384844 | T | G | 3 | 195399268 | A | C | 3 | 195436937 | A | C |
| 3 | 195437073 | A | G | 3 | 195437584 | G | C | 3 | 195437844 | T | G |
| 3 | 195437869 | C | T | 3 | 195437970 | T | C | 3 | 195437979 | T | G |
| 3 | 195437988 | T | C | 3 | 195437989 | C | G | 3 | 195437990 | A | C |
| 3 | 195437994 | A | G | 3 | 195438024 | T | G | 3 | 195438126 | T | G |
| 3 | 195438174 | A | G | 3 | 195438443 | T | C | 3 | 195438480 | A | C |
| 3 | 195692049 | G | T | 3 | 195692516 | C | T | 3 | 195692570 | G | A |
| 3 | 195700978 | A | C | 3 | 195701115 | A | G | 3 | 195709312 | G | C |
| 3 | 195709355 | C | G | 3 | 195717337 | C | G | 3 | 195872302 | C | T |
| 3 | 195872790 | T | G | 3 | 196379737 | C | G | 3 | 196759586 | C | A |
| 3 | 196759600 | A | G | 3 | 196759604 | T | G | 3 | 196759761 | A | C |
| 3 | 196759779 | A | G | 3 | 196760400 | C | T | 3 | 196760409 | G | A |
| 3 | 197110726 | C | T | 3 | 197112110 | C | A | 3 | 197114790 | A | G |
| 3 | 197114791 | G | C | 3 | 197114962 | T | C | 3 | 197115282 | A | C |
| 3 | 197115557 | T | G | 3 | 197115558 | C | T | 3 | 197115602 | A | G |
| 3 | 197116179 | G | A | 3 | 197116267 | A | G | 3 | 197116296 | T | C |
| 3 | 197116307 | G | C | 3 | 197116710 | G | T | 3 | 197118688 | T | C |
| 3 | 197354725 | C | T | 3 | 197354733 | T | G | 3 | 197354857 | C | A |
| 3 | 197354909 | C | A | 3 | 197825954 | G | A | 3 | 197825956 | G | A |
| 3 | 197826004 | G | A | 3 | 197826013 | C | A | 3 | 197826025 | G | A |
| 3 | 197826185 | G | A | 3 | 197832131 | C | T | 3 | 197832181 | C | T |
| 3 | 197832551 | A | G | 3 | 197832937 | G | C | 3 | 197832951 | G | A |
| 3 | 197851897 | G | A | 4 | 329501 | G | T | 4 | 1266462 | G | A |
| 4 | 1267160 | T | C | 4 | 1267203 | G | T | 4 | 1732410 | C | T |
| 4 | 1736737 | T | C | 4 | 1738732 | C | T | 4 | 1740539 | T | C |
| 4 | 2432104 | G | A | 4 | 2699972 | T | C | 4 | 3536882 | A | G |
| 4 | 3547346 | T | C | 4 | 3903137 | G | A | 4 | 3903256 | C | T |
| 4 | 3903553 | G | A | 4 | 3903604 | A | C | 4 | 3952705 | A | T |
| 4 | 3952730 | T | C | 4 | 3953310 | A | G | 4 | 3953330 | A | G |
| 4 | 3953359 | A | G | 4 | 3953364 | C | T | 4 | 3954873 | A | T |
| 4 | 3954878 | T | C | 4 | 3974295 | G | T | 4 | 3974296 | A | T |
| 4 | 3974455 | C | A | 4 | 3974546 | A | G | 4 | 3974559 | T | C |
| 4 | 3974583 | G | C | 4 | 4060689 | G | A | 4 | 4061299 | T | C |
| 4 | 4099959 | G | A | 4 | 4112309 | A | G | 4 | 4112604 | A | G |
| 4 | 5189645 | G | A | 4 | 5189717 | A | T | 4 | 5652298 | G | C |
| 4 | 6200768 | C | T | 4 | 6881739 | G | C | 4 | 6881785 | G | C |
| 4 | 8999303 | T | A | 4 | 8999512 | C | T | 4 | 9001121 | G | A |
| 4 | 9001176 | G | C | 4 | 9001703 | T | C | 4 | 9012383 | A | T |
| 4 | 9052653 | G | C | 4 | 9137789 | G | C | 4 | 9137790 | A | G |
| 4 | 9137990 | G | A | 4 | 9158848 | G | A | 4 | 9159079 | T | G |
| 4 | 9159219 | C | T | 4 | 9159256 | C | G | 4 | 9163190 | A | T |
| 4 | 9271895 | T | G | 4 | 9531098 | A | G | 4 | 9532659 | G | T |
| 4 | 9584058 | G | A | 4 | 9673458 | T | C | 4 | 9673980 | C | T |
| 4 | 9696823 | G | A | 4 | 9700910 | A | T | 4 | 9757407 | G | A |
| 4 | 9757485 | G | A | 4 | 10180622 | T | C | 4 | 10180643 | T | C |
| 4 | 10726609 | G | A | 4 | 10726610 | C | T | 4 | 10726707 | C | T |
| 4 | 12056196 | T | C | 4 | 13031210 | T | G | 4 | 14232397 | C | T |
| 4 | 14837656 | C | T | 4 | 15117509 | G | A | 4 | 15733093 | C | A |
| 4 | 15733108 | G | T | 4 | 15733159 | A | G | 4 | 17348027 | A | G |
| 4 | 17503839 | G | A | 4 | 18056140 | C | T | 4 | 18798633 | C | A |
| 4 | 19250760 | C | T | 4 | 21551721 | T | C | 4 | 21552230 | A | G |
| 4 | 23715644 | C | T | 4 | 24142003 | A | C | 4 | 25721273 | C | T |
| 4 | 27128768 | C | T | 4 | 27683108 | T | A | 4 | 28247387 | G | A |
| 4 | 28247910 | G | A | 4 | 28551237 | G | A | 4 | 29333030 | C | T |
| 4 | 29333037 | C | T | 4 | 29707479 | G | A | 4 | 30290545 | A | G |
| 4 | 30290549 | T | A | 4 | 31017144 | C | G | 4 | 31017221 | C | T |
| 4 | 31761297 | G | A | 4 | 32592869 | C | G | 4 | 32592949 | C | T |
| 4 | 33642926 | T | C | 4 | 34985401 | T | G | 4 | 34985806 | G | A |
| 4 | 35510086 | A | C | 4 | 35510132 | C | T | 4 | 35510659 | A | G |
| 4 | 35510681 | A | G | 4 | 36329799 | A | C | 4 | 36330306 | T | A |
| 4 | 37002365 | T | C | 4 | 37002380 | A | G | 4 | 38512602 | T | C |
| 4 | 39482627 | T | C | 4 | 39482684 | A | C | 4 | 39483115 | A | G |
| 4 | 39483162 | G | A | 4 | 40022837 | A | G | 4 | 40022870 | C | T |
| 4 | 40023258 | T | C | 4 | 40835384 | T | A | 4 | 40835854 | C | A |
| 4 | 41748511 | T | G | 4 | 42608665 | T | C | 4 | 46241769 | G | A |
| 4 | 46763980 | A | C | 4 | 47493210 | C | G | 4 | 49292437 | T | G |
| 4 | 49298634 | A | C | 4 | 49304353 | T | G | 4 | 49304428 | A | C |
| 4 | 49304465 | T | C | 4 | 49304469 | T | A | 4 | 49314901 | C | T |
| 4 | 49321018 | A | G | 4 | 49321104 | G | T | 4 | 49617655 | C | T |
| 4 | 49628621 | C | T | 4 | 49628869 | T | A | 4 | 49628914 | A | C |
| 4 | 49629091 | T | C | 4 | 49629144 | G | A | 4 | 49629145 | T | C |
| 4 | 52978719 | C | A | 4 | 52978741 | T | C | 4 | 52979321 | T | C |
| 4 | 53454588 | T | G | 4 | 54430834 | T | C | 4 | 55136027 | G | C |
| 4 | 55136245 | A | G | 4 | 55137183 | T | C | 4 | 55143321 | C | T |
| 4 | 55146926 | T | A | 4 | 55146927 | T | A | 4 | 55161047 | C | G |
| 4 | 55523938 | G | A | 4 | 55524533 | C | G | 4 | 55573693 | G | A |
| 4 | 55602451 | C | T | 4 | 55962165 | A | T | 4 | 55962166 | C | T |
| 4 | 55967806 | A | G | 4 | 55971400 | G | A | 4 | 55974290 | C | G |
| 4 | 55985142 | A | G | 4 | 55985149 | A | G | 4 | 55985209 | C | G |
| 4 | 55991731 | T | C | 4 | 57917346 | G | T | 4 | 58969178 | C | G |
| 4 | 58969622 | A | C | 4 | 59117179 | C | A | 4 | 59685846 | A | G |
| 4 | 63895205 | T | A | 4 | 64379405 | A | G | 4 | 64931039 | T | C |
| 4 | 66270412 | C | T | 4 | 66355888 | T | C | 4 | 66355901 | G | T |
| 4 | 67970457 | T | C | 4 | 69901970 | C | T | 4 | 70354884 | G | A |
| 4 | 72347419 | G | T | 4 | 73292973 | C | T | 4 | 74322255 | T | C |
| 4 | 74738851 | G | A | 4 | 75394764 | A | G | 4 | 75395312 | G | A |
| 4 | 75395322 | C | A | 4 | 78990229 | C | A | 4 | 78990900 | C | A |
| 4 | 79482186 | G | A | 4 | 79602718 | G | A | 4 | 80386263 | T | G |
| 4 | 80801388 | T | A | 4 | 84013033 | C | T | 4 | 86340131 | C | G |
| 4 | 86340164 | G | A | 4 | 87264746 | T | A | 4 | 87264750 | G | A |
| 4 | 87692129 | T | G | 4 | 87695876 | T | A | 4 | 87718279 | G | A |
| 4 | 87732483 | C | T | 4 | 87791437 | C | T | 4 | 89777379 | C | G |
| 4 | 90228238 | C | T | 4 | 93487149 | A | T | 4 | 93487465 | C | A |
| 4 | 95088301 | T | C | 4 | 95606136 | T | C | 4 | 99443068 | A | G |
| 4 | 99443113 | A | G | 4 | 99443127 | T | C | 4 | 99443569 | T | C |
| 4 | 99443585 | G | A | 4 | 99443611 | A | T | 4 | 100238998 | T | C |
| 4 | 101572039 | G | A | 4 | 102854264 | C | T | 4 | 104452689 | T | A |
| 4 | 106164284 | T | G | 4 | 106193588 | A | G | 4 | 106914301 | C | T |
| 4 | 106914306 | A | T | 4 | 106914338 | C | T | 4 | 106914855 | G | A |
| 4 | 108124872 | G | A | 4 | 108125157 | G | A | 4 | 108125170 | C | A |
| 4 | 108565609 | G | A | 4 | 111944989 | A | G | 4 | 113180779 | C | T |
| 4 | 113181343 | T | C | 4 | 113486769 | T | C | 4 | 115241715 | C | G |
| 4 | 115987887 | A | T | 4 | 116002140 | T | C | 4 | 117957242 | T | G |
| 4 | 118496905 | A | G | 4 | 118496919 | T | C | 4 | 118497508 | C | G |
| 4 | 118503372 | C | T | 4 | 119252323 | C | T | 4 | 119252368 | G | A |
| 4 | 119526740 | C | G | 4 | 119527206 | C | T | 4 | 119944319 | A | G |
| 4 | 119944900 | A | G | 4 | 119944903 | C | A | 4 | 120133549 | T | G |
| 4 | 120324707 | T | C | 4 | 120325264 | C | T | 4 | 120325273 | T | C |
| 4 | 120355709 | T | C | 4 | 120751902 | T | A | 4 | 121089766 | G | C |
| 4 | 121738049 | T | C | 4 | 122290719 | G | A | 4 | 122290979 | G | C |
| 4 | 122291131 | T | G | 4 | 122431338 | T | C | 4 | 122683623 | C | T |
| 4 | 124946414 | C | T | 4 | 124946423 | A | G | 4 | 125173747 | C | T |
| 4 | 125725127 | A | G | 4 | 127856273 | T | G | 4 | 128518960 | C | T |
| 4 | 132832700 | T | G | 4 | 132832731 | A | G | 4 | 133045796 | A | G |
| 4 | 133824971 | A | G | 4 | 133825066 | G | A | 4 | 133825500 | C | T |
| 4 | 133825533 | A | G | 4 | 133885960 | T | G | 4 | 134784133 | G | A |
| 4 | 137489544 | C | A | 4 | 137985807 | C | T | 4 | 138698629 | T | A |
| 4 | 139796813 | G | A | 4 | 140619075 | T | C | 4 | 143003526 | A | G |
| 4 | 143034019 | A | T | 4 | 143034029 | G | A | 4 | 143044829 | A | G |
| 4 | 143045482 | G | A | 4 | 143066775 | A | G | 4 | 143095131 | C | T |
| 4 | 143095139 | C | T | 4 | 143095201 | C | T | 4 | 143114003 | T | C |
| 4 | 143114011 | A | G | 4 | 143114040 | A | C | 4 | 143114482 | T | C |
| 4 | 143114548 | A | G | 4 | 143114556 | A | G | 4 | 143114558 | G | A |
| 4 | 143114598 | G | C | 4 | 143129292 | G | A | 4 | 143236139 | T | C |
| 4 | 143236158 | T | C | 4 | 143323915 | T | G | 4 | 143326120 | G | T |
| 4 | 143326714 | T | G | 4 | 143352065 | T | C | 4 | 143926811 | A | G |
| 4 | 144754667 | A | G | 4 | 145214743 | G | A | 4 | 145568682 | A | C |
| 4 | 146656399 | T | C | 4 | 147206992 | G | A | 4 | 147207075 | G | C |
| 4 | 147208378 | C | G | 4 | 147970517 | G | A | 4 | 148024671 | G | C |
| 4 | 149067352 | C | G | 4 | 149870390 | T | C | 4 | 150167697 | A | C |
| 4 | 150674206 | A | T | 4 | 151259617 | T | C | 4 | 153254121 | C | T |
| 4 | 153960301 | T | A | 4 | 155298773 | T | G | 4 | 155299346 | T | C |
| 4 | 155851079 | C | A | 4 | 156433308 | G | A | 4 | 156607373 | A | G |
| 4 | 157616767 | G | A | 4 | 159194870 | G | A | 4 | 159931005 | G | C |
| 4 | 160823765 | G | C | 4 | 161285415 | G | A | 4 | 161956269 | G | A |
| 4 | 161956317 | C | T | 4 | 161956363 | T | A | 4 | 161956376 | C | T |
| 4 | 161956767 | A | G | 4 | 161956781 | A | C | 4 | 162129559 | A | G |
| 4 | 163086385 | A | C | 4 | 164465973 | A | T | 4 | 164890625 | T | C |
| 4 | 165933477 | C | A | 4 | 167125325 | C | G | 4 | 167654337 | C | T |
| 4 | 167654397 | C | G | 4 | 167654397 | C | T | 4 | 167654427 | C | A |
| 4 | 168292643 | G | A | 4 | 168292723 | A | G | 4 | 168292832 | T | C |
| 4 | 168292836 | A | T | 4 | 168292853 | T | A | 4 | 168292864 | T | C |
| 4 | 168292887 | A | G | 4 | 169633306 | C | T | 4 | 170812566 | C | T |
| 4 | 171078557 | T | A | 4 | 171774610 | C | T | 4 | 178128578 | G | C |
| 4 | 180339049 | A | G | 4 | 181286425 | C | T | 4 | 181509274 | G | C |
| 4 | 183032807 | A | G | 4 | 184807301 | A | G | 4 | 185311508 | T | C |
| 4 | 185311570 | G | A | 4 | 185329052 | G | A | 4 | 185350307 | T | C |
| 4 | 185350422 | A | G | 4 | 185779168 | T | C | 4 | 185779461 | C | T |
| 4 | 185779481 | G | A | 4 | 185780254 | G | A | 4 | 186485037 | G | A |
| 4 | 187353180 | A | C | 4 | 187353188 | A | G | 4 | 187353199 | G | A |
| 4 | 187353362 | G | A | 4 | 187353388 | C | T | 4 | 187353390 | C | A |
| 4 | 187353437 | G | A | 4 | 187353459 | C | T | 4 | 187353485 | C | T |
| 4 | 187353486 | C | G | 4 | 187353487 | G | A | 4 | 187353491 | C | T |
| 4 | 187353497 | T | C | 4 | 187353500 | T | A | 4 | 187353508 | A | G |
| 4 | 187353510 | G | C | 4 | 187353510 | G | T | 4 | 187353518 | C | G |
| 4 | 187353520 | A | G | 4 | 187353550 | T | C | 4 | 187353554 | A | G |
| 4 | 187353557 | G | C | 4 | 187353610 | C | T | 4 | 187353658 | A | G |
| 4 | 187353818 | A | G | 4 | 187354048 | A | G | 4 | 187354174 | G | A |
| 4 | 187354186 | G | A | 4 | 187354197 | T | C | 4 | 187354312 | A | G |
| 4 | 187354335 | T | C | 4 | 187354416 | G | A | 4 | 187354429 | A | G |
| 4 | 187354521 | G | A | 4 | 187354542 | A | G | 4 | 187354828 | T | C |
| 4 | 187355027 | G | A | 4 | 187356475 | G | C | 4 | 187356484 | G | A |
| 4 | 187356497 | G | A | 4 | 187356955 | T | C | 4 | 187356957 | A | G |
| 4 | 187357003 | G | A | 4 | 187357027 | G | C | 4 | 187357036 | G | A |
| 4 | 187357363 | G | A | 4 | 187357369 | T | C | 4 | 187357387 | C | T |
| 4 | 187357415 | T | C | 4 | 187357667 | T | C | 4 | 187357686 | G | C |
| 4 | 187357798 | A | G | 4 | 187522112 | C | G | 4 | 187522789 | T | C |
| 4 | 187525910 | A | T | 4 | 187525922 | G | A | 4 | 187525940 | A | C |
| 4 | 187525941 | G | T | 4 | 187525946 | A | G | 4 | 187525986 | G | A |
| 4 | 187526002 | C | A | 4 | 187533153 | C | T | 4 | 187534689 | G | T |
| 4 | 187535063 | T | C | 4 | 187535765 | T | C | 4 | 187631223 | C | T |
| 4 | 189426841 | A | G | 4 | 189426857 | G | T | 4 | 189426863 | C | T |
| 4 | 189426864 | G | A | 4 | 189426871 | C | A | 4 | 189426888 | G | C |
| 4 | 189427370 | G | A | 4 | 189427435 | A | T | 4 | 189972360 | A | G |
| 4 | 189972363 | G | A | 4 | 189972366 | A | G | 4 | 189972384 | G | A |
| 4 | 189972492 | A | G | 4 | 189972498 | G | A | 4 | 189972501 | A | G |
| 4 | 189972516 | G | C | 4 | 189972897 | G | A | 4 | 189972919 | A | G |
| 4 | 189972930 | A | G | 4 | 189972933 | G | A | 4 | 189973230 | A | G |
| 4 | 189973257 | G | A | 4 | 189973266 | A | G | 4 | 189973278 | G | A |
| 4 | 189973949 | T | C | 4 | 190197957 | C | A | 4 | 190198002 | A | G |
| 4 | 190198077 | A | G | 4 | 190198080 | T | C | 4 | 190198100 | C | G |
| 4 | 190198107 | G | A | 4 | 190198111 | G | A | 4 | 190198121 | C | G |
| 4 | 190198127 | A | G | 4 | 190198142 | T | G | 4 | 190198193 | G | C |
| 4 | 190198201 | G | A | 4 | 190198206 | T | C | 4 | 190198214 | C | T |
| 4 | 190198221 | A | C | 4 | 190198225 | T | C | 4 | 190198226 | G | A |
| 4 | 190198377 | C | T | 4 | 190198703 | G | A | 4 | 190198707 | G | A |
| 4 | 190198717 | C | G | 4 | 190198844 | T | C | 4 | 190199033 | T | C |
| 4 | 190199199 | T | C | 4 | 190859230 | C | G | 4 | 190859289 | A | C |
| 4 | 190859333 | A | G | 4 | 190878827 | T | G | 4 | 190899555 | T | C |
| 5 | 19602 | A | G | 5 | 223415 | C | T | 5 | 225267 | C | A |
| 5 | 226453 | G | A | 5 | 233325 | G | A | 5 | 233362 | T | G |
| 5 | 233371 | A | G | 5 | 236932 | T | A | 5 | 254442 | C | T |
| 5 | 254861 | C | G | 5 | 649405 | A | G | 5 | 649749 | T | C |
| 5 | 650037 | T | C | 5 | 650266 | G | A | 5 | 650846 | C | T |
| 5 | 651295 | T | G | 5 | 1261052 | G | A | 5 | 1266226 | T | C |
| 5 | 1266310 | A | G | 5 | 1271524 | T | C | 5 | 1572585 | A | G |
| 5 | 1594664 | C | G | 5 | 1600771 | A | G | 5 | 1600785 | T | C |
| 5 | 1858062 | T | C | 5 | 1858062 | T | G | 5 | 1858821 | A | T |
| 5 | 1858834 | G | C | 5 | 2145174 | T | C | 5 | 2145508 | T | C |
| 5 | 2146102 | G | A | 5 | 2146276 | C | G | 5 | 2146338 | T | G |
| 5 | 2146345 | T | G | 5 | 2146527 | C | A | 5 | 2146527 | C | G |
| 5 | 2825568 | C | A | 5 | 3659894 | C | T | 5 | 3659913 | G | A |
| 5 | 3659989 | C | T | 5 | 3660487 | A | G | 5 | 4225004 | A | G |
| 5 | 4941345 | A | G | 5 | 6272411 | C | A | 5 | 6273004 | G | T |
| 5 | 7447405 | A | G | 5 | 7927332 | C | T | 5 | 7927348 | C | T |
| 5 | 7927356 | C | A | 5 | 7927414 | T | G | 5 | 7927952 | G | C |
| 5 | 8061222 | G | A | 5 | 8061279 | G | A | 5 | 8061284 | C | G |
| 5 | 10151085 | T | C | 5 | 10151123 | A | G | 5 | 10912625 | T | C |
| 5 | 12056802 | A | G | 5 | 12056837 | C | T | 5 | 12056923 | A | C |
| 5 | 12057204 | A | G | 5 | 12058227 | A | G | 5 | 13190185 | T | C |
| 5 | 13847794 | C | T | 5 | 14634747 | A | G | 5 | 14634756 | G | A |
| 5 | 15955155 | C | A | 5 | 16535341 | T | C | 5 | 17025201 | G | T |
| 5 | 17353828 | C | T | 5 | 17354234 | C | T | 5 | 20166781 | T | C |
| 5 | 20167136 | T | G | 5 | 21293117 | T | C | 5 | 21381238 | G | A |
| 5 | 21381264 | T | G | 5 | 21497311 | C | T | 5 | 21497344 | T | G |
| 5 | 22227645 | A | T | 5 | 23242291 | A | G | 5 | 23242685 | T | C |
| 5 | 23304111 | C | T | 5 | 23304211 | G | T | 5 | 23304235 | T | C |
| 5 | 23304261 | G | A | 5 | 23304368 | A | C | 5 | 23305356 | G | A |
| 5 | 23305374 | G | A | 5 | 23305484 | T | C | 5 | 23527239 | C | G |
| 5 | 23527919 | A | G | 5 | 23528007 | G | A | 5 | 24844076 | G | A |
| 5 | 25007293 | A | G | 5 | 25759642 | G | A | 5 | 27795250 | T | A |
| 5 | 28213790 | A | G | 5 | 29312232 | G | T | 5 | 29312728 | G | A |
| 5 | 29312755 | A | C | 5 | 29438592 | G | A | 5 | 29438662 | A | C |
| 5 | 29438664 | T | C | 5 | 30972823 | C | T | 5 | 31383964 | A | G |
| 5 | 31384055 | G | A | 5 | 31384509 | C | A | 5 | 31840606 | T | C |
| 5 | 32828846 | T | C | 5 | 33466421 | A | G | 5 | 33633582 | G | A |
| 5 | 34206776 | C | T | 5 | 35867204 | A | T | 5 | 36170237 | T | C |
| 5 | 36171420 | G | A | 5 | 36172105 | C | T | 5 | 36172115 | G | A |
| 5 | 36176874 | G | A | 5 | 36181617 | A | G | 5 | 36181627 | A | G |
| 5 | 36184284 | C | G | 5 | 38943260 | G | T | 5 | 38996638 | A | G |
| 5 | 39021369 | G | A | 5 | 39074696 | C | T | 5 | 40320944 | G | C |
| 5 | 40320945 | G | T | 5 | 43053497 | A | T | 5 | 43767400 | A | G |
| 5 | 43803258 | T | C | 5 | 44392401 | G | A | 5 | 44585369 | G | C |
| 5 | 44585815 | C | G | 5 | 50058043 | T | C | 5 | 51105168 | G | T |
| 5 | 53835771 | G | A | 5 | 54154481 | A | T | 5 | 55781646 | T | C |
| 5 | 55781772 | T | C | 5 | 55782130 | T | C | 5 | 56152795 | G | A |
| 5 | 56161975 | C | T | 5 | 56171391 | G | T | 5 | 56175226 | A | G |
| 5 | 56176306 | T | C | 5 | 56183637 | G | C | 5 | 56184444 | T | C |
| 5 | 56803613 | G | A | 5 | 58478599 | C | A | 5 | 58478692 | A | G |
| 5 | 58711707 | G | A | 5 | 60860146 | C | T | 5 | 62085252 | G | C |
| 5 | 64957686 | A | G | 5 | 64957718 | T | C | 5 | 65349061 | T | A |
| 5 | 65867918 | C | T | 5 | 66219033 | T | C | 5 | 66219117 | T | G |
| 5 | 67569602 | A | G | 5 | 67577051 | G | A | 5 | 67584357 | A | G |
| 5 | 67589914 | G | A | 5 | 67590140 | C | A | 5 | 67593016 | T | C |
| 5 | 68417945 | G | T | 5 | 68927608 | G | A | 5 | 68927676 | T | C |
| 5 | 69674512 | G | A | 5 | 69927417 | G | T | 5 | 71198806 | C | T |
| 5 | 71542000 | A | G | 5 | 72125176 | A | G | 5 | 73231521 | C | A |
| 5 | 73524903 | A | G | 5 | 73524939 | G | A | 5 | 73525404 | G | A |
| 5 | 75041362 | T | G | 5 | 76986580 | C | G | 5 | 77991568 | C | T |
| 5 | 80655266 | T | C | 5 | 82205681 | A | G | 5 | 82206152 | G | A |
| 5 | 83378803 | T | C | 5 | 83378919 | G | A | 5 | 84637681 | T | C |
| 5 | 85577284 | C | T | 5 | 85586579 | T | C | 5 | 85586584 | C | T |
| 5 | 86865094 | G | A | 5 | 89418018 | A | T | 5 | 93798480 | T | C |
| 5 | 93798817 | A | C | 5 | 94572738 | C | A | 5 | 95477855 | A | G |
| 5 | 97441644 | A | T | 5 | 97441664 | A | G | 5 | 98851779 | C | T |
| 5 | 98861253 | A | G | 5 | 98913692 | C | A | 5 | 98913961 | T | C |
| 5 | 99069979 | G | A | 5 | 99724591 | C | A | 5 | 99733040 | G | A |
| 5 | 99733085 | A | G | 5 | 100695270 | T | C | 5 | 102089175 | G | C |
| 5 | 107267081 | C | T | 5 | 107962193 | C | T | 5 | 108535800 | T | C |
| 5 | 110491533 | C | T | 5 | 111184331 | T | C | 5 | 111184434 | A | G |
| 5 | 111699038 | T | C | 5 | 111699088 | A | G | 5 | 111699115 | C | A |
| 5 | 111699641 | C | T | 5 | 112043170 | A | C | 5 | 112101793 | C | A |
| 5 | 112151551 | G | T | 5 | 112164862 | G | A | 5 | 112171097 | A | G |
| 5 | 112821283 | A | C | 5 | 113797914 | G | A | 5 | 114685700 | T | C |
| 5 | 115106332 | C | G | 5 | 115394388 | A | C | 5 | 115394396 | C | G |
| 5 | 115394857 | A | T | 5 | 115394859 | A | G | 5 | 115394869 | T | C |
| 5 | 115394881 | C | A | 5 | 115394908 | C | A | 5 | 116051351 | C | T |
| 5 | 117955311 | T | C | 5 | 118952055 | C | T | 5 | 122456440 | T | A |
| 5 | 122609817 | T | C | 5 | 124243030 | C | T | 5 | 124243078 | C | T |
| 5 | 125874804 | G | T | 5 | 127638759 | A | G | 5 | 128428722 | A | G |
| 5 | 128428723 | T | G | 5 | 128428851 | C | T | 5 | 128500520 | G | A |
| 5 | 131911847 | G | C | 5 | 131925174 | A | C | 5 | 131925187 | T | C |
| 5 | 131944129 | G | A | 5 | 131951996 | T | C | 5 | 131953623 | G | A |
| 5 | 131973177 | A | G | 5 | 133784798 | C | T | 5 | 134356380 | G | A |
| 5 | 134583301 | A | G | 5 | 134583302 | C | T | 5 | 134583937 | T | C |
| 5 | 135336981 | A | G | 5 | 135979386 | C | T | 5 | 137136265 | A | G |
| 5 | 137787827 | T | A | 5 | 140476395 | T | C | 5 | 140476396 | G | T |
| 5 | 140476653 | G | A | 5 | 140476654 | C | A | 5 | 140482102 | A | G |
| 5 | 140482483 | G | A | 5 | 140503443 | C | G | 5 | 140531746 | C | G |
| 5 | 140554189 | G | C | 5 | 140554808 | T | A | 5 | 140559532 | G | C |
| 5 | 140564048 | G | C | 5 | 140564088 | C | T | 5 | 140568796 | A | G |
| 5 | 140574045 | C | G | 5 | 140581220 | C | A | 5 | 140590766 | A | G |
| 5 | 140595625 | G | A | 5 | 140605030 | T | G | 5 | 141392706 | T | C |
| 5 | 143146391 | C | T | 5 | 144135790 | T | G | 5 | 144135871 | T | C |
| 5 | 144604975 | C | T | 5 | 145296020 | C | T | 5 | 146320823 | G | T |
| 5 | 146562965 | A | C | 5 | 146562965 | A | G | 5 | 148838021 | G | A |
| 5 | 149433322 | G | T | 5 | 149440644 | C | T | 5 | 149440645 | C | T |
| 5 | 149440723 | G | A | 5 | 149440739 | A | G | 5 | 149448060 | G | A |
| 5 | 149457493 | A | G | 5 | 149458110 | C | T | 5 | 149458128 | T | C |
| 5 | 149495770 | G | A | 5 | 149497672 | C | T | 5 | 149498759 | A | T |
| 5 | 149498821 | C | T | 5 | 149501803 | C | T | 5 | 149512042 | G | A |
| 5 | 149512781 | G | A | 5 | 149780952 | A | G | 5 | 150277909 | A | G |
| 5 | 151474068 | A | T | 5 | 151474626 | A | G | 5 | 151900065 | C | T |
| 5 | 151900374 | G | T | 5 | 152861322 | G | A | 5 | 154703595 | G | C |
| 5 | 155580762 | G | A | 5 | 155580831 | G | T | 5 | 156449748 | G | A |
| 5 | 158052650 | G | A | 5 | 159622823 | C | T | 5 | 160357140 | C | T |
| 5 | 161154595 | C | G | 5 | 163479999 | G | T | 5 | 165061117 | T | G |
| 5 | 167958419 | C | G | 5 | 168395021 | T | C | 5 | 169872183 | A | G |
| 5 | 170215778 | C | T | 5 | 170215820 | T | A | 5 | 170817415 | A | G |
| 5 | 170818969 | A | G | 5 | 170828170 | C | T | 5 | 170832637 | C | T |
| 5 | 172750436 | G | A | 5 | 174177551 | T | C | 5 | 174177552 | C | T |
| 5 | 174177562 | G | A | 5 | 174969438 | G | C | 5 | 174969467 | T | C |
| 5 | 174969879 | G | A | 5 | 174969908 | G | A | 5 | 175007464 | T | C |
| 5 | 175007481 | A | G | 5 | 175007493 | T | C | 5 | 175774865 | G | T |
| 5 | 176287029 | A | G | 5 | 176710547 | C | G | 5 | 177163580 | A | G |
| 5 | 177380181 | A | T | 5 | 177380281 | C | T | 5 | 177380537 | C | T |
| 5 | 177380562 | A | C | 5 | 177380624 | T | G | 5 | 178012176 | A | G |
| 5 | 178012696 | T | C | 5 | 178012728 | A | C | 5 | 179430530 | C | T |
| 5 | 180029879 | G | A | 5 | 180036239 | G | A | 5 | 180036318 | A | G |
| 5 | 180037317 | C | T | 5 | 180037318 | G | A | 5 | 180039187 | A | G |
| 5 | 180041274 | T | C | 5 | 180043093 | T | C | 5 | 180045453 | C | T |
| 5 | 180046209 | C | T | 5 | 180049152 | C | T | 5 | 180049181 | C | T |
| 5 | 180049525 | T | C | 5 | 180049907 | G | A | 5 | 180049928 | A | C |
| 5 | 180050066 | T | C | 5 | 180051296 | A | G | 5 | 180051305 | A | G |
| 5 | 180058008 | C | A | 5 | 180058963 | G | A | 5 | 180076129 | G | A |
| 5 | 180076130 | C | A | 5 | 180076240 | A | G | 5 | 180698413 | T | C |
| 5 | 180698428 | G | C | 6 | 675548 | A | C | 6 | 675553 | C | T |
| 6 | 675712 | C | T | 6 | 676129 | A | C | 6 | 676727 | T | G |
| 6 | 676780 | G | T | 6 | 676918 | A | G | 6 | 677120 | T | C |
| 6 | 2777264 | G | A | 6 | 2777334 | A | G | 6 | 3154871 | A | C |
| 6 | 3177590 | C | A | 6 | 3177601 | C | T | 6 | 3177658 | A | T |
| 6 | 3402411 | G | A | 6 | 3402435 | C | G | 6 | 3402520 | T | C |
| 6 | 4255068 | A | G | 6 | 4716150 | C | T | 6 | 4716185 | T | C |
| 6 | 5148454 | G | A | 6 | 6970420 | T | C | 6 | 6970890 | A | G |
| 6 | 7387462 | T | C | 6 | 7516700 | T | A | 6 | 7516833 | G | T |
| 6 | 7517131 | A | T | 6 | 7517239 | G | A | 6 | 7618813 | G | A |
| 6 | 10575131 | T | C | 6 | 11214092 | A | T | 6 | 11969231 | C | T |
| 6 | 11969262 | C | T | 6 | 15497649 | T | C | 6 | 17271896 | C | T |
| 6 | 18139450 | T | A | 6 | 18140175 | T | C | 6 | 18147780 | G | A |
| 6 | 18528399 | C | T | 6 | 21808894 | A | G | 6 | 22117590 | G | C |
| 6 | 24077986 | A | C | 6 | 24968393 | G | A | 6 | 24976616 | C | T |
| 6 | 24976637 | G | A | 6 | 24976739 | G | C | 6 | 25262087 | A | G |
| 6 | 26126524 | A | G | 6 | 26127090 | A | G | 6 | 26202337 | G | A |
| 6 | 26202605 | G | A | 6 | 26202768 | C | T | 6 | 26671135 | T | C |
| 6 | 26702489 | G | A | 6 | 26798264 | G | A | 6 | 26798312 | G | T |
| 6 | 26851448 | G | A | 6 | 26864205 | T | C | 6 | 26864281 | T | C |
| 6 | 26864327 | G | A | 6 | 27689939 | T | C | 6 | 27690453 | G | T |
| 6 | 28447915 | T | C | 6 | 29691857 | C | G | 6 | 29692622 | C | A |
| 6 | 29692622 | C | G | 6 | 29693499 | A | G | 6 | 29759811 | C | G |
| 6 | 29759823 | T | C | 6 | 29759824 | T | C | 6 | 29759876 | G | A |
| 6 | 29759885 | G | A | 6 | 29759923 | T | C | 6 | 29759996 | T | C |
| 6 | 29760029 | G | C | 6 | 29760111 | G | C | 6 | 29760149 | G | T |
| 6 | 29760188 | G | A | 6 | 29760510 | G | A | 6 | 29760523 | C | T |
| 6 | 29760536 | G | A | 6 | 29760590 | G | A | 6 | 29760605 | G | T |
| 6 | 29795636 | G | A | 6 | 29795657 | G | A | 6 | 29796115 | A | C |
| 6 | 29796152 | G | C | 6 | 29796257 | C | T | 6 | 29796306 | G | A |
| 6 | 29796327 | C | T | 6 | 29796749 | C | A | 6 | 29796838 | A | G |
| 6 | 29797173 | A | G | 6 | 29797553 | G | C | 6 | 29797639 | C | T |
| 6 | 29855320 | T | G | 6 | 29855386 | C | T | 6 | 29855392 | T | C |
| 6 | 29855392 | T | G | 6 | 29855401 | C | G | 6 | 29855409 | A | G |
| 6 | 29855677 | G | A | 6 | 29855682 | T | G | 6 | 29855706 | C | G |
| 6 | 29855733 | C | T | 6 | 29855849 | A | C | 6 | 29855856 | T | C |
| 6 | 29855858 | C | A | 6 | 29855901 | A | G | 6 | 29855902 | G | A |
| 6 | 29855940 | G | A | 6 | 29855945 | A | G | 6 | 29856092 | A | T |
| 6 | 29856109 | C | T | 6 | 29856199 | A | G | 6 | 29856204 | G | T |
| 6 | 29856655 | G | C | 6 | 29856686 | C | G | 6 | 29856693 | C | T |
| 6 | 29856718 | G | C | 6 | 29856965 | G | C | 6 | 29857013 | A | G |
| 6 | 29857021 | C | A | 6 | 29857753 | T | C | 6 | 29858218 | A | G |
| 6 | 29858256 | T | G | 6 | 29858294 | A | G | 6 | 29858475 | C | A |
| 6 | 29858476 | T | C | 6 | 29858477 | G | A | 6 | 29858517 | A | G |
| 6 | 29858660 | A | C | 6 | 29894128 | C | T | 6 | 29894139 | G | A |
| 6 | 29894205 | T | C | 6 | 29894211 | C | T | 6 | 29894226 | G | T |
| 6 | 29894243 | C | T | 6 | 29894250 | T | A | 6 | 29894269 | T | C |
| 6 | 29894299 | C | A | 6 | 29894332 | T | A | 6 | 29894334 | G | A |
| 6 | 29894351 | G | C | 6 | 29894389 | A | C | 6 | 29894392 | G | T |
| 6 | 29894406 | C | A | 6 | 29894407 | G | A | 6 | 29894410 | C | T |
| 6 | 29894439 | A | T | 6 | 29894456 | T | G | 6 | 29894523 | G | A |
| 6 | 29894567 | G | A | 6 | 29894580 | T | G | 6 | 29894603 | A | G |
| 6 | 29894756 | A | G | 6 | 29894763 | T | C | 6 | 29894798 | T | C |
| 6 | 29894851 | C | A | 6 | 29894853 | C | T | 6 | 29894874 | C | A |
| 6 | 29894966 | C | T | 6 | 29894991 | T | C | 6 | 29895002 | G | C |
| 6 | 29895005 | C | T | 6 | 29895039 | A | C | 6 | 29895215 | C | T |
| 6 | 29895250 | C | T | 6 | 29895334 | T | C | 6 | 29895399 | G | C |
| 6 | 29895472 | T | C | 6 | 29895862 | G | A | 6 | 29895888 | G | C |
| 6 | 29895913 | G | A | 6 | 29895929 | C | G | 6 | 29895944 | G | A |
| 6 | 29896001 | C | A | 6 | 29896054 | C | A | 6 | 29896055 | C | T |
| 6 | 29896084 | A | G | 6 | 29896105 | G | A | 6 | 29896114 | G | A |
| 6 | 29896120 | G | T | 6 | 29896125 | G | A | 6 | 29896128 | G | T |
| 6 | 29896257 | C | T | 6 | 29896268 | T | C | 6 | 29896275 | G | A |
| 6 | 29896291 | T | C | 6 | 29896484 | G | C | 6 | 29896523 | A | C |
| 6 | 29896925 | C | G | 6 | 29896967 | C | T | 6 | 29897017 | C | A |
| 6 | 29897030 | G | A | 6 | 29897061 | C | T | 6 | 29897259 | T | A |
| 6 | 29897301 | C | T | 6 | 29897322 | G | T | 6 | 29897350 | C | T |
| 6 | 29897379 | G | C | 6 | 29897408 | T | A | 6 | 29897412 | G | C |
| 6 | 29897416 | A | G | 6 | 29897417 | G | T | 6 | 29897422 | A | G |
| 6 | 29901640 | C | A | 6 | 29901783 | A | T | 6 | 29901806 | C | T |
| 6 | 29901865 | G | C | 6 | 29901898 | C | G | 6 | 29901987 | C | G |
| 6 | 29902005 | G | C | 6 | 29902016 | T | A | 6 | 29902040 | A | G |
| 6 | 29902050 | C | T | 6 | 29902051 | A | G | 6 | 29902059 | A | G |
| 6 | 29902063 | C | T | 6 | 29902064 | T | G | 6 | 29902074 | A | G |
| 6 | 29902129 | T | G | 6 | 29902130 | G | A | 6 | 29902168 | C | T |
| 6 | 29902198 | A | G | 6 | 29902240 | A | C | 6 | 29902293 | C | T |
| 6 | 29902294 | G | T | 6 | 29902327 | G | T | 6 | 29902332 | C | G |
| 6 | 29902333 | A | G | 6 | 29902334 | C | T | 6 | 29910057 | C | A |
| 6 | 29910176 | C | T | 6 | 29910189 | A | T | 6 | 29911571 | G | C |
| 6 | 29911604 | C | A | 6 | 29911684 | C | T | 6 | 29911685 | C | T |
| 6 | 29911727 | T | C | 6 | 29911790 | T | C | 6 | 29911803 | G | A |
| 6 | 29912614 | C | A | 6 | 29912649 | G | C | 6 | 29912656 | C | T |
| 6 | 29912657 | C | T | 6 | 29912668 | T | G | 6 | 29912669 | A | G |
| 6 | 29912680 | A | G | 6 | 29912687 | C | T | 6 | 29912713 | C | T |
| 6 | 29913495 | G | A | 6 | 29913518 | C | A | 6 | 29913526 | A | G |
| 6 | 29925127 | G | A | 6 | 29925134 | G | A | 6 | 29925438 | C | T |
| 6 | 29974346 | G | C | 6 | 29974500 | A | G | 6 | 29975047 | G | T |
| 6 | 29975453 | C | A | 6 | 29975840 | G | A | 6 | 29975950 | C | T |
| 6 | 29975994 | C | A | 6 | 29976024 | C | G | 6 | 29976499 | C | G |
| 6 | 29976741 | A | G | 6 | 29977145 | G | C | 6 | 30227397 | C | T |
| 6 | 30227583 | C | G | 6 | 30228652 | C | A | 6 | 30228721 | G | A |
| 6 | 30228879 | C | T | 6 | 30228881 | T | C | 6 | 30228938 | T | C |
| 6 | 30229306 | A | G | 6 | 30229457 | C | T | 6 | 30229463 | G | A |
| 6 | 30229504 | G | A | 6 | 30229583 | G | C | 6 | 30434900 | T | C |
| 6 | 30457732 | T | C | 6 | 30692230 | G | T | 6 | 30692231 | C | T |
| 6 | 30799399 | T | A | 6 | 31197074 | C | T | 6 | 31197514 | C | G |
| 6 | 31197528 | A | G | 6 | 31197589 | G | A | 6 | 31237858 | T | C |
| 6 | 31237862 | T | C | 6 | 31237876 | A | G | 6 | 31237897 | C | T |
| 6 | 31237917 | C | T | 6 | 31237918 | G | A | 6 | 31237950 | T | C |
| 6 | 31237955 | T | G | 6 | 31237960 | G | C | 6 | 31237962 | T | C |
| 6 | 31237963 | G | A | 6 | 31237965 | G | C | 6 | 31237971 | C | T |
| 6 | 31237975 | T | C | 6 | 31237987 | C | T | 6 | 31237991 | G | T |
| 6 | 31238009 | T | C | 6 | 31238010 | T | G | 6 | 31238027 | C | A |
| 6 | 31238029 | T | C | 6 | 31238053 | G | C | 6 | 31238068 | C | T |
| 6 | 31238126 | G | A | 6 | 31238135 | G | A | 6 | 31238138 | C | T |
| 6 | 31238147 | C | G | 6 | 31238155 | G | A | 6 | 31238230 | G | C |
| 6 | 31238234 | G | A | 6 | 31238259 | G | T | 6 | 31238267 | C | T |
| 6 | 31238297 | C | T | 6 | 31238308 | G | C | 6 | 31238318 | G | A |
| 6 | 31238322 | C | T | 6 | 31238345 | T | C | 6 | 31238372 | C | G |
| 6 | 31238613 | A | G | 6 | 31238632 | C | T | 6 | 31238661 | G | A |
| 6 | 31238662 | A | G | 6 | 31238728 | G | C | 6 | 31239802 | C | G |
| 6 | 31239821 | G | T | 6 | 31239827 | C | T | 6 | 31239855 | C | G |
| 6 | 31239868 | G | C | 6 | 31239869 | T | C | 6 | 31239896 | T | C |
| 6 | 31239937 | C | T | 6 | 31323012 | G | A | 6 | 31323020 | T | C |
| 6 | 31323065 | G | C | 6 | 31323567 | G | A | 6 | 31323677 | C | T |
| 6 | 31323707 | G | A | 6 | 31323721 | G | C | 6 | 31323725 | T | C |
| 6 | 31323745 | C | A | 6 | 31323766 | T | C | 6 | 31323838 | C | G |
| 6 | 31324003 | T | A | 6 | 31324003 | T | G | 6 | 31324004 | C | G |
| 6 | 31324176 | C | G | 6 | 31324184 | C | G | 6 | 31324194 | G | A |
| 6 | 31324200 | G | C | 6 | 31324201 | C | G | 6 | 31324206 | G | A |
| 6 | 31324208 | G | T | 6 | 31324210 | G | A | 6 | 31324227 | C | A |
| 6 | 31324229 | G | C | 6 | 31324489 | C | A | 6 | 31324489 | C | G |
| 6 | 31324491 | C | A | 6 | 31324499 | C | G | 6 | 31324523 | T | C |
| 6 | 31324530 | G | C | 6 | 31324531 | C | G | 6 | 31324531 | C | T |
| 6 | 31324536 | T | A | 6 | 31324536 | T | C | 6 | 31324536 | T | G |
| 6 | 31324538 | G | C | 6 | 31324539 | A | T | 6 | 31324542 | T | C |
| 6 | 31324547 | G | C | 6 | 31324829 | G | C | 6 | 31324830 | G | A |
| 6 | 31324847 | C | G | 6 | 31324864 | G | A | 6 | 31324887 | G | C |
| 6 | 31324888 | G | T | 6 | 31324891 | C | T | 6 | 31324892 | G | C |
| 6 | 31324895 | G | C | 6 | 31324911 | C | G | 6 | 31324921 | C | T |
| 6 | 31324925 | A | G | 6 | 31324931 | A | C | 6 | 31324941 | C | T |
| 6 | 31324953 | C | T | 6 | 31324955 | C | T | 6 | 31324996 | G | A |
| 6 | 31324996 | G | C | 6 | 31325004 | G | A | 6 | 31333610 | C | T |
| 6 | 31333731 | T | C | 6 | 31333751 | G | A | 6 | 31333820 | A | G |
| 6 | 31333874 | G | A | 6 | 31333911 | C | T | 6 | 31333939 | A | G |
| 6 | 31333963 | T | C | 6 | 31334080 | T | A | 6 | 31334120 | A | G |
| 6 | 31487954 | G | C | 6 | 31488038 | C | T | 6 | 31488145 | A | G |
| 6 | 32455520 | G | C | 6 | 32455544 | A | G | 6 | 32455560 | G | C |
| 6 | 32455565 | T | G | 6 | 32455566 | T | A | 6 | 32455569 | C | T |
| 6 | 32455577 | A | T | 6 | 32455580 | T | G | 6 | 32455585 | T | G |
| 6 | 32455586 | G | A | 6 | 32455588 | T | C | 6 | 32455629 | T | C |
| 6 | 32455644 | T | A | 6 | 32455645 | T | C | 6 | 32455651 | A | G |
| 6 | 32455662 | G | A | 6 | 32455668 | A | G | 6 | 32455672 | C | T |
| 6 | 32455680 | G | A | 6 | 32455692 | C | A | 6 | 32455718 | T | G |
| 6 | 32455720 | C | G | 6 | 32455725 | A | G | 6 | 32455725 | A | T |
| 6 | 32455726 | C | G | 6 | 32455737 | A | G | 6 | 32455742 | T | A |
| 6 | 32455743 | T | A | 6 | 32455743 | T | C | 6 | 32455748 | C | T |
| 6 | 32455750 | C | T | 6 | 32455756 | T | A | 6 | 32455757 | A | C |
| 6 | 32455757 | A | G | 6 | 32455777 | C | T | 6 | 32455795 | T | G |
| 6 | 32455801 | C | T | 6 | 32455810 | G | T | 6 | 32455812 | C | A |
| 6 | 32455827 | A | G | 6 | 32455828 | T | C | 6 | 32455838 | G | A |
| 6 | 32455839 | T | C | 6 | 32455843 | A | G | 6 | 32455847 | A | G |
| 6 | 32455859 | C | G | 6 | 32455877 | A | T | 6 | 32455887 | A | G |
| 6 | 32455901 | T | C | 6 | 32455906 | A | G | 6 | 32455909 | T | A |
| 6 | 32455912 | G | A | 6 | 32455917 | C | A | 6 | 32455917 | C | G |
| 6 | 32455928 | T | C | 6 | 32455943 | C | T | 6 | 32455952 | T | C |
| 6 | 32455965 | G | A | 6 | 32456028 | G | A | 6 | 32796346 | T | C |
| 6 | 32796480 | T | C | 6 | 32798789 | C | T | 6 | 32798795 | C | A |
| 6 | 32805049 | T | C | 6 | 32817130 | A | G | 6 | 32819642 | C | T |
| 6 | 32821859 | C | A | 6 | 33290402 | G | A | 6 | 33290825 | A | G |
| 6 | 33290872 | G | A | 6 | 34711946 | C | T | 6 | 34712232 | C | T |
| 6 | 35389999 | G | T | 6 | 35420923 | C | T | 6 | 35960390 | C | T |
| 6 | 37001592 | G | A | 6 | 37002182 | G | A | 6 | 37709919 | A | T |
| 6 | 38960253 | A | G | 6 | 38960677 | C | T | 6 | 38960750 | C | T |
| 6 | 40419999 | C | T | 6 | 41296173 | C | T | 6 | 41296679 | C | T |
| 6 | 41635569 | G | T | 6 | 42407020 | G | A | 6 | 43739327 | C | T |
| 6 | 43742419 | G | C | 6 | 43746949 | T | C | 6 | 44228162 | G | A |
| 6 | 44678620 | A | C | 6 | 45969983 | T | G | 6 | 46856100 | G | A |
| 6 | 46856581 | T | G | 6 | 47130819 | T | C | 6 | 47130910 | G | A |
| 6 | 47130988 | A | G | 6 | 47713939 | T | A | 6 | 47714073 | G | A |
| 6 | 48151808 | G | A | 6 | 48928482 | A | G | 6 | 48928544 | T | C |
| 6 | 49327632 | T | C | 6 | 49328214 | A | G | 6 | 51492164 | T | A |
| 6 | 51514205 | T | C | 6 | 51611344 | A | G | 6 | 51619921 | T | C |
| 6 | 51619972 | A | G | 6 | 51637240 | G | C | 6 | 51640787 | A | G |
| 6 | 51655796 | T | C | 6 | 51656388 | A | G | 6 | 51696056 | A | G |
| 6 | 51696074 | G | A | 6 | 51701484 | G | A | 6 | 51713012 | G | C |
| 6 | 51735613 | T | C | 6 | 51750961 | G | A | 6 | 51752241 | C | T |
| 6 | 51889099 | A | T | 6 | 51897565 | C | T | 6 | 51909525 | T | C |
| 6 | 51919253 | G | A | 6 | 51927666 | T | C | 6 | 51927683 | T | C |
| 6 | 51929476 | T | G | 6 | 51934976 | C | T | 6 | 51934978 | A | C |
| 6 | 51936717 | C | T | 6 | 51936723 | G | A | 6 | 51940816 | T | C |
| 6 | 51949862 | A | G | 6 | 52529666 | A | C | 6 | 52529714 | C | G |
| 6 | 52530205 | C | T | 6 | 53047564 | C | T | 6 | 54610711 | C | T |
| 6 | 54610742 | A | C | 6 | 54610783 | C | T | 6 | 54611146 | A | G |
| 6 | 56291176 | G | A | 6 | 58199238 | T | C | 6 | 58250570 | A | T |
| 6 | 58263871 | T | C | 6 | 58269738 | G | A | 6 | 58270343 | C | G |
| 6 | 58403634 | T | C | 6 | 58404193 | C | G | 6 | 58522197 | C | T |
| 6 | 61993928 | G | A | 6 | 62864433 | A | G | 6 | 62864516 | G | A |
| 6 | 63083740 | T | G | 6 | 63083748 | G | T | 6 | 63083774 | A | G |
| 6 | 65331854 | T | C | 6 | 65589020 | G | C | 6 | 66446642 | A | G |
| 6 | 67044197 | G | T | 6 | 67044321 | C | T | 6 | 67044663 | T | A |
| 6 | 67804122 | G | T | 6 | 67804186 | T | C | 6 | 67804194 | A | C |
| 6 | 67804202 | G | A | 6 | 71721412 | G | A | 6 | 71721445 | T | C |
| 6 | 72563207 | C | T | 6 | 72563211 | G | T | 6 | 72563750 | C | T |
| 6 | 73888146 | C | T | 6 | 75056542 | G | A | 6 | 75877044 | A | C |
| 6 | 76051990 | A | G | 6 | 77478497 | C | G | 6 | 78345710 | C | T |
| 6 | 79426986 | G | A | 6 | 79427089 | A | G | 6 | 81484692 | G | C |
| 6 | 81669149 | G | C | 6 | 82074113 | T | C | 6 | 83480639 | C | T |
| 6 | 83480720 | T | C | 6 | 83545840 | C | A | 6 | 83546246 | G | A |
| 6 | 85070849 | C | T | 6 | 85071341 | A | G | 6 | 85140178 | A | C |
| 6 | 85140313 | C | A | 6 | 85140399 | G | A | 6 | 88608057 | A | G |
| 6 | 90012435 | G | A | 6 | 91210640 | A | G | 6 | 91682066 | C | T |
| 6 | 92363395 | A | C | 6 | 92838614 | C | T | 6 | 93100978 | G | A |
| 6 | 93100980 | T | C | 6 | 93901330 | A | G | 6 | 94555470 | A | G |
| 6 | 96348615 | T | G | 6 | 97077504 | C | A | 6 | 101332215 | T | C |
| 6 | 101849962 | C | A | 6 | 105089423 | A | G | 6 | 108028709 | T | C |
| 6 | 108028752 | T | A | 6 | 108028757 | T | G | 6 | 108774766 | C | G |
| 6 | 109051552 | C | T | 6 | 109051652 | T | C | 6 | 109507842 | A | T |
| 6 | 110252356 | G | C | 6 | 110252919 | C | A | 6 | 110252987 | A | G |
| 6 | 110911774 | C | T | 6 | 111871080 | T | C | 6 | 112375307 | A | G |
| 6 | 112375889 | A | G | 6 | 112524506 | T | C | 6 | 112677375 | C | A |
| 6 | 113203779 | G | A | 6 | 113940257 | G | T | 6 | 114262015 | A | C |
| 6 | 114265754 | A | G | 6 | 114405657 | G | C | 6 | 114405704 | A | G |
| 6 | 115322919 | G | A | 6 | 115398931 | G | C | 6 | 116787050 | C | T |
| 6 | 116787688 | C | A | 6 | 117609387 | T | C | 6 | 117622521 | T | A |
| 6 | 117629650 | A | T | 6 | 117629663 | T | C | 6 | 117631666 | T | C |
| 6 | 117639104 | T | C | 6 | 117639153 | T | C | 6 | 117639620 | G | T |
| 6 | 117657400 | A | G | 6 | 117657686 | A | G | 6 | 117662944 | C | T |
| 6 | 117663364 | A | G | 6 | 117665635 | G | C | 6 | 117683505 | G | A |
| 6 | 117699988 | A | G | 6 | 117725168 | G | A | 6 | 117725789 | C | T |
| 6 | 117725885 | C | A | 6 | 117725890 | T | A | 6 | 117731107 | G | C |
| 6 | 118103565 | G | A | 6 | 119463313 | G | A | 6 | 119870312 | A | G |
| 6 | 120173251 | C | T | 6 | 121850570 | G | A | 6 | 121851118 | A | G |
| 6 | 122015474 | A | G | 6 | 122585885 | C | T | 6 | 123823950 | T | C |
| 6 | 124236107 | C | G | 6 | 124236196 | G | A | 6 | 124236507 | C | T |
| 6 | 124569808 | G | A | 6 | 125553174 | C | T | 6 | 127432657 | A | G |
| 6 | 127909778 | T | C | 6 | 127909784 | C | T | 6 | 128323940 | G | A |
| 6 | 128324030 | G | C | 6 | 128990229 | T | C | 6 | 128990286 | G | A |
| 6 | 128990824 | A | G | 6 | 130007597 | C | A | 6 | 130507274 | A | G |
| 6 | 131047028 | A | C | 6 | 132030037 | T | C | 6 | 132283588 | C | T |
| 6 | 132284167 | A | T | 6 | 132726450 | C | T | 6 | 132840195 | G | C |
| 6 | 132840197 | T | A | 6 | 133019682 | A | G | 6 | 133607088 | A | G |
| 6 | 134496112 | G | A | 6 | 134528280 | C | T | 6 | 135375762 | G | A |
| 6 | 135376379 | T | G | 6 | 135376409 | G | T | 6 | 135989483 | C | T |
| 6 | 137288483 | T | G | 6 | 137288522 | T | C | 6 | 137521772 | C | T |
| 6 | 138027115 | C | A | 6 | 138027145 | C | T | 6 | 140101549 | A | G |
| 6 | 140849815 | T | G | 6 | 142025855 | T | C | 6 | 144476023 | T | A |
| 6 | 144601920 | C | A | 6 | 144601921 | G | A | 6 | 144602478 | C | T |
| 6 | 145806936 | C | T | 6 | 145807010 | G | A | 6 | 146350563 | C | A |
| 6 | 147729477 | T | G | 6 | 148516642 | T | G | 6 | 149237845 | A | C |
| 6 | 149237889 | A | G | 6 | 149237931 | G | A | 6 | 149700805 | G | A |
| 6 | 151364186 | A | G | 6 | 151646048 | G | A | 6 | 152129746 | G | T |
| 6 | 152201624 | G | A | 6 | 153065996 | G | A | 6 | 153066078 | T | G |
| 6 | 153523383 | A | G | 6 | 153523425 | G | A | 6 | 154478704 | C | T |
| 6 | 154629327 | C | T | 6 | 156301661 | G | T | 6 | 156301726 | C | T |
| 6 | 156938505 | T | C | 6 | 156938506 | A | G | 6 | 156938539 | T | C |
| 6 | 157150145 | G | A | 6 | 157256310 | C | T | 6 | 158024313 | T | C |
| 6 | 158024403 | C | G | 6 | 158024861 | C | A | 6 | 158024868 | C | T |
| 6 | 159190019 | T | G | 6 | 159947789 | C | T | 6 | 160398330 | C | T |
| 6 | 160547249 | G | T | 6 | 160548433 | A | G | 6 | 161771412 | T | C |
| 6 | 161970231 | T | A | 6 | 161990678 | C | T | 6 | 162206577 | T | G |
| 6 | 162474893 | A | G | 6 | 162475434 | A | C | 6 | 162622550 | C | T |
| 6 | 165404863 | A | C | 6 | 165404917 | A | G | 6 | 165722345 | T | C |
| 6 | 166204195 | T | C | 6 | 166204288 | G | A | 6 | 166695194 | T | C |
| 6 | 167411008 | T | C | 6 | 167411334 | C | T | 6 | 167411337 | G | A |
| 6 | 167411388 | C | G | 6 | 167411401 | C | T | 6 | 167411616 | C | T |
| 6 | 167589680 | C | T | 6 | 167792111 | G | A | 6 | 167792114 | G | A |
| 6 | 167792651 | C | G | 6 | 167792671 | C | G | 6 | 167792694 | G | C |
| 6 | 167792699 | G | A | 6 | 168687802 | C | G | 6 | 168687815 | A | C |
| 6 | 168687819 | C | A | 6 | 168687828 | G | A | 6 | 168735290 | A | G |
| 6 | 168735309 | A | C | 6 | 168735311 | T | A | 6 | 169313243 | A | G |
| 6 | 169795962 | G | T | 6 | 170470548 | T | A | 6 | 170470568 | A | C |
| 6 | 170470778 | C | A | 6 | 170528622 | A | G | 6 | 170529546 | A | G |
| 6 | 170529549 | G | A | 6 | 170529566 | C | T | 6 | 170529587 | T | C |
| 6 | 170529638 | T | C | 6 | 170530453 | T | C | 6 | 170530506 | C | T |
| 6 | 170530518 | A | G | 6 | 170530559 | T | C | 6 | 170530838 | C | T |
| 6 | 170530873 | C | T | 6 | 171026382 | T | C | 6 | 171026394 | T | C |
| 7 | 746314 | A | G | 7 | 746705 | A | G | 7 | 746706 | C | A |
| 7 | 1883274 | T | C | 7 | 2004124 | C | T | 7 | 3155087 | T | C |
| 7 | 3155089 | T | G | 7 | 3155579 | C | T | 7 | 3155651 | C | G |
| 7 | 4320984 | A | G | 7 | 4321571 | A | G | 7 | 5156760 | G | A |
| 7 | 5946901 | G | C | 7 | 6013049 | C | G | 7 | 6018444 | A | G |
| 7 | 6018592 | G | A | 7 | 6022178 | G | C | 7 | 6029748 | C | T |
| 7 | 6035428 | A | C | 7 | 6035507 | C | T | 7 | 6038483 | G | A |
| 7 | 6039129 | T | C | 7 | 6041897 | T | C | 7 | 6042522 | C | T |
| 7 | 6043057 | A | G | 7 | 6043065 | C | T | 7 | 6043112 | C | T |
| 7 | 6045881 | C | T | 7 | 6045934 | T | C | 7 | 6048359 | C | T |
| 7 | 6048804 | G | C | 7 | 6048845 | A | G | 7 | 6048973 | C | A |
| 7 | 6414545 | C | T | 7 | 6431404 | G | T | 7 | 6431420 | C | A |
| 7 | 6431933 | C | G | 7 | 6441258 | G | A | 7 | 6442371 | G | A |
| 7 | 6776537 | G | A | 7 | 6776585 | C | T | 7 | 6777726 | T | C |
| 7 | 6777752 | T | C | 7 | 6777768 | T | G | 7 | 6777780 | T | G |
| 7 | 6777784 | A | G | 7 | 6777820 | T | G | 7 | 6777821 | G | A |
| 7 | 6785956 | A | G | 7 | 6786731 | C | T | 7 | 6790900 | T | C |
| 7 | 6791040 | G | C | 7 | 6919565 | T | C | 7 | 6974986 | G | A |
| 7 | 6975272 | G | T | 7 | 6975316 | G | T | 7 | 6975366 | C | A |
| 7 | 8549008 | G | A | 7 | 8549058 | G | A | 7 | 8549477 | C | T |
| 7 | 10013659 | G | A | 7 | 10897044 | T | C | 7 | 10897594 | C | A |
| 7 | 10897594 | C | G | 7 | 11349162 | C | T | 7 | 11349705 | G | A |
| 7 | 11349733 | A | G | 7 | 11349735 | T | C | 7 | 12122025 | C | T |
| 7 | 12510929 | A | C | 7 | 12511312 | G | A | 7 | 14868954 | C | T |
| 7 | 16297567 | C | T | 7 | 17006944 | G | C | 7 | 17007526 | G | T |
| 7 | 17514653 | T | C | 7 | 18318662 | C | T | 7 | 18624673 | G | A |
| 7 | 18625440 | C | T | 7 | 19184257 | C | G | 7 | 21206385 | A | G |
| 7 | 21764077 | A | G | 7 | 21764081 | G | A | 7 | 22327627 | G | A |
| 7 | 22328109 | C | T | 7 | 22947858 | A | G | 7 | 22948438 | G | C |
| 7 | 24353194 | A | C | 7 | 24353195 | C | T | 7 | 24353267 | C | T |
| 7 | 25233745 | C | G | 7 | 25233789 | G | C | 7 | 26627952 | G | T |
| 7 | 26627954 | G | A | 7 | 29464865 | T | C | 7 | 30177268 | G | A |
| 7 | 30177278 | C | T | 7 | 30855318 | G | A | 7 | 30855926 | G | C |
| 7 | 32133424 | G | A | 7 | 32512110 | A | T | 7 | 33101967 | C | T |
| 7 | 33102514 | G | A | 7 | 33193313 | A | C | 7 | 33193314 | T | C |
| 7 | 34540399 | G | T | 7 | 35083870 | T | C | 7 | 35084552 | C | T |
| 7 | 35084572 | C | T | 7 | 35084601 | T | C | 7 | 36186371 | G | C |
| 7 | 36858622 | G | A | 7 | 36858643 | A | G | 7 | 37415057 | G | A |
| 7 | 37708712 | A | G | 7 | 39807352 | G | A | 7 | 40320747 | G | C |
| 7 | 43874751 | A | G | 7 | 44026116 | T | G | 7 | 44056757 | C | T |
| 7 | 44057222 | C | G | 7 | 45256549 | A | G | 7 | 45256975 | A | G |
| 7 | 45828285 | C | T | 7 | 45847880 | A | G | 7 | 45856902 | T | C |
| 7 | 45856916 | G | A | 7 | 45857319 | G | A | 7 | 45857848 | G | A |
| 7 | 46028450 | C | T | 7 | 46028459 | C | T | 7 | 46643720 | A | C |
| 7 | 50470256 | G | A | 7 | 50745025 | G | A | 7 | 51450917 | G | A |
| 7 | 51450951 | A | G | 7 | 51451225 | T | C | 7 | 51451275 | G | A |
| 7 | 51451291 | C | T | 7 | 51451376 | A | G | 7 | 51451655 | G | A |
| 7 | 51451775 | T | C | 7 | 51451925 | C | T | 7 | 51451928 | T | C |
| 7 | 51451955 | G | T | 7 | 51452002 | A | G | 7 | 51452050 | G | A |
| 7 | 51452088 | T | C | 7 | 51452279 | A | G | 7 | 51824545 | C | T |
| 7 | 51824890 | T | C | 7 | 51824990 | A | G | 7 | 52225367 | T | C |
| 7 | 52758842 | T | C | 7 | 52759429 | A | G | 7 | 53189193 | A | G |
| 7 | 53189634 | G | A | 7 | 53190434 | G | T | 7 | 53190453 | A | G |
| 7 | 53190508 | A | G | 7 | 53190917 | C | T | 7 | 53203065 | G | A |
| 7 | 53718464 | T | C | 7 | 53718485 | G | T | 7 | 53854735 | T | A |
| 7 | 53854969 | T | C | 7 | 54618943 | A | G | 7 | 55210733 | G | C |
| 7 | 55219290 | T | C | 7 | 55219307 | C | G | 7 | 55221447 | C | G |
| 7 | 55225140 | G | C | 7 | 55232726 | G | T | 7 | 55248787 | T | C |
| 7 | 55259948 | C | G | 7 | 55658927 | T | C | 7 | 55659085 | T | C |
| 7 | 55806087 | G | T | 7 | 55806093 | G | T | 7 | 56405132 | T | C |
| 7 | 56430969 | C | G | 7 | 56430977 | G | C | 7 | 56430980 | C | A |
| 7 | 56431109 | A | G | 7 | 56431170 | A | G | 7 | 56431807 | G | A |
| 7 | 56432242 | T | C | 7 | 56635995 | C | A | 7 | 57289345 | C | T |
| 7 | 57603890 | T | G | 7 | 57603918 | G | A | 7 | 57669433 | G | T |
| 7 | 57669986 | A | G | 7 | 57809151 | C | A | 7 | 57832849 | T | C |
| 7 | 57938126 | A | G | 7 | 57938210 | T | C | 7 | 61058809 | T | G |
| 7 | 61059081 | A | G | 7 | 61063424 | G | A | 7 | 61063551 | A | G |
| 7 | 61071095 | A | T | 7 | 61071103 | C | T | 7 | 61071188 | G | T |
| 7 | 61071203 | C | T | 7 | 63206179 | A | G | 7 | 63206611 | A | G |
| 7 | 63206623 | G | T | 7 | 63229281 | G | A | 7 | 63229377 | C | T |
| 7 | 63229524 | T | C | 7 | 63230106 | C | T | 7 | 63230147 | C | T |
| 7 | 63230155 | G | A | 7 | 63602429 | G | A | 7 | 63602520 | A | G |
| 7 | 63602542 | T | C | 7 | 63635236 | A | C | 7 | 63635239 | G | C |
| 7 | 63635241 | A | G | 7 | 63635248 | T | C | 7 | 63635271 | C | G |
| 7 | 63635272 | T | C | 7 | 63635286 | C | T | 7 | 64583576 | C | G |
| 7 | 64583585 | C | T | 7 | 64583587 | C | T | 7 | 64583615 | G | C |
| 7 | 64583628 | C | T | 7 | 64652748 | C | G | 7 | 64652982 | T | G |
| 7 | 64653060 | G | A | 7 | 65037658 | C | G | 7 | 65037683 | T | C |
| 7 | 65059071 | A | G | 7 | 65082869 | G | A | 7 | 65163375 | C | G |
| 7 | 65163587 | T | C | 7 | 65432962 | G | A | 7 | 65552792 | A | T |
| 7 | 65971428 | G | T | 7 | 66355416 | C | T | 7 | 66457975 | T | C |
| 7 | 66458627 | A | G | 7 | 66458631 | C | T | 7 | 66458903 | T | C |
| 7 | 66459513 | C | T | 7 | 66459553 | G | C | 7 | 66760853 | T | C |
| 7 | 66760969 | G | A | 7 | 66760975 | C | T | 7 | 66761773 | T | C |
| 7 | 66762050 | A | T | 7 | 66762277 | C | T | 7 | 66762340 | A | C |
| 7 | 66762461 | T | A | 7 | 67635408 | C | T | 7 | 67635412 | G | A |
| 7 | 69444431 | G | C | 7 | 70047405 | G | A | 7 | 71309437 | C | T |
| 7 | 73809454 | T | C | 7 | 74178770 | C | T | 7 | 74193668 | G | A |
| 7 | 74582543 | T | G | 7 | 76072643 | C | T | 7 | 76072644 | A | G |
| 7 | 76237814 | C | T | 7 | 76237885 | A | C | 7 | 76364904 | C | A |
| 7 | 76621710 | C | T | 7 | 76641042 | T | C | 7 | 76641340 | A | T |
| 7 | 76669334 | G | A | 7 | 78690736 | C | T | 7 | 78690794 | G | A |
| 7 | 79293866 | A | G | 7 | 79293877 | G | T | 7 | 80387473 | G | C |
| 7 | 80387977 | A | G | 7 | 80826781 | T | C | 7 | 81346925 | G | A |
| 7 | 81358712 | G | A | 7 | 81381731 | T | C | 7 | 81858825 | G | T |
| 7 | 82925988 | A | G | 7 | 83407638 | A | C | 7 | 84345772 | C | G |
| 7 | 84707925 | C | T | 7 | 86856261 | G | T | 7 | 87031809 | A | G |
| 7 | 87133947 | C | A | 7 | 87133993 | T | C | 7 | 87149922 | C | T |
| 7 | 87161049 | T | C | 7 | 87174504 | C | T | 7 | 87182882 | T | C |
| 7 | 87190953 | A | G | 7 | 87195106 | C | T | 7 | 87195850 | T | C |
| 7 | 87199264 | C | A | 7 | 87199265 | T | A | 7 | 87604085 | A | T |
| 7 | 88195330 | G | A | 7 | 88454877 | G | C | 7 | 89641818 | T | C |
| 7 | 89641833 | G | A | 7 | 90158020 | C | T | 7 | 90158026 | C | G |
| 7 | 91509551 | T | C | 7 | 92247112 | C | T | 7 | 92300587 | G | A |
| 7 | 92301040 | C | T | 7 | 93194505 | G | A | 7 | 93194506 | C | G |
| 7 | 93194590 | C | A | 7 | 97264891 | C | A | 7 | 97264891 | C | G |
| 7 | 97560579 | G | A | 7 | 97561099 | A | C | 7 | 97561130 | C | A |
| 7 | 98648365 | C | G | 7 | 99314986 | A | G | 7 | 99738322 | G | C |
| 7 | 101977547 | A | G | 7 | 101977597 | G | A | 7 | 102117789 | G | A |
| 7 | 102539438 | A | T | 7 | 102800500 | A | T | 7 | 102800583 | T | C |
| 7 | 102801333 | C | T | 7 | 104309273 | G | A | 7 | 105460274 | C | A |
| 7 | 105626070 | A | G | 7 | 106848955 | G | T | 7 | 107468001 | C | T |
| 7 | 109660709 | T | C | 7 | 111633858 | T | C | 7 | 113250814 | T | G |
| 7 | 113250857 | C | T | 7 | 114352126 | T | C | 7 | 114352682 | T | A |
| 7 | 115334606 | A | C | 7 | 116340582 | A | C | 7 | 116399131 | G | A |
| 7 | 116423161 | G | A | 7 | 116435416 | G | A | 7 | 116894865 | T | A |
| 7 | 116894915 | A | T | 7 | 116894926 | A | G | 7 | 118211698 | G | A |
| 7 | 118211801 | T | C | 7 | 121419326 | C | T | 7 | 121595833 | G | A |
| 7 | 122184921 | C | T | 7 | 123010356 | A | C | 7 | 123881827 | T | G |
| 7 | 123881905 | G | A | 7 | 124465097 | A | T | 7 | 124469587 | G | A |
| 7 | 124480869 | T | C | 7 | 124481403 | T | A | 7 | 124481420 | T | A |
| 7 | 124483222 | T | C | 7 | 124483272 | A | T | 7 | 124492763 | A | G |
| 7 | 124499388 | G | T | 7 | 124499402 | C | G | 7 | 124503189 | A | G |
| 7 | 124503196 | C | T | 7 | 124537476 | A | C | 7 | 125088151 | A | G |
| 7 | 125640459 | C | T | 7 | 125640469 | A | C | 7 | 125640469 | A | T |
| 7 | 125640499 | C | T | 7 | 125640501 | A | C | 7 | 127239815 | C | T |
| 7 | 128147625 | A | G | 7 | 128210320 | C | T | 7 | 128210643 | T | C |
| 7 | 128210789 | G | A | 7 | 128266315 | A | G | 7 | 128266517 | C | T |
| 7 | 128266627 | G | T | 7 | 128297916 | G | A | 7 | 128766463 | G | T |
| 7 | 128766505 | A | C | 7 | 128767969 | A | G | 7 | 128768048 | C | A |
| 7 | 128846716 | A | G | 7 | 130575897 | T | G | 7 | 131628225 | C | T |
| 7 | 132907794 | C | T | 7 | 133345471 | A | T | 7 | 134945970 | T | C |
| 7 | 134945999 | C | T | 7 | 134946000 | T | G | 7 | 134946430 | A | G |
| 7 | 134946432 | G | C | 7 | 135129961 | A | G | 7 | 135519437 | T | G |
| 7 | 135519460 | A | G | 7 | 136023238 | C | T | 7 | 136023288 | C | T |
| 7 | 138503130 | T | C | 7 | 138503690 | C | T | 7 | 139174728 | T | A |
| 7 | 139595823 | G | C | 7 | 140439987 | T | A | 7 | 140492411 | T | C |
| 7 | 141035361 | G | A | 7 | 141814946 | G | A | 7 | 141815374 | T | G |
| 7 | 142457132 | A | C | 7 | 142457674 | G | A | 7 | 142470290 | T | C |
| 7 | 142478761 | C | A | 7 | 142478986 | A | G | 7 | 142480034 | C | A |
| 7 | 142480148 | G | C | 7 | 143720970 | G | C | 7 | 143720987 | G | T |
| 7 | 143964267 | A | G | 7 | 143964486 | T | C | 7 | 144060802 | C | T |
| 7 | 144670310 | G | A | 7 | 146238505 | T | A | 7 | 147194606 | A | G |
| 7 | 147194697 | C | T | 7 | 147720477 | T | A | 7 | 147720992 | T | C |
| 7 | 148034568 | T | A | 7 | 148035121 | A | G | 7 | 148508481 | A | G |
| 7 | 149031681 | A | G | 7 | 149450425 | G | A | 7 | 149450439 | T | C |
| 7 | 149687249 | G | C | 7 | 149687453 | G | A | 7 | 150485864 | G | A |
| 7 | 151407771 | T | G | 7 | 152372816 | C | G | 7 | 152372924 | G | A |
| 7 | 152752607 | T | C | 7 | 152752612 | C | T | 7 | 153404512 | C | A |
| 7 | 154452587 | C | T | 7 | 154452720 | A | G | 7 | 154452734 | A | G |
| 7 | 154453511 | C | T | 7 | 154453688 | G | A | 7 | 154453864 | A | G |
| 7 | 154453902 | T | C | 7 | 154453921 | C | T | 7 | 154453928 | G | A |
| 7 | 154454135 | T | C | 7 | 154454180 | G | T | 7 | 154454264 | A | G |
| 7 | 154454268 | G | T | 7 | 154454356 | A | G | 7 | 154454435 | A | G |
| 7 | 154454522 | T | C | 7 | 154454523 | G | T | 7 | 154454546 | A | G |
| 7 | 154454616 | C | G | 7 | 154454786 | T | C | 7 | 154454939 | A | G |
| 7 | 154454972 | G | A | 7 | 154455363 | T | C | 7 | 154455500 | A | G |
| 7 | 154455674 | C | A | 7 | 154915559 | G | T | 7 | 154915599 | A | G |
| 7 | 154915632 | C | T | 7 | 154915894 | T | C | 7 | 154916158 | C | T |
| 7 | 154916681 | G | C | 7 | 155126014 | C | T | 7 | 155126074 | G | A |
| 7 | 155126346 | A | G | 7 | 155126421 | C | A | 7 | 155126422 | C | T |
| 7 | 155126626 | C | T | 7 | 155126697 | T | C | 7 | 155126714 | A | G |
| 7 | 155126852 | C | T | 7 | 155126903 | C | T | 7 | 155127026 | T | C |
| 7 | 155127061 | A | C | 7 | 155127063 | C | T | 7 | 155127076 | G | C |
| 7 | 155127111 | A | C | 7 | 155127142 | T | C | 7 | 155127988 | A | G |
| 7 | 155127991 | G | C | 7 | 155128004 | G | C | 7 | 155128005 | A | C |
| 7 | 155128006 | T | C | 7 | 155947955 | G | A | 7 | 156781685 | G | A |
| 7 | 157459940 | T | C | 7 | 157459941 | G | A | 7 | 157950927 | T | C |
| 7 | 157950935 | A | T | 7 | 157951445 | A | G | 7 | 157951812 | A | G |
| 7 | 157952182 | G | A | 7 | 158248001 | G | A | 7 | 158249548 | C | T |
| 7 | 159001392 | A | G | 7 | 159001393 | G | A | 7 | 159001396 | G | T |
| 7 | 159001399 | A | T | 8 | 11386 | T | C | 8 | 40370 | A | G |
| 8 | 40565 | C | T | 8 | 143930 | T | C | 8 | 339174 | G | A |
| 8 | 339319 | T | C | 8 | 339338 | G | A | 8 | 339668 | G | A |
| 8 | 339702 | C | T | 8 | 580564 | T | C | 8 | 1292447 | A | C |
| 8 | 1550886 | C | G | 8 | 1550939 | C | A | 8 | 1552096 | A | G |
| 8 | 1552099 | T | G | 8 | 2676199 | C | A | 8 | 3415423 | A | C |
| 8 | 3415633 | G | A | 8 | 3415649 | C | T | 8 | 3415650 | C | G |
| 8 | 3848580 | T | C | 8 | 3848627 | C | T | 8 | 3848630 | G | C |
| 8 | 3849139 | C | G | 8 | 3849147 | T | A | 8 | 4113196 | G | A |
| 8 | 4650270 | C | T | 8 | 5995309 | A | G | 8 | 5995912 | G | A |
| 8 | 6141844 | T | C | 8 | 6141879 | C | T | 8 | 6141887 | C | T |
| 8 | 6946100 | A | G | 8 | 6946189 | A | G | 8 | 6946639 | G | T |
| 8 | 6946666 | C | A | 8 | 6946666 | C | G | 8 | 6948229 | C | T |
| 8 | 7000182 | T | G | 8 | 7000202 | C | T | 8 | 7000210 | T | C |
| 8 | 7000238 | A | G | 8 | 7070133 | T | C | 8 | 7070315 | A | T |
| 8 | 7192428 | G | A | 8 | 7913763 | C | G | 8 | 7913833 | A | G |
| 8 | 7913965 | A | T | 8 | 7913966 | A | G | 8 | 8088230 | T | C |
| 8 | 8094555 | T | C | 8 | 8737464 | T | G | 8 | 9165380 | G | T |
| 8 | 11189344 | C | G | 8 | 11607330 | C | T | 8 | 11607352 | C | T |
| 8 | 11607372 | T | C | 8 | 11607377 | A | G | 8 | 11607396 | G | A |
| 8 | 11612315 | C | T | 8 | 11612381 | T | C | 8 | 11612929 | A | C |
| 8 | 11614175 | G | T | 8 | 11614225 | G | T | 8 | 11993433 | G | A |
| 8 | 11993450 | G | A | 8 | 11993553 | C | T | 8 | 11993854 | C | T |
| 8 | 12285564 | G | A | 8 | 12285923 | C | T | 8 | 12466022 | C | T |
| 8 | 12507122 | G | C | 8 | 12517750 | A | C | 8 | 12517757 | G | A |
| 8 | 12517789 | A | G | 8 | 12517792 | A | C | 8 | 12517824 | G | T |
| 8 | 12518207 | T | A | 8 | 12518209 | A | G | 8 | 12525951 | T | A |
| 8 | 12526441 | C | G | 8 | 12553656 | A | G | 8 | 12553833 | A | G |
| 8 | 12554019 | C | G | 8 | 13282491 | C | G | 8 | 14085219 | C | G |
| 8 | 14085280 | A | T | 8 | 14085785 | T | C | 8 | 14085790 | G | A |
| 8 | 15229407 | G | C | 8 | 15230000 | G | A | 8 | 15786119 | A | G |
| 8 | 16421564 | G | C | 8 | 16701281 | T | G | 8 | 18074243 | A | T |
| 8 | 18074261 | G | A | 8 | 18080644 | A | T | 8 | 18080651 | A | C |
| 8 | 18080747 | T | G | 8 | 18257795 | C | T | 8 | 19319046 | G | C |
| 8 | 19319062 | G | A | 8 | 19319620 | G | A | 8 | 19319625 | G | A |
| 8 | 20918631 | A | G | 8 | 21170053 | A | G | 8 | 21171077 | A | C |
| 8 | 21171170 | T | G | 8 | 21696395 | T | G | 8 | 21696441 | G | A |
| 8 | 22068081 | A | G | 8 | 24287864 | G | C | 8 | 24593635 | G | A |
| 8 | 26519881 | G | A | 8 | 27325191 | G | T | 8 | 27831553 | T | C |
| 8 | 29398520 | C | T | 8 | 29884966 | C | T | 8 | 29886613 | T | C |
| 8 | 30210844 | A | G | 8 | 30939016 | C | G | 8 | 30974348 | G | A |
| 8 | 30989690 | C | T | 8 | 31498589 | G | A | 8 | 31948864 | A | G |
| 8 | 32405979 | T | C | 8 | 32406602 | C | T | 8 | 32432753 | G | T |
| 8 | 32474622 | A | G | 8 | 32599851 | C | T | 8 | 32617494 | G | A |
| 8 | 32620467 | A | G | 8 | 33282335 | G | A | 8 | 33571231 | G | A |
| 8 | 36952867 | C | T | 8 | 36952918 | A | G | 8 | 36953041 | A | G |
| 8 | 38272542 | C | G | 8 | 38272582 | C | A | 8 | 38284994 | C | T |
| 8 | 38286225 | G | A | 8 | 38319729 | A | G | 8 | 38804069 | A | G |
| 8 | 39015793 | T | G | 8 | 40574679 | T | G | 8 | 48405604 | A | T |
| 8 | 48649894 | G | A | 8 | 49540066 | G | T | 8 | 50732260 | A | G |
| 8 | 51133527 | G | T | 8 | 52152983 | C | T | 8 | 53031404 | A | G |
| 8 | 53851462 | T | C | 8 | 53851506 | A | G | 8 | 54068560 | G | A |
| 8 | 54069239 | G | A | 8 | 54446265 | T | C | 8 | 54452476 | C | A |
| 8 | 54573305 | G | T | 8 | 54573308 | G | A | 8 | 55379994 | T | G |
| 8 | 55918430 | T | C | 8 | 56866780 | T | C | 8 | 56879113 | C | G |
| 8 | 58085212 | G | T | 8 | 58711370 | G | A | 8 | 58711450 | C | G |
| 8 | 59257315 | G | C | 8 | 59337563 | C | T | 8 | 59338856 | G | C |
| 8 | 59736647 | T | A | 8 | 62114861 | G | A | 8 | 63055668 | C | T |
| 8 | 63516587 | G | A | 8 | 64183260 | C | A | 8 | 64321899 | T | C |
| 8 | 64322357 | G | A | 8 | 66081707 | G | T | 8 | 66081733 | A | G |
| 8 | 66081764 | G | T | 8 | 66502666 | G | T | 8 | 67380377 | C | T |
| 8 | 67380863 | T | C | 8 | 68934648 | A | T | 8 | 68965782 | T | A |
| 8 | 68967824 | A | G | 8 | 68968466 | T | G | 8 | 68981646 | T | C |
| 8 | 68981979 | T | C | 8 | 68995205 | A | G | 8 | 68995849 | T | G |
| 8 | 69006214 | A | G | 8 | 69046742 | G | A | 8 | 69104278 | C | T |
| 8 | 69104993 | A | G | 8 | 69129636 | A | T | 8 | 69130238 | G | A |
| 8 | 69143417 | C | T | 8 | 70174276 | G | A | 8 | 71340692 | T | A |
| 8 | 71974737 | G | T | 8 | 72466982 | T | C | 8 | 72530703 | C | T |
| 8 | 73795324 | A | C | 8 | 73795889 | C | G | 8 | 75205025 | T | A |
| 8 | 78756303 | G | T | 8 | 78756753 | A | T | 8 | 79457863 | C | T |
| 8 | 79457991 | C | G | 8 | 79673847 | C | T | 8 | 79673952 | T | C |
| 8 | 79776230 | C | T | 8 | 79776231 | A | G | 8 | 79776242 | C | T |
| 8 | 80432897 | C | T | 8 | 82698092 | A | G | 8 | 82698105 | A | G |
| 8 | 83531397 | G | A | 8 | 84739570 | T | C | 8 | 84740129 | G | A |
| 8 | 85148956 | G | A | 8 | 85950696 | G | A | 8 | 87754395 | G | T |
| 8 | 90255638 | G | A | 8 | 90255693 | A | G | 8 | 90255910 | A | C |
| 8 | 90255921 | A | G | 8 | 90255944 | A | T | 8 | 90372173 | G | A |
| 8 | 90947537 | C | T | 8 | 90955253 | T | G | 8 | 90968041 | A | G |
| 8 | 90976405 | C | T | 8 | 90976906 | G | A | 8 | 90982425 | C | T |
| 8 | 90993395 | C | T | 8 | 96216940 | T | G | 8 | 96939758 | T | A |
| 8 | 97548048 | C | A | 8 | 98232612 | G | A | 8 | 101467885 | G | C |
| 8 | 101467929 | T | C | 8 | 102145463 | A | G | 8 | 102998489 | G | C |
| 8 | 102999087 | A | C | 8 | 104137028 | C | T | 8 | 104137029 | A | C |
| 8 | 107401311 | G | A | 8 | 108671073 | C | T | 8 | 109145249 | G | A |
| 8 | 110482900 | T | C | 8 | 110703134 | C | T | 8 | 111149366 | C | T |
| 8 | 114582521 | C | T | 8 | 115803653 | A | G | 8 | 116980628 | T | C |
| 8 | 117915471 | G | A | 8 | 117915557 | A | G | 8 | 117916015 | G | A |
| 8 | 118250570 | C | T | 8 | 118824909 | A | G | 8 | 118825354 | C | T |
| 8 | 118830894 | C | T | 8 | 118831654 | T | C | 8 | 118834484 | A | C |
| 8 | 118842184 | G | A | 8 | 118849146 | T | C | 8 | 119122037 | T | C |
| 8 | 121244377 | A | G | 8 | 121244406 | A | G | 8 | 121926352 | G | A |
| 8 | 121926410 | T | C | 8 | 122272500 | G | A | 8 | 123314293 | C | T |
| 8 | 125753569 | T | C | 8 | 125753604 | G | A | 8 | 125754151 | A | G |
| 8 | 127239272 | C | T | 8 | 131498869 | A | T | 8 | 132926989 | A | G |
| 8 | 133512139 | C | T | 8 | 134828280 | G | C | 8 | 135318241 | G | T |
| 8 | 135510319 | G | T | 8 | 135510321 | G | T | 8 | 135510329 | T | G |
| 8 | 136215771 | G | A | 8 | 138475155 | T | C | 8 | 138475156 | T | A |
| 8 | 138475196 | G | T | 8 | 139218369 | G | A | 8 | 139219088 | A | C |
| 8 | 139219223 | C | A | 8 | 139219307 | G | A | 8 | 140264369 | G | A |
| 8 | 141167195 | G | A | 8 | 141684170 | T | C | 8 | 141685774 | A | G |
| 8 | 141711343 | G | A | 8 | 141762659 | G | A | 8 | 141774576 | A | G |
| 8 | 141799813 | T | C | 8 | 141828264 | G | A | 8 | 141874677 | C | T |
| 8 | 141890008 | T | C | 8 | 142640521 | G | T | 8 | 142640547 | A | C |
| 8 | 142640675 | T | C | 8 | 142640905 | G | A | 8 | 142641198 | C | A |
| 8 | 142642198 | T | A | 8 | 142642202 | G | A | 8 | 143130734 | T | C |
| 8 | 143956693 | G | A | 8 | 143957163 | C | G | 8 | 143994321 | T | C |
| 8 | 143994349 | T | C | 8 | 143994361 | T | A | 8 | 143994388 | G | C |
| 8 | 143994391 | G | C | 8 | 143994396 | A | T | 8 | 143994398 | A | G |
| 8 | 143994399 | T | C | 8 | 143994470 | G | A | 8 | 143994473 | A | C |
| 8 | 143994480 | A | G | 8 | 143994517 | T | C | 8 | 143994702 | G | T |
| 8 | 144286690 | T | G | 8 | 144286716 | T | C | 8 | 144286717 | T | G |
| 8 | 144286759 | G | C | 8 | 144286859 | G | A | 8 | 144287116 | T | G |
| 8 | 144287144 | G | A | 8 | 144287269 | A | G | 8 | 144287286 | C | T |
| 8 | 144743886 | T | C | 8 | 144743892 | A | G | 8 | 144743959 | A | G |
| 8 | 144744194 | C | T | 8 | 144744512 | C | T | 8 | 145180748 | G | A |
| 8 | 145180769 | G | C | 9 | 85392 | G | C | 9 | 118535 | T | C |
| 9 | 118653 | T | C | 9 | 147988 | G | A | 9 | 1200477 | T | C |
| 9 | 1200491 | A | C | 9 | 1200494 | C | G | 9 | 1200508 | T | C |
| 9 | 1201091 | T | C | 9 | 1798525 | G | A | 9 | 1799027 | C | T |
| 9 | 2120825 | G | A | 9 | 2121371 | G | T | 9 | 2624186 | A | G |
| 9 | 4090177 | T | A | 9 | 4090290 | T | C | 9 | 4835827 | C | G |
| 9 | 5021738 | G | A | 9 | 5055434 | C | T | 9 | 5056037 | A | G |
| 9 | 5072846 | G | A | 9 | 5078117 | T | C | 9 | 5081585 | A | G |
| 9 | 5082106 | G | T | 9 | 5465262 | G | A | 9 | 5549080 | T | C |
| 9 | 5549099 | G | A | 9 | 6033278 | G | A | 9 | 6930845 | C | T |
| 9 | 6930896 | G | A | 9 | 6930901 | T | G | 9 | 6931468 | T | A |
| 9 | 7273796 | C | G | 9 | 7274258 | G | C | 9 | 7597988 | C | T |
| 9 | 7798435 | C | T | 9 | 7798518 | G | C | 9 | 7994043 | A | G |
| 9 | 7994044 | T | A | 9 | 7994129 | A | T | 9 | 8578261 | T | C |
| 9 | 9394902 | A | G | 9 | 9651978 | G | C | 9 | 11167895 | C | T |
| 9 | 11610136 | C | A | 9 | 14473276 | A | C | 9 | 14518676 | G | C |
| 9 | 14519301 | T | C | 9 | 17014049 | C | T | 9 | 18936179 | C | G |
| 9 | 19789029 | G | A | 9 | 20414692 | C | G | 9 | 20973968 | T | A |
| 9 | 20974346 | T | C | 9 | 20975006 | C | A | 9 | 21166004 | A | G |
| 9 | 21166077 | T | C | 9 | 21187121 | T | A | 9 | 21201703 | T | C |
| 9 | 21206763 | G | T | 9 | 21216728 | T | C | 9 | 21350050 | T | C |
| 9 | 21350078 | A | T | 9 | 21440490 | T | C | 9 | 21974218 | A | G |
| 9 | 21975141 | A | T | 9 | 25475165 | T | A | 9 | 27168315 | G | A |
| 9 | 27169190 | A | T | 9 | 27185249 | C | A | 9 | 27185251 | C | T |
| 9 | 27190949 | T | C | 9 | 27190992 | C | T | 9 | 27202662 | C | T |
| 9 | 27206307 | A | C | 9 | 27208857 | G | T | 9 | 27209422 | G | A |
| 9 | 27209469 | A | C | 9 | 27219058 | G | A | 9 | 27228022 | A | G |
| 9 | 27228058 | T | G | 9 | 27228532 | G | A | 9 | 27228564 | G | A |
| 9 | 27584899 | T | C | 9 | 27608585 | G | C | 9 | 27608667 | T | C |
| 9 | 29706139 | G | A | 9 | 31020442 | A | C | 9 | 32264612 | G | A |
| 9 | 32264652 | T | C | 9 | 32853813 | G | A | 9 | 33524684 | G | A |
| 9 | 33524725 | G | T | 9 | 33524775 | C | A | 9 | 33525041 | T | A |
| 9 | 33602938 | T | G | 9 | 33602956 | G | A | 9 | 33603177 | G | A |
| 9 | 33613678 | C | A | 9 | 33613770 | T | A | 9 | 33613903 | A | G |
| 9 | 33613912 | C | G | 9 | 33613913 | T | G | 9 | 33613915 | A | G |
| 9 | 33613920 | C | A | 9 | 33613927 | G | T | 9 | 33613986 | C | A |
| 9 | 33796912 | A | G | 9 | 34724786 | G | A | 9 | 34724796 | T | A |
| 9 | 34724832 | G | A | 9 | 34834103 | T | C | 9 | 34834109 | T | C |
| 9 | 35657741 | A | G | 9 | 36847139 | G | A | 9 | 37003075 | A | G |
| 9 | 37020361 | G | A | 9 | 37020388 | T | C | 9 | 37033693 | A | G |
| 9 | 37490307 | C | A | 9 | 37490422 | G | A | 9 | 37490577 | C | G |
| 9 | 37512984 | G | A | 9 | 37513061 | C | G | 9 | 38344773 | T | A |
| 9 | 39029031 | T | C | 9 | 39029036 | C | A | 9 | 40072440 | C | A |
| 9 | 41237632 | C | G | 9 | 42847566 | G | A | 9 | 42847573 | C | A |
| 9 | 42847654 | A | C | 9 | 43541016 | C | T | 9 | 43940526 | T | G |
| 9 | 44212144 | T | G | 9 | 44245084 | C | T | 9 | 44761941 | T | C |
| 9 | 44768417 | G | A | 9 | 44768485 | A | C | 9 | 44768659 | C | T |
| 9 | 44791283 | G | T | 9 | 44791365 | T | C | 9 | 44841887 | G | A |
| 9 | 44841907 | T | G | 9 | 44842094 | G | A | 9 | 44842252 | A | C |
| 9 | 44842293 | T | G | 9 | 44847374 | G | A | 9 | 44847446 | A | C |
| 9 | 44847490 | A | G | 9 | 44847927 | C | A | 9 | 44847977 | T | C |
| 9 | 44865971 | G | T | 9 | 44866028 | C | T | 9 | 44866421 | C | T |
| 9 | 44904433 | A | G | 9 | 44904612 | A | C | 9 | 45079401 | T | C |
| 9 | 45079451 | C | G | 9 | 45079535 | T | G | 9 | 45079548 | G | A |
| 9 | 45079638 | G | A | 9 | 45079671 | T | G | 9 | 45415790 | G | C |
| 9 | 45741414 | G | A | 9 | 45741417 | A | G | 9 | 45741583 | A | G |
| 9 | 45758158 | C | T | 9 | 45758167 | T | A | 9 | 46125082 | T | C |
| 9 | 46125086 | G | A | 9 | 46125090 | G | A | 9 | 46200382 | G | T |
| 9 | 46200485 | C | T | 9 | 46200696 | G | A | 9 | 46200729 | T | G |
| 9 | 46877337 | A | G | 9 | 46877500 | A | C | 9 | 47165813 | A | T |
| 9 | 47165846 | C | A | 9 | 66260870 | A | C | 9 | 66675478 | A | G |
| 9 | 66675670 | T | C | 9 | 66675721 | C | T | 9 | 66675793 | G | A |
| 9 | 67020555 | G | A | 9 | 67020584 | A | C | 9 | 67020641 | A | C |
| 9 | 67683148 | C | A | 9 | 67683187 | A | C | 9 | 67683290 | A | G |
| 9 | 67683344 | C | T | 9 | 67683345 | A | G | 9 | 68233761 | T | C |
| 9 | 68672364 | G | T | 9 | 68724152 | G | C | 9 | 68760967 | C | T |
| 9 | 68761034 | G | C | 9 | 68761231 | A | G | 9 | 69450156 | C | T |
| 9 | 69450371 | C | T | 9 | 69563020 | T | C | 9 | 69563070 | A | C |
| 9 | 69784204 | C | T | 9 | 69784585 | C | T | 9 | 69787544 | T | C |
| 9 | 69787624 | C | T | 9 | 69787627 | C | T | 9 | 69787636 | A | T |
| 9 | 69787642 | G | A | 9 | 69787650 | A | G | 9 | 69788026 | G | A |
| 9 | 70152508 | C | T | 9 | 70888221 | C | T | 9 | 70888239 | G | A |
| 9 | 70888295 | T | C | 9 | 71020205 | A | G | 9 | 71020288 | C | G |
| 9 | 71020391 | G | C | 9 | 71020509 | T | C | 9 | 71020669 | T | C |
| 9 | 71628207 | G | C | 9 | 72137536 | A | G | 9 | 73469948 | C | T |
| 9 | 74245426 | C | T | 9 | 78133661 | C | T | 9 | 79150662 | A | G |
| 9 | 80409138 | C | G | 9 | 80795416 | T | C | 9 | 82962978 | G | A |
| 9 | 84935618 | T | C | 9 | 85346339 | C | T | 9 | 87316868 | A | T |
| 9 | 87322540 | T | C | 9 | 87325994 | A | G | 9 | 87338267 | T | A |
| 9 | 87425206 | T | G | 9 | 87481852 | G | A | 9 | 87570004 | C | T |
| 9 | 87570705 | C | T | 9 | 87635540 | C | T | 9 | 90268337 | T | C |
| 9 | 90631191 | A | G | 9 | 90631497 | T | C | 9 | 90650554 | C | T |
| 9 | 90650578 | T | C | 9 | 90651113 | A | G | 9 | 90940058 | G | A |
| 9 | 91154126 | A | G | 9 | 92280487 | A | G | 9 | 92280986 | T | C |
| 9 | 92995579 | C | T | 9 | 93359638 | A | G | 9 | 93504722 | T | C |
| 9 | 93714276 | T | C | 9 | 93714912 | C | G | 9 | 94518604 | T | C |
| 9 | 94768542 | A | G | 9 | 95484063 | A | G | 9 | 96375217 | T | C |
| 9 | 96375464 | G | A | 9 | 96581893 | A | G | 9 | 96582360 | T | C |
| 9 | 97078732 | A | G | 9 | 97078831 | C | A | 9 | 97078905 | A | G |
| 9 | 97078981 | A | G | 9 | 98009494 | C | T | 9 | 98209958 | G | A |
| 9 | 98231908 | G | A | 9 | 98238860 | T | C | 9 | 98248328 | A | G |
| 9 | 98248387 | A | G | 9 | 98270168 | A | G | 9 | 98270171 | A | G |
| 9 | 98278644 | C | A | 9 | 98279370 | T | A | 9 | 99703218 | G | T |
| 9 | 100437487 | G | T | 9 | 100437518 | G | C | 9 | 100447477 | A | C |
| 9 | 100452141 | T | G | 9 | 100462763 | G | C | 9 | 104565163 | G | T |
| 9 | 105915548 | G | A | 9 | 106693503 | T | C | 9 | 107361111 | T | C |
| 9 | 107361129 | C | T | 9 | 107361312 | T | C | 9 | 107361439 | G | C |
| 9 | 107361449 | T | C | 9 | 107361451 | G | A | 9 | 107361452 | C | G |
| 9 | 107361460 | G | A | 9 | 107361490 | A | T | 9 | 107361599 | G | C |
| 9 | 107361642 | G | A | 9 | 107361710 | G | A | 9 | 107367431 | A | T |
| 9 | 107367841 | A | T | 9 | 107379895 | G | T | 9 | 107380396 | T | G |
| 9 | 107380414 | C | A | 9 | 107839521 | G | A | 9 | 107839542 | A | G |
| 9 | 108654829 | G | A | 9 | 109863693 | G | A | 9 | 112508341 | G | A |
| 9 | 113336508 | C | T | 9 | 114371047 | T | C | 9 | 114371072 | G | A |
| 9 | 114371590 | G | A | 9 | 114371624 | A | C | 9 | 114680414 | C | T |
| 9 | 114680447 | G | A | 9 | 115386827 | A | T | 9 | 115821854 | T | C |
| 9 | 116025982 | A | C | 9 | 116026034 | A | G | 9 | 116026040 | C | T |
| 9 | 116026041 | G | A | 9 | 116816581 | G | A | 9 | 116816582 | A | T |
| 9 | 117092020 | G | A | 9 | 117750232 | T | C | 9 | 117750277 | T | C |
| 9 | 119127244 | T | C | 9 | 120437366 | C | T | 9 | 124570006 | C | T |
| 9 | 124570555 | A | G | 9 | 125288893 | C | T | 9 | 126202375 | C | T |
| 9 | 126214331 | G | C | 9 | 126219431 | T | C | 9 | 126319637 | C | G |
| 9 | 126414074 | T | C | 9 | 126555072 | G | C | 9 | 127956805 | C | G |
| 9 | 127956819 | A | G | 9 | 127957393 | T | C | 9 | 128026278 | G | T |
| 9 | 131993190 | C | T | 9 | 132200320 | G | A | 9 | 132524895 | C | G |
| 9 | 133440976 | C | T | 9 | 135271536 | T | C | 9 | 135271577 | C | T |
| 9 | 135271591 | T | C | 9 | 135273266 | A | G | 9 | 135273288 | A | G |
| 9 | 135276546 | G | A | 9 | 135276606 | A | G | 9 | 135777711 | T | A |
| 9 | 135779530 | T | C | 9 | 135781709 | G | C | 9 | 135782968 | C | T |
| 9 | 135802298 | A | G | 9 | 136768563 | C | A | 9 | 136768575 | A | G |
| 9 | 137415294 | T | C | 9 | 137415319 | C | T | 9 | 137415342 | T | C |
| 9 | 137415554 | A | G | 9 | 137415932 | A | G | 9 | 137416041 | A | C |
| 9 | 137416082 | T | C | 9 | 137416186 | C | T | 9 | 137416192 | C | T |
| 9 | 137416251 | C | T | 9 | 138364601 | T | C | 9 | 138604936 | C | T |
| 9 | 138604947 | C | G | 9 | 139403770 | G | A | 9 | 139411374 | A | G |
| 9 | 139411452 | G | A | 9 | 139411479 | G | A | 9 | 139413533 | C | T |
| 9 | 139413539 | C | T | 9 | 139418661 | A | G | 9 | 139997934 | A | G |
| 9 | 139997971 | C | T | 9 | 139998042 | C | T | 9 | 140242925 | C | T |
| 9 | 140242995 | G | A | 9 | 140242998 | G | T | 9 | 140243000 | T | C |
| 9 | 140675590 | T | C | 9 | 141031520 | C | T | 9 | 141031567 | G | C |
| 9 | 141031898 | T | G | 9 | 141031991 | T | C | 9 | 141075634 | G | A |
| 9 | 141076109 | C | A | 10 | 392593 | T | C | 10 | 392722 | G | A |
| 10 | 392735 | G | A | 10 | 392765 | G | A | 10 | 393501 | C | T |
| 10 | 393577 | C | T | 10 | 393669 | G | A | 10 | 393674 | T | C |
| 10 | 1482550 | C | T | 10 | 1582749 | T | C | 10 | 1582934 | T | C |
| 10 | 1582958 | A | G | 10 | 1583176 | T | C | 10 | 1583692 | T | C |
| 10 | 1584399 | A | G | 10 | 1584802 | G | A | 10 | 2565603 | A | T |
| 10 | 2565607 | G | T | 10 | 2565611 | G | A | 10 | 2565621 | T | C |
| 10 | 2565636 | T | G | 10 | 2566085 | C | T | 10 | 3498982 | A | G |
| 10 | 3499603 | C | T | 10 | 3499625 | T | C | 10 | 3499710 | A | G |
| 10 | 3499787 | T | C | 10 | 3499972 | A | G | 10 | 3500517 | C | T |
| 10 | 5018132 | A | G | 10 | 5018146 | T | A | 10 | 5018174 | C | T |
| 10 | 5018178 | G | A | 10 | 5018204 | A | G | 10 | 5018224 | C | T |
| 10 | 5018310 | T | C | 10 | 5018311 | G | T | 10 | 5033837 | C | A |
| 10 | 5033838 | A | G | 10 | 5034016 | T | C | 10 | 6182251 | C | T |
| 10 | 6182642 | G | A | 10 | 7535307 | A | G | 10 | 8097368 | G | A |
| 10 | 8098185 | A | G | 10 | 8105745 | G | A | 10 | 8106353 | G | A |
| 10 | 8111168 | C | T | 10 | 8562125 | A | T | 10 | 8562634 | A | G |
| 10 | 10779423 | C | T | 10 | 11769964 | G | T | 10 | 12201176 | C | T |
| 10 | 13000296 | G | A | 10 | 13000315 | C | T | 10 | 13000321 | G | C |
| 10 | 13978012 | G | A | 10 | 15009109 | T | C | 10 | 15009164 | G | T |
| 10 | 15009590 | A | G | 10 | 16416773 | A | G | 10 | 16416894 | A | G |
| 10 | 16417376 | T | C | 10 | 16969923 | A | C | 10 | 17419872 | G | A |
| 10 | 17419920 | C | G | 10 | 17591919 | C | G | 10 | 18440733 | T | C |
| 10 | 18790620 | A | G | 10 | 19007587 | C | T | 10 | 19007616 | A | G |
| 10 | 19764840 | T | C | 10 | 19765360 | G | A | 10 | 19765370 | C | T |
| 10 | 20932917 | C | T | 10 | 20933397 | G | A | 10 | 21063302 | G | C |
| 10 | 22493554 | T | A | 10 | 23400393 | T | C | 10 | 26158386 | A | C |
| 10 | 26158422 | A | C | 10 | 26930489 | G | C | 10 | 27227955 | T | C |
| 10 | 27539507 | C | T | 10 | 27539528 | G | A | 10 | 27539598 | G | C |
| 10 | 27539619 | T | C | 10 | 27539928 | A | G | 10 | 27539934 | G | A |
| 10 | 27692062 | T | C | 10 | 27692099 | A | G | 10 | 27692561 | T | G |
| 10 | 27692581 | G | T | 10 | 28192872 | C | T | 10 | 28193276 | G | T |
| 10 | 29491074 | A | T | 10 | 29491536 | G | A | 10 | 29783813 | G | A |
| 10 | 29783885 | A | G | 10 | 29784003 | G | A | 10 | 29784072 | G | C |
| 10 | 29784114 | T | C | 10 | 29784176 | G | A | 10 | 29784177 | T | C |
| 10 | 29784178 | G | C | 10 | 29784188 | A | G | 10 | 29784190 | A | G |
| 10 | 29784218 | G | T | 10 | 29784297 | T | A | 10 | 29784333 | A | G |
| 10 | 29784363 | C | T | 10 | 30213011 | C | T | 10 | 30213059 | G | A |
| 10 | 30213561 | C | T | 10 | 30977801 | T | C | 10 | 30978282 | G | T |
| 10 | 30978377 | G | C | 10 | 30978388 | C | T | 10 | 30978433 | A | G |
| 10 | 34451892 | G | A | 10 | 34451933 | T | C | 10 | 36910716 | G | A |
| 10 | 37430500 | C | A | 10 | 38539751 | C | T | 10 | 38597731 | G | A |
| 10 | 38711591 | C | G | 10 | 38711618 | C | A | 10 | 38743039 | G | A |
| 10 | 38743615 | G | A | 10 | 38743659 | A | G | 10 | 38743802 | G | T |
| 10 | 38752939 | A | C | 10 | 38770349 | C | A | 10 | 38893425 | C | T |
| 10 | 38893430 | C | T | 10 | 38893448 | T | C | 10 | 38893449 | G | A |
| 10 | 38893451 | C | T | 10 | 38893471 | C | T | 10 | 39006616 | G | T |
| 10 | 39011495 | C | T | 10 | 39012502 | A | G | 10 | 39023235 | C | G |
| 10 | 39023315 | T | C | 10 | 42607378 | T | C | 10 | 42607892 | A | G |
| 10 | 42607909 | C | T | 10 | 42607937 | G | A | 10 | 42607955 | G | T |
| 10 | 42607956 | C | T | 10 | 42738375 | C | G | 10 | 42821007 | A | G |
| 10 | 43247120 | G | A | 10 | 43247625 | C | G | 10 | 43572507 | A | G |
| 10 | 43572511 | C | A | 10 | 43572950 | A | G | 10 | 43572951 | A | C |
| 10 | 43601559 | G | A | 10 | 43604950 | A | G | 10 | 43614230 | G | A |
| 10 | 43614694 | G | A | 10 | 43620079 | G | A | 10 | 43620134 | C | T |
| 10 | 43691691 | C | T | 10 | 45406608 | T | C | 10 | 45652138 | T | C |
| 10 | 45652447 | A | G | 10 | 45652504 | T | G | 10 | 45652579 | A | G |
| 10 | 45922190 | G | A | 10 | 46195321 | T | G | 10 | 47152194 | G | A |
| 10 | 47152364 | A | G | 10 | 47170635 | A | T | 10 | 47707447 | G | A |
| 10 | 47707459 | T | C | 10 | 47729649 | C | T | 10 | 47739153 | A | G |
| 10 | 47750099 | G | T | 10 | 47768664 | T | C | 10 | 47768722 | T | C |
| 10 | 49218034 | G | A | 10 | 49218414 | C | A | 10 | 49218451 | G | A |
| 10 | 49218561 | G | C | 10 | 49240255 | A | G | 10 | 49240259 | C | A |
| 10 | 49872724 | C | A | 10 | 50013531 | A | G | 10 | 50013580 | C | T |
| 10 | 50013594 | C | T | 10 | 50534599 | G | A | 10 | 50534646 | C | T |
| 10 | 51486999 | G | A | 10 | 52438586 | G | A | 10 | 52438599 | G | A |
| 10 | 52439039 | G | A | 10 | 52467072 | G | A | 10 | 52537198 | C | T |
| 10 | 53615000 | T | C | 10 | 54171342 | G | T | 10 | 54171478 | A | T |
| 10 | 54171706 | G | T | 10 | 54228504 | G | T | 10 | 54228515 | A | G |
| 10 | 55422826 | G | T | 10 | 56915486 | C | T | 10 | 56915523 | G | A |
| 10 | 56915531 | A | T | 10 | 58941173 | C | T | 10 | 59342734 | T | A |
| 10 | 59343246 | A | C | 10 | 60228964 | G | A | 10 | 61956912 | C | A |
| 10 | 63723336 | T | C | 10 | 63723440 | A | C | 10 | 63723909 | C | T |
| 10 | 64703524 | C | T | 10 | 66488391 | G | T | 10 | 67598587 | C | T |
| 10 | 68498439 | A | G | 10 | 70342096 | G | A | 10 | 70836869 | T | G |
| 10 | 71582636 | T | A | 10 | 72359787 | A | G | 10 | 72691384 | C | T |
| 10 | 72691997 | C | T | 10 | 73542342 | A | G | 10 | 73542839 | T | C |
| 10 | 75434461 | G | A | 10 | 75458179 | C | A | 10 | 78649399 | C | T |
| 10 | 78649930 | A | G | 10 | 79540127 | C | T | 10 | 81282125 | G | A |
| 10 | 81282738 | T | C | 10 | 81632385 | G | C | 10 | 83187807 | T | A |
| 10 | 83187909 | C | T | 10 | 84158423 | G | A | 10 | 84158433 | A | G |
| 10 | 84158436 | G | A | 10 | 84158972 | T | C | 10 | 84473715 | A | G |
| 10 | 86281312 | G | C | 10 | 88651750 | C | T | 10 | 89128565 | C | G |
| 10 | 90232512 | T | C | 10 | 91142901 | T | C | 10 | 91570905 | C | T |
| 10 | 96541373 | A | G | 10 | 96602398 | T | A | 10 | 96741426 | C | T |
| 10 | 97759583 | T | C | 10 | 97759626 | A | G | 10 | 97760182 | G | A |
| 10 | 97949901 | C | T | 10 | 97990491 | C | T | 10 | 98319855 | A | G |
| 10 | 98320693 | G | C | 10 | 98320932 | A | G | 10 | 98510073 | A | G |
| 10 | 99439541 | G | C | 10 | 99439556 | A | C | 10 | 99439901 | G | A |
| 10 | 99972123 | T | G | 10 | 100508702 | C | A | 10 | 101341813 | G | A |
| 10 | 104263675 | T | C | 10 | 104268626 | T | C | 10 | 104269301 | G | A |
| 10 | 104374815 | G | C | 10 | 104378563 | G | T | 10 | 104378750 | G | A |
| 10 | 107980644 | T | G | 10 | 108496447 | G | A | 10 | 108496455 | C | T |
| 10 | 108497048 | C | T | 10 | 109397974 | A | G | 10 | 109398465 | T | C |
| 10 | 110281465 | C | T | 10 | 110998926 | G | C | 10 | 111333728 | A | G |
| 10 | 113752957 | C | T | 10 | 113753002 | T | C | 10 | 113753096 | T | C |
| 10 | 114488065 | G | A | 10 | 114591047 | C | G | 10 | 115475804 | A | G |
| 10 | 115548915 | A | T | 10 | 115548971 | T | A | 10 | 116006120 | C | T |
| 10 | 116006247 | T | C | 10 | 116180554 | C | T | 10 | 117486391 | G | T |
| 10 | 117810868 | G | A | 10 | 118147645 | A | G | 10 | 118147665 | T | C |
| 10 | 118148153 | A | C | 10 | 118525028 | A | T | 10 | 118525526 | T | C |
| 10 | 119243364 | C | T | 10 | 119243810 | T | A | 10 | 120403436 | C | T |
| 10 | 120766000 | A | G | 10 | 120766163 | T | A | 10 | 121050776 | C | T |
| 10 | 121050852 | G | A | 10 | 121050965 | G | A | 10 | 121162095 | T | C |
| 10 | 121631812 | C | G | 10 | 122520364 | C | T | 10 | 123246612 | G | A |
| 10 | 123345664 | C | T | 10 | 123358170 | C | T | 10 | 123358273 | C | T |
| 10 | 123862546 | C | T | 10 | 123862547 | A | G | 10 | 124573552 | T | C |
| 10 | 124573574 | C | T | 10 | 125310002 | C | T | 10 | 125310499 | G | A |
| 10 | 125757093 | C | T | 10 | 126195810 | G | A | 10 | 126195824 | C | G |
| 10 | 126196558 | G | C | 10 | 126741574 | C | T | 10 | 127349999 | A | C |
| 10 | 127846669 | C | T | 10 | 127846703 | C | T | 10 | 127846720 | T | C |
| 10 | 127847387 | T | A | 10 | 128227832 | T | C | 10 | 129339809 | G | A |
| 10 | 129340335 | T | C | 10 | 129650290 | G | A | 10 | 129650996 | A | G |
| 10 | 131265852 | C | T | 10 | 131506546 | G | A | 10 | 131557867 | A | G |
| 10 | 131557933 | G | A | 10 | 131564809 | G | A | 10 | 131565509 | G | A |
| 10 | 132295030 | A | G | 10 | 132295056 | T | C | 10 | 132557571 | T | C |
| 10 | 132557592 | A | G | 10 | 132557593 | T | A | 10 | 132559128 | T | C |
| 10 | 133566682 | G | A | 10 | 134318895 | G | T | 10 | 134926554 | A | G |
| 10 | 134926776 | T | C | 11 | 179894 | T | A | 11 | 2157044 | C | G |
| 11 | 2161159 | G | A | 11 | 3435481 | T | C | 11 | 3435668 | A | G |
| 11 | 3435671 | C | G | 11 | 3439299 | C | T | 11 | 3439368 | G | T |
| 11 | 3439925 | C | T | 11 | 3463826 | T | C | 11 | 3463827 | G | A |
| 11 | 3553733 | T | C | 11 | 3592585 | A | G | 11 | 3603224 | G | A |
| 11 | 3605382 | T | G | 11 | 3605415 | T | C | 11 | 3605624 | A | T |
| 11 | 3605644 | T | C | 11 | 3605771 | G | A | 11 | 3605773 | C | T |
| 11 | 3913738 | A | G | 11 | 4115974 | C | A | 11 | 4128486 | A | G |
| 11 | 4130621 | A | G | 11 | 4132640 | C | A | 11 | 4139978 | T | C |
| 11 | 4142592 | T | C | 11 | 4143692 | T | A | 11 | 4143721 | G | T |
| 11 | 4144842 | A | G | 11 | 4144867 | C | G | 11 | 4150101 | T | C |
| 11 | 4153891 | T | C | 11 | 4155132 | C | T | 11 | 4156040 | T | A |
| 11 | 4156106 | G | A | 11 | 4159202 | T | C | 11 | 4159225 | A | G |
| 11 | 4159247 | G | A | 11 | 4159252 | C | T | 11 | 4159285 | T | C |
| 11 | 5232146 | G | A | 11 | 5232212 | T | A | 11 | 6324678 | T | C |
| 11 | 6325216 | T | C | 11 | 6807468 | A | T | 11 | 7449501 | G | A |
| 11 | 7505088 | C | A | 11 | 7505089 | T | C | 11 | 8285124 | A | C |
| 11 | 9682401 | C | T | 11 | 10754380 | T | G | 11 | 11331170 | A | G |
| 11 | 11504981 | C | T | 11 | 14274550 | T | G | 11 | 14957663 | G | A |
| 11 | 16334778 | A | T | 11 | 17074520 | G | A | 11 | 18288806 | C | G |
| 11 | 18288948 | G | A | 11 | 18289166 | T | C | 11 | 18977418 | A | G |
| 11 | 19865934 | C | G | 11 | 20062121 | T | A | 11 | 20062277 | T | C |
| 11 | 20062704 | C | A | 11 | 21484515 | C | T | 11 | 21562629 | G | C |
| 11 | 21563066 | T | C | 11 | 22211249 | A | G | 11 | 22646025 | G | A |
| 11 | 22647550 | A | G | 11 | 23039122 | T | C | 11 | 23539200 | C | G |
| 11 | 23539342 | A | G | 11 | 23539753 | C | T | 11 | 24396319 | T | C |
| 11 | 25041687 | T | C | 11 | 25041755 | T | G | 11 | 25041811 | A | C |
| 11 | 25041812 | A | G | 11 | 25042243 | G | A | 11 | 25042257 | C | T |
| 11 | 27354842 | C | G | 11 | 29754392 | A | G | 11 | 32410337 | C | G |
| 11 | 32457138 | A | G | 11 | 33394460 | T | C | 11 | 34251360 | T | C |
| 11 | 34251855 | G | A | 11 | 35425747 | T | A | 11 | 36472800 | A | G |
| 11 | 37145360 | T | C | 11 | 38527379 | G | A | 11 | 42748900 | A | T |
| 11 | 42748986 | T | C | 11 | 42748998 | G | A | 11 | 43160505 | T | G |
| 11 | 43536288 | A | G | 11 | 44129086 | A | T | 11 | 44151480 | G | T |
| 11 | 44855872 | G | C | 11 | 45123270 | G | A | 11 | 46166591 | G | T |
| 11 | 46450781 | C | G | 11 | 46450821 | G | A | 11 | 48453678 | G | A |
| 11 | 48485877 | T | C | 11 | 48599491 | A | G | 11 | 48985441 | T | C |
| 11 | 48985508 | G | C | 11 | 49009877 | T | C | 11 | 49080616 | T | C |
| 11 | 49102685 | G | A | 11 | 49103176 | C | T | 11 | 49829781 | G | A |
| 11 | 49829847 | G | T | 11 | 49844161 | C | G | 11 | 49844296 | T | G |
| 11 | 49844746 | G | C | 11 | 49844747 | T | C | 11 | 49867489 | A | C |
| 11 | 49867490 | A | C | 11 | 49868030 | A | G | 11 | 49868050 | G | C |
| 11 | 49868051 | G | A | 11 | 49878757 | G | T | 11 | 49878885 | T | C |
| 11 | 49878894 | G | A | 11 | 49878896 | A | G | 11 | 49878899 | G | C |
| 11 | 49908279 | G | C | 11 | 49908382 | G | A | 11 | 49908392 | C | A |
| 11 | 49908581 | T | C | 11 | 49908583 | C | T | 11 | 49908586 | C | A |
| 11 | 49908592 | A | G | 11 | 49908840 | A | G | 11 | 49908856 | A | C |
| 11 | 51483215 | C | T | 11 | 51483747 | G | C | 11 | 51515460 | C | T |
| 11 | 51515640 | A | G | 11 | 51516257 | A | T | 11 | 54934563 | C | T |
| 11 | 55035985 | G | A | 11 | 55036233 | C | T | 11 | 55049986 | C | A |
| 11 | 55050558 | T | C | 11 | 55071967 | T | G | 11 | 55072428 | C | T |
| 11 | 55532244 | T | C | 11 | 55532303 | T | C | 11 | 55532334 | A | G |
| 11 | 55532908 | A | T | 11 | 56517716 | G | A | 11 | 60264755 | G | A |
| 11 | 60265322 | T | C | 11 | 60804962 | G | A | 11 | 61456160 | C | T |
| 11 | 61731977 | G | A | 11 | 61773679 | C | T | 11 | 62098295 | T | A |
| 11 | 62914125 | G | A | 11 | 63817799 | G | C | 11 | 64574200 | G | A |
| 11 | 65793121 | C | T | 11 | 65793797 | G | A | 11 | 67564437 | A | G |
| 11 | 67564738 | G | A | 11 | 67568487 | C | T | 11 | 67570639 | C | T |
| 11 | 67571091 | T | A | 11 | 67714122 | C | G | 11 | 67726415 | G | A |
| 11 | 67931735 | A | G | 11 | 67931761 | G | A | 11 | 69457547 | C | T |
| 11 | 69459046 | A | G | 11 | 69465681 | A | G | 11 | 70424188 | G | A |
| 11 | 70857741 | A | G | 11 | 71347185 | T | C | 11 | 71349215 | G | A |
| 11 | 71349372 | G | A | 11 | 71349527 | T | G | 11 | 71349654 | G | A |
| 11 | 71349696 | T | C | 11 | 71360446 | G | A | 11 | 71399927 | C | T |
| 11 | 71400609 | T | C | 11 | 71477298 | T | C | 11 | 71477335 | C | G |
| 11 | 71502424 | G | A | 11 | 71502531 | G | C | 11 | 71502886 | G | A |
| 11 | 71502930 | A | G | 11 | 71506814 | A | T | 11 | 71615069 | G | A |
| 11 | 75432864 | A | G | 11 | 75432886 | G | C | 11 | 75432903 | G | A |
| 11 | 76169644 | A | G | 11 | 76170687 | C | T | 11 | 76239148 | G | A |
| 11 | 76239150 | C | T | 11 | 76239160 | A | G | 11 | 76250374 | G | A |
| 11 | 76515087 | G | C | 11 | 76515105 | C | A | 11 | 77776590 | G | C |
| 11 | 77777188 | C | T | 11 | 78447534 | T | C | 11 | 80555975 | G | C |
| 11 | 80556380 | T | C | 11 | 81258169 | T | C | 11 | 81258185 | C | T |
| 11 | 83261268 | C | T | 11 | 83261334 | C | T | 11 | 83261851 | A | G |
| 11 | 83580202 | A | C | 11 | 87079863 | A | G | 11 | 88211600 | T | C |
| 11 | 89487612 | G | A | 11 | 89609220 | T | C | 11 | 89609260 | G | A |
| 11 | 89795691 | G | A | 11 | 90372636 | G | A | 11 | 90972900 | C | A |
| 11 | 90973362 | T | C | 11 | 90973418 | G | A | 11 | 91680278 | T | C |
| 11 | 92623608 | A | G | 11 | 93754508 | G | A | 11 | 94180115 | G | C |
| 11 | 94200690 | C | G | 11 | 94203353 | G | A | 11 | 94213155 | C | G |
| 11 | 94759059 | A | G | 11 | 94782500 | C | G | 11 | 94782501 | A | G |
| 11 | 94782515 | C | A | 11 | 95040632 | C | A | 11 | 95041189 | C | A |
| 11 | 95041230 | C | T | 11 | 95532166 | A | G | 11 | 95550810 | C | G |
| 11 | 95561488 | T | G | 11 | 95562771 | T | C | 11 | 96852915 | A | T |
| 11 | 97332219 | G | A | 11 | 98998719 | G | C | 11 | 99216940 | T | G |
| 11 | 99217421 | T | C | 11 | 99865248 | A | G | 11 | 99865266 | C | A |
| 11 | 99865868 | T | C | 11 | 100921941 | T | A | 11 | 101217386 | A | T |
| 11 | 101217962 | A | G | 11 | 101465270 | A | G | 11 | 101981463 | G | T |
| 11 | 101984628 | C | T | 11 | 102079986 | G | A | 11 | 102100199 | C | T |
| 11 | 103416881 | T | C | 11 | 103416913 | T | G | 11 | 103542664 | A | G |
| 11 | 104013175 | A | G | 11 | 104013263 | A | T | 11 | 105724696 | C | T |
| 11 | 106318163 | T | C | 11 | 106692416 | G | A | 11 | 107669817 | C | T |
| 11 | 107669894 | C | T | 11 | 107670383 | C | G | 11 | 107670446 | A | G |
| 11 | 107670459 | A | G | 11 | 108106782 | C | T | 11 | 108158134 | T | C |
| 11 | 108163733 | A | G | 11 | 108165406 | G | A | 11 | 108165407 | C | G |
| 11 | 108170271 | G | A | 11 | 108196509 | G | C | 11 | 108204881 | C | T |
| 11 | 110016032 | A | G | 11 | 110016519 | T | G | 11 | 110712255 | A | T |
| 11 | 110712847 | C | G | 11 | 114092153 | C | A | 11 | 116732856 | A | G |
| 11 | 117343197 | T | C | 11 | 117956263 | C | T | 11 | 117956338 | G | T |
| 11 | 117956409 | T | C | 11 | 117956943 | A | G | 11 | 118362869 | T | C |
| 11 | 118567444 | A | G | 11 | 118567486 | T | C | 11 | 119145274 | A | G |
| 11 | 119167448 | T | G | 11 | 119168370 | G | A | 11 | 120440043 | A | G |
| 11 | 120508093 | C | T | 11 | 124455318 | T | C | 11 | 125497247 | T | C |
| 11 | 125497342 | A | G | 11 | 125505095 | C | G | 11 | 125507676 | C | T |
| 11 | 126666122 | C | T | 11 | 128240874 | A | T | 11 | 128240927 | G | C |
| 11 | 130416523 | C | T | 11 | 130417076 | G | A | 11 | 130417573 | A | G |
| 11 | 131350351 | C | G | 11 | 131350968 | G | A | 11 | 134916463 | G | A |
| 11 | 134916479 | C | T | 11 | 134916495 | T | C | 11 | 134917197 | G | A |
| 12 | 420429 | G | A | 12 | 427077 | G | A | 12 | 433151 | A | G |
| 12 | 442354 | T | C | 12 | 443085 | C | A | 12 | 443795 | T | C |
| 12 | 460226 | C | T | 12 | 461637 | C | T | 12 | 464123 | A | G |
| 12 | 471826 | C | T | 12 | 474797 | T | C | 12 | 494762 | G | C |
| 12 | 528452 | A | T | 12 | 528865 | A | C | 12 | 528887 | A | T |
| 12 | 530660 | T | A | 12 | 530660 | T | G | 12 | 1194236 | A | G |
| 12 | 1194372 | A | G | 12 | 1194777 | A | G | 12 | 1196938 | A | G |
| 12 | 1200335 | G | A | 12 | 1899291 | T | A | 12 | 3379076 | G | A |
| 12 | 3567611 | T | C | 12 | 4445722 | G | A | 12 | 4553184 | C | T |
| 12 | 5082261 | A | G | 12 | 5082274 | C | T | 12 | 5082302 | A | G |
| 12 | 5082835 | A | G | 12 | 5748011 | T | C | 12 | 5748052 | C | T |
| 12 | 5748078 | A | G | 12 | 5748079 | A | G | 12 | 6697842 | A | G |
| 12 | 6704213 | T | A | 12 | 7808545 | A | G | 12 | 8387026 | G | A |
| 12 | 8387543 | C | T | 12 | 8391144 | C | G | 12 | 8391162 | T | C |
| 12 | 8391761 | C | T | 12 | 8393255 | G | A | 12 | 8393268 | A | G |
| 12 | 8393274 | T | C | 12 | 8393314 | G | A | 12 | 8393803 | C | T |
| 12 | 8544062 | A | G | 12 | 8544083 | G | A | 12 | 8544541 | C | T |
| 12 | 8550134 | C | T | 12 | 8552680 | T | C | 12 | 9451473 | G | A |
| 12 | 9451541 | C | A | 12 | 9451818 | T | C | 12 | 9585578 | C | T |
| 12 | 9585658 | A | G | 12 | 9848883 | A | G | 12 | 10854878 | A | G |
| 12 | 11174753 | G | A | 12 | 11174795 | T | G | 12 | 11201629 | G | A |
| 12 | 11230403 | T | C | 12 | 11230436 | G | A | 12 | 11230437 | A | C |
| 12 | 11230452 | C | A | 12 | 11230457 | C | G | 12 | 11230465 | C | T |
| 12 | 11230496 | G | A | 12 | 11230497 | G | A | 12 | 11230520 | G | A |
| 12 | 11230542 | A | C | 12 | 11230544 | C | T | 12 | 11230554 | G | A |
| 12 | 11230566 | G | A | 12 | 11230578 | T | C | 12 | 11230583 | T | C |
| 12 | 11230592 | C | G | 12 | 11230597 | G | A | 12 | 11230598 | C | T |
| 12 | 11230601 | C | T | 12 | 11230609 | G | A | 12 | 11230636 | T | G |
| 12 | 11230662 | C | T | 12 | 11230665 | G | A | 12 | 11230700 | G | C |
| 12 | 11230701 | G | A | 12 | 11230712 | C | T | 12 | 11230726 | C | T |
| 12 | 11230744 | A | T | 12 | 11230752 | T | A | 12 | 11230754 | C | T |
| 12 | 11230775 | A | C | 12 | 11230778 | A | C | 12 | 11230832 | A | C |
| 12 | 11230834 | T | C | 12 | 11230867 | T | A | 12 | 11230873 | T | G |
| 12 | 11230890 | C | G | 12 | 11230895 | A | T | 12 | 11230909 | A | G |
| 12 | 11230936 | A | T | 12 | 11230948 | A | G | 12 | 11230968 | A | G |
| 12 | 11231002 | C | A | 12 | 11231008 | T | C | 12 | 11231018 | G | A |
| 12 | 11231042 | A | G | 12 | 11231052 | G | A | 12 | 11611304 | G | A |
| 12 | 11611331 | C | T | 12 | 11611336 | G | A | 12 | 11611368 | C | T |
| 12 | 11611377 | T | C | 12 | 12027745 | A | C | 12 | 13370236 | A | C |
| 12 | 13555619 | G | T | 12 | 13555639 | C | T | 12 | 13556252 | G | C |
| 12 | 15138281 | A | G | 12 | 16397411 | A | G | 12 | 17757557 | C | A |
| 12 | 20177190 | C | T | 12 | 21616558 | G | A | 12 | 21616598 | C | T |
| 12 | 21617144 | A | G | 12 | 21617145 | A | G | 12 | 22193228 | G | A |
| 12 | 22957461 | A | G | 12 | 22957519 | G | A | 12 | 23192806 | T | C |
| 12 | 24358865 | C | T | 12 | 25363085 | C | T | 12 | 25368084 | T | C |
| 12 | 26504062 | C | G | 12 | 29688497 | T | C | 12 | 30359821 | T | C |
| 12 | 31242271 | G | A | 12 | 31508019 | G | C | 12 | 32741558 | A | G |
| 12 | 33074174 | A | G | 12 | 33074256 | T | G | 12 | 37971347 | G | C |
| 12 | 38600609 | G | A | 12 | 40879387 | A | G | 12 | 40880558 | A | T |
| 12 | 40880586 | A | G | 12 | 40880587 | T | A | 12 | 40880831 | A | T |
| 12 | 40880924 | A | G | 12 | 40881916 | T | A | 12 | 40881964 | G | A |
| 12 | 40881967 | C | T | 12 | 40881986 | G | A | 12 | 40882000 | C | G |
| 12 | 40882006 | T | C | 12 | 40882016 | G | T | 12 | 40882026 | A | G |
| 12 | 40882071 | C | G | 12 | 40882121 | G | T | 12 | 40882450 | G | C |
| 12 | 40882490 | A | G | 12 | 40882522 | C | A | 12 | 40882649 | G | T |
| 12 | 40882701 | G | T | 12 | 40883092 | C | A | 12 | 40883745 | C | A |
| 12 | 40883775 | C | T | 12 | 40883781 | G | A | 12 | 40883786 | T | A |
| 12 | 40883904 | G | A | 12 | 40884061 | C | T | 12 | 42477389 | T | A |
| 12 | 42477390 | A | C | 12 | 42477397 | A | G | 12 | 42477402 | T | C |
| 12 | 42477404 | T | C | 12 | 42477426 | A | G | 12 | 42477431 | C | T |
| 12 | 42477930 | C | T | 12 | 42477945 | A | G | 12 | 42477948 | T | G |
| 12 | 42477951 | T | C | 12 | 42477952 | G | A | 12 | 42477962 | C | T |
| 12 | 42477979 | G | C | 12 | 42478001 | T | G | 12 | 43965011 | A | T |
| 12 | 43965108 | T | A | 12 | 45228464 | C | T | 12 | 46665157 | G | A |
| 12 | 46955423 | C | T | 12 | 51213765 | T | C | 12 | 52698224 | C | T |
| 12 | 52698226 | A | G | 12 | 52698228 | T | G | 12 | 52698230 | C | T |
| 12 | 52698235 | G | T | 12 | 52698236 | C | T | 12 | 53346750 | C | T |
| 12 | 53512756 | A | T | 12 | 53512770 | G | T | 12 | 54087418 | C | T |
| 12 | 54476772 | G | A | 12 | 54501012 | G | C | 12 | 55828540 | C | T |
| 12 | 56473808 | T | C | 12 | 56474480 | A | G | 12 | 56478002 | A | C |
| 12 | 56481118 | G | A | 12 | 56488913 | G | A | 12 | 57489828 | C | T |
| 12 | 57544620 | C | T | 12 | 58141602 | C | T | 12 | 60103232 | G | A |
| 12 | 60553810 | C | T | 12 | 61969925 | T | C | 12 | 62524923 | A | G |
| 12 | 62525409 | A | T | 12 | 63503650 | T | G | 12 | 64798118 | C | G |
| 12 | 68551409 | C | T | 12 | 68946720 | C | T | 12 | 69202580 | T | G |
| 12 | 69203312 | G | A | 12 | 69207162 | G | A | 12 | 69222357 | C | A |
| 12 | 69230189 | G | T | 12 | 70074807 | G | C | 12 | 71520477 | A | C |
| 12 | 71520600 | T | C | 12 | 71521068 | G | T | 12 | 72372601 | C | T |
| 12 | 72979069 | T | A | 12 | 72979099 | T | G | 12 | 73501997 | T | G |
| 12 | 76633769 | T | C | 12 | 77056710 | A | G | 12 | 79575315 | T | A |
| 12 | 79575325 | C | A | 12 | 82670236 | C | T | 12 | 83471875 | G | A |
| 12 | 83545935 | C | T | 12 | 84356493 | G | A | 12 | 85165573 | A | T |
| 12 | 86084416 | G | T | 12 | 87490519 | G | T | 12 | 87490552 | T | C |
| 12 | 88925973 | T | C | 12 | 88939790 | T | C | 12 | 88973732 | A | G |
| 12 | 88974212 | A | G | 12 | 89490962 | T | C | 12 | 89491013 | G | A |
| 12 | 89864432 | T | A | 12 | 90270430 | G | C | 12 | 91851148 | T | A |
| 12 | 92997562 | C | A | 12 | 94372056 | C | G | 12 | 95012735 | A | G |
| 12 | 95602672 | C | T | 12 | 96161299 | T | C | 12 | 96161309 | G | T |
| 12 | 96161410 | A | T | 12 | 96161868 | G | C | 12 | 96161887 | G | C |
| 12 | 96848128 | C | T | 12 | 96848651 | C | T | 12 | 96848682 | A | G |
| 12 | 97799632 | G | A | 12 | 97799634 | C | T | 12 | 97800208 | T | C |
| 12 | 97800239 | C | T | 12 | 98984651 | A | G | 12 | 98985056 | T | C |
| 12 | 99265981 | A | G | 12 | 99266022 | A | T | 12 | 101215628 | G | A |
| 12 | 102361086 | T | A | 12 | 104959816 | T | A | 12 | 105079958 | T | C |
| 12 | 105673808 | G | A | 12 | 106114632 | G | A | 12 | 106620432 | A | G |
| 12 | 107381892 | C | T | 12 | 108168706 | G | A | 12 | 109330915 | G | A |
| 12 | 112241402 | G | A | 12 | 112843998 | A | T | 12 | 112856643 | G | A |
| 12 | 112890776 | A | G | 12 | 112942353 | G | A | 12 | 114501664 | T | C |
| 12 | 114501692 | T | C | 12 | 114501693 | G | A | 12 | 114501708 | T | A |
| 12 | 114501726 | C | T | 12 | 114501852 | A | T | 12 | 114501869 | A | C |
| 12 | 114501990 | A | G | 12 | 114502105 | T | A | 12 | 114502125 | T | C |
| 12 | 114502197 | T | C | 12 | 114502242 | A | G | 12 | 114502276 | A | C |
| 12 | 114502702 | G | T | 12 | 115184053 | G | C | 12 | 115866519 | G | T |
| 12 | 116050988 | C | T | 12 | 117193050 | T | C | 12 | 117692072 | T | C |
| 12 | 118256945 | G | C | 12 | 118256995 | T | A | 12 | 121426478 | C | T |
| 12 | 121433816 | C | T | 12 | 121433844 | A | G | 12 | 121434833 | G | A |
| 12 | 121876257 | G | T | 12 | 121876385 | G | C | 12 | 121876866 | G | A |
| 12 | 122849392 | A | C | 12 | 123186539 | A | G | 12 | 123186610 | G | A |
| 12 | 123186611 | C | G | 12 | 123199745 | T | C | 12 | 123199755 | A | G |
| 12 | 124804371 | G | C | 12 | 124804373 | C | T | 12 | 124804522 | A | C |
| 12 | 124804538 | G | A | 12 | 124804550 | C | A | 12 | 124804566 | G | A |
| 12 | 124804578 | C | A | 12 | 124804593 | G | C | 12 | 124804644 | A | G |
| 12 | 125177433 | C | T | 12 | 125177454 | G | A | 12 | 125177986 | C | T |
| 12 | 127490326 | A | C | 12 | 128958147 | A | G | 12 | 129299134 | T | C |
| 12 | 130876953 | T | C | 12 | 130876961 | G | T | 12 | 130876987 | T | C |
| 12 | 130877140 | G | T | 12 | 130877166 | T | C | 12 | 130877287 | G | A |
| 12 | 130877325 | A | G | 12 | 130877332 | G | A | 12 | 130877408 | C | T |
| 12 | 130877506 | A | G | 12 | 130877521 | G | A | 12 | 131017991 | C | G |
| 12 | 131966057 | T | C | 12 | 131966293 | C | T | 12 | 132286104 | C | T |
| 12 | 132286105 | G | A | 12 | 132286113 | T | C | 12 | 132286133 | G | A |
| 12 | 132957443 | T | C | 12 | 132958490 | A | G | 12 | 132959437 | G | A |
| 12 | 132959439 | G | A | 12 | 132959472 | T | A | 12 | 132959689 | C | T |
| 12 | 132959693 | T | C | 12 | 132959732 | T | C | 12 | 132961351 | G | T |
| 12 | 133201947 | T | C | 12 | 133208611 | A | G | 12 | 133209593 | T | C |
| 12 | 133209653 | C | T | 12 | 133209660 | G | A | 12 | 133212165 | A | G |
| 12 | 133235577 | A | C | 12 | 133241144 | C | A | 12 | 133243788 | T | A |
| 12 | 133243817 | C | T | 12 | 133250569 | T | G | 12 | 133250571 | C | T |
| 12 | 133254529 | C | G | 12 | 133255792 | A | C | 12 | 133258088 | C | T |
| 12 | 133818115 | C | T | 12 | 133818696 | G | T | 13 | 19098169 | G | A |
| 13 | 19098189 | T | C | 13 | 19196854 | T | C | 13 | 19196952 | A | G |
| 13 | 19196957 | A | G | 13 | 19196999 | A | G | 13 | 19197003 | T | C |
| 13 | 19197028 | A | G | 13 | 19197050 | A | C | 13 | 19197108 | G | T |
| 13 | 19197161 | T | C | 13 | 19197162 | G | A | 13 | 19312986 | G | A |
| 13 | 19312997 | T | C | 13 | 20006231 | A | C | 13 | 21517666 | C | T |
| 13 | 21535954 | C | G | 13 | 22638746 | G | A | 13 | 23269969 | T | A |
| 13 | 23516337 | C | T | 13 | 23533834 | T | G | 13 | 23534184 | T | C |
| 13 | 23534203 | G | A | 13 | 23534230 | C | G | 13 | 23534298 | G | A |
| 13 | 24958840 | C | G | 13 | 25244216 | C | G | 13 | 25244691 | C | T |
| 13 | 25535764 | C | G | 13 | 25535765 | A | G | 13 | 25535868 | T | C |
| 13 | 26923565 | T | G | 13 | 26974482 | T | G | 13 | 27487175 | G | A |
| 13 | 27487180 | C | T | 13 | 27827811 | C | T | 13 | 27945465 | T | G |
| 13 | 28597187 | C | T | 13 | 28601651 | T | C | 13 | 28602604 | T | C |
| 13 | 28609396 | T | C | 13 | 28609399 | G | C | 13 | 28610355 | T | A |
| 13 | 28611630 | T | C | 13 | 28644359 | G | C | 13 | 28886388 | A | G |
| 13 | 28893341 | G | C | 13 | 28893870 | C | G | 13 | 28896097 | G | A |
| 13 | 28919330 | G | A | 13 | 29007730 | A | G | 13 | 30015342 | A | G |
| 13 | 30015805 | T | C | 13 | 30888324 | T | A | 13 | 31276032 | G | C |
| 13 | 31276586 | G | T | 13 | 32421423 | C | T | 13 | 32899567 | G | C |
| 13 | 32900933 | T | A | 13 | 32907767 | C | T | 13 | 32921306 | G | A |
| 13 | 32921362 | T | C | 13 | 32945537 | A | T | 13 | 32953182 | A | G |
| 13 | 32968591 | G | A | 13 | 32968607 | A | G | 13 | 34723653 | T | G |
| 13 | 34723667 | C | T | 13 | 34724105 | C | T | 13 | 34724194 | C | T |
| 13 | 36108037 | A | C | 13 | 37377469 | T | A | 13 | 40733509 | C | A |
| 13 | 40733531 | A | T | 13 | 42300323 | T | C | 13 | 43517113 | C | T |
| 13 | 43517127 | A | G | 13 | 43517700 | G | A | 13 | 43517736 | G | T |
| 13 | 44772757 | A | G | 13 | 45626937 | C | T | 13 | 47199611 | T | C |
| 13 | 48273085 | A | G | 13 | 48881154 | G | C | 13 | 48919064 | A | G |
| 13 | 48934575 | A | T | 13 | 48954021 | G | C | 13 | 50466615 | G | C |
| 13 | 50466749 | C | G | 13 | 50466754 | T | C | 13 | 50466766 | C | T |
| 13 | 50466771 | A | G | 13 | 50466774 | G | A | 13 | 50466780 | C | T |
| 13 | 50466781 | A | G | 13 | 50466783 | C | G | 13 | 50935593 | C | T |
| 13 | 51230621 | A | G | 13 | 51231117 | C | T | 13 | 51231132 | G | C |
| 13 | 51685479 | C | T | 13 | 51685541 | C | T | 13 | 51686109 | G | A |
| 13 | 52482451 | G | A | 13 | 52602919 | A | C | 13 | 60909992 | G | A |
| 13 | 61266375 | T | C | 13 | 61651695 | T | A | 13 | 61652128 | T | C |
| 13 | 62884880 | A | G | 13 | 62885397 | T | C | 13 | 62885404 | C | G |
| 13 | 63984445 | G | A | 13 | 63984474 | C | A | 13 | 63984474 | C | T |
| 13 | 63984519 | T | C | 13 | 68406302 | A | G | 13 | 68406306 | A | G |
| 13 | 68406761 | G | A | 13 | 68820749 | C | T | 13 | 68820783 | A | G |
| 13 | 69675780 | G | A | 13 | 69675935 | T | C | 13 | 70884365 | C | G |
| 13 | 70884372 | T | C | 13 | 70884412 | G | A | 13 | 70884937 | T | C |
| 13 | 70885012 | C | T | 13 | 71158022 | T | C | 13 | 71498321 | T | C |
| 13 | 71498338 | C | T | 13 | 71498343 | G | T | 13 | 71498360 | G | C |
| 13 | 71498385 | C | T | 13 | 71498894 | A | G | 13 | 72477265 | C | T |
| 13 | 74077481 | C | T | 13 | 74549292 | T | C | 13 | 76333853 | G | A |
| 13 | 76583319 | A | T | 13 | 77614202 | G | A | 13 | 77614206 | A | T |
| 13 | 77614213 | C | T | 13 | 78800795 | G | A | 13 | 79015424 | G | A |
| 13 | 79775160 | C | T | 13 | 80384592 | G | A | 13 | 80384594 | A | T |
| 13 | 82485701 | G | A | 13 | 82641070 | G | T | 13 | 83720107 | C | G |
| 13 | 85714107 | T | C | 13 | 87209806 | G | T | 13 | 88275166 | G | T |
| 13 | 90617118 | T | C | 13 | 90617599 | C | T | 13 | 91023515 | C | A |
| 13 | 91779179 | A | C | 13 | 93036880 | A | C | 13 | 94062095 | C | A |
| 13 | 94996400 | G | A | 13 | 95538615 | C | G | 13 | 97843293 | G | T |
| 13 | 98419205 | T | C | 13 | 98522674 | A | T | 13 | 99464351 | T | A |
| 13 | 100477134 | C | A | 13 | 100477148 | A | G | 13 | 100545068 | C | T |
| 13 | 101182148 | A | G | 13 | 101987034 | A | C | 13 | 101987047 | A | G |
| 13 | 101987123 | A | G | 13 | 102057883 | A | C | 13 | 102058361 | G | A |
| 13 | 103498375 | T | C | 13 | 103520251 | T | C | 13 | 107853969 | G | T |
| 13 | 109006433 | A | G | 13 | 109006441 | T | C | 13 | 110418135 | C | T |
| 13 | 110418136 | G | A | 13 | 110419404 | C | T | 13 | 110976837 | A | C |
| 13 | 111132947 | G | A | 13 | 111133395 | C | T | 13 | 111133399 | A | G |
| 13 | 111133489 | A | G | 13 | 111133579 | G | A | 13 | 111133625 | T | A |
| 13 | 111133639 | G | A | 13 | 111133665 | C | T | 13 | 111133669 | A | G |
| 13 | 111133835 | A | T | 13 | 111134015 | A | T | 13 | 111134119 | A | G |
| 13 | 111589152 | G | A | 13 | 111589204 | G | C | 13 | 111589278 | G | A |
| 13 | 111590028 | A | G | 13 | 111590550 | A | G | 13 | 111590930 | C | T |
| 13 | 111591170 | G | A | 13 | 111591265 | G | A | 13 | 111591283 | T | C |
| 13 | 111591853 | G | A | 13 | 111592285 | A | G | 13 | 112802383 | G | A |
| 13 | 112802389 | G | A | 13 | 112803722 | T | C | 13 | 112803822 | G | C |
| 13 | 112803840 | A | T | 13 | 112803842 | A | G | 13 | 112803853 | T | G |
| 13 | 113498860 | C | T | 13 | 114089046 | C | T | 13 | 114089082 | C | T |
| 13 | 114089113 | A | G | 13 | 114089128 | T | C | 13 | 114089164 | C | T |
| 13 | 114089330 | G | A | 13 | 114089440 | G | C | 13 | 114089675 | G | T |
| 13 | 114089876 | A | G | 13 | 114089888 | T | C | 13 | 114089891 | A | G |
| 13 | 114089897 | C | T | 13 | 114090025 | C | T | 13 | 114090109 | C | T |
| 13 | 114090253 | T | C | 13 | 114090689 | G | A | 13 | 114091321 | T | C |
| 13 | 114838339 | C | T | 14 | 19491761 | T | C | 14 | 19629734 | C | G |
| 14 | 19688223 | G | A | 14 | 19688480 | G | C | 14 | 20084306 | C | T |
| 14 | 20084444 | G | C | 14 | 20086576 | G | A | 14 | 20140628 | C | G |
| 14 | 20140803 | G | C | 14 | 20140905 | C | T | 14 | 20414407 | C | A |
| 14 | 20414410 | T | C | 14 | 20414737 | C | T | 14 | 20414884 | G | A |
| 14 | 20811577 | A | C | 14 | 20811577 | A | G | 14 | 20811578 | A | G |
| 14 | 20811588 | T | C | 14 | 20812951 | G | A | 14 | 20815313 | A | G |
| 14 | 20822772 | C | T | 14 | 20826243 | A | C | 14 | 21236526 | T | C |
| 14 | 21237137 | G | A | 14 | 21630781 | C | T | 14 | 21860360 | G | A |
| 14 | 21863716 | C | T | 14 | 21871999 | T | G | 14 | 21875487 | T | C |
| 14 | 21894526 | T | C | 14 | 22320158 | C | A | 14 | 22320728 | T | C |
| 14 | 22371221 | G | T | 14 | 22371246 | A | G | 14 | 22746792 | C | T |
| 14 | 23029407 | T | C | 14 | 23029409 | T | C | 14 | 23029512 | G | C |
| 14 | 23029525 | A | T | 14 | 23652708 | T | A | 14 | 24436511 | T | C |
| 14 | 24436706 | G | C | 14 | 24436798 | G | T | 14 | 24473786 | C | A |
| 14 | 24526287 | G | C | 14 | 24526895 | G | A | 14 | 25515815 | A | G |
| 14 | 25515822 | T | C | 14 | 26605655 | T | C | 14 | 27314804 | C | T |
| 14 | 27855023 | T | C | 14 | 27855025 | C | T | 14 | 31919287 | T | C |
| 14 | 36351085 | G | A | 14 | 36417424 | G | A | 14 | 36417426 | T | A |
| 14 | 36417451 | G | A | 14 | 36417453 | T | A | 14 | 36417478 | A | G |
| 14 | 36417480 | A | T | 14 | 36417498 | T | C | 14 | 36417545 | C | T |
| 14 | 36417546 | A | G | 14 | 36417559 | G | A | 14 | 36417572 | T | C |
| 14 | 36417640 | G | A | 14 | 36417696 | A | T | 14 | 36417716 | T | C |
| 14 | 36417723 | A | T | 14 | 36417741 | T | C | 14 | 36417743 | T | C |
| 14 | 36417761 | C | T | 14 | 36417777 | A | T | 14 | 36417802 | A | G |
| 14 | 36417804 | A | T | 14 | 36417842 | T | C | 14 | 36417883 | A | G |
| 14 | 36417957 | T | C | 14 | 36417959 | T | C | 14 | 36418031 | T | C |
| 14 | 36418355 | T | C | 14 | 36419122 | C | A | 14 | 36419133 | T | G |
| 14 | 36989596 | A | G | 14 | 36989696 | G | T | 14 | 38639257 | C | T |
| 14 | 39354900 | C | G | 14 | 39858941 | A | G | 14 | 41413712 | C | T |
| 14 | 42835190 | G | A | 14 | 45027200 | G | T | 14 | 46316764 | A | G |
| 14 | 47492399 | C | G | 14 | 47562483 | T | A | 14 | 48921178 | T | C |
| 14 | 50520782 | A | G | 14 | 52229607 | C | G | 14 | 52229678 | T | C |
| 14 | 52230158 | T | G | 14 | 52519254 | A | G | 14 | 52519839 | G | A |
| 14 | 55880730 | A | G | 14 | 55880918 | A | G | 14 | 55921280 | A | G |
| 14 | 56232970 | T | C | 14 | 56233561 | G | A | 14 | 56548918 | C | A |
| 14 | 57463704 | G | A | 14 | 57586697 | C | T | 14 | 57586731 | T | G |
| 14 | 59170024 | C | G | 14 | 62003902 | C | T | 14 | 62983672 | A | T |
| 14 | 65473196 | G | A | 14 | 65568563 | C | T | 14 | 65733329 | A | T |
| 14 | 65733701 | A | G | 14 | 65734110 | C | G | 14 | 65734181 | C | G |
| 14 | 65734600 | G | A | 14 | 65934921 | T | C | 14 | 68291907 | G | A |
| 14 | 68352323 | A | G | 14 | 68354152 | C | T | 14 | 68758317 | T | C |
| 14 | 68934690 | A | G | 14 | 68944147 | T | G | 14 | 68944245 | T | C |
| 14 | 70440081 | T | C | 14 | 71632622 | C | T | 14 | 71632640 | C | T |
| 14 | 72266238 | T | A | 14 | 72736899 | G | A | 14 | 73047284 | A | G |
| 14 | 73997686 | G | A | 14 | 74025873 | A | T | 14 | 75506875 | G | A |
| 14 | 77101088 | A | G | 14 | 77704974 | G | C | 14 | 78149486 | T | C |
| 14 | 78149563 | G | C | 14 | 79217473 | C | T | 14 | 80002042 | A | G |
| 14 | 80002096 | T | C | 14 | 80002632 | A | G | 14 | 80874274 | A | T |
| 14 | 81554617 | A | G | 14 | 81558640 | C | G | 14 | 81559221 | G | A |
| 14 | 81562808 | A | G | 14 | 81606422 | G | T | 14 | 81606503 | T | C |
| 14 | 81610869 | C | A | 14 | 81610942 | C | T | 14 | 82053802 | T | C |
| 14 | 82054300 | G | T | 14 | 82602590 | C | T | 14 | 82603182 | C | T |
| 14 | 86166491 | A | G | 14 | 88199938 | T | C | 14 | 91507280 | T | C |
| 14 | 94339436 | C | G | 14 | 94845040 | G | A | 14 | 95389462 | A | G |
| 14 | 95582544 | A | C | 14 | 95600006 | G | C | 14 | 95600072 | T | G |
| 14 | 96092282 | C | T | 14 | 96592735 | C | G | 14 | 97107413 | G | A |
| 14 | 98485583 | T | C | 14 | 98486120 | G | A | 14 | 98868717 | G | A |
| 14 | 100595278 | T | A | 14 | 100595880 | G | A | 14 | 101134984 | G | A |
| 14 | 103050106 | G | A | 14 | 103050397 | C | G | 14 | 103840494 | G | A |
| 14 | 104168701 | C | T | 14 | 104563874 | C | T | 14 | 104563932 | G | T |
| 14 | 104564283 | T | C | 14 | 104564345 | G | A | 14 | 104564410 | T | C |
| 14 | 104564412 | G | A | 14 | 104945518 | C | G | 14 | 104945694 | A | T |
| 14 | 105243435 | G | T | 14 | 105246872 | A | G | 14 | 106049449 | T | C |
| 14 | 106167913 | T | A | 14 | 106725276 | G | A | 14 | 106725315 | G | A |
| 14 | 106725329 | C | T | 14 | 106725339 | A | G | 14 | 106725346 | G | A |
| 14 | 106725515 | A | C | 14 | 106725525 | C | A | 14 | 106725526 | T | A |
| 14 | 106725543 | A | T | 14 | 107046720 | T | A | 14 | 107048702 | G | A |
| 14 | 107048749 | C | G | 14 | 107048929 | T | C | 14 | 107112165 | C | G |
| 14 | 107112665 | A | C | 14 | 107130943 | G | A | 14 | 107130971 | C | T |
| 14 | 107131063 | A | G | 14 | 107131164 | A | C | 14 | 107131246 | C | T |
| 14 | 107131290 | T | C | 14 | 107131300 | T | C | 14 | 107131301 | C | G |
| 14 | 107131349 | A | G | 14 | 107131357 | T | C | 14 | 107131410 | A | G |
| 15 | 20148695 | G | T | 15 | 20148878 | C | A | 15 | 20319405 | C | T |
| 15 | 20319448 | T | C | 15 | 20543982 | T | C | 15 | 20543988 | A | G |
| 15 | 20635860 | C | T | 15 | 20891413 | A | C | 15 | 20891478 | C | T |
| 15 | 20891489 | T | C | 15 | 20891547 | C | T | 15 | 20891566 | C | A |
| 15 | 20891566 | C | G | 15 | 20891651 | A | G | 15 | 20891867 | G | T |
| 15 | 20891893 | C | A | 15 | 20966938 | G | T | 15 | 20967029 | A | G |
| 15 | 21136727 | T | A | 15 | 21136951 | G | A | 15 | 21136968 | C | T |
| 15 | 21137321 | C | A | 15 | 21325716 | C | T | 15 | 21325857 | T | C |
| 15 | 21326120 | C | T | 15 | 21326788 | C | G | 15 | 21966878 | C | T |
| 15 | 21973528 | C | T | 15 | 21973529 | A | G | 15 | 21973572 | T | C |
| 15 | 21973587 | T | C | 15 | 21973588 | A | G | 15 | 21973592 | G | T |
| 15 | 21973797 | T | C | 15 | 21973842 | A | C | 15 | 21973843 | G | A |
| 15 | 22148127 | C | T | 15 | 22148775 | A | G | 15 | 22148845 | A | G |
| 15 | 22151298 | C | T | 15 | 22151704 | A | G | 15 | 22151876 | C | T |
| 15 | 22822510 | G | C | 15 | 22822568 | T | G | 15 | 22822751 | C | G |
| 15 | 22822755 | G | A | 15 | 22822854 | T | A | 15 | 22822955 | G | T |
| 15 | 22823001 | T | A | 15 | 22823078 | A | G | 15 | 22823113 | T | C |
| 15 | 22823116 | C | A | 15 | 22823137 | A | C | 15 | 22823185 | T | G |
| 15 | 22823255 | A | C | 15 | 22823692 | T | C | 15 | 22823783 | G | T |
| 15 | 22823838 | C | A | 15 | 22823855 | A | G | 15 | 22823949 | C | A |
| 15 | 22823963 | T | C | 15 | 22824021 | T | G | 15 | 22824030 | C | A |
| 15 | 22824086 | G | A | 15 | 22824376 | A | G | 15 | 22826375 | T | C |
| 15 | 22826634 | A | C | 15 | 22826679 | C | T | 15 | 22826764 | T | C |
| 15 | 22827002 | G | C | 15 | 22827233 | A | G | 15 | 22827239 | G | C |
| 15 | 22827251 | A | C | 15 | 22827293 | C | A | 15 | 22827319 | G | T |
| 15 | 22827322 | A | T | 15 | 22827437 | A | G | 15 | 22827816 | A | C |
| 15 | 22827928 | T | G | 15 | 22827930 | C | A | 15 | 22827939 | T | C |
| 15 | 22827984 | G | C | 15 | 22827985 | G | A | 15 | 22828037 | G | A |
| 15 | 22828041 | A | G | 15 | 22828139 | A | T | 15 | 22828145 | T | C |
| 15 | 22828147 | T | C | 15 | 22828275 | G | C | 15 | 22828390 | A | T |
| 15 | 22828465 | A | C | 15 | 22829674 | C | G | 15 | 22829687 | G | T |
| 15 | 22829695 | C | T | 15 | 22830185 | T | C | 15 | 22830340 | C | T |
| 15 | 22830453 | G | A | 15 | 22830504 | T | A | 15 | 22830510 | G | T |
| 15 | 22830571 | C | T | 15 | 22830577 | A | G | 15 | 22830592 | G | T |
| 15 | 22830847 | C | G | 15 | 22830891 | T | C | 15 | 22830899 | G | T |
| 15 | 22830903 | G | C | 15 | 22830990 | C | T | 15 | 22830999 | C | T |
| 15 | 22832977 | A | G | 15 | 23782545 | T | C | 15 | 24059444 | T | A |
| 15 | 24059493 | A | T | 15 | 24060123 | T | G | 15 | 24060127 | G | A |
| 15 | 24506371 | C | A | 15 | 24506572 | C | T | 15 | 24672192 | G | T |
| 15 | 24672267 | G | C | 15 | 24672440 | G | C | 15 | 24672460 | A | C |
| 15 | 24672460 | A | T | 15 | 24672504 | G | C | 15 | 24672554 | G | T |
| 15 | 24672804 | T | A | 15 | 24722807 | G | T | 15 | 24722823 | G | C |
| 15 | 24722829 | G | A | 15 | 25414653 | C | T | 15 | 26273690 | G | A |
| 15 | 26274261 | A | G | 15 | 26600681 | A | G | 15 | 26601246 | G | A |
| 15 | 26601264 | G | A | 15 | 29717391 | T | C | 15 | 31490709 | A | C |
| 15 | 31821275 | G | A | 15 | 32535526 | G | A | 15 | 34638030 | C | T |
| 15 | 34639823 | A | G | 15 | 34639826 | A | G | 15 | 34639879 | C | G |
| 15 | 34639904 | G | A | 15 | 35147136 | C | A | 15 | 35147592 | G | A |
| 15 | 35384908 | C | A | 15 | 37292823 | G | A | 15 | 37292836 | G | A |
| 15 | 38190654 | G | T | 15 | 38545725 | A | C | 15 | 38617282 | A | G |
| 15 | 38642971 | G | C | 15 | 38643009 | G | A | 15 | 38643031 | C | T |
| 15 | 39034864 | C | T | 15 | 39034914 | G | A | 15 | 40457042 | A | C |
| 15 | 40457635 | C | T | 15 | 40998305 | T | A | 15 | 41011311 | G | A |
| 15 | 41021280 | C | G | 15 | 42265586 | G | A | 15 | 43853068 | C | A |
| 15 | 43853127 | C | T | 15 | 43853145 | G | A | 15 | 43853167 | T | C |
| 15 | 43853196 | C | G | 15 | 43853216 | T | A | 15 | 45985719 | G | C |
| 15 | 47150907 | T | C | 15 | 49220457 | A | G | 15 | 49615149 | A | G |
| 15 | 51483996 | G | C | 15 | 53022863 | A | C | 15 | 53023345 | C | T |
| 15 | 53178383 | C | G | 15 | 54607264 | T | C | 15 | 55099758 | G | T |
| 15 | 59722664 | C | T | 15 | 59844728 | A | G | 15 | 59845138 | G | A |
| 15 | 62046728 | T | C | 15 | 66736741 | C | T | 15 | 66774559 | A | G |
| 15 | 67458930 | C | T | 15 | 67476735 | G | A | 15 | 67476756 | C | T |
| 15 | 67476768 | C | T | 15 | 67477450 | C | T | 15 | 68123700 | C | T |
| 15 | 69383465 | T | A | 15 | 69676000 | C | A | 15 | 70292710 | C | T |
| 15 | 70573650 | C | A | 15 | 71967893 | G | A | 15 | 74460734 | C | T |
| 15 | 74654146 | T | C | 15 | 76001037 | C | T | 15 | 76039262 | T | C |
| 15 | 78206482 | C | T | 15 | 78206514 | A | T | 15 | 78206928 | G | A |
| 15 | 78206970 | G | T | 15 | 78206973 | C | A | 15 | 78207027 | A | G |
| 15 | 79032355 | T | C | 15 | 79032469 | C | T | 15 | 79155412 | T | C |
| 15 | 79526411 | G | T | 15 | 80012735 | A | G | 15 | 81410106 | G | A |
| 15 | 82636963 | G | A | 15 | 82637357 | T | C | 15 | 83331487 | T | C |
| 15 | 83569832 | C | G | 15 | 84143244 | G | A | 15 | 84857013 | C | G |
| 15 | 84909385 | G | A | 15 | 84951508 | C | A | 15 | 84951578 | G | C |
| 15 | 85052414 | C | T | 15 | 85495400 | C | T | 15 | 85495950 | C | T |
| 15 | 85788238 | G | A | 15 | 85788607 | C | T | 15 | 85788735 | T | C |
| 15 | 86855520 | G | A | 15 | 88428702 | C | A | 15 | 88472170 | A | G |
| 15 | 88483594 | A | G | 15 | 88680100 | G | A | 15 | 88680987 | T | A |
| 15 | 88690951 | C | T | 15 | 88726405 | G | A | 15 | 88727751 | A | T |
| 15 | 89801677 | G | C | 15 | 89811482 | T | C | 15 | 89821732 | G | A |
| 15 | 89824258 | A | C | 15 | 89833165 | T | C | 15 | 89836793 | C | A |
| 15 | 89843950 | A | T | 15 | 89855998 | C | T | 15 | 89859377 | A | G |
| 15 | 90628777 | T | C | 15 | 90631247 | G | A | 15 | 90633416 | A | G |
| 15 | 90645265 | G | A | 15 | 91290366 | G | C | 15 | 91337208 | G | A |
| 15 | 91347135 | C | A | 15 | 91347755 | A | G | 15 | 91347784 | C | T |
| 15 | 91352774 | A | G | 15 | 91969961 | G | C | 15 | 91969970 | T | G |
| 15 | 91969976 | C | G | 15 | 92868324 | A | G | 15 | 94208914 | A | C |
| 15 | 95270619 | C | T | 15 | 95270626 | T | C | 15 | 95271164 | T | A |
| 15 | 95662895 | T | A | 15 | 96479346 | C | T | 15 | 96705724 | T | C |
| 15 | 97586395 | C | A | 15 | 98419797 | C | A | 15 | 98419885 | G | C |
| 15 | 99434357 | G | A | 15 | 99443139 | G | A | 15 | 99443161 | A | G |
| 15 | 99454245 | A | G | 15 | 99454962 | C | T | 15 | 99454994 | G | A |
| 15 | 99458902 | C | T | 15 | 99482903 | C | T | 15 | 99485931 | T | A |
| 15 | 99491540 | G | A | 15 | 99500064 | C | T | 15 | 100285424 | T | G |
| 15 | 100333593 | G | A | 15 | 100339833 | G | C | 15 | 100339897 | T | C |
| 15 | 100339938 | C | T | 15 | 100340316 | A | C | 15 | 100340375 | A | G |
| 15 | 100340413 | C | T | 15 | 100340484 | A | G | 15 | 101110096 | T | C |
| 15 | 102296008 | G | C | 15 | 102296140 | C | T | 15 | 102296231 | C | T |
| 15 | 102300354 | T | C | 15 | 102301640 | T | C | 15 | 102301662 | A | C |
| 15 | 102301669 | G | C | 15 | 102303340 | A | G | 15 | 102303423 | A | G |
| 15 | 102303495 | C | A | 15 | 102303519 | G | T | 15 | 102304191 | G | C |
| 15 | 102305129 | T | C | 15 | 102305169 | G | T | 15 | 102517868 | G | A |
| 16 | 326817 | C | A | 16 | 326826 | C | G | 16 | 900140 | A | G |
| 16 | 1872922 | C | G | 16 | 1873332 | G | A | 16 | 1873372 | C | T |
| 16 | 2107489 | C | T | 16 | 3020860 | C | G | 16 | 3799857 | T | C |
| 16 | 3828348 | A | G | 16 | 4060980 | T | C | 16 | 4985044 | C | T |
| 16 | 5139217 | C | G | 16 | 5143432 | C | G | 16 | 5143976 | C | G |
| 16 | 5145474 | C | T | 16 | 5145847 | G | A | 16 | 5145848 | A | G |
| 16 | 5145883 | A | G | 16 | 5146062 | C | G | 16 | 5166093 | A | G |
| 16 | 5338275 | A | G | 16 | 5338313 | C | G | 16 | 5527601 | T | A |
| 16 | 6553772 | G | A | 16 | 6554204 | A | C | 16 | 6554253 | C | A |
| 16 | 6554268 | C | T | 16 | 7495752 | C | T | 16 | 7496375 | G | T |
| 16 | 8603974 | A | G | 16 | 8603995 | G | C | 16 | 8604458 | A | G |
| 16 | 8604472 | C | T | 16 | 8604479 | A | C | 16 | 8604481 | C | T |
| 16 | 8604503 | A | G | 16 | 8604517 | G | A | 16 | 8604540 | A | G |
| 16 | 9928329 | C | T | 16 | 9943995 | A | G | 16 | 10273536 | G | T |
| 16 | 10632828 | A | G | 16 | 12019830 | G | A | 16 | 12019883 | A | C |
| 16 | 12019895 | A | G | 16 | 12297011 | C | G | 16 | 14781854 | T | A |
| 16 | 14781862 | T | A | 16 | 14782377 | C | A | 16 | 14821494 | C | A |
| 16 | 14864463 | G | A | 16 | 14864910 | G | A | 16 | 15047046 | C | G |
| 16 | 15452301 | C | T | 16 | 15460786 | T | C | 16 | 15867652 | T | G |
| 16 | 16789081 | C | T | 16 | 16789406 | C | T | 16 | 18450847 | C | T |
| 16 | 18450961 | G | A | 16 | 18543753 | C | T | 16 | 19479406 | C | T |
| 16 | 20575093 | G | A | 16 | 20575307 | G | A | 16 | 21844766 | G | A |
| 16 | 21844944 | A | G | 16 | 21844958 | G | C | 16 | 23016538 | G | A |
| 16 | 23632479 | C | T | 16 | 23634026 | A | G | 16 | 23637251 | G | C |
| 16 | 23698547 | G | T | 16 | 24888454 | C | T | 16 | 25668649 | C | T |
| 16 | 25668663 | A | C | 16 | 26131607 | A | G | 16 | 26132278 | G | T |
| 16 | 26798205 | G | T | 16 | 27947757 | G | A | 16 | 28785263 | T | C |
| 16 | 28871657 | C | A | 16 | 29059935 | G | A | 16 | 29060011 | T | A |
| 16 | 29065054 | C | T | 16 | 29314400 | C | T | 16 | 29344359 | C | T |
| 16 | 29344604 | C | T | 16 | 29344690 | C | T | 16 | 29398813 | G | A |
| 16 | 31771741 | T | C | 16 | 31771776 | A | G | 16 | 31771780 | A | G |
| 16 | 31771902 | A | G | 16 | 31771903 | A | C | 16 | 31771904 | C | T |
| 16 | 31771907 | A | G | 16 | 31771911 | C | A | 16 | 31771914 | C | T |
| 16 | 31771943 | G | T | 16 | 31771944 | C | A | 16 | 31771949 | G | A |
| 16 | 31771953 | A | G | 16 | 31772070 | A | G | 16 | 32823016 | C | A |
| 16 | 32823158 | G | A | 16 | 32823526 | C | A | 16 | 33070217 | T | C |
| 16 | 48996185 | C | A | 16 | 48996189 | C | A | 16 | 48996630 | C | G |
| 16 | 50809902 | C | G | 16 | 50816660 | C | T | 16 | 52671770 | C | T |
| 16 | 53391457 | G | C | 16 | 53564401 | G | C | 16 | 55173502 | T | G |
| 16 | 55692499 | G | A | 16 | 56046208 | A | T | 16 | 57045159 | T | C |
| 16 | 57720488 | G | A | 16 | 57720563 | T | C | 16 | 57721127 | G | T |
| 16 | 57721146 | G | A | 16 | 58690676 | G | C | 16 | 58691267 | A | G |
| 16 | 61461212 | A | G | 16 | 62213143 | C | T | 16 | 63217610 | G | C |
| 16 | 65540174 | C | T | 16 | 68771034 | C | A | 16 | 68771122 | A | C |
| 16 | 68771547 | G | C | 16 | 68820718 | A | G | 16 | 68821271 | G | C |
| 16 | 68847044 | C | T | 16 | 68847712 | G | A | 16 | 68849904 | A | G |
| 16 | 68861864 | G | A | 16 | 68863314 | T | C | 16 | 70009172 | G | C |
| 16 | 70224457 | C | G | 16 | 70224541 | T | C | 16 | 70235983 | T | C |
| 16 | 71105150 | A | G | 16 | 72007232 | G | A | 16 | 72763955 | G | T |
| 16 | 72991057 | G | T | 16 | 73141028 | G | A | 16 | 74422136 | T | C |
| 16 | 74422332 | A | G | 16 | 74427116 | G | A | 16 | 76418228 | C | T |
| 16 | 76907109 | C | A | 16 | 77694411 | T | G | 16 | 77694421 | T | A |
| 16 | 78082862 | C | T | 16 | 78082906 | G | A | 16 | 78761364 | A | G |
| 16 | 78761917 | G | C | 16 | 79161532 | A | C | 16 | 79704152 | T | C |
| 16 | 81636255 | A | G | 16 | 81636851 | G | A | 16 | 82150582 | A | G |
| 16 | 82527841 | C | T | 16 | 83459041 | C | T | 16 | 83691187 | G | A |
| 16 | 85437616 | T | C | 16 | 85437625 | T | C | 16 | 85437629 | G | A |
| 16 | 85437709 | G | A | 16 | 85437722 | T | C | 16 | 85437742 | T | C |
| 16 | 85437752 | A | T | 16 | 85437996 | A | T | 16 | 85438220 | G | A |
| 16 | 85438556 | T | A | 16 | 85438649 | A | G | 16 | 85438680 | C | T |
| 16 | 85438700 | C | T | 16 | 85441380 | C | G | 16 | 85441405 | G | C |
| 16 | 85441424 | T | C | 16 | 85441812 | A | C | 16 | 85442570 | T | C |
| 16 | 85442574 | A | G | 16 | 85443843 | T | C | 16 | 85443876 | G | A |
| 16 | 85443989 | T | G | 16 | 85444082 | G | C | 16 | 85444102 | C | A |
| 16 | 85444107 | C | T | 16 | 85444130 | T | C | 16 | 85444204 | C | T |
| 16 | 85444618 | A | G | 16 | 85444655 | A | C | 16 | 85444705 | T | C |
| 16 | 85444849 | A | C | 16 | 85445141 | C | T | 16 | 85445267 | C | G |
| 16 | 85445365 | C | T | 16 | 86005933 | C | G | 16 | 86005964 | T | G |
| 16 | 86005984 | G | A | 16 | 86006286 | C | T | 16 | 87178517 | A | G |
| 16 | 87179026 | T | C | 16 | 87179050 | G | T | 16 | 87179063 | G | A |
| 16 | 87179102 | G | A | 16 | 87179142 | G | A | 16 | 87179180 | C | A |
| 16 | 87648305 | C | T | 16 | 87648315 | T | G | 16 | 87648341 | G | A |
| 16 | 87926558 | G | A | 16 | 87926696 | G | C | 16 | 88073139 | C | T |
| 16 | 88073194 | C | T | 16 | 88073218 | T | C | 16 | 88073296 | A | G |
| 16 | 88310744 | C | T | 16 | 88310829 | T | C | 16 | 88310830 | G | A |
| 16 | 88310910 | A | G | 16 | 88310955 | A | G | 16 | 88310963 | C | G |
| 16 | 88311269 | A | G | 16 | 88311279 | A | T | 16 | 88311285 | A | T |
| 16 | 88311291 | G | C | 16 | 88311355 | T | C | 16 | 88312367 | C | T |
| 16 | 88312750 | C | T | 16 | 88312754 | A | G | 16 | 88312962 | C | T |
| 16 | 88312973 | C | T | 16 | 88313227 | C | T | 16 | 88313229 | C | T |
| 16 | 88313255 | A | G | 16 | 88313383 | A | G | 16 | 88313402 | T | C |
| 16 | 88313449 | T | C | 16 | 88313454 | T | C | 16 | 88313493 | A | G |
| 16 | 88313505 | A | G | 16 | 88313530 | G | T | 16 | 88313585 | A | G |
| 16 | 88313677 | C | T | 16 | 88313845 | C | T | 16 | 88794959 | C | A |
| 16 | 88795086 | T | G | 16 | 88795431 | A | T | 16 | 88795433 | C | T |
| 16 | 88795718 | C | T | 16 | 89299506 | A | T | 16 | 89299519 | G | C |
| 16 | 89299899 | A | G | 16 | 89756171 | G | A | 16 | 89756185 | A | T |
| 16 | 89806732 | G | A | 16 | 89807543 | G | A | 16 | 89808939 | T | C |
| 16 | 89809548 | C | A | 16 | 89809549 | A | G | 16 | 89811151 | A | G |
| 16 | 89811209 | C | T | 16 | 89811663 | T | C | 16 | 89811751 | C | G |
| 16 | 89814818 | T | G | 16 | 89815321 | C | T | 16 | 89815824 | G | A |
| 16 | 89816569 | C | A | 16 | 89818340 | G | C | 16 | 89818908 | C | T |
| 16 | 89828043 | T | A | 16 | 89839976 | T | C | 16 | 89842479 | T | C |
| 16 | 89849735 | G | A | 16 | 89849740 | C | T | 16 | 89851004 | A | G |
| 16 | 89851033 | C | T | 16 | 89998794 | G | A | 16 | 90233312 | C | T |
| 17 | 164435 | C | T | 17 | 164955 | C | A | 17 | 164967 | T | C |
| 17 | 164969 | C | G | 17 | 164980 | C | T | 17 | 164997 | T | G |
| 17 | 164999 | C | G | 17 | 165010 | T | C | 17 | 165025 | T | C |
| 17 | 165071 | C | T | 17 | 165078 | C | A | 17 | 165085 | C | T |
| 17 | 165090 | G | T | 17 | 165147 | T | C | 17 | 165201 | C | G |
| 17 | 165240 | C | G | 17 | 165247 | C | T | 17 | 165256 | C | A |
| 17 | 165295 | T | C | 17 | 165357 | C | A | 17 | 165375 | A | C |
| 17 | 165386 | T | C | 17 | 165409 | C | T | 17 | 165419 | A | C |
| 17 | 165501 | T | C | 17 | 165515 | T | C | 17 | 165562 | C | T |
| 17 | 165565 | G | C | 17 | 165576 | T | C | 17 | 165585 | G | T |
| 17 | 165615 | G | T | 17 | 165643 | C | A | 17 | 165694 | A | G |
| 17 | 165695 | C | T | 17 | 165712 | T | A | 17 | 165732 | T | C |
| 17 | 165736 | C | T | 17 | 165742 | G | C | 17 | 165966 | T | C |
| 17 | 165976 | C | T | 17 | 166081 | T | C | 17 | 166186 | C | A |
| 17 | 166346 | A | G | 17 | 1020183 | G | A | 17 | 1020394 | G | T |
| 17 | 1022697 | C | T | 17 | 1022705 | G | T | 17 | 1022734 | G | A |
| 17 | 1022796 | G | C | 17 | 1022860 | G | C | 17 | 1022923 | C | T |
| 17 | 2257864 | A | G | 17 | 2258637 | A | C | 17 | 3315757 | T | C |
| 17 | 3315904 | G | A | 17 | 3771365 | A | C | 17 | 3771379 | G | A |
| 17 | 3771380 | T | C | 17 | 3771381 | G | A | 17 | 3771384 | C | A |
| 17 | 5033768 | C | A | 17 | 5034469 | G | A | 17 | 5043998 | C | T |
| 17 | 5603793 | C | T | 17 | 6461283 | A | G | 17 | 7569698 | A | C |
| 17 | 7573229 | C | T | 17 | 7576348 | C | T | 17 | 8107979 | G | T |
| 17 | 8109598 | G | A | 17 | 8113186 | A | G | 17 | 9756002 | G | T |
| 17 | 9756057 | T | C | 17 | 9756171 | A | T | 17 | 9756198 | T | C |
| 17 | 10095727 | T | G | 17 | 11355538 | C | T | 17 | 11356161 | A | C |
| 17 | 11356178 | G | A | 17 | 11924471 | C | T | 17 | 12042934 | T | A |
| 17 | 12968947 | T | C | 17 | 12969339 | G | T | 17 | 15486857 | G | A |
| 17 | 15486909 | A | G | 17 | 16012494 | A | T | 17 | 16068096 | C | T |
| 17 | 16528031 | C | T | 17 | 17120668 | G | C | 17 | 17625022 | A | G |
| 17 | 17625107 | T | C | 17 | 17625185 | G | T | 17 | 18297214 | C | G |
| 17 | 18299435 | C | T | 17 | 18299631 | C | G | 17 | 18393867 | C | T |
| 17 | 19486126 | C | T | 17 | 19486133 | T | C | 17 | 19486148 | T | C |
| 17 | 19486537 | A | G | 17 | 19486585 | C | A | 17 | 19486592 | T | C |
| 17 | 20451455 | A | C | 17 | 20453878 | G | T | 17 | 21262861 | G | A |
| 17 | 21408378 | T | C | 17 | 21503001 | G | C | 17 | 21825993 | T | G |
| 17 | 21826038 | T | C | 17 | 21826256 | A | G | 17 | 21859750 | A | G |
| 17 | 21901935 | C | G | 17 | 21901936 | C | T | 17 | 21901945 | G | C |
| 17 | 22213908 | C | T | 17 | 25746737 | C | A | 17 | 25747067 | C | T |
| 17 | 25747244 | C | T | 17 | 25748176 | C | A | 17 | 25748270 | G | T |
| 17 | 25982703 | C | T | 17 | 25983153 | G | A | 17 | 26079849 | T | C |
| 17 | 26087967 | T | A | 17 | 26087978 | A | T | 17 | 26088432 | C | G |
| 17 | 27536646 | T | C | 17 | 28043613 | G | A | 17 | 28883741 | C | T |
| 17 | 29541339 | A | T | 17 | 29558082 | T | C | 17 | 29592550 | T | C |
| 17 | 29675878 | T | G | 17 | 29684553 | C | T | 17 | 30389991 | G | A |
| 17 | 32084335 | G | T | 17 | 32980299 | C | T | 17 | 32980342 | C | A |
| 17 | 33520071 | C | T | 17 | 33520663 | C | T | 17 | 34416275 | T | A |
| 17 | 34416946 | G | A | 17 | 34488320 | G | A | 17 | 34587938 | T | C |
| 17 | 34587942 | C | T | 17 | 34754758 | G | C | 17 | 34754836 | A | C |
| 17 | 36097775 | G | A | 17 | 36102035 | G | C | 17 | 36103872 | C | G |
| 17 | 36221809 | T | A | 17 | 36578907 | C | T | 17 | 36579428 | C | T |
| 17 | 37230242 | T | C | 17 | 37626963 | G | A | 17 | 37646589 | T | C |
| 17 | 37650569 | G | A | 17 | 38561366 | T | C | 17 | 38568249 | C | G |
| 17 | 39383335 | C | G | 17 | 39394674 | A | G | 17 | 39394729 | C | A |
| 17 | 39394755 | C | A | 17 | 39394787 | C | A | 17 | 39394790 | T | C |
| 17 | 39394807 | C | A | 17 | 39394885 | C | T | 17 | 39394911 | A | G |
| 17 | 39406673 | T | G | 17 | 39604697 | A | G | 17 | 40490539 | T | G |
| 17 | 40490559 | G | T | 17 | 40498273 | T | C | 17 | 40498944 | T | C |
| 17 | 41199913 | T | C | 17 | 41205772 | G | A | 17 | 41212169 | C | T |
| 17 | 41213626 | G | T | 17 | 41213660 | T | C | 17 | 41222723 | T | C |
| 17 | 41230228 | G | A | 17 | 41232698 | C | T | 17 | 41248164 | C | T |
| 17 | 41250923 | T | C | 17 | 41257134 | T | C | 17 | 41276348 | T | C |
| 17 | 41310495 | T | C | 17 | 41623212 | G | A | 17 | 43057954 | G | A |
| 17 | 43626858 | C | T | 17 | 45095080 | T | G | 17 | 45095092 | G | A |
| 17 | 45095678 | G | A | 17 | 46319546 | G | A | 17 | 47696070 | T | C |
| 17 | 47696933 | C | T | 17 | 47696939 | G | A | 17 | 47700315 | G | A |
| 17 | 48899222 | A | G | 17 | 49451714 | A | G | 17 | 49451742 | G | C |
| 17 | 49452209 | A | G | 17 | 50762097 | A | G | 17 | 50762653 | G | T |
| 17 | 51901084 | A | G | 17 | 51901643 | C | T | 17 | 51901703 | C | G |
| 17 | 52941883 | G | C | 17 | 53573902 | C | T | 17 | 53574374 | G | A |
| 17 | 55426865 | T | C | 17 | 56447952 | G | C | 17 | 56773834 | T | C |
| 17 | 58082363 | C | G | 17 | 58082421 | G | C | 17 | 58082431 | G | C |
| 17 | 58174022 | G | A | 17 | 59761706 | A | G | 17 | 59878416 | G | C |
| 17 | 59886310 | T | C | 17 | 59934879 | G | C | 17 | 60341389 | G | C |
| 17 | 60341507 | A | G | 17 | 60341507 | A | T | 17 | 60350783 | T | C |
| 17 | 60350874 | C | A | 17 | 61963236 | C | A | 17 | 61963241 | A | G |
| 17 | 61963478 | T | C | 17 | 61963622 | A | C | 17 | 61978159 | T | C |
| 17 | 61978548 | A | G | 17 | 61978622 | G | A | 17 | 62790430 | G | A |
| 17 | 63532175 | A | G | 17 | 63537898 | T | C | 17 | 64914494 | C | T |
| 17 | 65854602 | C | T | 17 | 65855104 | T | A | 17 | 66180139 | C | G |
| 17 | 66180681 | G | C | 17 | 66211771 | G | C | 17 | 66211840 | T | C |
| 17 | 66211969 | T | A | 17 | 66212122 | A | T | 17 | 66511299 | C | T |
| 17 | 66518663 | A | G | 17 | 66520427 | C | T | 17 | 66520838 | A | G |
| 17 | 66523772 | G | A | 17 | 66524755 | C | T | 17 | 67528431 | G | A |
| 17 | 69034000 | A | T | 17 | 71276883 | C | T | 17 | 71978657 | A | T |
| 17 | 71978747 | G | A | 17 | 71979236 | T | G | 17 | 71979237 | T | G |
| 17 | 72005968 | A | G | 17 | 72005970 | A | T | 17 | 72623641 | G | A |
| 17 | 74442413 | T | C | 17 | 74442440 | C | G | 17 | 74442942 | A | G |
| 17 | 75557061 | G | T | 17 | 75558122 | T | C | 17 | 75558750 | A | G |
| 17 | 75558782 | A | T | 17 | 75558791 | A | C | 17 | 75558802 | C | T |
| 17 | 75558814 | C | T | 17 | 75559019 | C | T | 17 | 76168857 | G | T |
| 17 | 76608673 | A | G | 17 | 78299319 | T | C | 17 | 78519169 | G | A |
| 17 | 78704156 | T | C | 17 | 78728314 | G | A | 17 | 78765080 | A | C |
| 17 | 78795868 | A | G | 17 | 78820023 | T | C | 17 | 78820212 | G | A |
| 17 | 78829531 | C | T | 17 | 78831983 | C | T | 17 | 78854056 | A | G |
| 17 | 78854487 | A | G | 17 | 78854496 | C | T | 17 | 78867906 | G | A |
| 17 | 78867951 | G | C | 17 | 78882953 | G | A | 17 | 78882972 | A | C |
| 17 | 78897056 | C | G | 17 | 78897732 | C | T | 17 | 78897733 | T | A |
| 17 | 78919285 | A | G | 17 | 78919324 | G | A | 17 | 78919349 | A | G |
| 17 | 78919387 | G | A | 17 | 78919837 | G | C | 17 | 78919859 | G | C |
| 17 | 78920807 | A | G | 17 | 78922952 | C | G | 17 | 78923007 | T | G |
| 17 | 78935071 | G | A | 17 | 78937165 | G | A | 17 | 78937925 | C | T |
| 17 | 78938011 | C | A | 17 | 80107354 | G | A | 17 | 80108414 | T | C |
| 17 | 80108478 | A | G | 17 | 80108696 | C | T | 17 | 80108822 | C | T |
| 17 | 80108842 | T | C | 17 | 81024201 | C | T | 17 | 81024331 | G | A |
| 17 | 81024640 | C | A | 17 | 81024656 | G | A | 17 | 81024660 | T | C |
| 17 | 81024673 | C | T | 17 | 81024674 | C | G | 17 | 81024677 | A | C |
| 17 | 81024682 | T | G | 17 | 81024691 | A | G | 17 | 81024707 | C | T |
| 17 | 81024731 | A | G | 17 | 81024741 | G | A | 17 | 81024752 | A | C |
| 18 | 13754 | A | T | 18 | 43439 | A | T | 18 | 43535 | A | C |
| 18 | 43538 | T | C | 18 | 86837 | T | C | 18 | 86857 | T | C |
| 18 | 657352 | T | C | 18 | 657443 | A | C | 18 | 657458 | A | G |
| 18 | 657474 | T | C | 18 | 658035 | A | G | 18 | 1963905 | A | G |
| 18 | 2913896 | C | G | 18 | 2913918 | G | A | 18 | 3406591 | A | G |
| 18 | 4106340 | T | G | 18 | 4106917 | G | A | 18 | 4932957 | C | T |
| 18 | 4933515 | C | T | 18 | 4933516 | G | A | 18 | 4933518 | T | G |
| 18 | 6462081 | A | C | 18 | 7382800 | A | G | 18 | 7382881 | C | A |
| 18 | 8408675 | G | A | 18 | 8789233 | G | A | 18 | 11610464 | G | T |
| 18 | 11610468 | A | G | 18 | 11610624 | G | C | 18 | 11610630 | G | A |
| 18 | 11610763 | A | G | 18 | 11618008 | G | A | 18 | 11622851 | T | C |
| 18 | 11623045 | T | C | 18 | 11623100 | C | T | 18 | 11644581 | G | A |
| 18 | 11644667 | G | A | 18 | 11644679 | G | T | 18 | 11644723 | T | C |
| 18 | 11644746 | G | A | 18 | 12101808 | G | A | 18 | 12898367 | A | G |
| 18 | 13369940 | C | T | 18 | 13370544 | G | A | 18 | 13673746 | C | T |
| 18 | 13674057 | A | G | 18 | 13674212 | C | A | 18 | 13674777 | T | C |
| 18 | 13675072 | A | G | 18 | 13675437 | T | C | 18 | 13675449 | A | T |
| 18 | 13675457 | G | C | 18 | 13675494 | C | T | 18 | 13675799 | A | T |
| 18 | 14341320 | T | C | 18 | 14341763 | T | C | 18 | 14436696 | C | A |
| 18 | 14524300 | T | C | 18 | 14594781 | C | A | 18 | 14595105 | A | T |
| 18 | 15165336 | A | G | 18 | 15165362 | G | T | 18 | 15206879 | C | T |
| 18 | 19761756 | C | A | 18 | 19995753 | T | C | 18 | 21252592 | A | G |
| 18 | 21978687 | A | G | 18 | 22598696 | C | T | 18 | 23785015 | C | A |
| 18 | 23785095 | C | T | 18 | 23785115 | C | G | 18 | 23883026 | G | C |
| 18 | 23883058 | T | C | 18 | 24885804 | T | C | 18 | 26757855 | T | C |
| 18 | 27273833 | C | T | 18 | 27587457 | A | G | 18 | 28115656 | T | C |
| 18 | 28115679 | C | G | 18 | 28115710 | C | T | 18 | 28115783 | G | A |
| 18 | 28115955 | G | T | 18 | 28116070 | G | A | 18 | 29573525 | T | C |
| 18 | 31662955 | C | T | 18 | 33187675 | G | C | 18 | 33187676 | T | G |
| 18 | 33187692 | A | G | 18 | 34886895 | T | G | 18 | 34887449 | G | A |
| 18 | 34887505 | A | G | 18 | 35451056 | C | T | 18 | 36001103 | G | C |
| 18 | 38530356 | A | G | 18 | 39614110 | G | A | 18 | 39620376 | T | A |
| 18 | 39620932 | G | A | 18 | 39620950 | C | T | 18 | 40049623 | G | A |
| 18 | 40050171 | T | C | 18 | 43006049 | C | T | 18 | 44223365 | G | T |
| 18 | 44909077 | A | G | 18 | 44909081 | G | A | 18 | 45368587 | G | A |
| 18 | 45371445 | C | T | 18 | 45371509 | C | T | 18 | 45377882 | G | C |
| 18 | 45395111 | G | C | 18 | 45651984 | C | G | 18 | 46453156 | A | T |
| 18 | 46453754 | C | T | 18 | 47701550 | T | C | 18 | 48581601 | C | A |
| 18 | 48603403 | T | C | 18 | 49958403 | G | C | 18 | 49959036 | G | T |
| 18 | 51674135 | A | G | 18 | 54762024 | G | A | 18 | 55396242 | G | A |
| 18 | 55782772 | T | C | 18 | 56757385 | T | C | 18 | 56757399 | A | G |
| 18 | 56757422 | T | C | 18 | 59992577 | T | C | 18 | 59992591 | C | G |
| 18 | 60015207 | G | C | 18 | 60015750 | G | A | 18 | 60026926 | C | T |
| 18 | 60026931 | G | C | 18 | 60027005 | T | A | 18 | 60028762 | G | A |
| 18 | 60029238 | G | C | 18 | 60029281 | A | G | 18 | 60029292 | G | A |
| 18 | 60034276 | C | T | 18 | 60984964 | C | A | 18 | 61654297 | A | G |
| 18 | 63196837 | G | A | 18 | 65928322 | A | T | 18 | 66103401 | C | T |
| 18 | 67182508 | G | T | 18 | 67182512 | G | A | 18 | 67183000 | A | G |
| 18 | 67183043 | T | C | 18 | 68086329 | G | A | 18 | 68086404 | A | G |
| 18 | 68086457 | T | G | 18 | 68086460 | C | T | 18 | 68700027 | G | A |
| 18 | 69291422 | G | A | 18 | 70066366 | G | A | 18 | 70592333 | A | G |
| 18 | 70592333 | A | T | 18 | 70592352 | A | G | 18 | 70592858 | T | C |
| 18 | 71467330 | G | C | 18 | 71640426 | G | A | 18 | 72226809 | C | A |
| 18 | 72226857 | T | C | 18 | 73300822 | T | C | 18 | 74757060 | T | G |
| 18 | 74757768 | A | G | 18 | 76394226 | C | G | 18 | 76798551 | G | A |
| 18 | 76798812 | C | T | 18 | 76798870 | A | T | 18 | 76799194 | T | C |
| 18 | 76799227 | A | G | 18 | 76799231 | C | T | 18 | 76799232 | T | G |
| 18 | 76799233 | G | C | 18 | 76799233 | G | T | 18 | 76799237 | G | A |
| 18 | 76799276 | C | T | 18 | 76799300 | G | A | 18 | 76799500 | C | T |
| 18 | 77378236 | C | A | 18 | 77378238 | C | G | 18 | 77378242 | G | A |
| 18 | 77378897 | C | T | 18 | 77378974 | C | T | 18 | 77379017 | C | T |
| 18 | 77379036 | C | A | 18 | 77379117 | T | C | 18 | 77379177 | T | C |
| 18 | 77379214 | C | T | 18 | 77379461 | T | C | 18 | 77379462 | C | A |
| 18 | 77379462 | C | G | 18 | 77379473 | T | C | 18 | 77379566 | T | C |
| 18 | 77379577 | C | T | 18 | 77379578 | C | G | 18 | 77379589 | T | C |
| 18 | 77379649 | T | C | 18 | 77379742 | T | C | 18 | 77379754 | C | G |
| 18 | 77379765 | C | G | 18 | 77379814 | C | G | 18 | 77379845 | C | T |
| 18 | 77379922 | T | C | 18 | 77379994 | C | G | 18 | 77380025 | T | C |
| 18 | 77380053 | G | C | 18 | 77380053 | G | T | 18 | 77380442 | C | T |
| 18 | 77380579 | C | T | 18 | 77380810 | A | G | 19 | 1218219 | C | T |
| 19 | 1218771 | G | A | 19 | 1219129 | G | A | 19 | 1219702 | G | A |
| 19 | 1222316 | C | T | 19 | 1226901 | G | T | 19 | 2180515 | G | A |
| 19 | 2185651 | C | T | 19 | 2186237 | G | A | 19 | 2190774 | T | A |
| 19 | 2194896 | T | C | 19 | 2199576 | C | T | 19 | 2207041 | A | G |
| 19 | 2207452 | G | A | 19 | 2208859 | T | C | 19 | 2210269 | C | T |
| 19 | 2221792 | T | C | 19 | 2833802 | A | G | 19 | 2833864 | G | A |
| 19 | 3110585 | A | G | 19 | 3113770 | T | C | 19 | 3113780 | C | T |
| 19 | 3113839 | A | G | 19 | 3115402 | C | T | 19 | 3118638 | C | T |
| 19 | 3120739 | T | G | 19 | 3148819 | G | T | 19 | 4094110 | G | T |
| 19 | 4094775 | G | A | 19 | 4098947 | G | A | 19 | 4102132 | A | C |
| 19 | 5524721 | C | T | 19 | 6230241 | G | A | 19 | 6495064 | C | G |
| 19 | 6495079 | G | C | 19 | 6502137 | C | A | 19 | 6990495 | C | T |
| 19 | 6991043 | A | G | 19 | 7032902 | G | A | 19 | 7033122 | T | C |
| 19 | 7037737 | C | A | 19 | 7051588 | G | A | 19 | 7051637 | T | G |
| 19 | 7051643 | T | C | 19 | 7051909 | G | C | 19 | 7051942 | C | A |
| 19 | 7056116 | A | G | 19 | 7514901 | A | G | 19 | 7514901 | A | T |
| 19 | 7515546 | G | T | 19 | 7515550 | A | G | 19 | 7515569 | G | A |
| 19 | 7515658 | A | G | 19 | 7515659 | C | A | 19 | 7515661 | G | A |
| 19 | 7515716 | G | T | 19 | 7515742 | G | A | 19 | 7515750 | G | T |
| 19 | 7515772 | C | T | 19 | 7515788 | C | A | 19 | 7516210 | C | T |
| 19 | 7516243 | A | G | 19 | 7816166 | G | A | 19 | 7816206 | G | T |
| 19 | 7816216 | C | A | 19 | 8202601 | C | T | 19 | 8567500 | G | A |
| 19 | 8567548 | A | G | 19 | 10097667 | C | T | 19 | 10597746 | T | A |
| 19 | 10600753 | A | C | 19 | 11113475 | C | A | 19 | 11129429 | C | G |
| 19 | 11134090 | A | G | 19 | 11136857 | C | T | 19 | 11138226 | T | C |
| 19 | 11143811 | C | G | 19 | 11168798 | G | A | 19 | 11172299 | G | A |
| 19 | 12491498 | C | T | 19 | 12491779 | T | C | 19 | 13481531 | C | T |
| 19 | 13699891 | C | A | 19 | 13699931 | C | T | 19 | 14218491 | A | G |
| 19 | 14404337 | G | C | 19 | 15807884 | A | G | 19 | 15871215 | C | G |
| 19 | 15871285 | T | A | 19 | 15871360 | G | C | 19 | 15871498 | A | G |
| 19 | 16046478 | G | T | 19 | 17700647 | G | A | 19 | 17937300 | T | A |
| 19 | 17942927 | C | T | 19 | 17950674 | C | T | 19 | 18272190 | A | C |
| 19 | 18974059 | G | C | 19 | 19261179 | C | A | 19 | 19261833 | A | G |
| 19 | 19261851 | T | C | 19 | 19664077 | C | T | 19 | 20024912 | T | A |
| 19 | 20024959 | G | A | 19 | 20050037 | G | A | 19 | 20162695 | A | C |
| 19 | 20162824 | G | C | 19 | 20632050 | T | C | 19 | 20632172 | T | G |
| 19 | 20632400 | T | C | 19 | 20832195 | C | T | 19 | 20857905 | T | C |
| 19 | 21057335 | G | A | 19 | 21148018 | G | A | 19 | 21148051 | C | G |
| 19 | 21148086 | G | T | 19 | 21749035 | T | C | 19 | 21749580 | C | T |
| 19 | 22155757 | G | A | 19 | 22642684 | T | C | 19 | 23477341 | C | G |
| 19 | 23477350 | C | G | 19 | 28330492 | T | C | 19 | 28330539 | A | G |
| 19 | 29331628 | C | A | 19 | 29331703 | C | G | 19 | 29518038 | G | C |
| 19 | 30303352 | T | C | 19 | 30311372 | C | T | 19 | 30314371 | G | A |
| 19 | 34080511 | G | C | 19 | 34080579 | C | G | 19 | 34882578 | G | A |
| 19 | 36213072 | A | G | 19 | 36217957 | A | G | 19 | 36219420 | G | A |
| 19 | 36219525 | A | G | 19 | 36222017 | A | G | 19 | 36222023 | T | C |
| 19 | 36224973 | T | C | 19 | 36807142 | A | G | 19 | 36807249 | A | T |
| 19 | 36807297 | T | G | 19 | 36807306 | G | A | 19 | 36807345 | G | A |
| 19 | 36807347 | G | A | 19 | 37147049 | A | G | 19 | 37147639 | C | T |
| 19 | 37752858 | C | T | 19 | 37752861 | T | C | 19 | 37752862 | G | A |
| 19 | 37753071 | T | C | 19 | 37753073 | T | C | 19 | 37753239 | G | A |
| 19 | 38391602 | C | A | 19 | 38932157 | C | T | 19 | 39261999 | C | T |
| 19 | 39990995 | G | A | 19 | 39991023 | G | A | 19 | 39991614 | A | G |
| 19 | 39994023 | A | G | 19 | 39994513 | G | A | 19 | 39997472 | G | A |
| 19 | 40739513 | A | G | 19 | 40743523 | C | T | 19 | 40770860 | G | A |
| 19 | 41349550 | G | C | 19 | 41349640 | G | C | 19 | 41349978 | A | G |
| 19 | 41353107 | T | C | 19 | 41353849 | C | T | 19 | 41381310 | A | G |
| 19 | 41381334 | G | A | 19 | 41382180 | G | C | 19 | 41384067 | T | C |
| 19 | 41384084 | T | C | 19 | 41384102 | G | T | 19 | 41384385 | T | G |
| 19 | 41384386 | C | G | 19 | 41385734 | C | T | 19 | 41386527 | G | A |
| 19 | 41386544 | C | G | 19 | 41386547 | G | C | 19 | 41386584 | G | T |
| 19 | 41386622 | C | T | 19 | 41387241 | G | A | 19 | 41414363 | A | T |
| 19 | 41415087 | G | A | 19 | 41415112 | T | A | 19 | 41415818 | G | A |
| 19 | 41416072 | G | C | 19 | 41416143 | C | T | 19 | 41416260 | T | G |
| 19 | 41416263 | C | A | 19 | 41416264 | A | G | 19 | 41512415 | T | C |
| 19 | 41515018 | G | A | 19 | 41529956 | A | G | 19 | 41600800 | C | T |
| 19 | 41600808 | C | T | 19 | 41736661 | G | A | 19 | 41744831 | C | T |
| 19 | 41745930 | C | T | 19 | 41754217 | G | T | 19 | 41765229 | T | C |
| 19 | 43359392 | G | A | 19 | 43383004 | G | C | 19 | 43421318 | C | T |
| 19 | 43440900 | T | C | 19 | 43586183 | T | C | 19 | 43586267 | C | T |
| 19 | 43586408 | A | G | 19 | 43690192 | T | A | 19 | 43772788 | C | G |
| 19 | 43773287 | G | C | 19 | 43773303 | T | C | 19 | 43860030 | G | A |
| 19 | 43860048 | G | T | 19 | 43860053 | G | A | 19 | 43860076 | C | T |
| 19 | 43860192 | T | G | 19 | 43882832 | A | C | 19 | 43882836 | C | T |
| 19 | 43882895 | A | C | 19 | 43883013 | C | A | 19 | 43883124 | C | T |
| 19 | 43883126 | G | A | 19 | 44055898 | C | T | 19 | 44056052 | A | G |
| 19 | 44057227 | T | C | 19 | 44057276 | G | A | 19 | 44905282 | C | G |
| 19 | 44905683 | C | T | 19 | 44905779 | C | T | 19 | 45862449 | A | G |
| 19 | 45868649 | C | G | 19 | 45871606 | T | C | 19 | 45916587 | T | C |
| 19 | 45924038 | G | A | 19 | 46375779 | C | T | 19 | 46406242 | A | G |
| 19 | 46721467 | G | A | 19 | 48764695 | G | A | 19 | 48764721 | T | C |
| 19 | 48765117 | G | A | 19 | 49457938 | A | G | 19 | 49458570 | T | C |
| 19 | 49459876 | T | A | 19 | 50265230 | C | T | 19 | 50901893 | G | A |
| 19 | 50903004 | A | G | 19 | 50909107 | C | A | 19 | 50920959 | G | T |
| 19 | 51315061 | T | C | 19 | 51315132 | T | C | 19 | 51315175 | T | C |
| 19 | 51315251 | C | T | 19 | 51646187 | C | A | 19 | 52249947 | C | G |
| 19 | 52249959 | G | T | 19 | 52704961 | T | C | 19 | 52709064 | G | T |
| 19 | 52714353 | T | A | 19 | 52715716 | G | A | 19 | 52718799 | C | T |
| 19 | 52718816 | C | T | 19 | 52718884 | T | A | 19 | 52720262 | A | G |
| 19 | 52723930 | C | T | 19 | 52724027 | C | A | 19 | 53927121 | A | C |
| 19 | 53927131 | C | A | 19 | 53927136 | A | G | 19 | 54007749 | G | C |
| 19 | 54208058 | T | C | 19 | 54267981 | C | T | 19 | 54860836 | G | A |
| 19 | 54860845 | A | G | 19 | 54860961 | G | A | 19 | 54860964 | A | C |
| 19 | 54860970 | T | G | 19 | 54861346 | G | A | 19 | 54861759 | G | T |
| 19 | 55271654 | C | G | 19 | 55333096 | T | C | 19 | 55333246 | C | T |
| 19 | 55350666 | C | A | 19 | 55350713 | G | C | 19 | 55350754 | A | G |
| 19 | 55350948 | A | T | 19 | 55350979 | A | G | 19 | 55351022 | A | C |
| 19 | 55543973 | C | T | 19 | 55544430 | A | G | 19 | 55544687 | G | A |
| 19 | 55544750 | A | G | 19 | 55545139 | A | G | 19 | 55545191 | G | T |
| 19 | 56361176 | A | G | 19 | 56361263 | G | A | 19 | 56361279 | A | G |
| 19 | 56361479 | A | G | 19 | 56361733 | T | G | 19 | 56361758 | T | A |
| 19 | 56361933 | G | T | 19 | 57866239 | C | A | 19 | 58464742 | G | C |
| 19 | 59051326 | T | C | 19 | 59051337 | G | C | 20 | 402053 | G | A |
| 20 | 402440 | A | G | 20 | 928228 | T | C | 20 | 1496279 | A | G |
| 20 | 2367161 | C | T | 20 | 2367209 | T | C | 20 | 2545011 | T | C |
| 20 | 4040209 | G | T | 20 | 4040258 | T | C | 20 | 4040763 | T | C |
| 20 | 4040828 | T | C | 20 | 4040923 | T | G | 20 | 4040961 | C | G |
| 20 | 4155648 | C | T | 20 | 4155668 | C | T | 20 | 4776659 | G | A |
| 20 | 5889649 | T | C | 20 | 6922046 | T | A | 20 | 8475010 | A | G |
| 20 | 9318946 | A | G | 20 | 9353445 | C | T | 20 | 9365303 | T | C |
| 20 | 9371499 | G | A | 20 | 9374613 | G | T | 20 | 9440715 | C | G |
| 20 | 9448947 | G | A | 20 | 9449000 | T | G | 20 | 9457170 | C | T |
| 20 | 9459927 | A | G | 20 | 10882821 | C | T | 20 | 11821257 | G | A |
| 20 | 12626712 | G | T | 20 | 12626745 | A | G | 20 | 14706879 | T | A |
| 20 | 15313903 | T | A | 20 | 15314383 | G | A | 20 | 16239362 | C | T |
| 20 | 16239376 | C | T | 20 | 16239426 | C | T | 20 | 18190882 | C | T |
| 20 | 18191172 | T | C | 20 | 18191291 | G | T | 20 | 18466346 | G | A |
| 20 | 20336724 | C | T | 20 | 20336950 | G | T | 20 | 20337707 | A | G |
| 20 | 20337722 | A | G | 20 | 20337869 | G | T | 20 | 20338243 | A | G |
| 20 | 20338247 | T | A | 20 | 20338251 | A | G | 20 | 20338825 | A | G |
| 20 | 20338971 | C | G | 20 | 20817609 | G | C | 20 | 21142523 | C | G |
| 20 | 21885619 | A | C | 20 | 22297047 | A | G | 20 | 22563571 | G | A |
| 20 | 22892171 | G | T | 20 | 23456589 | T | G | 20 | 23456962 | A | T |
| 20 | 23456975 | C | G | 20 | 23457003 | C | G | 20 | 23757966 | G | C |
| 20 | 23758038 | C | A | 20 | 23840240 | T | C | 20 | 23840242 | T | C |
| 20 | 23975455 | C | T | 20 | 24649705 | C | T | 20 | 24650360 | G | A |
| 20 | 25845523 | C | T | 20 | 25845579 | C | A | 20 | 25845591 | C | A |
| 20 | 25845594 | G | T | 20 | 25943298 | G | A | 20 | 26073480 | T | C |
| 20 | 29517367 | G | A | 20 | 29517535 | C | T | 20 | 29520369 | G | A |
| 20 | 29520383 | A | G | 20 | 29551711 | G | T | 20 | 31015643 | T | C |
| 20 | 31019725 | T | C | 20 | 31938046 | C | T | 20 | 33581689 | T | C |
| 20 | 33582279 | C | A | 20 | 34101631 | C | A | 20 | 34854605 | T | C |
| 20 | 36012348 | C | A | 20 | 36022154 | T | C | 20 | 36030592 | G | T |
| 20 | 36030622 | T | C | 20 | 36838422 | T | C | 20 | 37900688 | C | G |
| 20 | 37900691 | C | T | 20 | 39690342 | T | C | 20 | 39710038 | T | G |
| 20 | 41426178 | C | T | 20 | 41677471 | G | C | 20 | 42196825 | T | C |
| 20 | 42733847 | A | G | 20 | 43334843 | G | A | 20 | 43962124 | C | T |
| 20 | 43964620 | C | T | 20 | 45659653 | A | G | 20 | 46456298 | C | T |
| 20 | 46456399 | C | A | 20 | 46749867 | G | A | 20 | 47128176 | G | A |
| 20 | 47201278 | C | G | 20 | 47201306 | C | T | 20 | 47833636 | T | C |
| 20 | 47833697 | T | C | 20 | 48574438 | G | A | 20 | 48574454 | G | A |
| 20 | 50477441 | C | T | 20 | 50677624 | T | A | 20 | 50677714 | G | T |
| 20 | 53503158 | C | T | 20 | 53503198 | C | A | 20 | 54066409 | G | A |
| 20 | 54961073 | A | G | 20 | 55462729 | T | C | 20 | 55462744 | G | A |
| 20 | 55463137 | C | T | 20 | 55991479 | G | A | 20 | 56169863 | C | A |
| 20 | 56994740 | A | G | 20 | 57479133 | A | G | 20 | 57483948 | G | A |
| 20 | 58057058 | C | A | 20 | 58057077 | T | G | 20 | 58057118 | C | A |
| 20 | 58057119 | T | A | 20 | 59495071 | G | A | 20 | 59848508 | G | A |
| 20 | 59848515 | G | T | 20 | 59848519 | A | G | 20 | 59848582 | T | C |
| 20 | 59848585 | A | G | 20 | 60495224 | G | A | 20 | 60495285 | G | A |
| 21 | 9483659 | T | C | 21 | 9910262 | A | C | 21 | 9954440 | C | A |
| 21 | 9954672 | C | A | 21 | 9954692 | C | G | 21 | 9954700 | T | A |
| 21 | 9954700 | T | C | 21 | 9954718 | C | G | 21 | 9954726 | T | C |
| 21 | 9954750 | C | A | 21 | 9954822 | C | T | 21 | 9954874 | G | T |
| 21 | 9954972 | T | G | 21 | 9954978 | G | T | 21 | 9954994 | G | A |
| 21 | 10029331 | A | T | 21 | 14457818 | G | A | 21 | 14513159 | G | A |
| 21 | 14524601 | A | T | 21 | 14900799 | C | T | 21 | 14900897 | A | G |
| 21 | 14900901 | T | C | 21 | 14936098 | T | G | 21 | 15090065 | G | A |
| 21 | 15090071 | T | C | 21 | 15090282 | T | G | 21 | 15090308 | A | G |
| 21 | 15090315 | G | A | 21 | 15090335 | C | T | 21 | 15090336 | G | A |
| 21 | 15090391 | A | G | 21 | 15216110 | T | C | 21 | 15216154 | G | A |
| 21 | 15216190 | T | A | 21 | 15219309 | G | A | 21 | 15219323 | T | C |
| 21 | 15373568 | G | A | 21 | 15475013 | C | T | 21 | 15475149 | C | G |
| 21 | 15475439 | G | A | 21 | 15475515 | T | C | 21 | 16599891 | C | G |
| 21 | 16599895 | T | C | 21 | 16600408 | G | A | 21 | 18236198 | T | A |
| 21 | 19322853 | T | A | 21 | 19322854 | T | C | 21 | 19799542 | C | T |
| 21 | 20144540 | G | C | 21 | 20230102 | C | T | 21 | 20230168 | G | A |
| 21 | 20230169 | T | C | 21 | 20230217 | A | C | 21 | 20230294 | C | A |
| 21 | 20230372 | T | C | 21 | 20230390 | G | A | 21 | 21201593 | G | T |
| 21 | 21201603 | A | G | 21 | 21561669 | C | G | 21 | 21561683 | A | G |
| 21 | 21562354 | A | T | 21 | 22519731 | A | T | 21 | 23687317 | A | G |
| 21 | 24465037 | A | T | 21 | 24563993 | G | T | 21 | 25330218 | A | G |
| 21 | 26832609 | G | T | 21 | 26833183 | C | T | 21 | 26833198 | G | A |
| 21 | 28389497 | A | C | 21 | 30115032 | C | T | 21 | 30115170 | T | G |
| 21 | 30115571 | C | T | 21 | 30115579 | T | A | 21 | 30115629 | C | G |
| 21 | 30115651 | A | G | 21 | 30115655 | G | T | 21 | 30506189 | A | G |
| 21 | 32114077 | A | T | 21 | 32965156 | T | G | 21 | 33471163 | C | T |
| 21 | 33805478 | A | G | 21 | 33805831 | T | C | 21 | 34361496 | G | A |
| 21 | 34776152 | T | G | 21 | 34787638 | C | A | 21 | 34793588 | A | G |
| 21 | 34798982 | G | A | 21 | 35602677 | T | C | 21 | 36232156 | C | A |
| 21 | 36252595 | G | A | 21 | 36252622 | T | C | 21 | 36253194 | T | A |
| 21 | 36261705 | G | A | 21 | 36357463 | C | T | 21 | 36358229 | G | A |
| 21 | 36420874 | C | T | 21 | 36508295 | A | C | 21 | 36972392 | A | G |
| 21 | 41506058 | A | G | 21 | 41506612 | C | T | 21 | 42861181 | G | C |
| 21 | 43528820 | A | G | 21 | 43528822 | C | T | 21 | 43528949 | C | T |
| 21 | 43528970 | C | A | 21 | 43528991 | T | C | 21 | 43529061 | T | G |
| 21 | 43529064 | C | T | 21 | 43529078 | A | G | 21 | 43529162 | C | T |
| 21 | 43529185 | C | A | 21 | 43529216 | G | C | 21 | 43529224 | A | G |
| 21 | 43529228 | C | A | 21 | 45615963 | T | G | 21 | 45616049 | G | A |
| 21 | 46338651 | C | T | 21 | 46339140 | C | T | 21 | 47419697 | C | T |
| 21 | 47419747 | A | G | 21 | 47419770 | G | A | 21 | 47419780 | G | A |
| 21 | 47419794 | T | C | 21 | 47419804 | C | T | 21 | 47419817 | A | G |
| 21 | 47419832 | G | A | 21 | 47419835 | A | G | 21 | 47419856 | G | A |
| 21 | 47419859 | G | A | 21 | 47419859 | G | C | 21 | 47419860 | T | C |
| 21 | 47419872 | G | A | 21 | 47419875 | A | G | 21 | 47419876 | G | A |
| 21 | 47420016 | G | A | 21 | 48021967 | A | T | 22 | 16366289 | C | T |
| 22 | 16366304 | G | A | 22 | 16866125 | G | T | 22 | 16867451 | T | C |
| 22 | 16867494 | T | C | 22 | 16867535 | C | T | 22 | 16867555 | G | A |
| 22 | 16867625 | G | A | 22 | 16867671 | T | G | 22 | 16867807 | G | C |
| 22 | 17000863 | G | A | 22 | 17001006 | G | A | 22 | 17001044 | G | A |
| 22 | 17001074 | T | C | 22 | 17001080 | C | T | 22 | 17001124 | A | G |
| 22 | 17001134 | C | T | 22 | 17001218 | G | C | 22 | 17023726 | T | C |
| 22 | 17349297 | C | G | 22 | 17391914 | A | G | 22 | 17513526 | A | C |
| 22 | 17514026 | G | A | 22 | 17514060 | C | T | 22 | 18465978 | C | T |
| 22 | 18845285 | A | T | 22 | 18845397 | T | C | 22 | 18863659 | C | T |
| 22 | 19486391 | C | T | 22 | 19621254 | A | G | 22 | 20361753 | C | T |
| 22 | 20361768 | A | C | 22 | 21050028 | A | G | 22 | 21050319 | G | A |
| 22 | 21051859 | G | A | 22 | 21051983 | C | A | 22 | 21051987 | T | C |
| 22 | 21051994 | C | A | 22 | 21052014 | A | G | 22 | 21052189 | A | G |
| 22 | 21052193 | T | C | 22 | 21052265 | G | C | 22 | 21052303 | C | T |
| 22 | 21287911 | A | G | 22 | 21303690 | G | T | 22 | 21303711 | G | A |
| 22 | 21304408 | A | G | 22 | 21304463 | T | C | 22 | 21339915 | T | G |
| 22 | 21342169 | G | C | 22 | 21342811 | C | T | 22 | 21343391 | G | C |
| 22 | 21344287 | C | T | 22 | 21344316 | C | T | 22 | 21347410 | G | T |
| 22 | 21347505 | C | T | 22 | 21349548 | A | G | 22 | 21349579 | C | T |
| 22 | 21349676 | T | A | 22 | 21349677 | C | T | 22 | 21351819 | G | A |
| 22 | 21470373 | C | T | 22 | 21644653 | G | A | 22 | 21644684 | T | A |
| 22 | 21644783 | C | T | 22 | 21647786 | T | C | 22 | 21648685 | G | C |
| 22 | 22401267 | G | A | 22 | 22612065 | C | T | 22 | 22625680 | A | C |
| 22 | 22978886 | T | C | 22 | 22978887 | G | A | 22 | 22978887 | G | T |
| 22 | 22980711 | C | G | 22 | 23240831 | T | C | 22 | 23246298 | C | A |
| 22 | 23246323 | C | T | 22 | 23657735 | G | A | 22 | 23659783 | C | G |
| 22 | 23659864 | C | T | 22 | 24129129 | G | T | 22 | 24134394 | T | C |
| 22 | 24136214 | T | C | 22 | 24136252 | C | T | 22 | 24145923 | G | A |
| 22 | 24158697 | G | C | 22 | 24158709 | A | G | 22 | 24159437 | G | T |
| 22 | 24159452 | A | G | 22 | 24167166 | G | A | 22 | 24167185 | C | T |
| 22 | 24167906 | T | C | 22 | 24376322 | G | A | 22 | 24377034 | A | G |
| 22 | 24383954 | C | T | 22 | 24654050 | T | C | 22 | 24655566 | A | G |
| 22 | 24659077 | A | G | 22 | 24659078 | T | C | 22 | 25043518 | C | T |
| 22 | 25068063 | C | T | 22 | 25068076 | C | T | 22 | 25798856 | G | A |
| 22 | 25799013 | T | C | 22 | 25799024 | T | A | 22 | 25832242 | A | G |
| 22 | 26179312 | C | T | 22 | 27863300 | G | A | 22 | 28007568 | G | A |
| 22 | 29090363 | G | A | 22 | 29090890 | G | A | 22 | 29099754 | T | C |
| 22 | 29108112 | A | C | 22 | 29108229 | A | G | 22 | 29129741 | T | C |
| 22 | 29999742 | C | G | 22 | 29999784 | C | A | 22 | 29999878 | G | C |
| 22 | 30032484 | A | C | 22 | 30053963 | A | G | 22 | 30053984 | A | G |
| 22 | 30054024 | G | A | 22 | 30056945 | G | A | 22 | 30064117 | C | T |
| 22 | 30064731 | A | G | 22 | 31071644 | A | C | 22 | 32813600 | T | C |
| 22 | 32813975 | C | G | 22 | 33171838 | G | T | 22 | 34225927 | C | A |
| 22 | 34226068 | G | A | 22 | 34226412 | C | G | 22 | 34226440 | T | C |
| 22 | 34842414 | G | C | 22 | 35843103 | C | T | 22 | 35843109 | C | T |
| 22 | 36678453 | C | G | 22 | 36678476 | C | A | 22 | 36679058 | T | C |
| 22 | 38054038 | A | G | 22 | 41514151 | C | T | 22 | 41514155 | G | A |
| 22 | 41536064 | T | C | 22 | 41543721 | G | A | 22 | 41544167 | C | T |
| 22 | 41552926 | T | G | 22 | 41554690 | C | T | 22 | 41565285 | A | G |
| 22 | 41565827 | A | G | 22 | 41566823 | A | G | 22 | 41570071 | T | C |
| 22 | 42232577 | C | T | 22 | 42232588 | G | A | 22 | 42522312 | T | C |
| 22 | 42522392 | G | A | 22 | 42523003 | A | G | 22 | 42523209 | T | C |
| 22 | 42523211 | T | C | 22 | 42526049 | C | G | 22 | 42536064 | T | C |
| 22 | 42536954 | A | G | 22 | 42537023 | T | C | 22 | 42537029 | C | T |
| 22 | 42537115 | C | T | 22 | 42537119 | C | T | 22 | 42537127 | A | G |
| 22 | 42537196 | T | C | 22 | 42538399 | C | T | 22 | 42539105 | T | C |
| 22 | 42539720 | C | G | 22 | 42540667 | C | T | 22 | 42540668 | A | G |
| 22 | 42547739 | G | A | 22 | 42547755 | C | G | 22 | 42548094 | A | G |
| 22 | 42548880 | G | A | 22 | 43735824 | T | C | 22 | 44510034 | T | A |
| 22 | 44510219 | T | C | 22 | 44510230 | T | C | 22 | 44510240 | A | G |
| 22 | 44510329 | C | T | 22 | 44510554 | G | A | 22 | 44510624 | A | G |
| 22 | 44510631 | G | T | 22 | 44511408 | G | C | 22 | 44511478 | T | C |
| 22 | 44511626 | T | C | 22 | 44511663 | G | C | 22 | 45676445 | C | G |
| 22 | 47002427 | T | C | 22 | 47566744 | G | A | 22 | 48301616 | T | C |
| 22 | 48301642 | C | T | 22 | 48301646 | G | A | 22 | 48301660 | C | T |
| 22 | 49258340 | G | C | 22 | 49258341 | G | C | 22 | 49258485 | A | G |
| 22 | 49258525 | A | G | 22 | 49258700 | G | C | 22 | 49258701 | G | C |
| 22 | 49551354 | A | G | 22 | 49551680 | G | A | 22 | 49551682 | G | A |
| 22 | 49551684 | A | G | 22 | 49551689 | C | A | 22 | 49551690 | C | T |
| 22 | 49551694 | G | A | 22 | 49551724 | G | C | 22 | 49551734 | T | C |
| 22 | 49551749 | G | A | 22 | 49551895 | G | A | 22 | 49551927 | G | A |
| 22 | 49551969 | C | T | 22 | 49551990 | T | C | 22 | 50017556 | G | A |
| 22 | 51115526 | T | C | 22 | 51115575 | C | T | X | 323851 | G | A |
| X | 323960 | C | T | X | 324085 | G | A | X | 324097 | T | C |
| X | 324135 | G | A | X | 324137 | A | C | X | 324137 | A | G |
| X | 324146 | G | A | X | 324224 | C | G | X | 324245 | G | A |
| X | 933046 | T | A | X | 933079 | A | T | X | 933848 | C | T |
| X | 1414936 | C | A | X | 1415582 | A | C | X | 1415726 | G | C |
| X | 1415754 | A | G | X | 1416082 | A | C | X | 1416494 | A | C |
| X | 1416825 | A | G | X | 1416859 | C | G | X | 1417722 | A | G |
| X | 1882476 | C | A | X | 1882644 | G | C | X | 1882671 | T | G |
| X | 1883661 | T | G | X | 2320736 | C | T | X | 2320768 | C | T |
| X | 2550218 | A | G | X | 2550271 | T | C | X | 3354064 | T | C |
| X | 3354073 | C | G | X | 3354304 | T | G | X | 3354320 | C | G |
| X | 3354323 | T | C | X | 3354489 | C | T | X | 3947844 | G | A |
| X | 3947940 | G | A | X | 3948055 | A | G | X | 3948056 | C | A |
| X | 3948072 | C | A | X | 3948325 | A | G | X | 5728321 | G | A |
| X | 5728524 | T | A | X | 6750277 | G | A | X | 6750665 | T | C |
| X | 7811259 | G | C | X | 7811504 | G | A | X | 7811645 | C | G |
| X | 7811747 | T | C | X | 7812017 | C | T | X | 7812081 | C | T |
| X | 7812197 | T | C | X | 8138080 | G | C | X | 8138160 | T | C |
| X | 8138165 | C | G | X | 8138170 | C | G | X | 8138171 | T | C |
| X | 8138182 | A | G | X | 8434256 | A | G | X | 8434367 | C | T |
| X | 8434432 | A | G | X | 8434433 | G | C | X | 8960281 | C | T |
| X | 9372403 | A | G | X | 9374934 | C | A | X | 9375163 | A | C |
| X | 9376287 | T | G | X | 9377057 | G | A | X | 9378063 | T | C |
| X | 9378595 | C | G | X | 9380687 | C | G | X | 9382151 | T | C |
| X | 9909935 | T | C | X | 9910404 | A | T | X | 9910483 | C | A |
| X | 11985475 | C | T | X | 11985898 | A | T | X | 12023301 | C | G |
| X | 12023305 | A | G | X | 12023314 | A | G | X | 12023535 | G | C |
| X | 13222032 | G | A | X | 13668792 | C | T | X | 13669007 | G | A |
| X | 14291097 | G | A | X | 14291174 | G | A | X | 14291313 | T | C |
| X | 14852765 | G | C | X | 15665327 | C | T | X | 16351394 | T | C |
| X | 16351704 | C | T | X | 16351825 | A | G | X | 17387641 | A | G |
| X | 17794293 | A | G | X | 17794390 | G | C | X | 18247605 | T | C |
| X | 21942691 | C | T | X | 22313514 | G | A | X | 22314058 | G | C |
| X | 22553123 | A | G | X | 22553314 | G | A | X | 24007345 | T | C |
| X | 26225293 | C | T | X | 26225571 | C | A | X | 26735568 | A | G |
| X | 26735679 | G | T | X | 27029285 | C | A | X | 28676918 | A | G |
| X | 28871324 | A | G | X | 28871343 | A | G | X | 28871801 | C | T |
| X | 29149454 | G | T | X | 29149490 | A | G | X | 30277764 | C | T |
| X | 30618867 | T | C | X | 31277151 | C | T | X | 32224435 | T | C |
| X | 32889854 | G | A | X | 33050164 | T | C | X | 34444321 | T | G |
| X | 35083559 | C | A | X | 35971670 | A | C | X | 35971684 | A | G |
| X | 36959988 | C | T | X | 39944288 | C | T | X | 40857040 | C | T |
| X | 41546554 | C | T | X | 43440829 | T | C | X | 43440856 | T | C |
| X | 44601065 | T | G | X | 45028722 | T | C | X | 45029140 | A | G |
| X | 45634577 | C | A | X | 47966001 | A | G | X | 48134195 | T | C |
| X | 48134267 | C | A | X | 48134283 | A | G | X | 48134352 | G | A |
| X | 48134363 | T | C | X | 48196483 | C | T | X | 48548091 | C | T |
| X | 49337861 | G | A | X | 49337915 | G | A | X | 50776337 | A | C |
| X | 51361920 | C | T | X | 54846603 | C | T | X | 54976385 | T | C |
| X | 55684550 | G | C | X | 55684552 | A | T | X | 55684553 | T | C |
| X | 57163476 | C | G | X | 62573853 | A | G | X | 67310673 | G | A |
| X | 67965273 | C | T | X | 70338399 | A | G | X | 70354080 | C | A |
| X | 70355942 | G | C | X | 70361608 | T | C | X | 70361619 | T | C |
| X | 70886761 | G | A | X | 70939147 | G | C | X | 72066630 | C | T |
| X | 74375460 | C | T | X | 74803555 | T | A | X | 75449353 | G | C |
| X | 75449473 | A | G | X | 75555810 | C | T | X | 76845060 | C | T |
| X | 76848940 | C | T | X | 76912391 | T | C | X | 76919991 | T | C |
| X | 77671395 | A | G | X | 78526387 | C | G | X | 79376181 | C | G |
| X | 79376359 | G | A | X | 79712450 | C | A | X | 81978259 | A | G |
| X | 82987871 | T | A | X | 82988286 | T | A | X | 83489042 | C | A |
| X | 84535451 | A | G | X | 85996514 | A | G | X | 86125877 | C | T |
| X | 86126034 | A | C | X | 86126184 | C | A | X | 86126214 | C | A |
| X | 88458887 | G | A | X | 88459088 | A | G | X | 88460086 | C | T |
| X | 88460163 | C | A | X | 88460234 | C | T | X | 88460693 | C | G |
| X | 88461023 | A | C | X | 88461098 | G | T | X | 88461193 | G | C |
| X | 88461257 | T | C | X | 88461306 | C | T | X | 88461381 | G | A |
| X | 88461454 | G | T | X | 88461489 | G | A | X | 89179384 | G | A |
| X | 89531825 | G | A | X | 90467751 | C | T | X | 90468268 | A | G |
| X | 90937645 | C | T | X | 90937839 | C | T | X | 91240753 | G | A |
| X | 91241043 | C | T | X | 91651454 | G | A | X | 91651560 | T | C |
| X | 91651589 | T | C | X | 91651601 | C | G | X | 94235658 | G | C |
| X | 97861548 | G | A | X | 97861806 | C | G | X | 100600966 | C | T |
| X | 100604681 | C | T | X | 100611432 | G | A | X | 100614597 | C | T |
| X | 100615025 | G | T | X | 100616989 | C | T | X | 100625293 | C | T |
| X | 100626864 | C | T | X | 100630500 | G | A | X | 101383199 | T | C |
| X | 102633394 | A | T | X | 105034364 | C | G | X | 106516125 | G | C |
| X | 107492960 | G | C | X | 108115548 | C | T | X | 111815818 | T | C |
| X | 114629506 | T | C | X | 114997566 | A | G | X | 115005861 | T | C |
| X | 115005934 | C | T | X | 115959641 | C | A | X | 116167250 | G | A |
| X | 117479815 | C | G | X | 119313694 | G | C | X | 122008986 | A | G |
| X | 122648454 | T | C | X | 122696563 | C | T | X | 122932585 | A | C |
| X | 123159474 | T | C | X | 123181551 | G | A | X | 123194867 | T | C |
| X | 123197557 | T | C | X | 123227604 | T | C | X | 124576157 | G | T |
| X | 125779449 | T | C | X | 125779655 | C | T | X | 126247979 | C | A |
| X | 127724972 | C | T | X | 128495450 | C | G | X | 130974130 | T | C |
| X | 130974165 | A | G | X | 130974206 | G | A | X | 130974276 | A | C |
| X | 130974277 | A | C | X | 130974284 | G | C | X | 130974290 | C | G |
| X | 130974310 | T | C | X | 130974312 | G | A | X | 132426862 | G | C |
| X | 135961796 | C | G | X | 137367771 | T | C | X | 138265862 | A | T |
| X | 138266294 | T | C | X | 139298295 | C | T | X | 139298601 | C | T |
| X | 139298612 | T | C | X | 139960824 | T | C | X | 140147409 | A | G |
| X | 140147500 | C | T | X | 140336615 | T | A | X | 140673061 | C | T |
| X | 140673198 | T | G | X | 140673372 | C | T | X | 140673423 | G | C |
| X | 140786796 | G | A | X | 140993642 | C | T | X | 140993905 | C | T |
| X | 140993912 | C | T | X | 140993913 | C | G | X | 140993945 | T | C |
| X | 140993947 | T | C | X | 140993989 | T | C | X | 140994031 | G | C |
| X | 140994037 | C | A | X | 142605437 | A | C | X | 142605455 | C | T |
| X | 142605483 | A | G | X | 142605491 | G | C | X | 142605583 | C | T |
| X | 142605628 | G | A | X | 142804302 | T | C | X | 142804326 | C | T |
| X | 143901920 | T | C | X | 143902298 | G | T | X | 144328894 | C | T |
| X | 144940393 | G | A | X | 146052064 | T | C | X | 146052294 | G | C |
| X | 146266206 | T | G | X | 146266521 | G | T | X | 146679744 | A | G |
| X | 146679975 | T | A | X | 146680129 | C | T | X | 149382872 | C | G |
| X | 149383074 | A | G | X | 150162380 | C | T | X | 152155921 | C | T |
| X | 152156110 | C | T | X | 152156144 | A | G | X | 152156176 | G | C |
| X | 152538286 | A | T | X | 152538627 | G | A | X | 152582526 | G | A |
| X | 153009797 | G | A | X | 153010224 | C | T | X | 153010267 | G | A |
| X | 154376753 | C | G | X | 155233098 | T | C | | | | |

What is claimed is:

1. Use of a reagent for detecting a marker in the preparation of a reagent for evaluating homologous recombination deficiency (HRD), the marker is composed of the SNP sites in which the site information is shown as follows:

| Chromo- | | Refer- | Varia- | Chromo- | | Refer- | Varia- | Chromo- | | Refer- | Varia- |
|---|---|---|---|---|---|---|---|---|---|---|---|
| some | Location | ence | tion | some | Location | ence | tion | some | Location | ence | tion |
| 1 | 105279 | G | C | 1 | 251663 | C | G | 1 | 649113 | C | T |
| 1 | 649192 | A | T | 1 | 743072 | C | A | 1 | 924528 | C | A |
| 1 | 1127507 | G | A | 1 | 1127523 | C | T | 1 | 1128429 | G | A |
| 1 | 1128521 | C | T | 1 | 1650410 | T | C | 1 | 2488930 | G | A |
| 1 | 2631804 | G | A | 1 | 2693121 | A | T | 1 | 3097439 | C | T |
| 1 | 3097459 | C | T | 1 | 3097464 | A | G | 1 | 3097498 | A | T |
| 1 | 3097509 | C | T | 1 | 3097522 | T | C | 1 | 3097532 | T | G |
| 1 | 3097534 | G | A | 1 | 3098219 | C | A | 1 | 3098360 | A | G |
| 1 | 3098386 | G | T | 1 | 4196428 | G | A | 1 | 4967739 | G | C |
| 1 | 4967744 | C | A | 1 | 5393906 | G | A | 1 | 5876714 | T | A |
| 1 | 5876956 | A | G | 1 | 5876977 | C | A | 1 | 5876988 | A | G |
| 1 | 5877088 | C | G | 1 | 5877112 | C | T | 1 | 5877121 | C | A |
| 1 | 5877138 | G | A | 1 | 5877145 | C | A | 1 | 5877169 | C | A |
| 1 | 5877171 | T | C | 1 | 5877327 | A | C | 1 | 5877329 | A | G |
| 1 | 5877337 | G | A | 1 | 5877367 | G | A | 1 | 5877384 | C | T |
| 1 | 6481623 | G | A | 1 | 9493601 | C | T | 1 | 9494062 | A | G |
| 1 | 9494142 | T | C | 1 | 10357406 | A | G | 1 | 11167760 | C | T |
| 1 | 11167829 | C | T | 1 | 11168529 | A | G | 1 | 11174067 | A | T |
| 1 | 11175176 | T | C | 1 | 11181630 | G | A | 1 | 11189191 | G | A |
| 1 | 11193401 | A | C | 1 | 11193406 | C | T | 1 | 11193408 | C | T |
| 1 | 11209899 | T | C | 1 | 11227224 | C | T | 1 | 11269796 | T | C |
| 1 | 11292753 | A | G | 1 | 11293200 | C | T | 1 | 11293216 | A | G |
| 1 | 11293792 | A | C | 1 | 11303610 | T | C | 1 | 11307412 | G | C |
| 1 | 11315853 | G | A | 1 | 11318763 | C | G | 1 | 11318983 | T | C |
| 1 | 12188028 | C | A | 1 | 12854713 | G | T | 1 | 12854716 | A | G |
| 1 | 12886052 | C | T | 1 | 12886098 | T | C | 1 | 12886436 | C | A |
| 1 | 12920067 | C | G | 1 | 12920132 | A | G | 1 | 12920191 | T | C |
| 1 | 12920200 | G | A | 1 | 12920622 | C | T | 1 | 12920708 | C | A |
| 1 | 12940618 | A | G | 1 | 12940822 | A | T | 1 | 12941059 | C | A |
| 1 | 12941086 | C | A | 1 | 13143375 | G | T | 1 | 13143405 | A | G |
| 1 | 13143441 | C | T | 1 | 13143453 | C | T | 1 | 13367310 | C | G |
| 1 | 13367310 | C | T | 1 | 14228227 | C | G | 1 | 14228236 | A | G |
| 1 | 14228340 | G | C | 1 | 14228356 | T | C | 1 | 15829457 | C | G |
| 1 | 16451413 | C | T | 1 | 16460541 | T | C | 1 | 17345113 | G | C |
| 1 | 17348847 | G | C | 1 | 17350243 | T | C | 1 | 17353988 | T | G |
| 1 | 17370970 | C | T | 1 | 17687161 | G | C | 1 | 18476868 | T | C |
| 1 | 18476876 | A | G | 1 | 18477394 | A | C | 1 | 18477450 | G | A |
| 1 | 20058523 | A | G | 1 | 20915418 | C | G | 1 | 20916080 | C | G |
| 1 | 20931774 | A | G | 1 | 20944721 | G | A | 1 | 22234424 | G | C |
| 1 | 22301165 | C | T | 1 | 22301180 | G | A | 1 | 22301199 | C | T |
| 1 | 22301755 | A | G | 1 | 22301769 | G | A | 1 | 22301770 | C | T |
| 1 | 22301824 | T | C | 1 | 22326109 | T | C | 1 | 22326455 | T | C |
| 1 | 22326488 | T | C | 1 | 22326548 | A | G | 1 | 22326587 | G | A |
| 1 | 23909324 | T | A | 1 | 24192453 | T | C | 1 | 26228409 | A | G |
| 1 | 28439964 | C | T | 1 | 28563391 | C | T | 1 | 29232578 | C | G |
| 1 | 31581492 | C | T | 1 | 31857847 | G | A | 1 | 31857869 | G | A |
| 1 | 31857916 | C | T | 1 | 33819830 | A | C | 1 | 34989237 | G | A |
| 1 | 37049763 | C | T | 1 | 37568312 | C | G | 1 | 38010651 | G | A |
| 1 | 38010751 | C | T | 1 | 39944488 | T | G | 1 | 40367607 | G | C |
| 1 | 40367625 | G | T | 1 | 40799427 | G | A | 1 | 41940980 | C | T |
| 1 | 42042317 | C | T | 1 | 42042915 | C | T | 1 | 43803222 | G | A |
| 1 | 44606254 | C | T | 1 | 44606565 | A | G | 1 | 45880669 | C | T |
| 1 | 45880751 | C | T | 1 | 46727304 | C | T | 1 | 46737961 | C | T |
| 1 | 47009757 | G | A | 1 | 47009799 | A | G | 1 | 47009829 | G | T |
| 1 | 47541531 | C | A | 1 | 48119989 | C | T | 1 | 48120087 | T | C |
| 1 | 50591705 | C | G | 1 | 50591743 | C | T | 1 | 53116251 | A | G |
| 1 | 55390669 | T | C | 1 | 55834642 | G | A | 1 | 55835253 | G | A |
| 1 | 57548321 | C | G | 1 | 59621601 | C | T | 1 | 59621636 | A | T |
| 1 | 59622219 | G | A | 1 | 59622225 | T | G | 1 | 61465647 | C | G |
| 1 | 62148611 | A | G | 1 | 63521709 | T | C | 1 | 63521755 | T | C |
| 1 | 64117913 | A | G | 1 | 65301317 | G | A | 1 | 65301490 | G | A |
| 1 | 65301611 | T | G | 1 | 65303338 | A | G | 1 | 65303390 | G | A |
| 1 | 65312583 | C | T | 1 | 65312949 | A | G | 1 | 65335343 | C | G |
| 1 | 65338745 | C | T | 1 | 65338775 | C | T | 1 | 67822377 | A | G |
| 1 | 68628372 | G | A | 1 | 71012395 | C | A | 1 | 72571656 | C | A |
| 1 | 73095148 | G | T | 1 | 77259478 | G | A | 1 | 77754620 | A | G |
| 1 | 80363334 | G | C | 1 | 82467045 | T | C | 1 | 82843604 | G | T |
| 1 | 83054685 | T | C | 1 | 83054799 | C | T | 1 | 83055055 | T | C |
| 1 | 83055137 | G | T | 1 | 83055238 | G | T | 1 | 84295861 | A | G |
| 1 | 85229792 | G | C | 1 | 86050077 | C | G | 1 | 86873847 | C | T |
| 1 | 87106420 | T | C | 1 | 89849268 | T | C | 1 | 90271586 | T | C |
| 1 | 90673701 | C | T | 1 | 90673754 | G | A | 1 | 91658629 | G | A |
| 1 | 91659249 | G | A | 1 | 91659289 | A | T | 1 | 92082962 | C | T |
| 1 | 96479066 | T | A | 1 | 96993374 | C | A | 1 | 96993449 | T | C |
| 1 | 97838854 | C | T | 1 | 97839413 | C | G | 1 | 97981706 | C | T |
| 1 | 99692027 | A | G | 1 | 100270805 | G | C | 1 | 102251801 | A | G |
| 1 | 102427608 | C | T | 1 | 103047194 | C | T | 1 | 105484566 | G | A |
| 1 | 106740553 | T | G | 1 | 107182505 | A | T | 1 | 110231592 | T | C |
| 1 | 110231777 | G | A | 1 | 110232087 | C | T | 1 | 110233138 | G | C |
| 1 | 110233147 | C | T | 1 | 110235607 | A | G | 1 | 110255174 | T | G |
| 1 | 110256473 | C | G | 1 | 111857685 | G | C | 1 | 111858291 | T | C |
| 1 | 112033285 | G | C | 1 | 113234456 | G | A | 1 | 117786992 | G | A |
| 1 | 117787502 | C | A | 1 | 118136824 | T | C | 1 | 118136861 | G | A |
| 1 | 118136912 | G | A | 1 | 119762037 | T | C | 1 | 119992928 | T | C |
| 1 | 119992995 | C | A | 1 | 119992999 | A | G | 1 | 120462468 | C | T |
| 1 | 120463230 | C | T | 1 | 120483921 | G | C | 1 | 120495934 | G | C |
| 1 | 120498086 | G | C | 1 | 120508803 | T | C | 1 | 120509783 | A | G |
| 1 | 120547695 | A | G | 1 | 144838618 | A | C | 1 | 145931852 | A | C |
| 1 | 146465784 | C | G | 1 | 146555193 | T | C | 1 | 147534441 | T | A |
| 1 | 148004440 | C | A | 1 | 148004899 | T | A | 1 | 148756363 | A | G |
| 1 | 148756397 | C | T | 1 | 148756432 | C | A | 1 | 148756499 | A | G |
| 1 | 148756515 | A | C | 1 | 148854651 | A | C | 1 | 148855855 | C | T |
| 1 | 149067299 | G | A | 1 | 149089962 | A | C | 1 | 149521316 | A | G |
| 1 | 149521391 | A | T | 1 | 149606839 | C | G | 1 | 149607021 | A | G |
| 1 | 149607027 | G | A | 1 | 152277345 | C | T | 1 | 152277396 | T | C |
| 1 | 152278689 | C | A | 1 | 152279406 | T | G | 1 | 152280032 | T | C |
| 1 | 152280170 | C | G | 1 | 152281007 | A | G | 1 | 152281008 | A | G |
| 1 | 152281039 | G | A | 1 | 152760075 | A | G | 1 | 152760093 | T | C |
| 1 | 152760096 | A | T | 1 | 152760628 | T | C | 1 | 152760642 | C | T |
| 1 | 152760644 | C | T | 1 | 152760647 | C | A | 1 | 152760651 | C | T |
| 1 | 152770521 | G | A | 1 | 152770533 | G | A | 1 | 152770589 | A | G |
| 1 | 152770828 | A | G | 1 | 153277768 | C | T | 1 | 154351073 | T | C |
| 1 | 155583937 | A | G | 1 | 156785356 | T | C | 1 | 158165578 | G | A |
| 1 | 158165601 | T | C | 1 | 158165661 | T | C | 1 | 158165722 | G | A |
| 1 | 158165742 | A | T | 1 | 158165938 | A | C | 1 | 158165984 | C | T |
| 1 | 158220870 | T | C | 1 | 158736215 | G | A | 1 | 159086711 | A | G |
| 1 | 160093648 | G | A | 1 | 160236470 | T | A | 1 | 161293193 | C | T |
| 1 | 161293215 | A | G | 1 | 161293646 | T | C | 1 | 161293708 | G | A |
| 1 | 161297993 | T | C | 1 | 161310719 | C | T | 1 | 161326201 | C | T |
| 1 | 161326274 | A | G | 1 | 161326293 | T | C | 1 | 161558725 | C | T |
| 1 | 161559082 | C | T | 1 | 162470503 | G | A | 1 | 163033342 | A | C |
| 1 | 166245169 | A | G | 1 | 166245170 | G | C | 1 | 166541921 | T | C |
| 1 | 167638203 | T | C | 1 | 167638251 | T | G | 1 | 168365929 | C | G |
| 1 | 168513243 | G | A | 1 | 168513692 | C | T | 1 | 168545758 | C | T |
| 1 | 177940341 | C | T | 1 | 177940348 | C | T | 1 | 178922201 | A | T |
| 1 | 179335158 | C | T | 1 | 179335162 | G | C | 1 | 180162293 | G | C |
| 1 | 180162364 | C | T | 1 | 181394677 | G | C | 1 | 181828178 | T | G |
| 1 | 186256808 | A | G | 1 | 188674740 | C | A | 1 | 189555573 | G | C |
| 1 | 191151643 | G | T | 1 | 191152225 | C | T | 1 | 191719275 | A | T |
| 1 | 192017093 | C | G | 1 | 194952546 | G | C | 1 | 195371632 | G | A |
| 1 | 196788601 | C | T | 1 | 196912415 | C | T | 1 | 198340898 | T | C |
| 1 | 199449355 | C | T | 1 | 199548706 | C | G | 1 | 201178470 | G | A |
| 1 | 201489285 | G | C | 1 | 201489286 | C | G | 1 | 201489302 | C | T |
| 1 | 201489565 | C | T | 1 | 201978049 | C | T | 1 | 201978068 | G | A |
| 1 | 202096562 | T | C | 1 | 203274536 | C | A | 1 | 203842900 | C | T |
| 1 | 203843484 | T | C | 1 | 204315839 | T | G | 1 | 204494436 | C | T |
| 1 | 204495253 | G | A | 1 | 204500257 | G | A | 1 | 204511704 | T | C |
| 1 | 204513402 | G | A | 1 | 205816893 | T | C | 1 | 205816923 | G | A |
| 1 | 206646274 | G | T | 1 | 206646311 | C | T | 1 | 206647387 | T | G |
| 1 | 206647450 | A | G | 1 | 206669136 | C | A | 1 | 207807908 | C | T |
| 1 | 207807921 | G | C | 1 | 207807928 | G | A | 1 | 207808385 | C | G |
| 1 | 208164511 | C | T | 1 | 208780934 | A | C | 1 | 210371949 | A | G |
| 1 | 210372455 | T | C | 1 | 210827321 | T | C | 1 | 210827322 | G | A |
| 1 | 210827379 | C | T | 1 | 211126407 | G | A | 1 | 211855564 | G | A |
| 1 | 211855593 | T | A | 1 | 212224761 | A | G | 1 | 212224946 | G | T |
| 1 | 212240533 | A | G | 1 | 212245792 | A | G | 1 | 214386346 | T | G |
| 1 | 214386351 | C | T | 1 | 214386365 | A | T | 1 | 214571527 | C | G |
| 1 | 215128241 | C | T | 1 | 216840332 | G | C | 1 | 216840371 | C | T |
| 1 | 216840798 | C | T | 1 | 218238495 | T | C | 1 | 218572004 | G | A |
| 1 | 219969405 | T | C | 1 | 222477115 | C | A | 1 | 222642478 | C | A |
| 1 | 222642700 | G | A | 1 | 222677791 | C | G | 1 | 223089124 | G | A |
| 1 | 223718462 | G | A | 1 | 223718518 | C | A | 1 | 224164610 | G | A |
| 1 | 224220492 | A | G | 1 | 224220734 | G | A | 1 | 224644312 | C | T |
| 1 | 226549498 | G | A | 1 | 226553934 | A | C | 1 | 226557878 | T | C |
| 1 | 226580187 | T | C | 1 | 227680600 | G | A | 1 | 227680783 | T | C |
| 1 | 227680868 | T | C | 1 | 227681213 | T | C | 1 | 227681563 | A | G |
| 1 | 227681567 | T | C | 1 | 227695408 | T | G | 1 | 227695429 | C | G |
| 1 | 227695486 | G | A | 1 | 227695818 | C | T | 1 | 227695830 | C | T |
| 1 | 227695868 | G | A | 1 | 227696002 | A | G | 1 | 227696033 | T | C |
| 1 | 227696110 | G | A | 1 | 228156074 | C | T | 1 | 228163615 | A | C |
| 1 | 228163691 | A | G | 1 | 228164114 | G | C | 1 | 228164190 | G | A |
| 1 | 228164584 | A | G | 1 | 228165010 | A | G | 1 | 228457036 | T | A |
| 1 | 228457543 | G | A | 1 | 228716894 | C | T | 1 | 229824770 | T | C |
| 1 | 231146762 | G | A | 1 | 231146770 | G | C | 1 | 232491583 | T | G |
| 1 | 233280999 | T | A | 1 | 234126736 | G | A | 1 | 234127282 | C | G |
| 1 | 234127319 | C | T | 1 | 234128435 | A | G | 1 | 234128441 | A | T |
| 1 | 234128967 | C | T | 1 | 234948160 | T | C | 1 | 235049923 | A | C |
| 1 | 235702712 | C | T | 1 | 236919092 | T | G | 1 | 236919146 | G | A |
| 1 | 237410497 | C | G | 1 | 237410504 | G | C | 1 | 237445099 | T | C |
| 1 | 238113949 | T | A | 1 | 239800851 | C | T | 1 | 240451555 | T | C |
| 1 | 240451568 | C | T | 1 | 241665510 | A | G | 1 | 241671638 | C | T |
| 1 | 242453104 | C | T | 1 | 242819974 | T | G | 1 | 243204262 | G | T |
| 1 | 243245426 | C | T | 1 | 243662773 | T | C | 1 | 243801190 | T | C |
| 1 | 243828306 | T | A | 1 | 244891367 | A | G | 1 | 244891415 | G | A |
| 1 | 245856763 | G | A | 1 | 246362962 | T | C | 1 | 246363094 | G | C |
| 1 | 246641904 | T | C | 1 | 246642046 | A | G | 1 | 246642078 | T | C |
| 1 | 246642155 | A | G | 1 | 247337911 | A | C | 1 | 247338427 | A | G |
| 1 | 247591141 | G | A | 1 | 248084854 | A | G | 1 | 248084909 | T | G |
| 1 | 248085341 | T | A | 1 | 248436611 | G | A | 1 | 248436616 | A | G |
| 1 | 248907914 | G | T | 1 | 248907917 | C | T | 1 | 248907919 | C | T |
| 2 | 197601 | A | G | 2 | 197712 | T | C | 2 | 1366783 | G | A |
| 2 | 1366859 | G | A | 2 | 1833356 | T | C | 2 | 1833781 | G | A |
| 2 | 2413270 | T | G | 2 | 2413783 | C | T | 2 | 2940325 | G | A |
| 2 | 2940329 | A | G | 2 | 3332735 | A | G | 2 | 3333243 | T | G |
| 2 | 3877140 | C | T | 2 | 3877747 | G | A | 2 | 4561867 | T | C |
| 2 | 5389739 | A | G | 2 | 5768841 | C | G | 2 | 6426700 | T | C |
| 2 | 6426703 | C | T | 2 | 6943743 | C | A | 2 | 7068710 | C | G |
| 2 | 7999597 | G | A | 2 | 9446167 | C | A | 2 | 9446205 | G | A |
| 2 | 10381717 | C | T | 2 | 10676227 | C | G | 2 | 10676309 | C | T |
| 2 | 10676315 | A | G | 2 | 10676317 | T | G | 2 | 10676320 | G | A |
| 2 | 10676368 | T | C | 2 | 10676498 | T | C | 2 | 10676610 | G | A |
| 2 | 11093401 | T | C | 2 | 11093430 | T | C | 2 | 11242610 | C | T |
| 2 | 11994573 | A | C | 2 | 11994575 | G | A | 2 | 11994645 | A | G |
| 2 | 11994813 | A | G | 2 | 11994822 | C | G | 2 | 11994829 | T | A |
| 2 | 11994840 | T | A | 2 | 11994849 | T | C | 2 | 11994863 | A | C |
| 2 | 11994865 | G | A | 2 | 11994907 | T | C | 2 | 13981557 | C | T |
| 2 | 14082251 | G | T | 2 | 15832606 | G | A | 2 | 16086469 | C | T |
| 2 | 19524172 | T | G | 2 | 21634473 | T | C | 2 | 24167417 | T | G |
| 2 | 24439048 | A | G | 2 | 25458379 | G | A | 2 | 25472784 | C | T |
| 2 | 25505793 | T | C | 2 | 25522795 | G | A | 2 | 26367863 | T | C |
| 2 | 26835397 | C | A | 2 | 26835419 | G | A | 2 | 27615389 | C | T |
| 2 | 28350922 | G | A | 2 | 29416946 | T | C | 2 | 29432982 | C | T |
| 2 | 29436633 | G | T | 2 | 29437182 | T | C | 2 | 29451511 | C | G |
| 2 | 29940820 | C | T | 2 | 30142547 | G | C | 2 | 31552688 | G | C |
| 2 | 31553158 | C | G | 2 | 33896721 | G | A | 2 | 35130065 | T | G |
| 2 | 36768719 | C | G | 2 | 37050374 | A | G | 2 | 37996061 | C | T |
| 2 | 38002857 | T | G | 2 | 40783500 | C | G | 2 | 40783501 | T | C |
| 2 | 41273446 | A | C | 2 | 41273450 | T | C | 2 | 43779665 | T | C |
| 2 | 43783241 | C | A | 2 | 43783273 | G | A | 2 | 43787758 | G | C |
| 2 | 43798616 | C | G | 2 | 43802347 | T | A | 2 | 43802380 | C | G |
| 2 | 43803897 | A | C | 2 | 44274227 | G | C | 2 | 44274729 | A | G |
| 2 | 44292977 | C | T | 2 | 44985166 | C | G | 2 | 45576533 | C | A |
| 2 | 45577081 | A | G | 2 | 46064330 | A | G | 2 | 46525391 | G | C |
| 2 | 46588488 | G | T | 2 | 46597296 | T | C | 2 | 46603260 | C | T |
| 2 | 46603438 | A | G | 2 | 46604857 | T | C | 2 | 46605659 | A | G |
| 2 | 46611471 | G | A | 2 | 47597029 | C | A | 2 | 47602241 | T | C |
| 2 | 47606683 | T | C | 2 | 47612156 | A | G | 2 | 47639871 | T | G |
| 2 | 47643191 | C | G | 2 | 47672475 | T | A | 2 | 47698384 | G | A |
| 2 | 47703241 | A | G | 2 | 47703984 | T | G | 2 | 47709704 | T | C |
| 2 | 48022900 | A | G | 2 | 48914615 | C | G | 2 | 48941428 | C | T |
| 2 | 48952574 | C | T | 2 | 48956475 | C | T | 2 | 48956512 | A | G |
| 2 | 48958595 | A | G | 2 | 48958626 | A | G | 2 | 48958647 | C | T |
| 2 | 48982358 | G | A | 2 | 49070593 | G | C | 2 | 49071094 | G | C |
| 2 | 49660621 | A | T | 2 | 50231869 | G | A | 2 | 50232465 | C | T |
| 2 | 50763570 | A | G | 2 | 51450722 | A | G | 2 | 52153976 | T | C |
| 2 | 53792407 | T | C | 2 | 55066617 | G | C | 2 | 56338148 | G | T |
| 2 | 58453585 | T | C | 2 | 58453620 | C | G | 2 | 59260307 | G | A |
| 2 | 61183901 | A | G | 2 | 62373915 | T | C | 2 | 64720254 | G | A |
| 2 | 64892858 | G | T | 2 | 65863984 | T | G | 2 | 65864061 | C | G |
| 2 | 66063089 | A | G | 2 | 66955742 | C | T | 2 | 68999640 | G | A |
| 2 | 71256898 | G | T | 2 | 71256905 | C | A | 2 | 71256965 | G | A |
| 2 | 71283017 | G | T | 2 | 71283128 | G | A | 2 | 71283247 | G | A |
| 2 | 71283425 | T | G | 2 | 73295583 | G | A | 2 | 73869120 | G | A |
| 2 | 73869558 | T | C | 2 | 75089569 | A | G | 2 | 75090155 | G | A |
| 2 | 76124531 | C | T | 2 | 76124535 | C | T | 2 | 76890353 | T | A |
| 2 | 77637227 | A | T | 2 | 79445642 | A | T | 2 | 80101025 | G | A |
| 2 | 80101040 | A | G | 2 | 81423752 | T | C | 2 | 83419234 | G | C |
| 2 | 84073752 | A | T | 2 | 84074312 | A | G | 2 | 85141323 | A | G |
| 2 | 85685734 | A | G | 2 | 87405001 | T | C | 2 | 87405397 | G | A |
| 2 | 87444116 | A | G | 2 | 87444177 | A | G | 2 | 87444438 | C | T |
| 2 | 87615504 | A | G | 2 | 88433305 | T | C | 2 | 88739133 | T | A |
| 2 | 88739134 | C | G | 2 | 88739141 | C | A | 2 | 88739644 | C | G |
| 2 | 89080379 | C | T | 2 | 89080394 | C | A | 2 | 89080854 | C | G |
| 2 | 90447633 | T | C | 2 | 90448948 | A | G | 2 | 90448956 | C | T |
| 2 | 90449010 | G | T | 2 | 91671081 | C | T | 2 | 91671100 | T | C |
| 2 | 91671122 | C | G | 2 | 91715572 | T | C | 2 | 92151397 | G | A |
| 2 | 95849653 | T | C | 2 | 96596710 | G | C | 2 | 96809331 | T | C |
| 2 | 96920993 | C | T | 2 | 97366088 | T | C | 2 | 97366599 | A | G |
| 2 | 98223750 | A | G | 2 | 99537295 | A | C | 2 | 101350715 | G | A |
| 2 | 102254291 | T | G | 2 | 102254307 | G | A | 2 | 102254364 | C | G |
| 2 | 102616745 | G | T | 2 | 102617204 | T | C | 2 | 104432433 | T | C |
| 2 | 104432494 | A | C | 2 | 104599371 | T | G | 2 | 105671951 | C | T |
| 2 | 105672240 | T | G | 2 | 106543154 | A | T | 2 | 107019359 | T | C |
| 2 | 107019383 | G | T | 2 | 107019457 | T | C | 2 | 107019874 | T | C |
| 2 | 107424428 | C | T | 2 | 108481932 | C | G | 2 | 109928180 | C | T |
| 2 | 109928207 | G | T | 2 | 110800798 | C | A | 2 | 110800846 | G | A |
| 2 | 112289514 | C | A | 2 | 112289579 | T | C | 2 | 112506112 | C | T |
| 2 | 112506288 | T | C | 2 | 112544651 | G | A | 2 | 113691060 | T | C |
| 2 | 113691288 | T | C | 2 | 113869803 | C | T | 2 | 113869843 | C | A |
| 2 | 114256098 | G | A | 2 | 114256343 | A | G | 2 | 114259673 | C | A |
| 2 | 114288352 | G | T | 2 | 119480974 | C | T | 2 | 120130317 | C | T |
| 2 | 120130337 | C | T | 2 | 120130347 | G | T | 2 | 121321830 | C | T |
| 2 | 122995587 | G | T | 2 | 122996267 | C | T | 2 | 123153811 | A | G |
| 2 | 123154175 | A | G | 2 | 123921841 | C | A | 2 | 124206212 | G | T |
| 2 | 124206727 | G | C | 2 | 124206743 | G | A | 2 | 125189819 | A | T |
| 2 | 127420931 | A | G | 2 | 127811810 | C | T | 2 | 127812457 | G | A |
| 2 | 127812468 | A | G | 2 | 128018522 | T | C | 2 | 129473008 | C | T |
| 2 | 130126515 | A | G | 2 | 130801047 | T | C | 2 | 130805820 | T | C |
| 2 | 131233331 | C | T | 2 | 131945811 | C | A | 2 | 131947586 | C | T |
| 2 | 131955247 | G | C | 2 | 132045098 | C | G | 2 | 132046355 | C | T |
| 2 | 132107348 | A | C | 2 | 132181110 | C | T | 2 | 132533478 | T | C |
| 2 | 132534075 | G | T | 2 | 132579856 | G | A | 2 | 132579930 | A | G |
| 2 | 132580131 | A | C | 2 | 132796086 | G | A | 2 | 132796335 | A | G |
| 2 | 132796339 | A | G | 2 | 132797478 | G | A | 2 | 132809855 | A | G |
| 2 | 132809875 | G | A | 2 | 132809905 | G | T | 2 | 132872466 | C | A |
| 2 | 132872691 | A | G | 2 | 132872847 | G | T | 2 | 132905500 | C | T |
| 2 | 132905513 | C | A | 2 | 132905976 | G | A | 2 | 133644992 | T | A |
| 2 | 133645369 | G | A | 2 | 133645476 | G | A | 2 | 134945535 | A | G |
| 2 | 134946060 | T | C | 2 | 137181641 | C | A | 2 | 137181641 | C | G |
| 2 | 137181661 | C | T | 2 | 137900678 | A | C | 2 | 140000027 | A | G |
| 2 | 140991087 | A | C | 2 | 140992063 | G | C | 2 | 141028164 | C | T |
| 2 | 141055080 | T | G | 2 | 141072930 | C | T | 2 | 141114235 | C | G |
| 2 | 141128001 | C | G | 2 | 141129072 | T | C | 2 | 141136007 | T | C |
| 2 | 141202412 | C | T | 2 | 141202499 | T | A | 2 | 141245486 | G | A |
| 2 | 141271959 | T | G | 2 | 141272545 | G | A | 2 | 141283130 | T | C |
| 2 | 141284090 | C | G | 2 | 141293912 | G | T | 2 | 141299730 | T | C |
| 2 | 141299775 | C | T | 2 | 141355954 | C | T | 2 | 141457603 | G | C |
| 2 | 141460317 | T | A | 2 | 141526501 | T | C | 2 | 141570937 | G | T |
| 2 | 141571590 | C | T | 2 | 141624902 | T | C | 2 | 141641859 | C | A |
| 2 | 141644076 | T | A | 2 | 141660215 | T | C | 2 | 141661007 | A | G |
| 2 | 141707558 | T | C | 2 | 141707610 | A | G | 2 | 141777212 | C | G |
| 2 | 141812434 | C | T | 2 | 141819422 | G | A | 2 | 142004512 | A | C |
| 2 | 142238325 | A | T | 2 | 143225895 | G | A | 2 | 143226379 | A | G |
| 2 | 143226439 | G | T | 2 | 143748793 | A | G | 2 | 144559694 | G | T |
| 2 | 147187335 | T | C | 2 | 148300099 | G | A | 2 | 148300132 | G | A |
| 2 | 149284724 | G | A | 2 | 150920975 | T | C | 2 | 152025225 | A | G |
| 2 | 152943265 | T | C | 2 | 154500721 | T | A | 2 | 155316251 | T | C |
| 2 | 155316904 | C | T | 2 | 156332987 | G | C | 2 | 157262577 | T | G |
| 2 | 160895559 | T | C | 2 | 160895602 | C | G | 2 | 161487015 | T | A |
| 2 | 161822563 | G | A | 2 | 162101078 | A | G | 2 | 166025675 | A | G |
| 2 | 167857455 | G | C | 2 | 170143494 | A | C | 2 | 170624543 | A | G |
| 2 | 171678379 | C | T | 2 | 172034684 | A | G | 2 | 172035195 | G | T |
| 2 | 173664873 | T | C | 2 | 174482931 | G | A | 2 | 175584721 | G | C |
| 2 | 176675940 | A | G | 2 | 177107092 | G | C | 2 | 178098502 | G | T |
| 2 | 178494520 | A | G | 2 | 178528351 | T | C | 2 | 178528360 | A | G |
| 2 | 178528874 | T | C | 2 | 178545736 | T | C | 2 | 178576681 | G | A |
| 2 | 178681294 | T | C | 2 | 178682329 | T | A | 2 | 178682901 | C | T |
| 2 | 178684720 | G | C | 2 | 178704709 | A | G | 2 | 178740334 | T | G |
| 2 | 178740393 | C | T | 2 | 178740398 | C | G | 2 | 178969434 | G | C |
| 2 | 179296821 | G | A | 2 | 179729081 | C | T | 2 | 180308028 | C | T |
| 2 | 180734989 | A | C | 2 | 181490053 | C | T | 2 | 181490633 | G | C |
| 2 | 182433865 | G | A | 2 | 182433873 | G | T | 2 | 182434468 | G | A |
| 2 | 182434507 | A | G | 2 | 183726713 | C | T | 2 | 184128825 | A | G |
| 2 | 184912216 | T | C | 2 | 184912648 | T | C | 2 | 187169224 | G | A |
| 2 | 187866471 | C | T | 2 | 187867057 | G | A | 2 | 190176234 | G | T |
| 2 | 190176235 | C | A | 2 | 190708982 | C | A | 2 | 190741733 | G | A |
| 2 | 190742374 | A | G | 2 | 192422875 | G | T | 2 | 193726153 | C | T |
| 2 | 193726212 | T | C | 2 | 193726268 | A | G | 2 | 194468850 | T | C |
| 2 | 195079687 | G | C | 2 | 196335442 | T | G | 2 | 197037730 | G | A |
| 2 | 198273591 | C | T | 2 | 198274929 | G | A | 2 | 198285554 | C | G |
| 2 | 198905073 | T | A | 2 | 201487990 | C | T | 2 | 212252955 | C | T |
| 2 | 212292929 | G | C | 2 | 212530466 | C | T | 2 | 212543510 | T | C |
| 2 | 212544193 | T | C | 2 | 212566477 | G | A | 2 | 212569828 | C | T |
| 2 | 213296501 | T | G | 2 | 214019110 | T | C | 2 | 215595440 | T | A |
| 2 | 215595445 | C | A | 2 | 215610777 | T | C | 2 | 215617500 | T | C |
| 2 | 215631987 | A | G | 2 | 215632629 | G | T | 2 | 215645030 | T | C |
| 2 | 215645046 | C | T | 2 | 215672347 | C | T | 2 | 215672827 | C | T |
| 2 | 215672833 | A | G | 2 | 216203296 | A | G | 2 | 216860550 | A | T |
| 2 | 217021055 | A | G | 2 | 217453875 | T | C | 2 | 217453939 | C | A |
| 2 | 220136059 | C | A | 2 | 220634227 | C | A | 2 | 220634291 | A | G |
| 2 | 222231318 | G | A | 2 | 222231319 | A | G | 2 | 222231322 | C | T |
| 2 | 222231334 | A | G | 2 | 222232311 | A | G | 2 | 222233697 | T | C |
| 2 | 222721065 | C | T | 2 | 223900314 | C | T | 2 | 224277502 | T | C |
| 2 | 225346377 | A | G | 2 | 225347043 | T | A | 2 | 225360975 | T | C |
| 2 | 225360980 | T | C | 2 | 225360985 | T | C | 2 | 225362798 | A | G |
| 2 | 225367995 | C | T | 2 | 225367996 | C | G | 2 | 225368094 | G | T |
| 2 | 225371246 | A | C | 2 | 225434256 | C | G | 2 | 225658104 | G | A |
| 2 | 228264500 | C | T | 2 | 228725134 | C | T | 2 | 229039040 | C | T |
| 2 | 230470702 | A | G | 2 | 230471160 | T | G | 2 | 233311776 | G | T |
| 2 | 233311824 | A | T | 2 | 234071665 | A | C | 2 | 234676118 | A | G |
| 2 | 234680679 | A | C | 2 | 234681416 | T | C | 2 | 235386951 | T | C |
| 2 | 236618069 | T | C | 2 | 236618494 | A | T | 2 | 236618499 | A | G |
| 2 | 237032523 | A | G | 2 | 237032837 | G | A | 2 | 237820187 | C | T |
| 2 | 238512001 | G | A | 2 | 239675968 | C | T | 2 | 239675977 | T | G |
| 2 | 240872090 | G | A | 2 | 241063584 | G | A | 2 | 241588811 | T | A |
| 2 | 242793849 | G | A | 2 | 243009529 | A | G | 2 | 243059290 | A | T |
| 2 | 243059293 | C | A | 2 | 243071262 | G | T | 2 | 243088710 | G | C |
| 3 | 142069 | A | C | 3 | 1947348 | A | G | 3 | 1947477 | T | C |
| 3 | 2409278 | A | G | 3 | 2409817 | T | C | 3 | 3142901 | T | C |
| 3 | 3142943 | T | C | 3 | 4025464 | A | G | 3 | 4025573 | T | C |
| 3 | 4665123 | A | G | 3 | 5131185 | C | G | 3 | 7888983 | T | C |
| 3 | 8730717 | G | A | 3 | 8730781 | A | C | 3 | 9877716 | G | A |
| 3 | 9877783 | G | A | 3 | 10030236 | T | C | 3 | 10030521 | T | C |
| 3 | 10034925 | G | C | 3 | 10035601 | C | T | 3 | 10075983 | T | C |
| 3 | 10075991 | A | G | 3 | 10078247 | A | G | 3 | 10078289 | G | C |
| 3 | 10080722 | T | C | 3 | 10083169 | C | T | 3 | 10085816 | G | T |
| 3 | 10094418 | T | C | 3 | 10102377 | T | A | 3 | 10104186 | A | T |
| 3 | 10106924 | G | A | 3 | 10108745 | G | C | 3 | 10119603 | C | A |
| 3 | 10122575 | T | C | 3 | 10131831 | C | T | 3 | 10133710 | G | A |
| 3 | 10134766 | C | T | 3 | 10140115 | G | A | 3 | 10183274 | C | T |
| 3 | 10183337 | G | A | 3 | 10191219 | C | G | 3 | 10191943 | G | A |
| 3 | 11041606 | G | A | 3 | 11042207 | T | C | 3 | 11767152 | A | G |
| 3 | 11902162 | G | A | 3 | 11911822 | C | A | 3 | 11912512 | A | C |
| 3 | 11912534 | T | C | 3 | 11912557 | G | A | 3 | 11912622 | G | A |
| 3 | 11912635 | C | T | 3 | 11913124 | A | G | 3 | 11913188 | A | G |
| 3 | 11915481 | C | T | 3 | 11924678 | C | G | 3 | 11925226 | G | A |
| 3 | 11925254 | T | C | 3 | 11926243 | C | G | 3 | 11926269 | G | A |
| 3 | 11926423 | A | G | 3 | 11926467 | A | G | 3 | 11926508 | A | G |
| 3 | 11926604 | A | G | 3 | 11926734 | T | G | 3 | 11928126 | T | G |
| 3 | 11928127 | A | T | 3 | 11928469 | G | T | 3 | 11932455 | T | G |
| 3 | 11933223 | T | C | 3 | 12299313 | A | G | 3 | 12632652 | G | C |
| 3 | 12633552 | T | C | 3 | 12829030 | A | G | 3 | 12829036 | C | T |
| 3 | 12829040 | G | T | 3 | 12829095 | A | C | 3 | 12829156 | A | G |
| 3 | 13152051 | T | C | 3 | 13152457 | C | T | 3 | 14189147 | A | G |
| 3 | 14212258 | C | A | 3 | 15516820 | C | T | 3 | 16408371 | C | T |
| 3 | 16408389 | G | T | 3 | 16408401 | G | C | 3 | 16408402 | G | A |
| 3 | 16408413 | G | A | 3 | 16408424 | C | T | 3 | 16408445 | G | A |
| 3 | 16408451 | C | T | 3 | 16408457 | C | T | 3 | 16408487 | C | G |
| 3 | 16408494 | T | C | 3 | 16408499 | C | T | 3 | 16408500 | A | G |
| 3 | 16408503 | G | T | 3 | 16408519 | G | A | 3 | 16408533 | C | T |
| 3 | 16408534 | A | G | 3 | 16408536 | G | A | 3 | 16408793 | T | G |
| 3 | 16408949 | T | C | 3 | 16408990 | G | T | 3 | 16740865 | A | G |
| 3 | 18412689 | G | T | 3 | 19405951 | T | A | 3 | 21805928 | G | A |
| 3 | 22485461 | T | C | 3 | 22998179 | C | T | 3 | 22998724 | G | T |
| 3 | 22998753 | C | T | 3 | 22998818 | T | C | 3 | 22998824 | A | G |
| 3 | 23128544 | T | C | 3 | 24355183 | A | T | 3 | 24355275 | G | A |
| 3 | 25619630 | G | C | 3 | 25620233 | T | A | 3 | 26445467 | T | C |
| 3 | 27432779 | C | T | 3 | 27432871 | G | A | 3 | 27756251 | T | C |
| 3 | 30686627 | T | C | 3 | 30686647 | T | C | 3 | 30715977 | C | T |
| 3 | 30729636 | G | A | 3 | 32071969 | G | A | 3 | 32072004 | T | C |
| 3 | 32195427 | T | C | 3 | 32232782 | G | T | 3 | 32301422 | C | A |
| 3 | 32301426 | C | T | 3 | 33086893 | T | C | 3 | 33841065 | A | G |
| 3 | 36148273 | C | T | 3 | 36148306 | T | G | 3 | 36148330 | C | T |
| 3 | 36148763 | G | C | 3 | 37067050 | A | G | 3 | 37627598 | T | C |
| 3 | 37790112 | C | T | 3 | 38737643 | T | C | 3 | 40439614 | G | A |
| 3 | 40440116 | G | A | 3 | 40537215 | C | A | 3 | 41269152 | A | T |
| 3 | 45251978 | G | A | 3 | 46511969 | G | A | 3 | 47083782 | C | G |
| 3 | 47103376 | C | T | 3 | 47108330 | C | T | 3 | 48486773 | G | C |
| 3 | 48487338 | G | A | 3 | 48487353 | A | C | 3 | 51102839 | G | A |
| 3 | 51755441 | G | A | 3 | 52597664 | G | A | 3 | 52621839 | C | T |
| 3 | 52659124 | T | C | 3 | 52678494 | G | A | 3 | 53884074 | G | C |
| 3 | 53999979 | A | G | 3 | 55738985 | T | G | 3 | 56235603 | C | A |
| 3 | 56737197 | C | T | 3 | 58036651 | T | C | 3 | 58594418 | C | A |
| 3 | 59701045 | C | T | 3 | 60439446 | T | A | 3 | 60545661 | T | A |
| 3 | 61557787 | G | A | 3 | 63442932 | A | T | 3 | 63443008 | A | C |
| 3 | 64184676 | G | A | 3 | 65289882 | C | T | 3 | 65734943 | T | C |
| 3 | 66077019 | T | C | 3 | 66077076 | A | T | 3 | 66077167 | G | A |
| 3 | 66077485 | G | A | 3 | 66746914 | T | C | 3 | 67145056 | T | C |
| 3 | 69392244 | G | T | 3 | 69392263 | T | C | 3 | 69392331 | C | A |
| 3 | 69392754 | G | A | 3 | 69392762 | G | A | 3 | 69392793 | T | C |
| 3 | 70013349 | A | G | 3 | 71847123 | A | G | 3 | 72978434 | T | C |
| 3 | 73433369 | G | A | 3 | 74513397 | A | G | 3 | 74514007 | T | C |
| 3 | 75365484 | T | C | 3 | 75366035 | C | T | 3 | 75397290 | A | G |
| 3 | 75475805 | A | G | 3 | 75572090 | T | C | 3 | 75572133 | C | G |
| 3 | 75572165 | G | C | 3 | 75572199 | C | T | 3 | 75572202 | T | C |
| 3 | 75572238 | G | A | 3 | 75572329 | G | C | 3 | 75572368 | A | C |
| 3 | 75572374 | G | C | 3 | 75875639 | A | C | 3 | 75875740 | G | A |
| 3 | 75875973 | G | A | 3 | 75876015 | T | G | 3 | 75876023 | A | G |
| 3 | 75876115 | G | A | 3 | 76779549 | C | G | 3 | 76779577 | T | G |
| 3 | 76780491 | A | G | 3 | 77338588 | A | C | 3 | 77338630 | G | T |
| 3 | 77338634 | T | C | 3 | 77338635 | A | C | 3 | 77338636 | A | C |
| 3 | 77338640 | T | C | 3 | 77338835 | G | C | 3 | 77338920 | T | C |
| 3 | 77339156 | G | C | 3 | 77339675 | T | G | 3 | 77340008 | T | C |
| 3 | 77345372 | T | G | 3 | 77345549 | G | A | 3 | 77345598 | G | A |
| 3 | 77459994 | T | C | 3 | 80457094 | G | A | 3 | 81833577 | T | C |
| 3 | 81833966 | G | A | 3 | 81834060 | G | A | 3 | 81834065 | T | A |
| 3 | 83497272 | A | G | 3 | 84924659 | T | C | 3 | 85870199 | A | T |
| 3 | 86702627 | G | T | 3 | 87386674 | T | C | 3 | 87695509 | C | T |
| 3 | 89176645 | A | G | 3 | 89444743 | G | T | 3 | 89448936 | T | A |
| 3 | 89498096 | C | A | 3 | 89521965 | T | G | 3 | 89528313 | T | G |
| 3 | 93903910 | G | A | 3 | 95886072 | A | G | 3 | 97940846 | C | T |
| 3 | 97957539 | C | T | 3 | 97957580 | G | C | 3 | 97957609 | C | T |
| 3 | 97957611 | G | A | 3 | 97983257 | C | G | 3 | 97983265 | C | A |
| 3 | 97983711 | T | C | 3 | 98840224 | C | T | 3 | 101620615 | G | A |
| 3 | 101620682 | A | G | 3 | 102650845 | G | C | 3 | 103705800 | A | T |
| 3 | 104106527 | G | A | 3 | 104106528 | G | A | 3 | 104106562 | C | T |
| 3 | 104639822 | T | C | 3 | 105377515 | C | T | 3 | 105400846 | A | G |
| 3 | 105420602 | C | T | 3 | 105422536 | T | C | 3 | 105439311 | T | A |
| 3 | 105452593 | A | G | 3 | 105459717 | A | G | 3 | 105571964 | C | T |
| 3 | 105586714 | T | C | 3 | 107805553 | C | G | 3 | 108034167 | A | C |
| 3 | 109128553 | G | T | 3 | 109128692 | C | T | 3 | 109775371 | G | A |
| 3 | 109775869 | G | A | 3 | 110496808 | T | C | 3 | 112243112 | A | C |
| 3 | 112352718 | A | G | 3 | 112933625 | A | G | 3 | 112933635 | T | C |
| 3 | 117156240 | G | A | 3 | 117156255 | C | T | 3 | 117156294 | C | A |
| 3 | 117156738 | T | C | 3 | 119272235 | T | C | 3 | 119419376 | G | A |
| 3 | 120402581 | G | T | 3 | 122380722 | C | A | 3 | 123957278 | C | T |
| 3 | 123957294 | C | G | 3 | 124410346 | A | G | 3 | 124840312 | T | A |
| 3 | 125431505 | A | C | 3 | 125431673 | A | T | 3 | 125453782 | G | T |
| 3 | 125453797 | C | T | 3 | 125453908 | G | T | 3 | 125453977 | A | T |
| 3 | 125454162 | C | G | 3 | 125454180 | A | C | 3 | 125482012 | T | G |
| 3 | 125482430 | C | T | 3 | 125484006 | G | C | 3 | 125484009 | C | T |
| 3 | 125494802 | T | C | 3 | 125494864 | T | C | 3 | 125495356 | A | G |
| 3 | 125495487 | T | C | 3 | 125495558 | A | G | 3 | 125639885 | C | A |
| 3 | 125639911 | C | T | 3 | 125643329 | G | A | 3 | 125643332 | A | G |
| 3 | 125643719 | A | G | 3 | 125643772 | G | A | 3 | 126254692 | G | A |
| 3 | 128201013 | A | C | 3 | 129309455 | G | A | 3 | 129722895 | G | A |
| 3 | 129724802 | C | T | 3 | 129804885 | C | G | 3 | 129805060 | C | A |
| 3 | 129805077 | C | T | 3 | 129805236 | A | G | 3 | 129821816 | C | G |
| 3 | 129821817 | C | T | 3 | 129822217 | C | T | 3 | 129822218 | A | G |
| 3 | 129822230 | T | C | 3 | 129825954 | T | G | 3 | 129825955 | A | G |
| 3 | 129826002 | G | T | 3 | 129826513 | T | C | 3 | 129828037 | C | A |
| 3 | 129828078 | T | G | 3 | 129919713 | C | T | 3 | 129920258 | C | T |
| 3 | 130745968 | C | T | 3 | 130745981 | A | G | 3 | 131138571 | C | T |
| 3 | 131138589 | A | G | 3 | 131138658 | C | T | 3 | 133124049 | C | T |
| 3 | 141485046 | T | C | 3 | 142232743 | A | T | 3 | 142233990 | T | C |
| 3 | 142234534 | G | A | 3 | 142234589 | C | A | 3 | 142271797 | A | C |
| 3 | 142273032 | T | C | 3 | 142274534 | C | T | 3 | 142279796 | T | C |
| 3 | 142659361 | A | G | 3 | 143426876 | C | T | 3 | 143427340 | C | G |
| 3 | 144276648 | A | G | 3 | 145139463 | T | C | 3 | 145139946 | A | T |
| 3 | 147955974 | G | T | 3 | 148649659 | C | A | 3 | 148649678 | C | T |
| 3 | 148650252 | G | A | 3 | 149181661 | C | T | 3 | 149271087 | T | C |
| 3 | 149657658 | C | T | 3 | 151053633 | T | C | 3 | 152685955 | T | C |
| 3 | 152686473 | A | G | 3 | 152686523 | G | A | 3 | 152686545 | C | T |
| 3 | 153099380 | T | C | 3 | 155829938 | A | C | 3 | 155830631 | C | T |
| 3 | 156174496 | G | A | 3 | 156880573 | T | C | 3 | 156880587 | A | G |
| 3 | 156880688 | A | G | 3 | 157559512 | A | G | 3 | 158780937 | A | G |
| 3 | 159490097 | C | T | 3 | 162693420 | C | T | 3 | 162693961 | T | A |
| 3 | 164940782 | A | G | 3 | 164941295 | G | A | 3 | 165796202 | A | G |
| 3 | 165796228 | A | T | 3 | 166033539 | G | A | 3 | 166580001 | C | T |
| 3 | 166749739 | T | C | 3 | 167800290 | T | C | 3 | 169200968 | C | A |
| 3 | 169201226 | G | A | 3 | 169427817 | A | G | 3 | 169977589 | T | C |
| 3 | 169978059 | A | G | 3 | 169981058 | G | A | 3 | 169985539 | C | A |
| 3 | 169986068 | T | G | 3 | 169988658 | G | A | 3 | 169998506 | A | G |
| 3 | 170009952 | G | A | 3 | 170021160 | G | A | 3 | 171071949 | T | G |
| 3 | 171684965 | G | A | 3 | 172126162 | C | T | 3 | 173114305 | A | G |
| 3 | 173874385 | G | A | 3 | 173874469 | G | T | 3 | 174056020 | C | G |
| 3 | 174562570 | G | T | 3 | 174562655 | G | T | 3 | 174562942 | A | T |
| 3 | 174563022 | G | A | 3 | 175937783 | T | C | 3 | 176405586 | C | T |
| 3 | 177744450 | G | A | 3 | 178927111 | C | A | 3 | 178927149 | T | C |
| 3 | 178935799 | T | C | 3 | 178938554 | A | T | 3 | 178948385 | A | T |
| 3 | 180424060 | C | T | 3 | 181994593 | C | T | 3 | 181994594 | G | A |
| 3 | 182332176 | T | A | 3 | 182735211 | T | G | 3 | 183204239 | A | G |
| 3 | 184220524 | G | T | 3 | 184220528 | T | C | 3 | 184220531 | G | A |
| 3 | 184220534 | T | C | 3 | 184220549 | A | G | 3 | 184220588 | C | T |
| 3 | 184220608 | G | C | 3 | 184220819 | G | A | 3 | 184220848 | G | C |
| 3 | 184220849 | G | A | 3 | 184220864 | C | T | 3 | 184220865 | A | G |
| 3 | 184220878 | G | C | 3 | 184220879 | G | A | 3 | 184220880 | T | C |
| 3 | 184220890 | T | C | 3 | 184220894 | T | C | 3 | 184220902 | A | C |
| 3 | 184220907 | A | T | 3 | 184220910 | T | C | 3 | 184220915 | A | G |
| 3 | 184220920 | T | C | 3 | 184220924 | C | T | 3 | 184220940 | C | T |
| 3 | 184220945 | G | A | 3 | 184220948 | T | A | 3 | 184220950 | T | C |
| 3 | 184928239 | T | C | 3 | 186508574 | G | C | 3 | 188293043 | T | C |
| 3 | 189382904 | T | C | 3 | 189383430 | C | T | 3 | 190093573 | G | C |
| 3 | 190093689 | G | T | 3 | 190093915 | T | C | 3 | 190094003 | T | C |
| 3 | 190094024 | G | A | 3 | 191093053 | T | C | 3 | 191093080 | A | G |
| 3 | 191741924 | C | T | 3 | 191742322 | G | A | 3 | 194426037 | C | A |
| 3 | 194712317 | C | A | 3 | 194723092 | G | T | 3 | 195384760 | G | C |
| 3 | 195384844 | T | G | 3 | 195399268 | A | C | 3 | 195436937 | A | C |
| 3 | 195437073 | A | G | 3 | 195437584 | G | C | 3 | 195437844 | T | G |
| 3 | 195437869 | C | T | 3 | 195437970 | T | C | 3 | 195437979 | T | G |
| 3 | 195437988 | T | C | 3 | 195437989 | C | G | 3 | 195437990 | A | C |
| 3 | 195437994 | A | G | 3 | 195438024 | T | G | 3 | 195438126 | T | G |
| 3 | 195438174 | A | G | 3 | 195438443 | T | C | 3 | 195438480 | A | C |
| 3 | 195692049 | G | T | 3 | 195692516 | C | T | 3 | 195692570 | G | A |
| 3 | 195700978 | A | C | 3 | 195701115 | A | G | 3 | 195709312 | G | C |
| 3 | 195709355 | C | G | 3 | 195717337 | C | G | 3 | 195872302 | C | T |
| 3 | 195872790 | T | G | 3 | 196379737 | C | G | 3 | 196759586 | C | A |
| 3 | 196759600 | A | G | 3 | 196759604 | T | G | 3 | 196759761 | A | C |
| 3 | 196759779 | A | G | 3 | 196760400 | C | T | 3 | 196760409 | G | A |
| 3 | 197110726 | C | T | 3 | 197112110 | C | A | 3 | 197114790 | A | G |
| 3 | 197114791 | G | C | 3 | 197114962 | T | C | 3 | 197115282 | A | C |
| 3 | 197115557 | T | G | 3 | 197115558 | C | T | 3 | 197115602 | A | G |
| 3 | 197116179 | G | A | 3 | 197116267 | A | G | 3 | 197116296 | T | C |
| 3 | 197116307 | G | C | 3 | 197116710 | G | T | 3 | 197118688 | T | C |
| 3 | 197354725 | C | T | 3 | 197354733 | T | G | 3 | 197354857 | C | A |
| 3 | 197354909 | C | A | 3 | 197825954 | G | A | 3 | 197825956 | G | A |
| 3 | 197826004 | G | A | 3 | 197826013 | C | A | 3 | 197826025 | G | A |
| 3 | 197826185 | G | A | 3 | 197832131 | C | T | 3 | 197832181 | C | T |
| 3 | 197832551 | A | G | 3 | 197832937 | G | C | 3 | 197832951 | G | A |
| 3 | 197851897 | G | A | 4 | 329501 | G | T | 4 | 1266462 | G | A |
| 4 | 1267160 | T | C | 4 | 1267203 | G | T | 4 | 1732410 | C | T |
| 4 | 1736737 | T | C | 4 | 1738732 | C | T | 4 | 1740539 | T | C |
| 4 | 2432104 | G | A | 4 | 2699972 | T | C | 4 | 3536882 | A | G |
| 4 | 3547346 | T | C | 4 | 3903137 | G | A | 4 | 3903256 | C | T |
| 4 | 3903553 | G | A | 4 | 3903604 | A | C | 4 | 3952705 | A | T |
| 4 | 3952730 | T | C | 4 | 3953310 | A | G | 4 | 3953330 | A | G |
| 4 | 3953359 | A | G | 4 | 3953364 | C | T | 4 | 3954873 | A | T |
| 4 | 3954878 | T | C | 4 | 3974295 | G | T | 4 | 3974296 | A | T |
| 4 | 3974455 | C | A | 4 | 3974546 | A | G | 4 | 3974559 | T | C |
| 4 | 3974583 | G | C | 4 | 4060689 | G | A | 4 | 4061299 | T | C |
| 4 | 4099959 | G | A | 4 | 4112309 | A | G | 4 | 4112604 | A | G |
| 4 | 5189645 | G | A | 4 | 5189717 | A | T | 4 | 5652298 | G | C |
| 4 | 6200768 | C | T | 4 | 6881739 | G | C | 4 | 6881785 | G | C |
| 4 | 8999303 | T | A | 4 | 8999512 | C | T | 4 | 9001121 | G | A |
| 4 | 9001176 | G | C | 4 | 9001703 | T | C | 4 | 9012383 | A | T |
| 4 | 9052653 | G | C | 4 | 9137789 | G | C | 4 | 9137790 | A | G |
| 4 | 9137990 | G | A | 4 | 9158848 | G | A | 4 | 9159079 | T | G |
| 4 | 9159219 | C | T | 4 | 9159256 | C | G | 4 | 9163190 | A | T |
| 4 | 9271895 | T | G | 4 | 9531098 | A | G | 4 | 9532659 | G | T |
| 4 | 9584058 | G | A | 4 | 9673458 | T | C | 4 | 9673980 | C | T |
| 4 | 9696823 | G | A | 4 | 9700910 | A | T | 4 | 9757407 | G | A |
| 4 | 9757485 | G | A | 4 | 10180622 | T | C | 4 | 10180643 | T | C |
| 4 | 10726609 | G | A | 4 | 10726610 | C | T | 4 | 10726707 | C | T |
| 4 | 12056196 | T | C | 4 | 13031210 | T | G | 4 | 14232397 | C | T |
| 4 | 14837656 | C | T | 4 | 15117509 | G | A | 4 | 15733093 | C | A |
| 4 | 15733108 | G | T | 4 | 15733159 | A | G | 4 | 17348027 | A | G |
| 4 | 17503839 | G | A | 4 | 18056140 | C | T | 4 | 18798633 | C | A |
| 4 | 19250760 | C | T | 4 | 21551721 | T | C | 4 | 21552230 | A | G |
| 4 | 23715644 | C | T | 4 | 24142003 | A | C | 4 | 25721273 | C | T |
| 4 | 27128768 | C | T | 4 | 27683108 | T | A | 4 | 28247387 | G | A |
| 4 | 28247910 | G | A | 4 | 28551237 | G | A | 4 | 29333030 | C | T |
| 4 | 29333037 | C | T | 4 | 29707479 | G | A | 4 | 30290545 | A | G |
| 4 | 30290549 | T | A | 4 | 31017144 | C | G | 4 | 31017221 | C | T |
| 4 | 31761297 | G | A | 4 | 32592869 | C | G | 4 | 32592949 | C | T |
| 4 | 33642926 | T | C | 4 | 34985401 | T | G | 4 | 34985806 | G | A |
| 4 | 35510086 | A | C | 4 | 35510132 | C | T | 4 | 35510659 | A | G |
| 4 | 35510681 | A | G | 4 | 36329799 | A | C | 4 | 36330306 | T | A |
| 4 | 37002365 | T | C | 4 | 37002380 | A | G | 4 | 38512602 | T | C |
| 4 | 39482627 | T | C | 4 | 39482684 | A | C | 4 | 39483115 | A | G |
| 4 | 39483162 | G | A | 4 | 40022837 | A | G | 4 | 40022870 | C | T |
| 4 | 40023258 | T | C | 4 | 40835384 | T | A | 4 | 40835854 | C | A |
| 4 | 41748511 | T | G | 4 | 42608665 | T | C | 4 | 46241769 | G | A |
| 4 | 46763980 | A | C | 4 | 47493210 | C | G | 4 | 49292437 | T | G |
| 4 | 49298634 | A | C | 4 | 49304353 | T | G | 4 | 49304428 | A | C |
| 4 | 49304465 | T | C | 4 | 49304469 | T | A | 4 | 49314901 | C | T |
| 4 | 49321018 | A | G | 4 | 49321104 | G | T | 4 | 49617655 | C | T |
| 4 | 49628621 | C | T | 4 | 49628869 | T | A | 4 | 49628914 | A | C |
| 4 | 49629091 | T | C | 4 | 49629144 | G | A | 4 | 49629145 | T | C |
| 4 | 52978719 | C | A | 4 | 52978741 | T | C | 4 | 52979321 | T | C |
| 4 | 53454588 | T | G | 4 | 54430834 | T | C | 4 | 55136027 | G | C |
| 4 | 55136245 | A | G | 4 | 55137183 | T | C | 4 | 55143321 | C | T |
| 4 | 55146926 | T | A | 4 | 55146927 | T | A | 4 | 55161047 | C | G |
| 4 | 55523938 | G | A | 4 | 55524533 | C | G | 4 | 55573693 | G | A |
| 4 | 55602451 | C | T | 4 | 55962165 | A | T | 4 | 55962166 | C | T |
| 4 | 55967806 | A | G | 4 | 55971400 | G | A | 4 | 55974290 | C | G |
| 4 | 55985142 | A | G | 4 | 55985149 | A | G | 4 | 55985209 | C | G |
| 4 | 55991731 | T | C | 4 | 57917346 | G | T | 4 | 58969178 | C | G |
| 4 | 58969622 | A | C | 4 | 59117179 | C | A | 4 | 59685846 | A | G |
| 4 | 63895205 | T | A | 4 | 64379405 | A | G | 4 | 64931039 | T | C |
| 4 | 66270412 | C | T | 4 | 66355888 | T | C | 4 | 66355901 | G | T |
| 4 | 67970457 | T | C | 4 | 69901970 | C | T | 4 | 70354884 | G | A |
| 4 | 72347419 | G | T | 4 | 73292973 | C | T | 4 | 74322255 | T | C |
| 4 | 74738851 | G | A | 4 | 75394764 | A | G | 4 | 75395312 | G | A |
| 4 | 75395322 | C | A | 4 | 78990229 | C | A | 4 | 78990900 | C | A |
| 4 | 79482186 | G | A | 4 | 79602718 | G | A | 4 | 80386263 | T | G |
| 4 | 80801388 | T | A | 4 | 84013033 | C | T | 4 | 86340131 | C | G |
| 4 | 86340164 | G | A | 4 | 87264746 | T | A | 4 | 87264750 | G | A |
| 4 | 87692129 | T | G | 4 | 87695876 | T | A | 4 | 87718279 | G | A |
| 4 | 87732483 | C | T | 4 | 87791437 | C | T | 4 | 89777379 | C | G |
| 4 | 90228238 | C | T | 4 | 93487149 | A | T | 4 | 93487465 | C | A |
| 4 | 95088301 | T | C | 4 | 95606136 | T | C | 4 | 99443068 | A | G |
| 4 | 99443113 | A | G | 4 | 99443127 | T | C | 4 | 99443569 | T | C |
| 4 | 99443585 | G | A | 4 | 99443611 | A | T | 4 | 100238998 | T | C |
| 4 | 101572039 | G | A | 4 | 102854264 | C | T | 4 | 104452689 | T | A |
| 4 | 106164284 | T | G | 4 | 106193588 | A | G | 4 | 106914301 | C | T |
| 4 | 106914306 | A | T | 4 | 106914338 | C | T | 4 | 106914855 | G | A |
| 4 | 108124872 | G | A | 4 | 108125157 | G | A | 4 | 108125170 | C | A |
| 4 | 108565609 | G | A | 4 | 111944989 | A | G | 4 | 113180779 | C | T |
| 4 | 113181343 | T | C | 4 | 113486769 | T | C | 4 | 115241715 | C | G |
| 4 | 115987887 | A | T | 4 | 116002140 | T | C | 4 | 117957242 | T | G |
| 4 | 118496905 | A | G | 4 | 118496919 | T | C | 4 | 118497508 | C | G |
| 4 | 118503372 | C | T | 4 | 119252323 | C | T | 4 | 119252368 | G | A |
| 4 | 119526740 | C | G | 4 | 119527206 | C | T | 4 | 119944319 | A | G |
| 4 | 119944900 | A | G | 4 | 119944903 | C | A | 4 | 120133549 | T | G |
| 4 | 120324707 | T | C | 4 | 120325264 | C | T | 4 | 120325273 | T | C |
| 4 | 120355709 | T | C | 4 | 120751902 | T | A | 4 | 121089766 | G | C |
| 4 | 121738049 | T | C | 4 | 122290719 | G | A | 4 | 122290979 | G | C |
| 4 | 122291131 | T | G | 4 | 122431338 | T | C | 4 | 122683623 | C | T |
| 4 | 124946414 | C | T | 4 | 124946423 | A | G | 4 | 125173747 | C | T |
| 4 | 125725127 | A | G | 4 | 127856273 | T | G | 4 | 128518960 | C | T |
| 4 | 132832700 | T | G | 4 | 132832731 | A | G | 4 | 133045796 | A | G |
| 4 | 133824971 | A | G | 4 | 133825066 | G | A | 4 | 133825500 | C | T |
| 4 | 133825533 | A | G | 4 | 133885960 | T | G | 4 | 134784133 | G | A |
| 4 | 137489544 | C | A | 4 | 137985807 | C | T | 4 | 138698629 | T | A |
| 4 | 139796813 | G | A | 4 | 140619075 | T | C | 4 | 143003526 | A | G |
| 4 | 143034019 | A | T | 4 | 143034029 | G | A | 4 | 143044829 | A | G |
| 4 | 143045482 | G | A | 4 | 143066775 | A | G | 4 | 143095131 | C | T |
| 4 | 143095139 | C | T | 4 | 143095201 | C | T | 4 | 143114003 | T | C |
| 4 | 143114011 | A | G | 4 | 143114040 | A | C | 4 | 143114482 | T | C |
| 4 | 143114548 | A | G | 4 | 143114556 | A | G | 4 | 143114558 | G | A |
| 4 | 143114598 | G | C | 4 | 143129292 | G | A | 4 | 143236139 | T | C |
| 4 | 143236158 | T | C | 4 | 143323915 | T | G | 4 | 143326120 | G | T |
| 4 | 143326714 | T | G | 4 | 143352065 | T | C | 4 | 143926811 | A | G |
| 4 | 144754667 | A | G | 4 | 145214743 | G | A | 4 | 145568682 | A | C |
| 4 | 146656399 | T | C | 4 | 147206992 | G | A | 4 | 147207075 | G | C |
| 4 | 147208378 | C | G | 4 | 147970517 | G | A | 4 | 148024671 | G | C |
| 4 | 149067352 | C | G | 4 | 149870390 | T | C | 4 | 150167697 | A | C |
| 4 | 150674206 | A | T | 4 | 151259617 | T | C | 4 | 153254121 | C | T |
| 4 | 153960301 | T | A | 4 | 155298773 | T | G | 4 | 155299346 | T | C |
| 4 | 155851079 | C | A | 4 | 156433308 | G | A | 4 | 156607373 | A | G |
| 4 | 157616767 | G | A | 4 | 159194870 | G | A | 4 | 159931005 | G | C |
| 4 | 160823765 | G | C | 4 | 161285415 | G | A | 4 | 161956269 | G | A |
| 4 | 161956317 | C | T | 4 | 161956363 | T | A | 4 | 161956376 | C | T |
| 4 | 161956767 | A | G | 4 | 161956781 | A | C | 4 | 162129559 | A | G |
| 4 | 163086385 | A | C | 4 | 164465973 | A | T | 4 | 164890625 | T | C |
| 4 | 165933477 | C | A | 4 | 167125325 | C | G | 4 | 167654337 | C | T |
| 4 | 167654397 | C | G | 4 | 167654397 | C | T | 4 | 167654427 | C | A |
| 4 | 168292643 | G | A | 4 | 168292723 | A | G | 4 | 168292832 | T | C |
| 4 | 168292836 | A | T | 4 | 168292853 | T | A | 4 | 168292864 | T | C |
| 4 | 168292887 | A | G | 4 | 169633306 | C | T | 4 | 170812566 | C | T |
| 4 | 171078557 | T | A | 4 | 171774610 | C | T | 4 | 178128578 | G | C |
| 4 | 180339049 | A | G | 4 | 181286425 | C | T | 4 | 181509274 | G | C |
| 4 | 183032807 | A | G | 4 | 184807301 | A | G | 4 | 185311508 | T | C |
| 4 | 185311570 | G | A | 4 | 185329052 | G | A | 4 | 185350307 | T | C |
| 4 | 185350422 | A | G | 4 | 185779168 | T | C | 4 | 185779461 | C | T |
| 4 | 185779481 | G | A | 4 | 185780254 | G | A | 4 | 186485037 | G | A |
| 4 | 187353180 | A | C | 4 | 187353188 | A | G | 4 | 187353199 | G | A |
| 4 | 187353362 | G | A | 4 | 187353388 | C | T | 4 | 187353390 | C | A |
| 4 | 187353437 | G | A | 4 | 187353459 | C | T | 4 | 187353485 | C | T |
| 4 | 187353486 | C | G | 4 | 187353487 | G | A | 4 | 187353491 | C | T |
| 4 | 187353497 | T | C | 4 | 187353500 | T | A | 4 | 187353508 | A | G |
| 4 | 187353510 | G | C | 4 | 187353510 | G | T | 4 | 187353518 | C | G |
| 4 | 187353520 | A | G | 4 | 187353550 | T | C | 4 | 187353554 | A | G |
| 4 | 187353557 | G | C | 4 | 187353610 | C | T | 4 | 187353658 | A | G |
| 4 | 187353818 | A | G | 4 | 187354048 | A | G | 4 | 187354174 | G | A |
| 4 | 187354186 | G | A | 4 | 187354197 | T | C | 4 | 187354312 | A | G |
| 4 | 187354335 | T | C | 4 | 187354416 | G | A | 4 | 187354429 | A | G |
| 4 | 187354521 | G | A | 4 | 187354542 | A | G | 4 | 187354828 | T | C |
| 4 | 187355027 | G | A | 4 | 187356475 | G | C | 4 | 187356484 | G | A |
| 4 | 187356497 | G | A | 4 | 187356955 | T | C | 4 | 187356957 | A | G |
| 4 | 187357003 | G | A | 4 | 187357027 | G | C | 4 | 187357036 | G | A |
| 4 | 187357363 | G | A | 4 | 187357369 | T | C | 4 | 187357387 | C | T |
| 4 | 187357415 | T | C | 4 | 187357667 | T | C | 4 | 187357686 | G | C |
| 4 | 187357798 | A | G | 4 | 187522112 | C | G | 4 | 187522789 | T | C |
| 4 | 187525910 | A | T | 4 | 187525922 | G | A | 4 | 187525940 | A | C |
| 4 | 187525941 | G | T | 4 | 187525946 | A | G | 4 | 187525986 | G | A |
| 4 | 187526002 | C | A | 4 | 187533153 | C | T | 4 | 187534689 | G | T |
| 4 | 187535063 | T | C | 4 | 187535765 | T | C | 4 | 187631223 | C | T |
| 4 | 189426841 | A | G | 4 | 189426857 | G | T | 4 | 189426863 | C | T |
| 4 | 189426864 | G | A | 4 | 189426871 | C | A | 4 | 189426888 | G | C |
| 4 | 189427370 | G | A | 4 | 189427435 | A | T | 4 | 189972360 | A | G |
| 4 | 189972363 | G | A | 4 | 189972366 | A | G | 4 | 189972384 | G | A |
| 4 | 189972492 | A | G | 4 | 189972498 | G | A | 4 | 189972501 | A | G |
| 4 | 189972516 | G | C | 4 | 189972897 | G | A | 4 | 189972919 | A | G |
| 4 | 189972930 | A | G | 4 | 189972933 | G | A | 4 | 189973230 | A | G |
| 4 | 189973257 | G | A | 4 | 189973266 | A | G | 4 | 189973278 | G | A |
| 4 | 189973949 | T | C | 4 | 190197957 | C | A | 4 | 190198002 | A | G |
| 4 | 190198077 | A | G | 4 | 190198080 | T | C | 4 | 190198100 | C | G |
| 4 | 190198107 | G | A | 4 | 190198111 | G | A | 4 | 190198121 | C | G |
| 4 | 190198127 | A | G | 4 | 190198142 | T | G | 4 | 190198193 | G | C |
| 4 | 190198201 | G | A | 4 | 190198206 | T | C | 4 | 190198214 | C | T |
| 4 | 190198221 | A | C | 4 | 190198225 | T | C | 4 | 190198226 | G | A |
| 4 | 190198377 | C | T | 4 | 190198703 | G | A | 4 | 190198707 | G | A |
| 4 | 190198717 | C | G | 4 | 190198844 | T | C | 4 | 190199033 | T | C |
| 4 | 190199199 | T | C | 4 | 190859230 | C | G | 4 | 190859289 | A | C |
| 4 | 190859333 | A | G | 4 | 190878827 | T | G | 4 | 190899555 | T | C |
| 5 | 19602 | A | G | 5 | 223415 | C | T | 5 | 225267 | C | A |
| 5 | 226453 | G | A | 5 | 233325 | G | A | 5 | 233362 | T | G |
| 5 | 233371 | A | G | 5 | 236932 | T | A | 5 | 254442 | C | T |
| 5 | 254861 | C | G | 5 | 649405 | A | G | 5 | 649749 | T | C |
| 5 | 650037 | T | C | 5 | 650266 | G | A | 5 | 650846 | C | T |
| 5 | 651295 | T | G | 5 | 1261052 | G | A | 5 | 1266226 | T | C |
| 5 | 1266310 | A | G | 5 | 1271524 | T | C | 5 | 1572585 | A | G |
| 5 | 1594664 | C | G | 5 | 1600771 | A | G | 5 | 1600785 | T | C |
| 5 | 1858062 | T | C | 5 | 1858062 | T | G | 5 | 1858821 | A | T |
| 5 | 1858834 | G | C | 5 | 2145174 | T | C | 5 | 2145508 | T | C |
| 5 | 2146102 | G | A | 5 | 2146276 | C | G | 5 | 2146338 | T | G |
| 5 | 2146345 | T | G | 5 | 2146527 | C | A | 5 | 2146527 | C | G |
| 5 | 2825568 | C | A | 5 | 3659894 | C | T | 5 | 3659913 | G | A |
| 5 | 3659989 | C | T | 5 | 3660487 | A | G | 5 | 4225004 | A | G |
| 5 | 4941345 | A | G | 5 | 6272411 | C | A | 5 | 6273004 | G | T |
| 5 | 7447405 | A | G | 5 | 7927332 | C | T | 5 | 7927348 | C | T |
| 5 | 7927356 | C | A | 5 | 7927414 | T | G | 5 | 7927952 | G | C |
| 5 | 8061222 | G | A | 5 | 8061279 | G | A | 5 | 8061284 | C | G |
| 5 | 10151085 | T | C | 5 | 10151123 | A | G | 5 | 10912625 | T | C |
| 5 | 12056802 | A | G | 5 | 12056837 | C | T | 5 | 12056923 | A | C |
| 5 | 12057204 | A | G | 5 | 12058227 | A | G | 5 | 13190185 | T | C |
| 5 | 13847794 | C | T | 5 | 14634747 | A | G | 5 | 14634756 | G | A |
| 5 | 15955155 | C | A | 5 | 16535341 | T | C | 5 | 17025201 | G | T |
| 5 | 17353828 | C | T | 5 | 17354234 | C | T | 5 | 20166781 | T | C |
| 5 | 20167136 | T | G | 5 | 21293117 | T | C | 5 | 21381238 | G | A |
| 5 | 21381264 | T | G | 5 | 21497311 | C | T | 5 | 21497344 | T | G |
| 5 | 22227645 | A | T | 5 | 23242291 | A | G | 5 | 23242685 | T | C |
| 5 | 23304111 | C | T | 5 | 23304211 | G | T | 5 | 23304235 | T | C |
| 5 | 23304261 | G | A | 5 | 23304368 | A | C | 5 | 23305356 | G | A |
| 5 | 23305374 | G | A | 5 | 23305484 | T | C | 5 | 23527239 | C | G |
| 5 | 23527919 | A | G | 5 | 23528007 | G | A | 5 | 24844076 | G | A |
| 5 | 25007293 | A | G | 5 | 25759642 | G | A | 5 | 27795250 | T | A |
| 5 | 28213790 | A | G | 5 | 29312232 | G | T | 5 | 29312728 | G | A |
| 5 | 29312755 | A | C | 5 | 29438592 | G | A | 5 | 29438662 | A | C |
| 5 | 29438664 | T | C | 5 | 30972823 | C | T | 5 | 31383964 | A | G |
| 5 | 31384055 | G | A | 5 | 31384509 | C | A | 5 | 31840606 | T | C |
| 5 | 32828846 | T | C | 5 | 33466421 | A | G | 5 | 33633582 | G | A |
| 5 | 34206776 | C | T | 5 | 35867204 | A | T | 5 | 36170237 | T | C |
| 5 | 36171420 | G | A | 5 | 36172105 | C | T | 5 | 36172115 | G | A |
| 5 | 36176874 | G | A | 5 | 36181617 | A | G | 5 | 36181627 | A | G |
| 5 | 36184284 | C | G | 5 | 38943260 | G | T | 5 | 38996638 | A | G |
| 5 | 39021369 | G | A | 5 | 39074696 | C | T | 5 | 40320944 | G | C |
| 5 | 40320945 | G | T | 5 | 43053497 | A | T | 5 | 43767400 | A | G |
| 5 | 43803258 | T | C | 5 | 44392401 | G | A | 5 | 44585369 | G | C |
| 5 | 44585815 | C | G | 5 | 50058043 | T | C | 5 | 51105168 | G | T |
| 5 | 53835771 | G | A | 5 | 54154481 | A | T | 5 | 55781646 | T | C |
| 5 | 55781772 | T | C | 5 | 55782130 | T | C | 5 | 56152795 | G | A |
| 5 | 56161975 | C | T | 5 | 56171391 | G | T | 5 | 56175226 | A | G |
| 5 | 56176306 | T | C | 5 | 56183637 | G | C | 5 | 56184444 | T | C |
| 5 | 56803613 | G | A | 5 | 58478599 | C | A | 5 | 58478692 | A | G |
| 5 | 58711707 | G | A | 5 | 60860146 | C | T | 5 | 62085252 | G | C |
| 5 | 64957686 | A | G | 5 | 64957718 | T | C | 5 | 65349061 | T | A |
| 5 | 65867918 | C | T | 5 | 66219033 | T | C | 5 | 66219117 | T | G |
| 5 | 67569602 | A | G | 5 | 67577051 | G | A | 5 | 67584357 | A | G |
| 5 | 67589914 | G | A | 5 | 67590140 | C | A | 5 | 67593016 | T | C |
| 5 | 68417945 | G | T | 5 | 68927608 | G | A | 5 | 68927676 | T | C |
| 5 | 69674512 | G | A | 5 | 69927417 | G | T | 5 | 71198806 | C | T |
| 5 | 71542000 | A | G | 5 | 72125176 | A | G | 5 | 73231521 | C | A |
| 5 | 73524903 | A | G | 5 | 73524939 | G | A | 5 | 73525404 | G | A |
| 5 | 75041362 | T | G | 5 | 76986580 | C | G | 5 | 77991568 | C | T |
| 5 | 80655266 | T | C | 5 | 82205681 | A | G | 5 | 82206152 | G | A |
| 5 | 83378803 | T | C | 5 | 83378919 | G | A | 5 | 84637681 | T | C |
| 5 | 85577284 | C | T | 5 | 85586579 | T | C | 5 | 85586584 | C | T |
| 5 | 86865094 | G | A | 5 | 89418018 | A | T | 5 | 93798480 | T | C |
| 5 | 93798817 | A | C | 5 | 94572738 | C | A | 5 | 95477855 | A | G |
| 5 | 97441644 | A | T | 5 | 97441664 | A | G | 5 | 98851779 | C | T |
| 5 | 98861253 | A | G | 5 | 98913692 | C | A | 5 | 98913961 | T | C |
| 5 | 99069979 | G | A | 5 | 99724591 | C | A | 5 | 99733040 | G | A |
| 5 | 99733085 | A | G | 5 | 100695270 | T | C | 5 | 102089175 | G | C |
| 5 | 107267081 | C | T | 5 | 107962193 | C | T | 5 | 108535800 | T | C |
| 5 | 110491533 | C | T | 5 | 111184331 | T | C | 5 | 111184434 | A | G |
| 5 | 111699038 | T | C | 5 | 111699088 | A | G | 5 | 111699115 | C | A |
| 5 | 111699641 | C | T | 5 | 112043170 | A | C | 5 | 112101793 | C | A |
| 5 | 112151551 | G | T | 5 | 112164862 | G | A | 5 | 112171097 | A | G |
| 5 | 112821283 | A | C | 5 | 113797914 | G | A | 5 | 114685700 | T | C |
| 5 | 115106332 | C | G | 5 | 115394388 | A | C | 5 | 115394396 | C | G |
| 5 | 115394857 | A | T | 5 | 115394859 | A | G | 5 | 115394869 | T | C |
| 5 | 115394881 | C | A | 5 | 115394908 | C | A | 5 | 116051351 | C | T |
| 5 | 117955311 | T | C | 5 | 118952055 | C | T | 5 | 122456440 | T | A |
| 5 | 122609817 | T | C | 5 | 124243030 | C | T | 5 | 124243078 | C | T |
| 5 | 125874804 | G | T | 5 | 127638759 | A | G | 5 | 128428722 | A | G |
| 5 | 128428723 | T | G | 5 | 128428851 | C | T | 5 | 128500520 | G | A |
| 5 | 131911847 | G | C | 5 | 131925174 | A | C | 5 | 131925187 | T | C |
| 5 | 131944129 | G | A | 5 | 131951996 | T | C | 5 | 131953623 | G | A |
| 5 | 131973177 | A | G | 5 | 133784798 | C | T | 5 | 134356380 | G | A |
| 5 | 134583301 | A | G | 5 | 134583302 | C | T | 5 | 134583937 | T | C |
| 5 | 135336981 | A | G | 5 | 135979386 | C | T | 5 | 137136265 | A | G |
| 5 | 137787827 | T | A | 5 | 140476395 | T | C | 5 | 140476396 | G | T |
| 5 | 140476653 | G | A | 5 | 140476654 | C | A | 5 | 140482102 | A | G |
| 5 | 140482483 | G | A | 5 | 140503443 | C | G | 5 | 140531746 | C | G |
| 5 | 140554189 | G | C | 5 | 140554808 | T | A | 5 | 140559532 | G | C |
| 5 | 140564048 | G | C | 5 | 140564088 | C | T | 5 | 140568796 | A | G |
| 5 | 140574045 | C | G | 5 | 140581220 | C | A | 5 | 140590766 | A | G |
| 5 | 140595625 | G | A | 5 | 140605030 | T | G | 5 | 141392706 | T | C |
| 5 | 143146391 | C | T | 5 | 144135790 | T | G | 5 | 144135871 | T | C |
| 5 | 144604975 | C | T | 5 | 145296020 | C | T | 5 | 146320823 | G | T |
| 5 | 146562965 | A | C | 5 | 146562965 | A | G | 5 | 148838021 | G | A |
| 5 | 149433322 | G | T | 5 | 149440644 | C | T | 5 | 149440645 | C | T |
| 5 | 149440723 | G | A | 5 | 149440739 | A | G | 5 | 149448060 | G | A |
| 5 | 149457493 | A | G | 5 | 149458110 | C | T | 5 | 149458128 | T | C |
| 5 | 149495770 | G | A | 5 | 149497672 | C | T | 5 | 149498759 | A | T |
| 5 | 149498821 | C | T | 5 | 149501803 | C | T | 5 | 149512042 | G | A |
| 5 | 149512781 | G | A | 5 | 149780952 | A | G | 5 | 150277909 | A | G |
| 5 | 151474068 | A | T | 5 | 151474626 | A | G | 5 | 151900065 | C | T |
| 5 | 151900374 | G | T | 5 | 152861322 | G | A | 5 | 154703595 | G | C |
| 5 | 155580762 | G | A | 5 | 155580831 | G | T | 5 | 156449748 | G | A |
| 5 | 158052650 | G | A | 5 | 159622823 | C | T | 5 | 160357140 | C | T |
| 5 | 161154595 | C | G | 5 | 163479999 | G | T | 5 | 165061117 | T | G |
| 5 | 167958419 | C | G | 5 | 168395021 | T | C | 5 | 169872183 | A | G |
| 5 | 170215778 | C | T | 5 | 170215820 | T | A | 5 | 170817415 | A | G |
| 5 | 170818969 | A | G | 5 | 170828170 | C | T | 5 | 170832637 | C | T |
| 5 | 172750436 | G | A | 5 | 174177551 | T | C | 5 | 174177552 | C | T |
| 5 | 174177562 | G | A | 5 | 174969438 | G | C | 5 | 174969467 | T | C |
| 5 | 174969879 | G | A | 5 | 174969908 | G | A | 5 | 175007464 | T | C |
| 5 | 175007481 | A | G | 5 | 175007493 | T | C | 5 | 175774865 | G | T |
| 5 | 176287029 | A | G | 5 | 176710547 | C | G | 5 | 177163580 | A | G |
| 5 | 177380181 | A | T | 5 | 177380281 | C | T | 5 | 177380537 | C | T |
| 5 | 177380562 | A | C | 5 | 177380624 | T | G | 5 | 178012176 | A | G |
| 5 | 178012696 | T | C | 5 | 178012728 | A | C | 5 | 179430530 | C | T |
| 5 | 180029879 | G | A | 5 | 180036239 | G | A | 5 | 180036318 | A | G |
| 5 | 180037317 | C | T | 5 | 180037318 | G | A | 5 | 180039187 | A | G |
| 5 | 180041274 | T | C | 5 | 180043093 | T | C | 5 | 180045453 | C | T |
| 5 | 180046209 | C | T | 5 | 180049152 | C | T | 5 | 180049181 | C | T |
| 5 | 180049525 | T | C | 5 | 180049907 | G | A | 5 | 180049928 | A | C |
| 5 | 180050066 | T | C | 5 | 180051296 | A | G | 5 | 180051305 | A | G |
| 5 | 180058008 | C | A | 5 | 180058963 | G | A | 5 | 180076129 | G | A |
| 5 | 180076130 | C | A | 5 | 180076240 | A | G | 5 | 180698413 | T | C |
| 5 | 180698428 | G | C | 6 | 675548 | A | C | 6 | 675553 | C | T |
| 6 | 675712 | C | T | 6 | 676129 | A | C | 6 | 676727 | T | G |
| 6 | 676780 | G | T | 6 | 676918 | A | G | 6 | 677120 | T | C |
| 6 | 2777264 | G | A | 6 | 2777334 | A | G | 6 | 3154871 | A | C |
| 6 | 3177590 | C | A | 6 | 3177601 | C | T | 6 | 3177658 | A | T |
| 6 | 3402411 | G | A | 6 | 3402435 | C | G | 6 | 3402520 | T | C |
| 6 | 4255068 | A | G | 6 | 4716150 | C | T | 6 | 4716185 | T | C |
| 6 | 5148454 | G | A | 6 | 6970420 | T | C | 6 | 6970890 | A | G |
| 6 | 7387462 | T | C | 6 | 7516700 | T | A | 6 | 7516833 | G | T |
| 6 | 7517131 | A | T | 6 | 7517239 | G | A | 6 | 7618813 | G | A |
| 6 | 10575131 | T | C | 6 | 11214092 | A | T | 6 | 11969231 | C | T |
| 6 | 11969262 | C | T | 6 | 15497649 | T | C | 6 | 17271896 | C | T |
| 6 | 18139450 | T | A | 6 | 18140175 | T | C | 6 | 18147780 | G | A |
| 6 | 18528399 | C | T | 6 | 21808894 | A | G | 6 | 22117590 | G | C |
| 6 | 24077986 | A | C | 6 | 24968393 | G | A | 6 | 24976616 | C | T |
| 6 | 24976637 | G | A | 6 | 24976739 | G | C | 6 | 25262087 | A | G |
| 6 | 26126524 | A | G | 6 | 26127090 | A | G | 6 | 26202337 | G | A |
| 6 | 26202605 | G | A | 6 | 26202768 | C | T | 6 | 26671135 | T | C |
| 6 | 26702489 | G | A | 6 | 26798264 | G | A | 6 | 26798312 | G | T |
| 6 | 26851448 | G | A | 6 | 26864205 | T | C | 6 | 26864281 | T | C |
| 6 | 26864327 | G | A | 6 | 27689939 | T | C | 6 | 27690453 | G | T |
| 6 | 28447915 | T | C | 6 | 29691857 | C | G | 6 | 29692622 | C | A |
| 6 | 29692622 | C | G | 6 | 29693499 | A | G | 6 | 29759811 | C | G |
| 6 | 29759823 | T | C | 6 | 29759824 | T | C | 6 | 29759876 | G | A |
| 6 | 29759885 | G | A | 6 | 29759923 | T | C | 6 | 29759996 | T | C |
| 6 | 29760029 | G | C | 6 | 29760111 | G | C | 6 | 29760149 | G | T |
| 6 | 29760188 | G | A | 6 | 29760510 | G | A | 6 | 29760523 | C | T |
| 6 | 29760536 | G | A | 6 | 29760590 | G | A | 6 | 29760605 | G | T |
| 6 | 29795636 | G | A | 6 | 29795657 | G | A | 6 | 29796115 | A | C |
| 6 | 29796152 | G | C | 6 | 29796257 | C | T | 6 | 29796306 | G | A |
| 6 | 29796327 | C | T | 6 | 29796749 | C | A | 6 | 29796838 | A | G |
| 6 | 29797173 | A | G | 6 | 29797553 | G | C | 6 | 29797639 | C | T |
| 6 | 29855320 | T | G | 6 | 29855386 | C | T | 6 | 29855392 | T | C |
| 6 | 29855392 | T | G | 6 | 29855401 | C | G | 6 | 29855409 | A | G |
| 6 | 29855677 | G | A | 6 | 29855682 | T | G | 6 | 29855706 | C | G |
| 6 | 29855733 | C | T | 6 | 29855849 | A | C | 6 | 29855856 | T | C |
| 6 | 29855858 | C | A | 6 | 29855901 | A | G | 6 | 29855902 | G | A |
| 6 | 29855940 | G | A | 6 | 29855945 | A | G | 6 | 29856092 | A | T |
| 6 | 29856109 | C | T | 6 | 29856199 | A | G | 6 | 29856204 | G | T |
| 6 | 29856655 | G | C | 6 | 29856686 | C | G | 6 | 29856693 | C | T |
| 6 | 29856718 | G | C | 6 | 29856965 | G | C | 6 | 29857013 | A | G |
| 6 | 29857021 | C | A | 6 | 29857753 | T | C | 6 | 29858218 | A | G |
| 6 | 29858256 | T | G | 6 | 29858294 | A | G | 6 | 29858475 | C | A |
| 6 | 29858476 | T | C | 6 | 29858477 | G | A | 6 | 29858517 | A | G |
| 6 | 29858660 | A | C | 6 | 29894128 | C | T | 6 | 29894139 | G | A |
| 6 | 29894205 | T | C | 6 | 29894211 | C | T | 6 | 29894226 | G | T |
| 6 | 29894243 | C | T | 6 | 29894250 | T | A | 6 | 29894269 | T | C |
| 6 | 29894299 | C | A | 6 | 29894332 | T | A | 6 | 29894334 | G | A |
| 6 | 29894351 | G | C | 6 | 29894389 | A | C | 6 | 29894392 | G | T |
| 6 | 29894406 | C | A | 6 | 29894407 | G | A | 6 | 29894410 | C | T |
| 6 | 29894439 | A | T | 6 | 29894456 | T | G | 6 | 29894523 | G | A |
| 6 | 29894567 | G | A | 6 | 29894580 | T | G | 6 | 29894603 | A | G |
| 6 | 29894756 | A | G | 6 | 29894763 | T | C | 6 | 29894798 | T | C |
| 6 | 29894851 | C | A | 6 | 29894853 | C | T | 6 | 29894874 | C | A |
| 6 | 29894966 | C | T | 6 | 29894991 | T | C | 6 | 29895002 | G | C |
| 6 | 29895005 | C | T | 6 | 29895039 | A | C | 6 | 29895215 | C | T |
| 6 | 29895250 | C | T | 6 | 29895334 | T | C | 6 | 29895399 | G | C |
| 6 | 29895472 | T | C | 6 | 29895862 | G | A | 6 | 29895888 | G | C |
| 6 | 29895913 | G | A | 6 | 29895929 | C | G | 6 | 29895944 | G | A |
| 6 | 29896001 | C | A | 6 | 29896054 | C | A | 6 | 29896055 | C | T |
| 6 | 29896084 | A | G | 6 | 29896105 | G | A | 6 | 29896114 | G | A |
| 6 | 29896120 | G | T | 6 | 29896125 | G | A | 6 | 29896128 | G | T |
| 6 | 29896257 | C | T | 6 | 29896268 | T | C | 6 | 29896275 | G | A |
| 6 | 29896291 | T | C | 6 | 29896484 | G | C | 6 | 29896523 | A | C |
| 6 | 29896925 | C | G | 6 | 29896967 | C | T | 6 | 29897017 | C | A |
| 6 | 29897030 | G | A | 6 | 29897061 | C | T | 6 | 29897259 | T | A |
| 6 | 29897301 | C | T | 6 | 29897322 | G | T | 6 | 29897350 | C | T |
| 6 | 29897379 | G | C | 6 | 29897408 | T | A | 6 | 29897412 | G | C |
| 6 | 29897416 | A | G | 6 | 29897417 | G | T | 6 | 29897422 | A | G |
| 6 | 29901640 | C | A | 6 | 29901783 | A | T | 6 | 29901806 | C | T |
| 6 | 29901865 | G | C | 6 | 29901898 | C | G | 6 | 29901987 | C | G |
| 6 | 29902005 | G | C | 6 | 29902016 | T | A | 6 | 29902040 | A | G |
| 6 | 29902050 | C | T | 6 | 29902051 | A | G | 6 | 29902059 | A | G |
| 6 | 29902063 | C | T | 6 | 29902064 | T | G | 6 | 29902074 | A | G |
| 6 | 29902129 | T | G | 6 | 29902130 | G | A | 6 | 29902168 | C | T |
| 6 | 29902198 | A | G | 6 | 29902240 | A | C | 6 | 29902293 | C | T |
| 6 | 29902294 | G | T | 6 | 29902327 | G | T | 6 | 29902332 | C | G |
| 6 | 29902333 | A | G | 6 | 29902334 | C | T | 6 | 29910057 | C | A |
| 6 | 29910176 | C | T | 6 | 29910189 | A | T | 6 | 29911571 | G | C |
| 6 | 29911604 | C | A | 6 | 29911684 | C | T | 6 | 29911685 | C | T |
| 6 | 29911727 | T | C | 6 | 29911790 | T | C | 6 | 29911803 | G | A |
| 6 | 29912614 | C | A | 6 | 29912649 | G | C | 6 | 29912656 | C | T |
| 6 | 29912657 | C | T | 6 | 29912668 | T | G | 6 | 29912669 | A | G |
| 6 | 29912680 | A | G | 6 | 29912687 | C | T | 6 | 29912713 | C | T |
| 6 | 29913495 | G | A | 6 | 29913518 | C | A | 6 | 29913526 | A | G |
| 6 | 29925127 | G | A | 6 | 29925134 | G | A | 6 | 29925438 | C | T |
| 6 | 29974346 | G | C | 6 | 29974500 | A | G | 6 | 29975047 | G | T |
| 6 | 29975453 | C | A | 6 | 29975840 | G | A | 6 | 29975950 | C | T |
| 6 | 29975994 | C | A | 6 | 29976024 | C | G | 6 | 29976499 | C | G |
| 6 | 29976741 | A | G | 6 | 29977145 | G | C | 6 | 30227397 | C | T |
| 6 | 30227583 | C | G | 6 | 30228652 | C | A | 6 | 30228721 | G | A |
| 6 | 30228879 | C | T | 6 | 30228881 | T | C | 6 | 30228938 | T | C |
| 6 | 30229306 | A | G | 6 | 30229457 | C | T | 6 | 30229463 | G | A |
| 6 | 30229504 | G | A | 6 | 30229583 | G | C | 6 | 30434900 | T | C |
| 6 | 30457732 | T | C | 6 | 30692230 | G | T | 6 | 30692231 | C | T |
| 6 | 30799399 | T | A | 6 | 31197074 | C | T | 6 | 31197514 | C | G |
| 6 | 31197528 | A | G | 6 | 31197589 | G | A | 6 | 31237858 | T | C |
| 6 | 31237862 | T | C | 6 | 31237876 | A | G | 6 | 31237897 | C | T |
| 6 | 31237917 | C | T | 6 | 31237918 | G | A | 6 | 31237950 | T | C |
| 6 | 31237955 | T | G | 6 | 31237960 | G | C | 6 | 31237962 | T | C |
| 6 | 31237963 | G | A | 6 | 31237965 | G | C | 6 | 31237971 | C | T |
| 6 | 31237975 | T | C | 6 | 31237987 | C | T | 6 | 31237991 | G | T |
| 6 | 31238009 | T | C | 6 | 31238010 | T | G | 6 | 31238027 | C | A |
| 6 | 31238029 | T | C | 6 | 31238053 | G | C | 6 | 31238068 | C | T |
| 6 | 31238126 | G | A | 6 | 31238135 | G | A | 6 | 31238138 | C | T |
| 6 | 31238147 | C | G | 6 | 31238155 | G | A | 6 | 31238230 | G | C |
| 6 | 31238234 | G | A | 6 | 31238259 | G | T | 6 | 31238267 | C | T |
| 6 | 31238297 | C | T | 6 | 31238308 | G | C | 6 | 31238318 | G | A |
| 6 | 31238322 | C | T | 6 | 31238345 | T | C | 6 | 31238372 | C | G |
| 6 | 31238613 | A | G | 6 | 31238632 | C | T | 6 | 31238661 | G | A |
| 6 | 31238662 | A | G | 6 | 31238728 | G | C | 6 | 31239802 | C | G |
| 6 | 31239821 | G | T | 6 | 31239827 | C | T | 6 | 31239855 | C | G |
| 6 | 31239868 | G | C | 6 | 31239869 | T | C | 6 | 31239896 | T | C |
| 6 | 31239937 | C | T | 6 | 31323012 | G | A | 6 | 31323020 | T | C |
| 6 | 31323065 | G | C | 6 | 31323567 | G | A | 6 | 31323677 | C | T |
| 6 | 31323707 | G | A | 6 | 31323721 | G | C | 6 | 31323725 | T | C |
| 6 | 31323745 | C | A | 6 | 31323766 | T | C | 6 | 31323838 | C | G |
| 6 | 31324003 | T | A | 6 | 31324003 | T | G | 6 | 31324004 | C | G |
| 6 | 31324176 | C | G | 6 | 31324184 | C | G | 6 | 31324194 | G | A |
| 6 | 31324200 | G | C | 6 | 31324201 | C | G | 6 | 31324206 | G | A |
| 6 | 31324208 | G | T | 6 | 31324210 | G | A | 6 | 31324227 | C | A |
| 6 | 31324229 | G | C | 6 | 31324489 | C | A | 6 | 31324489 | C | G |
| 6 | 31324491 | C | A | 6 | 31324499 | C | G | 6 | 31324523 | T | C |
| 6 | 31324530 | G | C | 6 | 31324531 | C | G | 6 | 31324531 | C | T |
| 6 | 31324536 | T | A | 6 | 31324536 | T | C | 6 | 31324536 | T | G |
| 6 | 31324538 | G | C | 6 | 31324539 | A | T | 6 | 31324542 | T | C |
| 6 | 31324547 | G | C | 6 | 31324829 | G | C | 6 | 31324830 | G | A |
| 6 | 31324847 | C | G | 6 | 31324864 | G | A | 6 | 31324887 | G | C |
| 6 | 31324888 | G | T | 6 | 31324891 | C | T | 6 | 31324892 | G | C |
| 6 | 31324895 | G | C | 6 | 31324911 | C | G | 6 | 31324921 | C | T |
| 6 | 31324925 | A | G | 6 | 31324931 | A | C | 6 | 31324941 | C | T |
| 6 | 31324953 | C | T | 6 | 31324955 | C | T | 6 | 31324996 | G | A |
| 6 | 31324996 | G | C | 6 | 31325004 | G | A | 6 | 31333610 | C | T |
| 6 | 31333731 | T | C | 6 | 31333751 | G | A | 6 | 31333820 | A | G |
| 6 | 31333874 | G | A | 6 | 31333911 | C | T | 6 | 31333939 | A | G |
| 6 | 31333963 | T | C | 6 | 31334080 | T | A | 6 | 31334120 | A | G |
| 6 | 31487954 | G | C | 6 | 31488038 | C | T | 6 | 31488145 | A | G |
| 6 | 32455520 | G | C | 6 | 32455544 | A | G | 6 | 32455560 | G | C |
| 6 | 32455565 | T | G | 6 | 32455566 | T | A | 6 | 32455569 | C | T |
| 6 | 32455577 | A | T | 6 | 32455580 | T | G | 6 | 32455585 | T | G |
| 6 | 32455586 | G | A | 6 | 32455588 | T | C | 6 | 32455629 | T | C |
| 6 | 32455644 | T | A | 6 | 32455645 | T | C | 6 | 32455651 | A | G |
| 6 | 32455662 | G | A | 6 | 32455668 | A | G | 6 | 32455672 | C | T |
| 6 | 32455680 | G | A | 6 | 32455692 | C | A | 6 | 32455718 | T | G |
| 6 | 32455720 | C | G | 6 | 32455725 | A | G | 6 | 32455725 | A | T |
| 6 | 32455726 | C | G | 6 | 32455737 | A | G | 6 | 32455742 | T | A |
| 6 | 32455743 | T | A | 6 | 32455743 | T | C | 6 | 32455748 | C | T |
| 6 | 32455750 | C | T | 6 | 32455756 | T | A | 6 | 32455757 | A | C |
| 6 | 32455757 | A | G | 6 | 32455777 | C | T | 6 | 32455795 | T | G |
| 6 | 32455801 | C | T | 6 | 32455810 | G | T | 6 | 32455812 | C | A |
| 6 | 32455827 | A | G | 6 | 32455828 | T | C | 6 | 32455838 | G | A |
| 6 | 32455839 | T | C | 6 | 32455843 | A | G | 6 | 32455847 | A | G |
| 6 | 32455859 | C | G | 6 | 32455877 | A | T | 6 | 32455887 | A | G |
| 6 | 32455901 | T | C | 6 | 32455906 | A | G | 6 | 32455909 | T | A |
| 6 | 32455912 | G | A | 6 | 32455917 | C | A | 6 | 32455917 | C | G |
| 6 | 32455928 | T | C | 6 | 32455943 | C | T | 6 | 32455952 | T | C |
| 6 | 32455965 | G | A | 6 | 32456028 | G | A | 6 | 32796346 | T | C |
| 6 | 32796480 | T | C | 6 | 32798789 | C | T | 6 | 32798795 | C | A |
| 6 | 32805049 | T | C | 6 | 32817130 | A | G | 6 | 32819642 | C | T |
| 6 | 32821859 | C | A | 6 | 33290402 | G | A | 6 | 33290825 | A | G |
| 6 | 33290872 | G | A | 6 | 34711946 | C | T | 6 | 34712232 | C | T |
| 6 | 35389999 | G | T | 6 | 35420923 | C | T | 6 | 35960390 | C | T |
| 6 | 37001592 | G | A | 6 | 37002182 | G | A | 6 | 37709919 | A | T |
| 6 | 38960253 | A | G | 6 | 38960677 | C | T | 6 | 38960750 | C | T |
| 6 | 40419999 | C | T | 6 | 41296173 | C | T | 6 | 41296679 | C | T |
| 6 | 41635569 | G | T | 6 | 42407020 | G | A | 6 | 43739327 | C | T |
| 6 | 43742419 | G | C | 6 | 43746949 | T | C | 6 | 44228162 | G | A |
| 6 | 44678620 | A | C | 6 | 45969983 | T | G | 6 | 46856100 | G | A |
| 6 | 46856581 | T | G | 6 | 47130819 | T | C | 6 | 47130910 | G | A |
| 6 | 47130988 | A | G | 6 | 47713939 | T | A | 6 | 47714073 | G | A |
| 6 | 48151808 | G | A | 6 | 48928482 | A | G | 6 | 48928544 | T | C |
| 6 | 49327632 | T | C | 6 | 49328214 | A | G | 6 | 51492164 | T | A |
| 6 | 51514205 | T | C | 6 | 51611344 | A | G | 6 | 51619921 | T | C |
| 6 | 51619972 | A | G | 6 | 51637240 | G | C | 6 | 51640787 | A | G |
| 6 | 51655796 | T | C | 6 | 51656388 | A | G | 6 | 51696056 | A | G |
| 6 | 51696074 | G | A | 6 | 51701484 | G | A | 6 | 51713012 | G | C |
| 6 | 51735613 | T | C | 6 | 51750961 | G | A | 6 | 51752241 | C | T |
| 6 | 51889099 | A | T | 6 | 51897565 | C | T | 6 | 51909525 | T | C |
| 6 | 51919253 | G | A | 6 | 51927666 | T | C | 6 | 51927683 | T | C |
| 6 | 51929476 | T | G | 6 | 51934976 | C | T | 6 | 51934978 | A | C |
| 6 | 51936717 | C | T | 6 | 51936723 | G | A | 6 | 51940816 | T | C |
| 6 | 51949862 | A | G | 6 | 52529666 | A | C | 6 | 52529714 | C | G |
| 6 | 52530205 | C | T | 6 | 53047564 | C | T | 6 | 54610711 | C | T |
| 6 | 54610742 | A | C | 6 | 54610783 | C | T | 6 | 54611146 | A | G |
| 6 | 56291176 | G | A | 6 | 58199238 | T | C | 6 | 58250570 | A | T |
| 6 | 58263871 | T | C | 6 | 58269738 | G | A | 6 | 58270343 | C | G |
| 6 | 58403634 | T | C | 6 | 58404193 | C | G | 6 | 58522197 | C | T |
| 6 | 61993928 | G | A | 6 | 62864433 | A | G | 6 | 62864516 | G | A |
| 6 | 63083740 | T | G | 6 | 63083748 | G | T | 6 | 63083774 | A | G |
| 6 | 65331854 | T | C | 6 | 65589020 | G | C | 6 | 66446642 | A | G |
| 6 | 67044197 | G | T | 6 | 67044321 | C | T | 6 | 67044663 | T | A |
| 6 | 67804122 | G | T | 6 | 67804186 | T | C | 6 | 67804194 | A | C |
| 6 | 67804202 | G | A | 6 | 71721412 | G | A | 6 | 71721445 | T | C |
| 6 | 72563207 | C | T | 6 | 72563211 | G | T | 6 | 72563750 | C | T |
| 6 | 73888146 | C | T | 6 | 75056542 | G | A | 6 | 75877044 | A | C |
| 6 | 76051990 | A | G | 6 | 77478497 | C | G | 6 | 78345710 | C | T |
| 6 | 79426986 | G | A | 6 | 79427089 | A | G | 6 | 81484692 | G | C |
| 6 | 81669149 | G | C | 6 | 82074113 | T | C | 6 | 83480639 | C | T |
| 6 | 83480720 | T | C | 6 | 83545840 | C | A | 6 | 83546246 | G | A |
| 6 | 85070849 | C | T | 6 | 85071341 | A | G | 6 | 85140178 | A | C |
| 6 | 85140313 | C | A | 6 | 85140399 | G | A | 6 | 88608057 | A | G |
| 6 | 90012435 | G | A | 6 | 91210640 | A | G | 6 | 91682066 | C | T |
| 6 | 92363395 | A | C | 6 | 92838614 | C | T | 6 | 93100978 | G | A |
| 6 | 93100980 | T | C | 6 | 93901330 | A | G | 6 | 94555470 | A | G |
| 6 | 96348615 | T | G | 6 | 97077504 | C | A | 6 | 101332215 | T | C |
| 6 | 101849962 | C | A | 6 | 105089423 | A | G | 6 | 108028709 | T | C |
| 6 | 108028752 | T | A | 6 | 108028757 | T | G | 6 | 108774766 | C | G |
| 6 | 109051552 | C | T | 6 | 109051652 | T | C | 6 | 109507842 | A | T |
| 6 | 110252356 | G | C | 6 | 110252919 | C | A | 6 | 110252987 | A | G |
| 6 | 110911774 | C | T | 6 | 111871080 | T | C | 6 | 112375307 | A | G |
| 6 | 112375889 | A | G | 6 | 112524506 | T | C | 6 | 112677375 | C | A |
| 6 | 113203779 | G | A | 6 | 113940257 | G | T | 6 | 114262015 | A | C |
| 6 | 114265754 | A | G | 6 | 114405657 | G | C | 6 | 114405704 | A | G |
| 6 | 115322919 | G | A | 6 | 115398931 | G | C | 6 | 116787050 | C | T |
| 6 | 116787688 | C | A | 6 | 117609387 | T | C | 6 | 117622521 | T | A |
| 6 | 117629650 | A | T | 6 | 117629663 | T | C | 6 | 117631666 | T | C |
| 6 | 117639104 | T | C | 6 | 117639153 | T | C | 6 | 117639620 | G | T |
| 6 | 117657400 | A | G | 6 | 117657686 | A | G | 6 | 117662944 | C | T |
| 6 | 117663364 | A | G | 6 | 117665635 | G | C | 6 | 117683505 | G | A |
| 6 | 117699988 | A | G | 6 | 117725168 | G | A | 6 | 117725789 | C | T |
| 6 | 117725885 | C | A | 6 | 117725890 | T | A | 6 | 117731107 | G | C |
| 6 | 118103565 | G | A | 6 | 119463313 | G | A | 6 | 119870312 | A | G |
| 6 | 120173251 | C | T | 6 | 121850570 | G | A | 6 | 121851118 | A | G |
| 6 | 122015474 | A | G | 6 | 122585885 | C | T | 6 | 123823950 | T | C |
| 6 | 124236107 | C | G | 6 | 124236196 | G | A | 6 | 124236507 | C | T |
| 6 | 124569808 | G | A | 6 | 125553174 | C | T | 6 | 127432657 | A | G |
| 6 | 127909778 | T | C | 6 | 127909784 | C | T | 6 | 128323940 | G | A |
| 6 | 128324030 | G | C | 6 | 128990229 | T | C | 6 | 128990286 | G | A |
| 6 | 128990824 | A | G | 6 | 130007597 | C | A | 6 | 130507274 | A | G |
| 6 | 131047028 | A | C | 6 | 132030037 | T | C | 6 | 132283588 | C | T |
| 6 | 132284167 | A | T | 6 | 132726450 | C | T | 6 | 132840195 | G | C |
| 6 | 132840197 | T | A | 6 | 133019682 | A | G | 6 | 133607088 | A | G |
| 6 | 134496112 | G | A | 6 | 134528280 | C | T | 6 | 135375762 | G | A |
| 6 | 135376379 | T | G | 6 | 135376409 | G | T | 6 | 135989483 | C | T |
| 6 | 137288483 | T | G | 6 | 137288522 | T | C | 6 | 137521772 | C | T |
| 6 | 138027115 | C | A | 6 | 138027145 | C | T | 6 | 140101549 | A | G |
| 6 | 140849815 | T | G | 6 | 142025855 | T | C | 6 | 144476023 | T | A |
| 6 | 144601920 | C | A | 6 | 144601921 | G | A | 6 | 144602478 | C | T |
| 6 | 145806936 | C | T | 6 | 145807010 | G | A | 6 | 146350563 | C | A |
| 6 | 147729477 | T | G | 6 | 148516642 | T | G | 6 | 149237845 | A | C |
| 6 | 149237889 | A | G | 6 | 149237931 | G | A | 6 | 149700805 | G | A |
| 6 | 151364186 | A | G | 6 | 151646048 | G | A | 6 | 152129746 | G | T |
| 6 | 152201624 | G | A | 6 | 153065996 | G | A | 6 | 153066078 | T | G |
| 6 | 153523383 | A | G | 6 | 153523425 | G | A | 6 | 154478704 | C | T |
| 6 | 154629327 | C | T | 6 | 156301661 | G | T | 6 | 156301726 | C | T |
| 6 | 156938505 | T | C | 6 | 156938506 | A | G | 6 | 156938539 | T | C |
| 6 | 157150145 | G | A | 6 | 157256310 | C | T | 6 | 158024313 | T | C |
| 6 | 158024403 | C | G | 6 | 158024861 | C | A | 6 | 158024868 | C | T |
| 6 | 159190019 | T | G | 6 | 159947789 | C | T | 6 | 160398330 | C | T |
| 6 | 160547249 | G | T | 6 | 160548433 | A | G | 6 | 161771412 | T | C |
| 6 | 161970231 | T | A | 6 | 161990678 | C | T | 6 | 162206577 | T | G |
| 6 | 162474893 | A | G | 6 | 162475434 | A | C | 6 | 162622550 | C | T |
| 6 | 165404863 | A | C | 6 | 165404917 | A | G | 6 | 165722345 | T | C |
| 6 | 166204195 | T | C | 6 | 166204288 | G | A | 6 | 166695194 | T | C |
| 6 | 167411008 | T | C | 6 | 167411334 | C | T | 6 | 167411337 | G | A |
| 6 | 167411388 | C | G | 6 | 167411401 | C | T | 6 | 167411616 | C | T |
| 6 | 167589680 | C | T | 6 | 167792111 | G | A | 6 | 167792114 | G | A |
| 6 | 167792651 | C | G | 6 | 167792671 | C | G | 6 | 167792694 | G | C |
| 6 | 167792699 | G | A | 6 | 168687802 | C | G | 6 | 168687815 | A | C |
| 6 | 168687819 | C | A | 6 | 168687828 | G | A | 6 | 168735290 | A | G |
| 6 | 168735309 | A | C | 6 | 168735311 | T | A | 6 | 169313243 | A | G |
| 6 | 169795962 | G | T | 6 | 170470548 | T | A | 6 | 170470568 | A | C |
| 6 | 170470778 | C | A | 6 | 170528622 | A | G | 6 | 170529546 | A | G |
| 6 | 170529549 | G | A | 6 | 170529566 | C | T | 6 | 170529587 | T | C |
| 6 | 170529638 | T | C | 6 | 170530453 | T | C | 6 | 170530506 | C | T |
| 6 | 170530518 | A | G | 6 | 170530559 | T | C | 6 | 170530838 | C | T |
| 6 | 170530873 | C | T | 6 | 171026382 | T | C | 6 | 171026394 | T | C |
| 7 | 746314 | A | G | 7 | 746705 | A | G | 7 | 746706 | C | A |
| 7 | 1883274 | T | C | 7 | 2004124 | C | T | 7 | 3155087 | T | C |
| 7 | 3155089 | T | G | 7 | 3155579 | C | T | 7 | 3155651 | C | G |
| 7 | 4320984 | A | G | 7 | 4321571 | A | G | 7 | 5156760 | G | A |
| 7 | 5946901 | G | C | 7 | 6013049 | C | G | 7 | 6018444 | A | G |
| 7 | 6018592 | G | A | 7 | 6022178 | G | C | 7 | 6029748 | C | T |
| 7 | 6035428 | A | C | 7 | 6035507 | C | T | 7 | 6038483 | G | A |
| 7 | 6039129 | T | C | 7 | 6041897 | T | C | 7 | 6042522 | C | T |
| 7 | 6043057 | A | G | 7 | 6043065 | C | T | 7 | 6043112 | C | T |
| 7 | 6045881 | C | T | 7 | 6045934 | T | C | 7 | 6048359 | C | T |
| 7 | 6048804 | G | C | 7 | 6048845 | A | G | 7 | 6048973 | C | A |
| 7 | 6414545 | C | T | 7 | 6431404 | G | T | 7 | 6431420 | C | A |
| 7 | 6431933 | C | G | 7 | 6441258 | G | A | 7 | 6442371 | G | A |
| 7 | 6776537 | G | A | 7 | 6776585 | C | T | 7 | 6777726 | T | C |
| 7 | 6777752 | T | C | 7 | 6777768 | T | G | 7 | 6777780 | T | G |
| 7 | 6777784 | A | G | 7 | 6777820 | T | G | 7 | 6777821 | G | A |
| 7 | 6785956 | A | G | 7 | 6786731 | C | T | 7 | 6790900 | T | C |
| 7 | 6791040 | G | C | 7 | 6919565 | T | C | 7 | 6974986 | G | A |
| 7 | 6975272 | G | T | 7 | 6975316 | G | T | 7 | 6975366 | C | A |
| 7 | 8549008 | G | A | 7 | 8549058 | G | A | 7 | 8549477 | C | T |
| 7 | 10013659 | G | A | 7 | 10897044 | T | C | 7 | 10897594 | C | A |
| 7 | 10897594 | C | G | 7 | 11349162 | C | T | 7 | 11349705 | G | A |
| 7 | 11349733 | A | G | 7 | 11349735 | T | C | 7 | 12122025 | C | T |
| 7 | 12510929 | A | C | 7 | 12511312 | G | A | 7 | 14868954 | C | T |
| 7 | 16297567 | C | T | 7 | 17006944 | G | C | 7 | 17007526 | G | T |
| 7 | 17514653 | T | C | 7 | 18318662 | C | T | 7 | 18624673 | G | A |
| 7 | 18625440 | C | T | 7 | 19184257 | C | G | 7 | 21206385 | A | G |
| 7 | 21764077 | A | G | 7 | 21764081 | G | A | 7 | 22327627 | G | A |
| 7 | 22328109 | C | T | 7 | 22947858 | A | G | 7 | 22948438 | G | C |
| 7 | 24353194 | A | C | 7 | 24353195 | C | T | 7 | 24353267 | C | T |
| 7 | 25233745 | C | G | 7 | 25233789 | G | C | 7 | 26627952 | G | T |
| 7 | 26627954 | G | A | 7 | 29464865 | T | C | 7 | 30177268 | G | A |
| 7 | 30177278 | C | T | 7 | 30855318 | G | A | 7 | 30855926 | G | C |
| 7 | 32133424 | G | A | 7 | 32512110 | A | T | 7 | 33101967 | C | T |
| 7 | 33102514 | G | A | 7 | 33193313 | A | C | 7 | 33193314 | T | C |
| 7 | 34540399 | G | T | 7 | 35083870 | T | C | 7 | 35084552 | C | T |
| 7 | 35084572 | C | T | 7 | 35084601 | T | C | 7 | 36186371 | G | C |
| 7 | 36858622 | G | A | 7 | 36858643 | A | G | 7 | 37415057 | G | A |
| 7 | 37708712 | A | G | 7 | 39807352 | G | A | 7 | 40320747 | G | C |
| 7 | 43874751 | A | G | 7 | 44026116 | T | G | 7 | 44056757 | C | T |
| 7 | 44057222 | C | G | 7 | 45256549 | A | G | 7 | 45256975 | A | G |
| 7 | 45828285 | C | T | 7 | 45847880 | A | G | 7 | 45856902 | T | C |
| 7 | 45856916 | G | A | 7 | 45857319 | G | A | 7 | 45857848 | G | A |
| 7 | 46028450 | C | T | 7 | 46028459 | C | T | 7 | 46643720 | A | C |
| 7 | 50470256 | G | A | 7 | 50745025 | G | A | 7 | 51450917 | G | A |
| 7 | 51450951 | A | G | 7 | 51451225 | T | C | 7 | 51451275 | G | A |
| 7 | 51451291 | C | T | 7 | 51451376 | A | G | 7 | 51451655 | G | A |
| 7 | 51451775 | T | C | 7 | 51451925 | C | T | 7 | 51451928 | T | C |
| 7 | 51451955 | G | T | 7 | 51452002 | A | G | 7 | 51452050 | G | A |
| 7 | 51452088 | T | C | 7 | 51452279 | A | G | 7 | 51824545 | C | T |
| 7 | 51824890 | T | C | 7 | 51824990 | A | G | 7 | 52225367 | T | C |
| 7 | 52758842 | T | C | 7 | 52759429 | A | G | 7 | 53189193 | A | G |
| 7 | 53189634 | G | A | 7 | 53190434 | G | T | 7 | 53190453 | A | G |
| 7 | 53190508 | A | G | 7 | 53190917 | C | T | 7 | 53203065 | G | A |
| 7 | 53718464 | T | C | 7 | 53718485 | G | T | 7 | 53854735 | T | A |
| 7 | 53854969 | T | C | 7 | 54618943 | A | G | 7 | 55210733 | G | C |
| 7 | 55219290 | T | C | 7 | 55219307 | C | G | 7 | 55221447 | C | G |
| 7 | 55225140 | G | C | 7 | 55232726 | G | T | 7 | 55248787 | T | C |
| 7 | 55259948 | C | G | 7 | 55658927 | T | C | 7 | 55659085 | T | C |
| 7 | 55806087 | G | T | 7 | 55806093 | G | T | 7 | 56405132 | T | C |
| 7 | 56430969 | C | G | 7 | 56430977 | G | C | 7 | 56430980 | C | A |
| 7 | 56431109 | A | G | 7 | 56431170 | A | G | 7 | 56431807 | G | A |
| 7 | 56432242 | T | C | 7 | 56635995 | C | A | 7 | 57289345 | C | T |
| 7 | 57603890 | T | G | 7 | 57603918 | G | A | 7 | 57669433 | G | T |
| 7 | 57669986 | A | G | 7 | 57809151 | C | A | 7 | 57832849 | T | C |
| 7 | 57938126 | A | G | 7 | 57938210 | T | C | 7 | 61058809 | T | G |
| 7 | 61059081 | A | G | 7 | 61063424 | G | A | 7 | 61063551 | A | G |
| 7 | 61071095 | A | T | 7 | 61071103 | C | T | 7 | 61071188 | G | T |
| 7 | 61071203 | C | T | 7 | 63206179 | A | G | 7 | 63206611 | A | G |
| 7 | 63206623 | G | T | 7 | 63229281 | G | A | 7 | 63229377 | C | T |
| 7 | 63229524 | T | C | 7 | 63230106 | C | T | 7 | 63230147 | C | T |
| 7 | 63230155 | G | A | 7 | 63602429 | G | A | 7 | 63602520 | A | G |
| 7 | 63602542 | T | C | 7 | 63635236 | A | C | 7 | 63635239 | G | C |
| 7 | 63635241 | A | G | 7 | 63635248 | T | C | 7 | 63635271 | C | G |
| 7 | 63635272 | T | C | 7 | 63635286 | C | T | 7 | 64583576 | C | G |
| 7 | 64583585 | C | T | 7 | 64583587 | C | T | 7 | 64583615 | G | C |
| 7 | 64583628 | C | T | 7 | 64652748 | C | G | 7 | 64652982 | T | G |
| 7 | 64653060 | G | A | 7 | 65037658 | C | G | 7 | 65037683 | T | C |
| 7 | 65059071 | A | G | 7 | 65082869 | G | A | 7 | 65163375 | C | G |
| 7 | 65163587 | T | C | 7 | 65432962 | G | A | 7 | 65552792 | A | T |
| 7 | 65971428 | G | T | 7 | 66355416 | C | T | 7 | 66457975 | T | C |
| 7 | 66458627 | A | G | 7 | 66458631 | C | T | 7 | 66458903 | T | C |
| 7 | 66459513 | C | T | 7 | 66459553 | G | C | 7 | 66760853 | T | C |
| 7 | 66760969 | G | A | 7 | 66760975 | C | T | 7 | 66761773 | T | C |
| 7 | 66762050 | A | T | 7 | 66762277 | C | T | 7 | 66762340 | A | C |
| 7 | 66762461 | T | A | 7 | 67635408 | C | T | 7 | 67635412 | G | A |
| 7 | 69444431 | G | C | 7 | 70047405 | G | A | 7 | 71309437 | C | T |
| 7 | 73809454 | T | C | 7 | 74178770 | C | T | 7 | 74193668 | G | A |
| 7 | 74582543 | T | G | 7 | 76072643 | C | T | 7 | 76072644 | A | G |
| 7 | 76237814 | C | T | 7 | 76237885 | A | C | 7 | 76364904 | C | A |
| 7 | 76621710 | C | T | 7 | 76641042 | T | C | 7 | 76641340 | A | T |
| 7 | 76669334 | G | A | 7 | 78690736 | C | T | 7 | 78690794 | G | A |
| 7 | 79293866 | A | G | 7 | 79293877 | G | T | 7 | 80387473 | G | C |
| 7 | 80387977 | A | G | 7 | 80826781 | T | C | 7 | 81346925 | G | A |
| 7 | 81358712 | G | A | 7 | 81381731 | T | C | 7 | 81858825 | G | T |
| 7 | 82925988 | A | G | 7 | 83407638 | A | C | 7 | 84345772 | C | G |
| 7 | 84707925 | C | T | 7 | 86856261 | G | T | 7 | 87031809 | A | G |
| 7 | 87133947 | C | A | 7 | 87133993 | T | C | 7 | 87149922 | C | T |
| 7 | 87161049 | T | C | 7 | 87174504 | C | T | 7 | 87182882 | T | C |
| 7 | 87190953 | A | G | 7 | 87195106 | C | T | 7 | 87195850 | T | C |
| 7 | 87199264 | C | A | 7 | 87199265 | T | A | 7 | 87604085 | A | T |
| 7 | 88195330 | G | A | 7 | 88454877 | G | C | 7 | 89641818 | T | C |
| 7 | 89641833 | G | A | 7 | 90158020 | C | T | 7 | 90158026 | C | G |
| 7 | 91509551 | T | C | 7 | 92247112 | C | T | 7 | 92300587 | G | A |
| 7 | 92301040 | C | T | 7 | 93194505 | G | A | 7 | 93194506 | C | G |
| 7 | 93194590 | C | A | 7 | 97264891 | C | A | 7 | 97264891 | C | G |
| 7 | 97560579 | G | A | 7 | 97561099 | A | C | 7 | 97561130 | C | A |
| 7 | 98648365 | C | G | 7 | 99314986 | A | G | 7 | 99738322 | G | C |
| 7 | 101977547 | A | G | 7 | 101977597 | G | A | 7 | 102117789 | G | A |
| 7 | 102539438 | A | T | 7 | 102800500 | A | T | 7 | 102800583 | T | C |
| 7 | 102801333 | C | T | 7 | 104309273 | G | A | 7 | 105460274 | C | A |
| 7 | 105626070 | A | G | 7 | 106848955 | G | T | 7 | 107468001 | C | T |
| 7 | 109660709 | T | C | 7 | 111633858 | T | C | 7 | 113250814 | T | G |
| 7 | 113250857 | C | T | 7 | 114352126 | T | C | 7 | 114352682 | T | A |
| 7 | 115334606 | A | C | 7 | 116340582 | A | C | 7 | 116399131 | G | A |
| 7 | 116423161 | G | A | 7 | 116435416 | G | A | 7 | 116894865 | T | A |
| 7 | 116894915 | A | T | 7 | 116894926 | A | G | 7 | 118211698 | G | A |
| 7 | 118211801 | T | C | 7 | 121419326 | C | T | 7 | 121595833 | G | A |
| 7 | 122184921 | C | T | 7 | 123010356 | A | C | 7 | 123881827 | T | G |
| 7 | 123881905 | G | A | 7 | 124465097 | A | T | 7 | 124469587 | G | A |
| 7 | 124480869 | T | C | 7 | 124481403 | T | A | 7 | 124481420 | T | A |
| 7 | 124483222 | T | C | 7 | 124483272 | A | T | 7 | 124492763 | A | G |
| 7 | 124499388 | G | T | 7 | 124499402 | C | G | 7 | 124503189 | A | G |
| 7 | 124503196 | C | T | 7 | 124537476 | A | C | 7 | 125088151 | A | G |
| 7 | 125640459 | C | T | 7 | 125640469 | A | C | 7 | 125640469 | A | T |
| 7 | 125640499 | C | T | 7 | 125640501 | A | C | 7 | 127239815 | C | T |
| 7 | 128147625 | A | G | 7 | 128210320 | C | T | 7 | 128210643 | T | C |
| 7 | 128210789 | G | A | 7 | 128266315 | A | G | 7 | 128266517 | C | T |
| 7 | 128266627 | G | T | 7 | 128297916 | G | A | 7 | 128766463 | G | T |
| 7 | 128766505 | A | C | 7 | 128767969 | A | G | 7 | 128768048 | C | A |
| 7 | 128846716 | A | G | 7 | 130575897 | T | G | 7 | 131628225 | C | T |
| 7 | 132907794 | C | T | 7 | 133345471 | A | T | 7 | 134945970 | T | C |
| 7 | 134945999 | C | T | 7 | 134946000 | T | G | 7 | 134946430 | A | G |
| 7 | 134946432 | G | C | 7 | 135129961 | A | G | 7 | 135519437 | T | G |
| 7 | 135519460 | A | G | 7 | 136023238 | C | T | 7 | 136023288 | C | T |
| 7 | 138503130 | T | C | 7 | 138503690 | C | T | 7 | 139174728 | T | A |
| 7 | 139595823 | G | C | 7 | 140439987 | T | A | 7 | 140492411 | T | C |
| 7 | 141035361 | G | A | 7 | 141814946 | G | A | 7 | 141815374 | T | G |
| 7 | 142457132 | A | C | 7 | 142457674 | G | A | 7 | 142470290 | T | C |
| 7 | 142478761 | C | A | 7 | 142478986 | A | G | 7 | 142480034 | C | A |
| 7 | 142480148 | G | C | 7 | 143720970 | G | C | 7 | 143720987 | G | T |
| 7 | 143964267 | A | G | 7 | 143964486 | T | C | 7 | 144060802 | C | T |
| 7 | 144670310 | G | A | 7 | 146238505 | T | A | 7 | 147194606 | A | G |
| 7 | 147194697 | C | T | 7 | 147720477 | T | A | 7 | 147720992 | T | C |
| 7 | 148034568 | T | A | 7 | 148035121 | A | G | 7 | 148508481 | A | G |
| 7 | 149031681 | A | G | 7 | 149450425 | G | A | 7 | 149450439 | T | C |
| 7 | 149687249 | G | C | 7 | 149687453 | G | A | 7 | 150485864 | G | A |
| 7 | 151407771 | T | G | 7 | 152372816 | C | G | 7 | 152372924 | G | A |
| 7 | 152752607 | T | C | 7 | 152752612 | C | T | 7 | 153404512 | C | A |
| 7 | 154452587 | C | T | 7 | 154452720 | A | G | 7 | 154452734 | A | G |
| 7 | 154453511 | C | T | 7 | 154453688 | G | A | 7 | 154453864 | A | G |
| 7 | 154453902 | T | C | 7 | 154453921 | C | T | 7 | 154453928 | G | A |
| 7 | 154454135 | T | C | 7 | 154454180 | G | T | 7 | 154454264 | A | G |
| 7 | 154454268 | G | T | 7 | 154454356 | A | G | 7 | 154454435 | A | G |
| 7 | 154454522 | T | C | 7 | 154454523 | G | T | 7 | 154454546 | A | G |
| 7 | 154454616 | C | G | 7 | 154454786 | T | C | 7 | 154454939 | A | G |
| 7 | 154454972 | G | A | 7 | 154455363 | T | C | 7 | 154455500 | A | G |
| 7 | 154455674 | C | A | 7 | 154915559 | G | T | 7 | 154915599 | A | G |
| 7 | 154915632 | C | T | 7 | 154915894 | T | C | 7 | 154916158 | C | T |
| 7 | 154916681 | G | C | 7 | 155126014 | C | T | 7 | 155126074 | G | A |
| 7 | 155126346 | A | G | 7 | 155126421 | C | A | 7 | 155126422 | C | T |
| 7 | 155126626 | C | T | 7 | 155126697 | T | C | 7 | 155126714 | A | G |
| 7 | 155126852 | C | T | 7 | 155126903 | C | T | 7 | 155127026 | T | C |
| 7 | 155127061 | A | C | 7 | 155127063 | C | T | 7 | 155127076 | G | C |
| 7 | 155127111 | A | C | 7 | 155127142 | T | C | 7 | 155127988 | A | G |
| 7 | 155127991 | G | C | 7 | 155128004 | G | C | 7 | 155128005 | A | C |
| 7 | 155128006 | T | C | 7 | 155947955 | G | A | 7 | 156781685 | G | A |
| 7 | 157459940 | T | C | 7 | 157459941 | G | A | 7 | 157950927 | T | C |
| 7 | 157950935 | A | T | 7 | 157951445 | A | G | 7 | 157951812 | A | G |
| 7 | 157952182 | G | A | 7 | 158248001 | G | A | 7 | 158249548 | C | T |
| 7 | 159001392 | A | G | 7 | 159001393 | G | A | 7 | 159001396 | G | T |
| 7 | 159001399 | A | T | 8 | 11386 | T | C | 8 | 40370 | A | G |
| 8 | 40565 | C | T | 8 | 143930 | T | C | 8 | 339174 | G | A |
| 8 | 339319 | T | C | 8 | 339338 | G | A | 8 | 339668 | G | A |
| 8 | 339702 | C | T | 8 | 580564 | T | C | 8 | 1292447 | A | C |
| 8 | 1550886 | C | G | 8 | 1550939 | C | A | 8 | 1552096 | A | G |
| 8 | 1552099 | T | G | 8 | 2676199 | C | A | 8 | 3415423 | A | C |
| 8 | 3415633 | G | A | 8 | 3415649 | C | T | 8 | 3415650 | C | G |
| 8 | 3848580 | T | C | 8 | 3848627 | C | T | 8 | 3848630 | G | C |
| 8 | 3849139 | C | G | 8 | 3849147 | T | A | 8 | 4113196 | G | A |
| 8 | 4650270 | C | T | 8 | 5995309 | A | G | 8 | 5995912 | G | A |
| 8 | 6141844 | T | C | 8 | 6141879 | C | T | 8 | 6141887 | C | T |
| 8 | 6946100 | A | G | 8 | 6946189 | A | G | 8 | 6946639 | G | T |
| 8 | 6946666 | C | A | 8 | 6946666 | C | G | 8 | 6948229 | C | T |
| 8 | 7000182 | T | G | 8 | 7000202 | C | T | 8 | 7000210 | T | C |
| 8 | 7000238 | A | G | 8 | 7070133 | T | C | 8 | 7070315 | A | T |
| 8 | 7192428 | G | A | 8 | 7913763 | C | G | 8 | 7913833 | A | G |
| 8 | 7913965 | A | T | 8 | 7913966 | A | G | 8 | 8088230 | T | C |
| 8 | 8094555 | T | C | 8 | 8737464 | T | G | 8 | 9165380 | G | T |
| 8 | 11189344 | C | G | 8 | 11607330 | C | T | 8 | 11607352 | C | T |
| 8 | 11607372 | T | C | 8 | 11607377 | A | G | 8 | 11607396 | G | A |
| 8 | 11612315 | C | T | 8 | 11612381 | T | C | 8 | 11612929 | A | C |
| 8 | 11614175 | G | T | 8 | 11614225 | G | T | 8 | 11993433 | G | A |
| 8 | 11993450 | G | A | 8 | 11993553 | C | T | 8 | 11993854 | C | T |
| 8 | 12285564 | G | A | 8 | 12285923 | C | T | 8 | 12466022 | C | T |
| 8 | 12507122 | G | C | 8 | 12517750 | A | C | 8 | 12517757 | G | A |
| 8 | 12517789 | A | G | 8 | 12517792 | A | C | 8 | 12517824 | G | T |
| 8 | 12518207 | T | A | 8 | 12518209 | A | G | 8 | 12525951 | T | A |
| 8 | 12526441 | C | G | 8 | 12553656 | A | G | 8 | 12553833 | A | G |
| 8 | 12554019 | C | G | 8 | 13282491 | C | G | 8 | 14085219 | C | G |
| 8 | 14085280 | A | T | 8 | 14085785 | T | C | 8 | 14085790 | G | A |
| 8 | 15229407 | G | C | 8 | 15230000 | G | A | 8 | 15786119 | A | G |
| 8 | 16421564 | G | C | 8 | 16701281 | T | G | 8 | 18074243 | A | T |
| 8 | 18074261 | G | A | 8 | 18080644 | A | T | 8 | 18080651 | A | C |
| 8 | 18080747 | T | G | 8 | 18257795 | C | T | 8 | 19319046 | G | C |
| 8 | 19319062 | G | A | 8 | 19319620 | G | A | 8 | 19319625 | G | A |
| 8 | 20918631 | A | G | 8 | 21170053 | A | G | 8 | 21171077 | A | C |
| 8 | 21171170 | T | G | 8 | 21696395 | T | G | 8 | 21696441 | G | A |
| 8 | 22068081 | A | G | 8 | 24287864 | G | C | 8 | 24593635 | G | A |
| 8 | 26519881 | G | A | 8 | 27325191 | G | T | 8 | 27831553 | T | C |
| 8 | 29398520 | C | T | 8 | 29884966 | C | T | 8 | 29886613 | T | C |
| 8 | 30210844 | A | G | 8 | 30939016 | C | G | 8 | 30974348 | G | A |
| 8 | 30989690 | C | T | 8 | 31498589 | G | A | 8 | 31948864 | A | G |
| 8 | 32405979 | T | C | 8 | 32406602 | C | T | 8 | 32432753 | G | T |
| 8 | 32474622 | A | G | 8 | 32599851 | C | T | 8 | 32617494 | G | A |
| 8 | 32620467 | A | G | 8 | 33282335 | G | A | 8 | 33571231 | G | A |
| 8 | 36952867 | C | T | 8 | 36952918 | A | G | 8 | 36953041 | A | G |
| 8 | 38272542 | C | G | 8 | 38272582 | C | A | 8 | 38284994 | C | T |
| 8 | 38286225 | G | A | 8 | 38319729 | A | G | 8 | 38804069 | A | G |
| 8 | 39015793 | T | G | 8 | 40574679 | T | G | 8 | 48405604 | A | T |
| 8 | 48649894 | G | A | 8 | 49540066 | G | T | 8 | 50732260 | A | G |
| 8 | 51133527 | G | T | 8 | 52152983 | C | T | 8 | 53031404 | A | G |
| 8 | 53851462 | T | C | 8 | 53851506 | A | G | 8 | 54068560 | G | A |
| 8 | 54069239 | G | A | 8 | 54446265 | T | C | 8 | 54452476 | C | A |
| 8 | 54573305 | G | T | 8 | 54573308 | G | A | 8 | 55379994 | T | G |
| 8 | 55918430 | T | C | 8 | 56866780 | T | C | 8 | 56879113 | C | G |
| 8 | 58085212 | G | T | 8 | 58711370 | G | A | 8 | 58711450 | C | G |
| 8 | 59257315 | G | C | 8 | 59337563 | C | T | 8 | 59338856 | G | C |
| 8 | 59736647 | T | A | 8 | 62114861 | G | A | 8 | 63055668 | C | T |
| 8 | 63516587 | G | A | 8 | 64183260 | C | A | 8 | 64321899 | T | C |
| 8 | 64322357 | G | A | 8 | 66081707 | G | T | 8 | 66081733 | A | G |
| 8 | 66081764 | G | T | 8 | 66502666 | G | T | 8 | 67380377 | C | T |
| 8 | 67380863 | T | C | 8 | 68934648 | A | T | 8 | 68965782 | T | A |
| 8 | 68967824 | A | G | 8 | 68968466 | T | G | 8 | 68981646 | T | C |
| 8 | 68981979 | T | C | 8 | 68995205 | A | G | 8 | 68995849 | T | G |
| 8 | 69006214 | A | G | 8 | 69046742 | G | A | 8 | 69104278 | C | T |
| 8 | 69104993 | A | G | 8 | 69129636 | A | T | 8 | 69130238 | G | A |
| 8 | 69143417 | C | T | 8 | 70174276 | G | A | 8 | 71340692 | T | A |
| 8 | 71974737 | G | T | 8 | 72466982 | T | C | 8 | 72530703 | C | T |
| 8 | 73795324 | A | C | 8 | 73795889 | C | G | 8 | 75205025 | T | A |
| 8 | 78756303 | G | T | 8 | 78756753 | A | T | 8 | 79457863 | C | T |
| 8 | 79457991 | C | G | 8 | 79673847 | C | T | 8 | 79673952 | T | C |
| 8 | 79776230 | C | T | 8 | 79776231 | A | G | 8 | 79776242 | C | T |
| 8 | 80432897 | C | T | 8 | 82698092 | A | G | 8 | 82698105 | A | G |
| 8 | 83531397 | G | A | 8 | 84739570 | T | C | 8 | 84740129 | G | A |
| 8 | 85148956 | G | A | 8 | 85950696 | G | A | 8 | 87754395 | G | T |
| 8 | 90255638 | G | A | 8 | 90255693 | A | G | 8 | 90255910 | A | C |
| 8 | 90255921 | A | G | 8 | 90255944 | A | T | 8 | 90372173 | G | A |
| 8 | 90947537 | C | T | 8 | 90955253 | T | G | 8 | 90968041 | A | G |
| 8 | 90976405 | C | T | 8 | 90976906 | G | A | 8 | 90982425 | C | T |
| 8 | 90993395 | C | T | 8 | 96216940 | T | G | 8 | 96939758 | T | A |
| 8 | 97548048 | C | A | 8 | 98232612 | G | A | 8 | 101467885 | G | C |
| 8 | 101467929 | T | C | 8 | 102145463 | A | G | 8 | 102998489 | G | C |
| 8 | 102999087 | A | C | 8 | 104137028 | C | T | 8 | 104137029 | A | C |
| 8 | 107401311 | G | A | 8 | 108671073 | C | T | 8 | 109145249 | G | A |
| 8 | 110482900 | T | C | 8 | 110703134 | C | T | 8 | 111149366 | C | T |
| 8 | 114582521 | C | T | 8 | 115803653 | A | G | 8 | 116980628 | T | C |
| 8 | 117915471 | G | A | 8 | 117915557 | A | G | 8 | 117916015 | G | A |
| 8 | 118250570 | C | T | 8 | 118824909 | A | G | 8 | 118825354 | C | T |
| 8 | 118830894 | C | T | 8 | 118831654 | T | C | 8 | 118834484 | A | C |
| 8 | 118842184 | G | A | 8 | 118849146 | T | C | 8 | 119122037 | T | C |
| 8 | 121244377 | A | G | 8 | 121244406 | A | G | 8 | 121926352 | G | A |
| 8 | 121926410 | T | C | 8 | 122272500 | G | A | 8 | 123314293 | C | T |
| 8 | 125753569 | T | C | 8 | 125753604 | G | A | 8 | 125754151 | A | G |
| 8 | 127239272 | C | T | 8 | 131498869 | A | T | 8 | 132926989 | A | G |
| 8 | 133512139 | C | T | 8 | 134828280 | G | C | 8 | 135318241 | G | T |
| 8 | 135510319 | G | T | 8 | 135510321 | G | T | 8 | 135510329 | T | G |
| 8 | 136215771 | G | A | 8 | 138475155 | T | C | 8 | 138475156 | T | A |
| 8 | 138475196 | G | T | 8 | 139218369 | G | A | 8 | 139219088 | A | C |
| 8 | 139219223 | C | A | 8 | 139219307 | G | A | 8 | 140264369 | G | A |
| 8 | 141167195 | G | A | 8 | 141684170 | T | C | 8 | 141685774 | A | G |
| 8 | 141711343 | G | A | 8 | 141762659 | G | A | 8 | 141774576 | A | G |
| 8 | 141799813 | T | C | 8 | 141828264 | G | A | 8 | 141874677 | C | T |
| 8 | 141890008 | T | C | 8 | 142640521 | G | T | 8 | 142640547 | A | C |
| 8 | 142640675 | T | C | 8 | 142640905 | G | A | 8 | 142641198 | C | A |
| 8 | 142642198 | T | A | 8 | 142642202 | G | A | 8 | 143130734 | T | C |
| 8 | 143956693 | G | A | 8 | 143957163 | C | G | 8 | 143994321 | T | C |
| 8 | 143994349 | T | C | 8 | 143994361 | T | A | 8 | 143994388 | G | C |
| 8 | 143994391 | G | C | 8 | 143994396 | A | T | 8 | 143994398 | A | G |
| 8 | 143994399 | T | C | 8 | 143994470 | G | A | 8 | 143994473 | A | C |
| 8 | 143994480 | A | G | 8 | 143994517 | T | C | 8 | 143994702 | G | T |
| 8 | 144286690 | T | G | 8 | 144286716 | T | C | 8 | 144286717 | T | G |
| 8 | 144286759 | G | C | 8 | 144286859 | G | A | 8 | 144287116 | T | G |
| 8 | 144287144 | G | A | 8 | 144287269 | A | G | 8 | 144287286 | C | T |
| 8 | 144743886 | T | C | 8 | 144743892 | A | G | 8 | 144743959 | A | G |
| 8 | 144744194 | C | T | 8 | 144744512 | C | T | 8 | 145180748 | G | A |
| 8 | 145180769 | G | C | 9 | 85392 | G | C | 9 | 118535 | T | C |
| 9 | 118653 | T | C | 9 | 147988 | G | A | 9 | 1200477 | T | C |
| 9 | 1200491 | A | C | 9 | 1200494 | C | G | 9 | 1200508 | T | C |
| 9 | 1201091 | T | C | 9 | 1798525 | G | A | 9 | 1799027 | C | T |
| 9 | 2120825 | G | A | 9 | 2121371 | G | T | 9 | 2624186 | A | G |
| 9 | 4090177 | T | A | 9 | 4090290 | T | C | 9 | 4835827 | C | G |
| 9 | 5021738 | G | A | 9 | 5055434 | C | T | 9 | 5056037 | A | G |
| 9 | 5072846 | G | A | 9 | 5078117 | T | C | 9 | 5081585 | A | G |
| 9 | 5082106 | G | T | 9 | 5465262 | G | A | 9 | 5549080 | T | C |
| 9 | 5549099 | G | A | 9 | 6033278 | G | A | 9 | 6930845 | C | T |
| 9 | 6930896 | G | A | 9 | 6930901 | T | G | 9 | 6931468 | T | A |
| 9 | 7273796 | C | G | 9 | 7274258 | G | C | 9 | 7597988 | C | T |
| 9 | 7798435 | C | T | 9 | 7798518 | G | C | 9 | 7994043 | A | G |
| 9 | 7994044 | T | A | 9 | 7994129 | A | T | 9 | 8578261 | T | C |
| 9 | 9394902 | A | G | 9 | 9651978 | G | C | 9 | 11167895 | C | T |
| 9 | 11610136 | C | A | 9 | 14473276 | A | C | 9 | 14518676 | G | C |
| 9 | 14519301 | T | C | 9 | 17014049 | C | T | 9 | 18936179 | C | G |
| 9 | 19789029 | G | A | 9 | 20414692 | C | G | 9 | 20973968 | T | A |
| 9 | 20974346 | T | C | 9 | 20975006 | C | A | 9 | 21166004 | A | G |
| 9 | 21166077 | T | C | 9 | 21187121 | T | A | 9 | 21201703 | T | C |
| 9 | 21206763 | G | T | 9 | 21216728 | T | C | 9 | 21350050 | T | C |
| 9 | 21350078 | A | T | 9 | 21440490 | T | C | 9 | 21974218 | A | G |
| 9 | 21975141 | A | T | 9 | 25475165 | T | A | 9 | 27168315 | G | A |
| 9 | 27169190 | A | T | 9 | 27185249 | C | A | 9 | 27185251 | C | T |
| 9 | 27190949 | T | C | 9 | 27190992 | C | T | 9 | 27202662 | C | T |
| 9 | 27206307 | A | C | 9 | 27208857 | G | T | 9 | 27209422 | G | A |
| 9 | 27209469 | A | C | 9 | 27219058 | G | A | 9 | 27228022 | A | G |
| 9 | 27228058 | T | G | 9 | 27228532 | G | A | 9 | 27228564 | G | A |
| 9 | 27584899 | T | C | 9 | 27608585 | G | C | 9 | 27608667 | T | C |
| 9 | 29706139 | G | A | 9 | 31020442 | A | C | 9 | 32264612 | G | A |
| 9 | 32264652 | T | C | 9 | 32853813 | G | A | 9 | 33524684 | G | A |
| 9 | 33524725 | G | T | 9 | 33524775 | C | A | 9 | 33525041 | T | A |
| 9 | 33602938 | T | G | 9 | 33602956 | G | A | 9 | 33603177 | G | A |
| 9 | 33613678 | C | A | 9 | 33613770 | T | A | 9 | 33613903 | A | G |
| 9 | 33613912 | C | G | 9 | 33613913 | T | G | 9 | 33613915 | A | G |
| 9 | 33613920 | C | A | 9 | 33613927 | G | T | 9 | 33613986 | C | A |
| 9 | 33796912 | A | G | 9 | 34724786 | G | A | 9 | 34724796 | T | A |
| 9 | 34724832 | G | A | 9 | 34834103 | T | C | 9 | 34834109 | T | C |
| 9 | 35657741 | A | G | 9 | 36847139 | G | A | 9 | 37003075 | A | G |
| 9 | 37020361 | G | A | 9 | 37020388 | T | C | 9 | 37033693 | A | G |
| 9 | 37490307 | C | A | 9 | 37490422 | G | A | 9 | 37490577 | C | G |
| 9 | 37512984 | G | A | 9 | 37513061 | C | G | 9 | 38344773 | T | A |
| 9 | 39029031 | T | C | 9 | 39029036 | C | A | 9 | 40072440 | C | A |
| 9 | 41237632 | C | G | 9 | 42847566 | G | A | 9 | 42847573 | C | A |
| 9 | 42847654 | A | C | 9 | 43541016 | C | T | 9 | 43940526 | T | G |
| 9 | 44212144 | T | G | 9 | 44245084 | C | T | 9 | 44761941 | T | C |
| 9 | 44768417 | G | A | 9 | 44768485 | A | C | 9 | 44768659 | C | T |
| 9 | 44791283 | G | T | 9 | 44791365 | T | C | 9 | 44841887 | G | A |
| 9 | 44841907 | T | G | 9 | 44842094 | G | A | 9 | 44842252 | A | C |
| 9 | 44842293 | T | G | 9 | 44847374 | G | A | 9 | 44847446 | A | C |
| 9 | 44847490 | A | G | 9 | 44847927 | C | A | 9 | 44847977 | T | C |
| 9 | 44865971 | G | T | 9 | 44866028 | C | T | 9 | 44866421 | C | T |
| 9 | 44904433 | A | G | 9 | 44904612 | A | C | 9 | 45079401 | T | C |
| 9 | 45079451 | C | G | 9 | 45079535 | T | G | 9 | 45079548 | G | A |
| 9 | 45079638 | G | A | 9 | 45079671 | T | G | 9 | 45415790 | G | C |
| 9 | 45741414 | G | A | 9 | 45741417 | A | G | 9 | 45741583 | A | G |
| 9 | 45758158 | C | T | 9 | 45758167 | T | A | 9 | 46125082 | T | C |
| 9 | 46125086 | G | A | 9 | 46125090 | G | A | 9 | 46200382 | G | T |
| 9 | 46200485 | C | T | 9 | 46200696 | G | A | 9 | 46200729 | T | G |
| 9 | 46877337 | A | G | 9 | 46877500 | A | C | 9 | 47165813 | A | T |
| 9 | 47165846 | C | A | 9 | 66260870 | A | C | 9 | 66675478 | A | G |
| 9 | 66675670 | T | C | 9 | 66675721 | C | T | 9 | 66675793 | G | A |
| 9 | 67020555 | G | A | 9 | 67020584 | A | C | 9 | 67020641 | A | C |
| 9 | 67683148 | C | A | 9 | 67683187 | A | C | 9 | 67683290 | A | G |
| 9 | 67683344 | C | T | 9 | 67683345 | A | G | 9 | 68233761 | T | C |
| 9 | 68672364 | G | T | 9 | 68724152 | G | C | 9 | 68760967 | C | T |
| 9 | 68761034 | G | C | 9 | 68761231 | A | G | 9 | 69450156 | C | T |
| 9 | 69450371 | C | T | 9 | 69563020 | T | C | 9 | 69563070 | A | C |
| 9 | 69784204 | C | T | 9 | 69784585 | C | T | 9 | 69787544 | T | C |
| 9 | 69787624 | C | T | 9 | 69787627 | C | T | 9 | 69787636 | A | T |
| 9 | 69787642 | G | A | 9 | 69787650 | A | G | 9 | 69788026 | G | A |
| 9 | 70152508 | C | T | 9 | 70888221 | C | T | 9 | 70888239 | G | A |
| 9 | 70888295 | T | C | 9 | 71020205 | A | G | 9 | 71020288 | C | G |
| 9 | 71020391 | G | C | 9 | 71020509 | T | C | 9 | 71020669 | T | C |
| 9 | 71628207 | G | C | 9 | 72137536 | A | G | 9 | 73469948 | C | T |
| 9 | 74245426 | C | T | 9 | 78133661 | C | T | 9 | 79150662 | A | G |
| 9 | 80409138 | C | G | 9 | 80795416 | T | C | 9 | 82962978 | G | A |
| 9 | 84935618 | T | C | 9 | 85346339 | C | T | 9 | 87316868 | A | T |
| 9 | 87322540 | T | C | 9 | 87325994 | A | G | 9 | 87338267 | T | A |
| 9 | 87425206 | T | G | 9 | 87481852 | G | A | 9 | 87570004 | C | T |
| 9 | 87570705 | C | T | 9 | 87635540 | C | T | 9 | 90268337 | T | C |
| 9 | 90631191 | A | G | 9 | 90631497 | T | C | 9 | 90650554 | C | T |
| 9 | 90650578 | T | C | 9 | 90651113 | A | G | 9 | 90940058 | G | A |
| 9 | 91154126 | A | G | 9 | 92280487 | A | G | 9 | 92280986 | T | C |
| 9 | 92995579 | C | T | 9 | 93359638 | A | G | 9 | 93504722 | T | C |
| 9 | 93714276 | T | C | 9 | 93714912 | C | G | 9 | 94518604 | T | C |
| 9 | 94768542 | A | G | 9 | 95484063 | A | G | 9 | 96375217 | T | C |
| 9 | 96375464 | G | A | 9 | 96581893 | A | G | 9 | 96582360 | T | C |
| 9 | 97078732 | A | G | 9 | 97078831 | C | A | 9 | 97078905 | A | G |
| 9 | 97078981 | A | G | 9 | 98009494 | C | T | 9 | 98209958 | G | A |
| 9 | 98231908 | G | A | 9 | 98238860 | T | C | 9 | 98248328 | A | G |
| 9 | 98248387 | A | G | 9 | 98270168 | A | G | 9 | 98270171 | A | G |
| 9 | 98278644 | C | A | 9 | 98279370 | T | A | 9 | 99703218 | G | T |
| 9 | 100437487 | G | T | 9 | 100437518 | G | C | 9 | 100447477 | A | C |
| 9 | 100452141 | T | G | 9 | 100462763 | G | C | 9 | 104565163 | G | T |
| 9 | 105915548 | G | A | 9 | 106693503 | T | C | 9 | 107361111 | T | C |
| 9 | 107361129 | C | T | 9 | 107361312 | T | C | 9 | 107361439 | G | C |
| 9 | 107361449 | T | C | 9 | 107361451 | G | A | 9 | 107361452 | C | G |
| 9 | 107361460 | G | A | 9 | 107361490 | A | T | 9 | 107361599 | G | C |
| 9 | 107361642 | G | A | 9 | 107361710 | G | A | 9 | 107367431 | A | T |
| 9 | 107367841 | A | T | 9 | 107379895 | G | T | 9 | 107380396 | T | G |
| 9 | 107380414 | C | A | 9 | 107839521 | G | A | 9 | 107839542 | A | G |
| 9 | 108654829 | G | A | 9 | 109863693 | G | A | 9 | 112508341 | G | A |
| 9 | 113336508 | C | T | 9 | 114371047 | T | C | 9 | 114371072 | G | A |
| 9 | 114371590 | G | A | 9 | 114371624 | A | C | 9 | 114680414 | C | T |
| 9 | 114680447 | G | A | 9 | 115386827 | A | T | 9 | 115821854 | T | C |
| 9 | 116025982 | A | C | 9 | 116026034 | A | G | 9 | 116026040 | C | T |
| 9 | 116026041 | G | A | 9 | 116816581 | G | A | 9 | 116816582 | A | T |
| 9 | 117092020 | G | A | 9 | 117750232 | T | C | 9 | 117750277 | T | C |
| 9 | 119127244 | T | C | 9 | 120437366 | C | T | 9 | 124570006 | C | T |
| 9 | 124570555 | A | G | 9 | 125288893 | C | T | 9 | 126202375 | C | T |
| 9 | 126214331 | G | C | 9 | 126219431 | T | C | 9 | 126319637 | C | G |
| 9 | 126414074 | T | C | 9 | 126555072 | G | C | 9 | 127956805 | C | G |
| 9 | 127956819 | A | G | 9 | 127957393 | T | C | 9 | 128026278 | G | T |
| 9 | 131993190 | C | T | 9 | 132200320 | G | A | 9 | 132524895 | C | G |
| 9 | 133440976 | C | T | 9 | 135271536 | T | C | 9 | 135271577 | C | T |
| 9 | 135271591 | T | C | 9 | 135273266 | A | G | 9 | 135273288 | A | G |
| 9 | 135276546 | G | A | 9 | 135276606 | A | G | 9 | 135777711 | T | A |
| 9 | 135779530 | T | C | 9 | 135781709 | G | C | 9 | 135782968 | C | T |
| 9 | 135802298 | A | G | 9 | 136768563 | C | A | 9 | 136768575 | A | G |
| 9 | 137415294 | T | C | 9 | 137415319 | C | T | 9 | 137415342 | T | C |
| 9 | 137415554 | A | G | 9 | 137415932 | A | G | 9 | 137416041 | A | C |
| 9 | 137416082 | T | C | 9 | 137416186 | C | T | 9 | 137416192 | C | T |
| 9 | 137416251 | C | T | 9 | 138364601 | T | C | 9 | 138604936 | C | T |
| 9 | 138604947 | C | G | 9 | 139403770 | G | A | 9 | 139411374 | A | G |
| 9 | 139411452 | G | A | 9 | 139411479 | G | A | 9 | 139413533 | C | T |
| 9 | 139413539 | C | T | 9 | 139418661 | A | G | 9 | 139997934 | A | G |
| 9 | 139997971 | C | T | 9 | 139998042 | C | T | 9 | 140242925 | C | T |
| 9 | 140242995 | G | A | 9 | 140242998 | G | T | 9 | 140243000 | T | C |
| 9 | 140675590 | T | C | 9 | 141031520 | C | T | 9 | 141031567 | G | C |
| 9 | 141031898 | T | G | 9 | 141031991 | T | C | 9 | 141075634 | G | A |
| 9 | 141076109 | C | A | 10 | 392593 | T | C | 10 | 392722 | G | A |
| 10 | 392735 | G | A | 10 | 392765 | G | A | 10 | 393501 | C | T |
| 10 | 393577 | C | T | 10 | 393669 | G | A | 10 | 393674 | T | C |
| 10 | 1482550 | C | T | 10 | 1582749 | T | C | 10 | 1582934 | T | C |
| 10 | 1582958 | A | G | 10 | 1583176 | T | C | 10 | 1583692 | T | C |
| 10 | 1584399 | A | G | 10 | 1584802 | G | A | 10 | 2565603 | A | T |
| 10 | 2565607 | G | T | 10 | 2565611 | G | A | 10 | 2565621 | T | C |
| 10 | 2565636 | T | G | 10 | 2566085 | C | T | 10 | 3498982 | A | G |
| 10 | 3499603 | C | T | 10 | 3499625 | T | C | 10 | 3499710 | A | G |
| 10 | 3499787 | T | C | 10 | 3499972 | A | G | 10 | 3500517 | C | T |
| 10 | 5018132 | A | G | 10 | 5018146 | T | A | 10 | 5018174 | C | T |
| 10 | 5018178 | G | A | 10 | 5018204 | A | G | 10 | 5018224 | C | T |
| 10 | 5018310 | T | C | 10 | 5018311 | G | T | 10 | 5033837 | C | A |
| 10 | 5033838 | A | G | 10 | 5034016 | T | C | 10 | 6182251 | C | T |
| 10 | 6182642 | G | A | 10 | 7535307 | A | G | 10 | 8097368 | G | A |
| 10 | 8098185 | A | G | 10 | 8105745 | G | A | 10 | 8106353 | G | A |
| 10 | 8111168 | C | T | 10 | 8562125 | A | T | 10 | 8562634 | A | G |
| 10 | 10779423 | C | T | 10 | 11769964 | G | T | 10 | 12201176 | C | T |
| 10 | 13000296 | G | A | 10 | 13000315 | C | T | 10 | 13000321 | G | C |
| 10 | 13978012 | G | A | 10 | 15009109 | T | C | 10 | 15009164 | G | T |
| 10 | 15009590 | A | G | 10 | 16416773 | A | G | 10 | 16416894 | A | G |
| 10 | 16417376 | T | C | 10 | 16969923 | A | C | 10 | 17419872 | G | A |
| 10 | 17419920 | C | G | 10 | 17591919 | C | G | 10 | 18440733 | T | C |
| 10 | 18790620 | A | G | 10 | 19007587 | C | T | 10 | 19007616 | A | G |
| 10 | 19764840 | T | C | 10 | 19765360 | G | A | 10 | 19765370 | C | T |
| 10 | 20932917 | C | T | 10 | 20933397 | G | A | 10 | 21063302 | G | C |
| 10 | 22493554 | T | A | 10 | 23400393 | T | C | 10 | 26158386 | A | C |
| 10 | 26158422 | A | C | 10 | 26930489 | G | C | 10 | 27227955 | T | C |
| 10 | 27539507 | C | T | 10 | 27539528 | G | A | 10 | 27539598 | G | C |
| 10 | 27539619 | T | C | 10 | 27539928 | A | G | 10 | 27539934 | G | A |
| 10 | 27692062 | T | C | 10 | 27692099 | A | G | 10 | 27692561 | T | G |
| 10 | 27692581 | G | T | 10 | 28192872 | C | T | 10 | 28193276 | G | T |
| 10 | 29491074 | A | T | 10 | 29491536 | G | A | 10 | 29783813 | G | A |
| 10 | 29783885 | A | G | 10 | 29784003 | G | A | 10 | 29784072 | G | C |
| 10 | 29784114 | T | C | 10 | 29784176 | G | A | 10 | 29784177 | T | C |
| 10 | 29784178 | G | C | 10 | 29784188 | A | G | 10 | 29784190 | A | G |
| 10 | 29784218 | G | T | 10 | 29784297 | T | A | 10 | 29784333 | A | G |
| 10 | 29784363 | C | T | 10 | 30213011 | C | T | 10 | 30213059 | G | A |
| 10 | 30213561 | C | T | 10 | 30977801 | T | C | 10 | 30978282 | G | T |
| 10 | 30978377 | G | C | 10 | 30978388 | C | T | 10 | 30978433 | A | G |
| 10 | 34451892 | G | A | 10 | 34451933 | T | C | 10 | 36910716 | G | A |
| 10 | 37430500 | C | A | 10 | 38539751 | C | T | 10 | 38597731 | G | A |
| 10 | 38711591 | C | G | 10 | 38711618 | C | A | 10 | 38743039 | G | A |
| 10 | 38743615 | G | A | 10 | 38743659 | A | G | 10 | 38743802 | G | T |
| 10 | 38752939 | A | C | 10 | 38770349 | C | A | 10 | 38893425 | C | T |
| 10 | 38893430 | C | T | 10 | 38893448 | T | C | 10 | 38893449 | G | A |
| 10 | 38893451 | C | T | 10 | 38893471 | C | T | 10 | 39006616 | G | T |
| 10 | 39011495 | C | T | 10 | 39012502 | A | G | 10 | 39023235 | C | G |
| 10 | 39023315 | T | C | 10 | 42607378 | T | C | 10 | 42607892 | A | G |
| 10 | 42607909 | C | T | 10 | 42607937 | G | A | 10 | 42607955 | G | T |
| 10 | 42607956 | C | T | 10 | 42738375 | C | G | 10 | 42821007 | A | G |
| 10 | 43247120 | G | A | 10 | 43247625 | C | G | 10 | 43572507 | A | G |
| 10 | 43572511 | C | A | 10 | 43572950 | A | G | 10 | 43572951 | A | C |
| 10 | 43601559 | G | A | 10 | 43604950 | A | G | 10 | 43614230 | G | A |
| 10 | 43614694 | G | A | 10 | 43620079 | G | A | 10 | 43620134 | C | T |
| 10 | 43691691 | C | T | 10 | 45406608 | T | C | 10 | 45652138 | T | C |
| 10 | 45652447 | A | G | 10 | 45652504 | T | G | 10 | 45652579 | A | G |
| 10 | 45922190 | G | A | 10 | 46195321 | T | G | 10 | 47152194 | G | A |
| 10 | 47152364 | A | G | 10 | 47170635 | A | T | 10 | 47707447 | G | A |
| 10 | 47707459 | T | C | 10 | 47729649 | C | T | 10 | 47739153 | A | G |
| 10 | 47750099 | G | T | 10 | 47768664 | T | C | 10 | 47768722 | T | C |
| 10 | 49218034 | G | A | 10 | 49218414 | C | A | 10 | 49218451 | G | A |
| 10 | 49218561 | G | C | 10 | 49240255 | A | G | 10 | 49240259 | C | A |
| 10 | 49872724 | C | A | 10 | 50013531 | A | G | 10 | 50013580 | C | T |
| 10 | 50013594 | C | T | 10 | 50534599 | G | A | 10 | 50534646 | C | T |
| 10 | 51486999 | G | A | 10 | 52438586 | G | A | 10 | 52438599 | G | A |
| 10 | 52439039 | G | A | 10 | 52467072 | G | A | 10 | 52537198 | C | T |
| 10 | 53615000 | T | C | 10 | 54171342 | G | T | 10 | 54171478 | A | T |
| 10 | 54171706 | G | T | 10 | 54228504 | G | T | 10 | 54228515 | A | G |
| 10 | 55422826 | G | T | 10 | 56915486 | C | T | 10 | 56915523 | G | A |
| 10 | 56915531 | A | T | 10 | 58941173 | C | T | 10 | 59342734 | T | A |
| 10 | 59343246 | A | C | 10 | 60228964 | G | A | 10 | 61956912 | C | A |
| 10 | 63723336 | T | C | 10 | 63723440 | A | C | 10 | 63723909 | C | T |
| 10 | 64703524 | C | T | 10 | 66488391 | G | T | 10 | 67598587 | C | T |
| 10 | 68498439 | A | G | 10 | 70342096 | G | A | 10 | 70836869 | T | G |
| 10 | 71582636 | T | A | 10 | 72359787 | A | G | 10 | 72691384 | C | T |
| 10 | 72691997 | C | T | 10 | 73542342 | A | G | 10 | 73542839 | T | C |
| 10 | 75434461 | G | A | 10 | 75458179 | C | A | 10 | 78649399 | C | T |
| 10 | 78649930 | A | G | 10 | 79540127 | C | T | 10 | 81282125 | G | A |
| 10 | 81282738 | T | C | 10 | 81632385 | G | C | 10 | 83187807 | T | A |
| 10 | 83187909 | C | T | 10 | 84158423 | G | A | 10 | 84158433 | A | G |
| 10 | 84158436 | G | A | 10 | 84158972 | T | C | 10 | 84473715 | A | G |
| 10 | 86281312 | G | C | 10 | 88651750 | C | T | 10 | 89128565 | C | G |
| 10 | 90232512 | T | C | 10 | 91142901 | T | C | 10 | 91570905 | C | T |
| 10 | 96541373 | A | G | 10 | 96602398 | T | A | 10 | 96741426 | C | T |
| 10 | 97759583 | T | C | 10 | 97759626 | A | G | 10 | 97760182 | G | A |
| 10 | 97949901 | C | T | 10 | 97990491 | C | T | 10 | 98319855 | A | G |
| 10 | 98320693 | G | C | 10 | 98320932 | A | G | 10 | 98510073 | A | G |
| 10 | 99439541 | G | C | 10 | 99439556 | A | C | 10 | 99439901 | G | A |
| 10 | 99972123 | T | G | 10 | 100508702 | C | A | 10 | 101341813 | G | A |
| 10 | 104263675 | T | C | 10 | 104268626 | T | C | 10 | 104269301 | G | A |
| 10 | 104374815 | G | C | 10 | 104378563 | G | T | 10 | 104378750 | G | A |
| 10 | 107980644 | T | G | 10 | 108496447 | G | A | 10 | 108496455 | C | T |
| 10 | 108497048 | C | T | 10 | 109397974 | A | G | 10 | 109398465 | T | C |
| 10 | 110281465 | C | T | 10 | 110998926 | G | C | 10 | 111333728 | A | G |
| 10 | 113752957 | C | T | 10 | 113753002 | T | C | 10 | 113753096 | T | C |
| 10 | 114488065 | G | A | 10 | 114591047 | C | G | 10 | 115475804 | A | G |
| 10 | 115548915 | A | T | 10 | 115548971 | T | A | 10 | 116006120 | C | T |
| 10 | 116006247 | T | C | 10 | 116180554 | C | T | 10 | 117486391 | G | T |
| 10 | 117810868 | G | A | 10 | 118147645 | A | G | 10 | 118147665 | T | C |
| 10 | 118148153 | A | C | 10 | 118525028 | A | T | 10 | 118525526 | T | C |
| 10 | 119243364 | C | T | 10 | 119243810 | T | A | 10 | 120403436 | C | T |
| 10 | 120766000 | A | G | 10 | 120766163 | T | A | 10 | 121050776 | C | T |
| 10 | 121050852 | G | A | 10 | 121050965 | G | A | 10 | 121162095 | T | C |
| 10 | 121631812 | C | G | 10 | 122520364 | C | T | 10 | 123246612 | G | A |
| 10 | 123345664 | C | T | 10 | 123358170 | C | T | 10 | 123358273 | C | T |
| 10 | 123862546 | C | T | 10 | 123862547 | A | G | 10 | 124573552 | T | C |
| 10 | 124573574 | C | T | 10 | 125310002 | C | T | 10 | 125310499 | G | A |
| 10 | 125757093 | C | T | 10 | 126195810 | G | A | 10 | 126195824 | C | G |
| 10 | 126196558 | G | C | 10 | 126741574 | C | T | 10 | 127349999 | A | C |
| 10 | 127846669 | C | T | 10 | 127846703 | C | T | 10 | 127846720 | T | C |
| 10 | 127847387 | T | A | 10 | 128227832 | T | C | 10 | 129339809 | G | A |
| 10 | 129340335 | T | C | 10 | 129650290 | G | A | 10 | 129650996 | A | G |
| 10 | 131265852 | C | T | 10 | 131506546 | G | A | 10 | 131557867 | A | G |
| 10 | 131557933 | G | A | 10 | 131564809 | G | A | 10 | 131565509 | G | A |
| 10 | 132295030 | A | G | 10 | 132295056 | T | C | 10 | 132557571 | T | C |
| 10 | 132557592 | A | G | 10 | 132557593 | T | A | 10 | 132559128 | T | C |
| 10 | 133566682 | G | A | 10 | 134318895 | G | T | 10 | 134926554 | A | G |
| 10 | 134926776 | T | C | 11 | 179894 | T | A | 11 | 2157044 | C | G |
| 11 | 2161159 | G | A | 11 | 3435481 | T | C | 11 | 3435668 | A | G |
| 11 | 3435671 | C | G | 11 | 3439299 | C | T | 11 | 3439368 | G | T |
| 11 | 3439925 | C | T | 11 | 3463826 | T | C | 11 | 3463827 | G | A |
| 11 | 3553733 | T | C | 11 | 3592585 | A | G | 11 | 3603224 | G | A |
| 11 | 3605382 | T | G | 11 | 3605415 | T | C | 11 | 3605624 | A | T |
| 11 | 3605644 | T | C | 11 | 3605771 | G | A | 11 | 3605773 | C | T |
| 11 | 3913738 | A | G | 11 | 4115974 | C | A | 11 | 4128486 | A | G |
| 11 | 4130621 | A | G | 11 | 4132640 | C | A | 11 | 4139978 | T | C |
| 11 | 4142592 | T | C | 11 | 4143692 | T | A | 11 | 4143721 | G | T |
| 11 | 4144842 | A | G | 11 | 4144867 | C | G | 11 | 4150101 | T | C |
| 11 | 4153891 | T | C | 11 | 4155132 | C | T | 11 | 4156040 | T | A |
| 11 | 4156106 | G | A | 11 | 4159202 | T | C | 11 | 4159225 | A | G |
| 11 | 4159247 | G | A | 11 | 4159252 | C | T | 11 | 4159285 | T | C |
| 11 | 5232146 | G | A | 11 | 5232212 | T | A | 11 | 6324678 | T | C |
| 11 | 6325216 | T | C | 11 | 6807468 | A | T | 11 | 7449501 | G | A |
| 11 | 7505088 | C | A | 11 | 7505089 | T | C | 11 | 8285124 | A | C |
| 11 | 9682401 | C | T | 11 | 10754380 | T | G | 11 | 11331170 | A | G |
| 11 | 11504981 | C | T | 11 | 14274550 | T | G | 11 | 14957663 | G | A |
| 11 | 16334778 | A | T | 11 | 17074520 | G | A | 11 | 18288806 | C | G |
| 11 | 18288948 | G | A | 11 | 18289166 | T | C | 11 | 18977418 | A | G |
| 11 | 19865934 | C | G | 11 | 20062121 | T | A | 11 | 20062277 | T | C |
| 11 | 20062704 | C | A | 11 | 21484515 | C | T | 11 | 21562629 | G | C |
| 11 | 21563066 | T | C | 11 | 22211249 | A | G | 11 | 22646025 | G | A |
| 11 | 22647550 | A | G | 11 | 23039122 | T | C | 11 | 23539200 | C | G |
| 11 | 23539342 | A | G | 11 | 23539753 | C | T | 11 | 24396319 | T | C |
| 11 | 25041687 | T | C | 11 | 25041755 | T | G | 11 | 25041811 | A | C |
| 11 | 25041812 | A | G | 11 | 25042243 | G | A | 11 | 25042257 | C | T |
| 11 | 27354842 | C | G | 11 | 29754392 | A | G | 11 | 32410337 | C | G |
| 11 | 32457138 | A | G | 11 | 33394460 | T | C | 11 | 34251360 | T | C |
| 11 | 34251855 | G | A | 11 | 35425747 | T | A | 11 | 36472800 | A | G |
| 11 | 37145360 | T | C | 11 | 38527379 | G | A | 11 | 42748900 | A | T |
| 11 | 42748986 | T | C | 11 | 42748998 | G | A | 11 | 43160505 | T | G |
| 11 | 43536288 | A | G | 11 | 44129086 | A | T | 11 | 44151480 | G | T |
| 11 | 44855872 | G | C | 11 | 45123270 | G | A | 11 | 46166591 | G | T |
| 11 | 46450781 | C | G | 11 | 46450821 | G | A | 11 | 48453678 | G | A |
| 11 | 48485877 | T | C | 11 | 48599491 | A | G | 11 | 48985441 | T | C |
| 11 | 48985508 | G | C | 11 | 49009877 | T | C | 11 | 49080616 | T | C |
| 11 | 49102685 | G | A | 11 | 49103176 | C | T | 11 | 49829781 | G | A |
| 11 | 49829847 | G | T | 11 | 49844161 | C | G | 11 | 49844296 | T | G |
| 11 | 49844746 | G | C | 11 | 49844747 | T | C | 11 | 49867489 | A | C |
| 11 | 49867490 | A | C | 11 | 49868030 | A | G | 11 | 49868050 | G | C |
| 11 | 49868051 | G | A | 11 | 49878757 | G | T | 11 | 49878885 | T | C |
| 11 | 49878894 | G | A | 11 | 49878896 | A | G | 11 | 49878899 | G | C |
| 11 | 49908279 | G | C | 11 | 49908382 | G | A | 11 | 49908392 | C | A |
| 11 | 49908581 | T | C | 11 | 49908583 | C | T | 11 | 49908586 | C | A |
| 11 | 49908592 | A | G | 11 | 49908840 | A | G | 11 | 49908856 | A | C |
| 11 | 51483215 | C | T | 11 | 51483747 | G | C | 11 | 51515460 | C | T |
| 11 | 51515640 | A | G | 11 | 51516257 | A | T | 11 | 54934563 | C | T |
| 11 | 55035985 | G | A | 11 | 55036233 | C | T | 11 | 55049986 | C | A |
| 11 | 55050558 | T | C | 11 | 55071967 | T | G | 11 | 55072428 | C | T |
| 11 | 55532244 | T | C | 11 | 55532303 | T | C | 11 | 55532334 | A | G |
| 11 | 55532908 | A | T | 11 | 56517716 | G | A | 11 | 60264755 | G | A |
| 11 | 60265322 | T | C | 11 | 60804962 | G | A | 11 | 61456160 | C | T |
| 11 | 61731977 | G | A | 11 | 61773679 | C | T | 11 | 62098295 | T | A |
| 11 | 62914125 | G | A | 11 | 63817799 | G | C | 11 | 64574200 | G | A |
| 11 | 65793121 | C | T | 11 | 65793797 | G | A | 11 | 67564437 | A | G |
| 11 | 67564738 | G | A | 11 | 67568487 | C | T | 11 | 67570639 | C | T |
| 11 | 67571091 | T | A | 11 | 67714122 | C | G | 11 | 67726415 | G | A |
| 11 | 67931735 | A | G | 11 | 67931761 | G | A | 11 | 69457547 | C | T |
| 11 | 69459046 | A | G | 11 | 69465681 | A | G | 11 | 70424188 | G | A |
| 11 | 70857741 | A | G | 11 | 71347185 | T | C | 11 | 71349215 | G | A |
| 11 | 71349372 | G | A | 11 | 71349527 | T | G | 11 | 71349654 | G | A |
| 11 | 71349696 | T | C | 11 | 71360446 | G | A | 11 | 71399927 | C | T |
| 11 | 71400609 | T | C | 11 | 71477298 | T | C | 11 | 71477335 | C | G |
| 11 | 71502424 | G | A | 11 | 71502531 | G | C | 11 | 71502886 | G | A |
| 11 | 71502930 | A | G | 11 | 71506814 | A | T | 11 | 71615069 | G | A |
| 11 | 75432864 | A | G | 11 | 75432886 | G | C | 11 | 75432903 | G | A |
| 11 | 76169644 | A | G | 11 | 76170687 | C | T | 11 | 76239148 | G | A |
| 11 | 76239150 | C | T | 11 | 76239160 | A | G | 11 | 76250374 | G | A |
| 11 | 76515087 | G | C | 11 | 76515105 | C | A | 11 | 77776590 | G | C |
| 11 | 77777188 | C | T | 11 | 78447534 | T | C | 11 | 80555975 | G | C |
| 11 | 80556380 | T | C | 11 | 81258169 | T | C | 11 | 81258185 | C | T |
| 11 | 83261268 | C | T | 11 | 83261334 | C | T | 11 | 83261851 | A | G |
| 11 | 83580202 | A | C | 11 | 87079863 | A | G | 11 | 88211600 | T | C |
| 11 | 89487612 | G | A | 11 | 89609220 | T | C | 11 | 89609260 | G | A |
| 11 | 89795691 | G | A | 11 | 90372636 | G | A | 11 | 90972900 | C | A |
| 11 | 90973362 | T | C | 11 | 90973418 | G | A | 11 | 91680278 | T | C |
| 11 | 92623608 | A | G | 11 | 93754508 | G | A | 11 | 94180115 | G | C |
| 11 | 94200690 | C | G | 11 | 94203353 | G | A | 11 | 94213155 | C | G |
| 11 | 94759059 | A | G | 11 | 94782500 | C | G | 11 | 94782501 | A | G |
| 11 | 94782515 | C | A | 11 | 95040632 | C | A | 11 | 95041189 | C | A |
| 11 | 95041230 | C | T | 11 | 95532166 | A | G | 11 | 95550810 | C | G |
| 11 | 95561488 | T | G | 11 | 95562771 | T | C | 11 | 96852915 | A | T |
| 11 | 97332219 | G | A | 11 | 98998719 | G | C | 11 | 99216940 | T | G |
| 11 | 99217421 | T | C | 11 | 99865248 | A | G | 11 | 99865266 | C | A |
| 11 | 99865868 | T | C | 11 | 100921941 | T | A | 11 | 101217386 | A | T |
| 11 | 101217962 | A | G | 11 | 101465270 | A | G | 11 | 101981463 | G | T |
| 11 | 101984628 | C | T | 11 | 102079986 | G | A | 11 | 102100199 | C | T |
| 11 | 103416881 | T | C | 11 | 103416913 | T | G | 11 | 103542664 | A | G |
| 11 | 104013175 | A | G | 11 | 104013263 | A | T | 11 | 105724696 | C | T |
| 11 | 106318163 | T | C | 11 | 106692416 | G | A | 11 | 107669817 | C | T |
| 11 | 107669894 | C | T | 11 | 107670383 | C | G | 11 | 107670446 | A | G |
| 11 | 107670459 | A | G | 11 | 108106782 | C | T | 11 | 108158134 | T | C |
| 11 | 108163733 | A | G | 11 | 108165406 | G | A | 11 | 108165407 | C | G |
| 11 | 108170271 | G | A | 11 | 108196509 | G | C | 11 | 108204881 | C | T |
| 11 | 110016032 | A | G | 11 | 110016519 | T | G | 11 | 110712255 | A | T |
| 11 | 110712847 | C | G | 11 | 114092153 | C | A | 11 | 116732856 | A | G |
| 11 | 117343197 | T | C | 11 | 117956263 | C | T | 11 | 117956338 | G | T |
| 11 | 117956409 | T | C | 11 | 117956943 | A | G | 11 | 118362869 | T | C |
| 11 | 118567444 | A | G | 11 | 118567486 | T | C | 11 | 119145274 | A | G |
| 11 | 119167448 | T | G | 11 | 119168370 | G | A | 11 | 120440043 | A | G |
| 11 | 120508093 | C | T | 11 | 124455318 | T | C | 11 | 125497247 | T | C |
| 11 | 125497342 | A | G | 11 | 125505095 | C | G | 11 | 125507676 | C | T |
| 11 | 126666122 | C | T | 11 | 128240874 | A | T | 11 | 128240927 | G | C |
| 11 | 130416523 | C | T | 11 | 130417076 | G | A | 11 | 130417573 | A | G |
| 11 | 131350351 | C | G | 11 | 131350968 | G | A | 11 | 134916463 | G | A |
| 11 | 134916479 | C | T | 11 | 134916495 | T | C | 11 | 134917197 | G | A |
| 12 | 420429 | G | A | 12 | 427077 | G | A | 12 | 433151 | A | G |
| 12 | 442354 | T | C | 12 | 443085 | C | A | 12 | 443795 | T | C |
| 12 | 460226 | C | T | 12 | 461637 | C | T | 12 | 464123 | A | G |
| 12 | 471826 | C | T | 12 | 474797 | T | C | 12 | 494762 | G | C |
| 12 | 528452 | A | T | 12 | 528865 | A | C | 12 | 528887 | A | T |
| 12 | 530660 | T | A | 12 | 530660 | T | G | 12 | 1194236 | A | G |
| 12 | 1194372 | A | G | 12 | 1194777 | A | G | 12 | 1196938 | A | G |
| 12 | 1200335 | G | A | 12 | 1899291 | T | A | 12 | 3379076 | G | A |
| 12 | 3567611 | T | C | 12 | 4445722 | G | A | 12 | 4553184 | C | T |
| 12 | 5082261 | A | G | 12 | 5082274 | C | T | 12 | 5082302 | A | G |
| 12 | 5082835 | A | G | 12 | 5748011 | T | C | 12 | 5748052 | C | T |
| 12 | 5748078 | A | G | 12 | 5748079 | A | G | 12 | 6697842 | A | G |
| 12 | 6704213 | T | A | 12 | 7808545 | A | G | 12 | 8387026 | G | A |
| 12 | 8387543 | C | T | 12 | 8391144 | C | G | 12 | 8391162 | T | C |
| 12 | 8391761 | C | T | 12 | 8393255 | G | A | 12 | 8393268 | A | G |
| 12 | 8393274 | T | C | 12 | 8393314 | G | A | 12 | 8393803 | C | T |
| 12 | 8544062 | A | G | 12 | 8544083 | G | A | 12 | 8544541 | C | T |
| 12 | 8550134 | C | T | 12 | 8552680 | T | C | 12 | 9451473 | G | A |
| 12 | 9451541 | C | A | 12 | 9451818 | T | C | 12 | 9585578 | C | T |
| 12 | 9585658 | A | G | 12 | 9848883 | A | G | 12 | 10854878 | A | G |
| 12 | 11174753 | G | A | 12 | 11174795 | T | G | 12 | 11201629 | G | A |
| 12 | 11230403 | T | C | 12 | 11230436 | G | A | 12 | 11230437 | A | C |
| 12 | 11230452 | C | A | 12 | 11230457 | C | G | 12 | 11230465 | C | T |
| 12 | 11230496 | G | A | 12 | 11230497 | G | A | 12 | 11230520 | G | A |
| 12 | 11230542 | A | C | 12 | 11230544 | C | T | 12 | 11230554 | G | A |
| 12 | 11230566 | G | A | 12 | 11230578 | T | C | 12 | 11230583 | T | C |
| 12 | 11230592 | C | G | 12 | 11230597 | G | A | 12 | 11230598 | C | T |
| 12 | 11230601 | C | T | 12 | 11230609 | G | A | 12 | 11230636 | T | G |
| 12 | 11230662 | C | T | 12 | 11230665 | G | A | 12 | 11230700 | G | C |
| 12 | 11230701 | G | A | 12 | 11230712 | C | T | 12 | 11230726 | C | T |
| 12 | 11230744 | A | T | 12 | 11230752 | T | A | 12 | 11230754 | C | T |
| 12 | 11230775 | A | C | 12 | 11230778 | A | C | 12 | 11230832 | A | C |
| 12 | 11230834 | T | C | 12 | 11230867 | T | A | 12 | 11230873 | T | G |
| 12 | 11230890 | C | G | 12 | 11230895 | A | T | 12 | 11230909 | A | G |
| 12 | 11230936 | A | T | 12 | 11230948 | A | G | 12 | 11230968 | A | G |
| 12 | 11231002 | C | A | 12 | 11231008 | T | C | 12 | 11231018 | G | A |
| 12 | 11231042 | A | G | 12 | 11231052 | G | A | 12 | 11611304 | G | A |
| 12 | 11611331 | C | T | 12 | 11611336 | G | A | 12 | 11611368 | C | T |
| 12 | 11611377 | T | C | 12 | 12027745 | A | C | 12 | 13370236 | A | C |
| 12 | 13555619 | G | T | 12 | 13555639 | C | T | 12 | 13556252 | G | C |
| 12 | 15138281 | A | G | 12 | 16397411 | A | G | 12 | 17757557 | C | A |
| 12 | 20177190 | C | T | 12 | 21616558 | G | A | 12 | 21616598 | C | T |
| 12 | 21617144 | A | G | 12 | 21617145 | A | G | 12 | 22193228 | G | A |
| 12 | 22957461 | A | G | 12 | 22957519 | G | A | 12 | 23192806 | T | C |
| 12 | 24358865 | C | T | 12 | 25363085 | C | T | 12 | 25368084 | T | C |
| 12 | 26504062 | C | G | 12 | 29688497 | T | C | 12 | 30359821 | T | C |
| 12 | 31242271 | G | A | 12 | 31508019 | G | C | 12 | 32741558 | A | G |
| 12 | 33074174 | A | G | 12 | 33074256 | T | G | 12 | 37971347 | G | C |
| 12 | 38600609 | G | A | 12 | 40879387 | A | G | 12 | 40880558 | A | T |
| 12 | 40880586 | A | G | 12 | 40880587 | T | A | 12 | 40880831 | A | T |
| 12 | 40880924 | A | G | 12 | 40881916 | T | A | 12 | 40881964 | G | A |
| 12 | 40881967 | C | T | 12 | 40881986 | G | A | 12 | 40882000 | C | G |
| 12 | 40882006 | T | C | 12 | 40882016 | G | T | 12 | 40882026 | A | G |
| 12 | 40882071 | C | G | 12 | 40882121 | G | T | 12 | 40882450 | G | C |
| 12 | 40882490 | A | G | 12 | 40882522 | C | A | 12 | 40882649 | G | T |
| 12 | 40882701 | G | T | 12 | 40883092 | C | A | 12 | 40883745 | C | A |
| 12 | 40883775 | C | T | 12 | 40883781 | G | A | 12 | 40883786 | T | A |
| 12 | 40883904 | G | A | 12 | 40884061 | C | T | 12 | 42477389 | T | A |
| 12 | 42477390 | A | C | 12 | 42477397 | A | G | 12 | 42477402 | T | C |
| 12 | 42477404 | T | C | 12 | 42477426 | A | G | 12 | 42477431 | C | T |
| 12 | 42477930 | C | T | 12 | 42477945 | A | G | 12 | 42477948 | T | G |
| 12 | 42477951 | T | C | 12 | 42477952 | G | A | 12 | 42477962 | C | T |
| 12 | 42477979 | G | C | 12 | 42478001 | T | G | 12 | 43965011 | A | T |
| 12 | 43965108 | T | A | 12 | 45228464 | C | T | 12 | 46665157 | G | A |
| 12 | 46955423 | C | T | 12 | 51213765 | T | C | 12 | 52698224 | C | T |
| 12 | 52698226 | A | G | 12 | 52698228 | T | G | 12 | 52698230 | C | T |
| 12 | 52698235 | G | T | 12 | 52698236 | C | T | 12 | 53346750 | C | T |
| 12 | 53512756 | A | T | 12 | 53512770 | G | T | 12 | 54087418 | C | T |
| 12 | 54476772 | G | A | 12 | 54501012 | G | C | 12 | 55828540 | C | T |
| 12 | 56473808 | T | C | 12 | 56474480 | A | G | 12 | 56478002 | A | C |
| 12 | 56481118 | G | A | 12 | 56488913 | G | A | 12 | 57489828 | C | T |
| 12 | 57544620 | C | T | 12 | 58141602 | C | T | 12 | 60103232 | G | A |
| 12 | 60553810 | C | T | 12 | 61969925 | T | C | 12 | 62524923 | A | G |
| 12 | 62525409 | A | T | 12 | 63503650 | T | G | 12 | 64798118 | C | G |
| 12 | 68551409 | C | T | 12 | 68946720 | C | T | 12 | 69202580 | T | G |
| 12 | 69203312 | G | A | 12 | 69207162 | G | A | 12 | 69222357 | C | A |
| 12 | 69230189 | G | T | 12 | 70074807 | G | C | 12 | 71520477 | A | C |
| 12 | 71520600 | T | C | 12 | 71521068 | G | T | 12 | 72372601 | C | T |
| 12 | 72979069 | T | A | 12 | 72979099 | T | G | 12 | 73501997 | T | G |
| 12 | 76633769 | T | C | 12 | 77056710 | A | G | 12 | 79575315 | T | A |
| 12 | 79575325 | C | A | 12 | 82670236 | C | T | 12 | 83471875 | G | A |
| 12 | 83545935 | C | T | 12 | 84356493 | G | A | 12 | 85165573 | A | T |
| 12 | 86084416 | G | T | 12 | 87490519 | G | T | 12 | 87490552 | T | C |
| 12 | 88925973 | T | C | 12 | 88939790 | T | C | 12 | 88973732 | A | G |
| 12 | 88974212 | A | G | 12 | 89490962 | T | C | 12 | 89491013 | G | A |
| 12 | 89864432 | T | A | 12 | 90270430 | G | C | 12 | 91851148 | T | A |
| 12 | 92997562 | C | A | 12 | 94372056 | C | G | 12 | 95012735 | A | G |
| 12 | 95602672 | C | T | 12 | 96161299 | T | C | 12 | 96161309 | G | T |
| 12 | 96161410 | A | T | 12 | 96161868 | G | C | 12 | 96161887 | G | C |
| 12 | 96848128 | C | T | 12 | 96848651 | C | T | 12 | 96848682 | A | G |
| 12 | 97799632 | G | A | 12 | 97799634 | C | T | 12 | 97800208 | T | C |
| 12 | 97800239 | C | T | 12 | 98984651 | A | G | 12 | 98985056 | T | C |
| 12 | 99265981 | A | G | 12 | 99266022 | A | T | 12 | 101215628 | G | A |
| 12 | 102361086 | T | A | 12 | 104959816 | T | A | 12 | 105079958 | T | C |
| 12 | 105673808 | G | A | 12 | 106114632 | G | A | 12 | 106620432 | A | G |
| 12 | 107381892 | C | T | 12 | 108168706 | G | A | 12 | 109330915 | G | A |
| 12 | 112241402 | G | A | 12 | 112843998 | A | T | 12 | 112856643 | G | A |
| 12 | 112890776 | A | G | 12 | 112942353 | G | A | 12 | 114501664 | T | C |
| 12 | 114501692 | T | C | 12 | 114501693 | G | A | 12 | 114501708 | T | A |
| 12 | 114501726 | C | T | 12 | 114501852 | A | T | 12 | 114501869 | A | C |
| 12 | 114501990 | A | G | 12 | 114502105 | T | A | 12 | 114502125 | T | C |
| 12 | 114502197 | T | C | 12 | 114502242 | A | G | 12 | 114502276 | A | C |
| 12 | 114502702 | G | T | 12 | 115184053 | G | C | 12 | 115866519 | G | T |
| 12 | 116050988 | C | T | 12 | 117193050 | T | C | 12 | 117692072 | T | C |
| 12 | 118256945 | G | C | 12 | 118256995 | T | A | 12 | 121426478 | C | T |
| 12 | 121433816 | C | T | 12 | 121433844 | A | G | 12 | 121434833 | G | A |
| 12 | 121876257 | G | T | 12 | 121876385 | G | C | 12 | 121876866 | G | A |
| 12 | 122849392 | A | C | 12 | 123186539 | A | G | 12 | 123186610 | G | A |
| 12 | 123186611 | C | G | 12 | 123199745 | T | C | 12 | 123199755 | A | G |
| 12 | 124804371 | G | C | 12 | 124804373 | C | T | 12 | 124804522 | A | C |
| 12 | 124804538 | G | A | 12 | 124804550 | C | A | 12 | 124804566 | G | A |
| 12 | 124804578 | C | A | 12 | 124804593 | G | C | 12 | 124804644 | A | G |
| 12 | 125177433 | C | T | 12 | 125177454 | G | A | 12 | 125177986 | C | T |
| 12 | 127490326 | A | C | 12 | 128958147 | A | G | 12 | 129299134 | T | C |
| 12 | 130876953 | T | C | 12 | 130876961 | G | T | 12 | 130876987 | T | C |
| 12 | 130877140 | G | T | 12 | 130877166 | T | C | 12 | 130877287 | G | A |
| 12 | 130877325 | A | G | 12 | 130877332 | G | A | 12 | 130877408 | C | T |
| 12 | 130877506 | A | G | 12 | 130877521 | G | A | 12 | 131017991 | C | G |
| 12 | 131966057 | T | C | 12 | 131966293 | C | T | 12 | 132286104 | C | T |
| 12 | 132286105 | G | A | 12 | 132286113 | T | C | 12 | 132286133 | G | A |
| 12 | 132957443 | T | C | 12 | 132958490 | A | G | 12 | 132959437 | G | A |
| 12 | 132959439 | G | A | 12 | 132959472 | T | A | 12 | 132959689 | C | T |
| 12 | 132959693 | T | C | 12 | 132959732 | T | C | 12 | 132961351 | G | T |
| 12 | 133201947 | T | C | 12 | 133208611 | A | G | 12 | 133209593 | T | C |
| 12 | 133209653 | C | T | 12 | 133209660 | G | A | 12 | 133212165 | A | G |
| 12 | 133235577 | A | C | 12 | 133241144 | C | A | 12 | 133243788 | T | A |
| 12 | 133243817 | C | T | 12 | 133250569 | T | G | 12 | 133250571 | C | T |
| 12 | 133254529 | C | G | 12 | 133255792 | A | C | 12 | 133258088 | C | T |
| 12 | 133818115 | C | T | 12 | 133818696 | G | T | 13 | 19098169 | G | A |
| 13 | 19098189 | T | C | 13 | 19196854 | T | C | 13 | 19196952 | A | G |
| 13 | 19196957 | A | G | 13 | 19196999 | A | G | 13 | 19197003 | T | C |
| 13 | 19197028 | A | G | 13 | 19197050 | A | C | 13 | 19197108 | G | T |
| 13 | 19197161 | T | C | 13 | 19197162 | G | A | 13 | 19312986 | G | A |
| 13 | 19312997 | T | C | 13 | 20006231 | A | C | 13 | 21517666 | C | T |
| 13 | 21535954 | C | G | 13 | 22638746 | G | A | 13 | 23269969 | T | A |
| 13 | 23516337 | C | T | 13 | 23533834 | T | G | 13 | 23534184 | T | C |
| 13 | 23534203 | G | A | 13 | 23534230 | C | G | 13 | 23534298 | G | A |
| 13 | 24958840 | C | G | 13 | 25244216 | C | G | 13 | 25244691 | C | T |
| 13 | 25535764 | C | G | 13 | 25535765 | A | G | 13 | 25535868 | T | C |
| 13 | 26923565 | T | G | 13 | 26974482 | T | G | 13 | 27487175 | G | A |
| 13 | 27487180 | C | T | 13 | 27827811 | C | T | 13 | 27945465 | T | G |
| 13 | 28597187 | C | T | 13 | 28601651 | T | C | 13 | 28602604 | T | C |
| 13 | 28609396 | T | C | 13 | 28609399 | G | C | 13 | 28610355 | T | A |
| 13 | 28611630 | T | C | 13 | 28644359 | G | C | 13 | 28886388 | A | G |
| 13 | 28893341 | G | C | 13 | 28893870 | C | G | 13 | 28896097 | G | A |
| 13 | 28919330 | G | A | 13 | 29007730 | A | G | 13 | 30015342 | A | G |
| 13 | 30015805 | T | C | 13 | 30888324 | T | A | 13 | 31276032 | G | C |
| 13 | 31276586 | G | T | 13 | 32421423 | C | T | 13 | 32899567 | G | C |
| 13 | 32900933 | T | A | 13 | 32907767 | C | T | 13 | 32921306 | G | A |
| 13 | 32921362 | T | C | 13 | 32945537 | A | T | 13 | 32953182 | A | G |
| 13 | 32968591 | G | A | 13 | 32968607 | A | G | 13 | 34723653 | T | G |
| 13 | 34723667 | C | T | 13 | 34724105 | C | T | 13 | 34724194 | C | T |
| 13 | 36108037 | A | C | 13 | 37377469 | T | A | 13 | 40733509 | C | A |
| 13 | 40733531 | A | T | 13 | 42300323 | T | C | 13 | 43517113 | C | T |
| 13 | 43517127 | A | G | 13 | 43517700 | G | A | 13 | 43517736 | G | T |
| 13 | 44772757 | A | G | 13 | 45626937 | C | T | 13 | 47199611 | T | C |
| 13 | 48273085 | A | G | 13 | 48881154 | G | C | 13 | 48919064 | A | G |
| 13 | 48934575 | A | T | 13 | 48954021 | G | C | 13 | 50466615 | G | C |
| 13 | 50466749 | C | G | 13 | 50466754 | T | C | 13 | 50466766 | C | T |
| 13 | 50466771 | A | G | 13 | 50466774 | G | A | 13 | 50466780 | C | T |
| 13 | 50466781 | A | G | 13 | 50466783 | C | G | 13 | 50935593 | C | T |
| 13 | 51230621 | A | G | 13 | 51231117 | C | T | 13 | 51231132 | G | C |
| 13 | 51685479 | C | T | 13 | 51685541 | C | T | 13 | 51686109 | G | A |
| 13 | 52482451 | G | A | 13 | 52602919 | A | C | 13 | 60909992 | G | A |
| 13 | 61266375 | T | C | 13 | 61651695 | T | A | 13 | 61652128 | T | C |
| 13 | 62884880 | A | G | 13 | 62885397 | T | C | 13 | 62885404 | C | G |
| 13 | 63984445 | G | A | 13 | 63984474 | C | A | 13 | 63984474 | C | T |
| 13 | 63984519 | T | C | 13 | 68406302 | A | G | 13 | 68406306 | A | G |
| 13 | 68406761 | G | A | 13 | 68820749 | C | T | 13 | 68820783 | A | G |
| 13 | 69675780 | G | A | 13 | 69675935 | T | C | 13 | 70884365 | C | G |
| 13 | 70884372 | T | C | 13 | 70884412 | G | A | 13 | 70884937 | T | C |
| 13 | 70885012 | C | T | 13 | 71158022 | T | C | 13 | 71498321 | T | C |
| 13 | 71498338 | C | T | 13 | 71498343 | G | T | 13 | 71498360 | G | C |
| 13 | 71498385 | C | T | 13 | 71498894 | A | G | 13 | 72477265 | C | T |
| 13 | 74077481 | C | T | 13 | 74549292 | T | C | 13 | 76333853 | G | A |
| 13 | 76583319 | A | T | 13 | 77614202 | G | A | 13 | 77614206 | A | T |
| 13 | 77614213 | C | T | 13 | 78800795 | G | A | 13 | 79015424 | G | A |
| 13 | 79775160 | C | T | 13 | 80384592 | G | A | 13 | 80384594 | A | T |
| 13 | 82485701 | G | A | 13 | 82641070 | G | T | 13 | 83720107 | C | G |
| 13 | 85714107 | T | C | 13 | 87209806 | G | T | 13 | 88275166 | G | T |
| 13 | 90617118 | T | C | 13 | 90617599 | C | T | 13 | 91023515 | C | A |
| 13 | 91779179 | A | C | 13 | 93036880 | A | C | 13 | 94062095 | C | A |
| 13 | 94996400 | G | A | 13 | 95538615 | C | G | 13 | 97843293 | G | T |
| 13 | 98419205 | T | C | 13 | 98522674 | A | T | 13 | 99464351 | T | A |
| 13 | 100477134 | C | A | 13 | 100477148 | A | G | 13 | 100545068 | C | T |
| 13 | 101182148 | A | G | 13 | 101987034 | A | C | 13 | 101987047 | A | G |
| 13 | 101987123 | A | G | 13 | 102057883 | A | C | 13 | 102058361 | G | A |
| 13 | 103498375 | T | C | 13 | 103520251 | T | C | 13 | 107853969 | G | T |
| 13 | 109006433 | A | G | 13 | 109006441 | T | C | 13 | 110418135 | C | T |
| 13 | 110418136 | G | A | 13 | 110419404 | C | T | 13 | 110976837 | A | C |
| 13 | 111132947 | G | A | 13 | 111133395 | C | T | 13 | 111133399 | A | G |
| 13 | 111133489 | A | G | 13 | 111133579 | G | A | 13 | 111133625 | T | A |
| 13 | 111133639 | G | A | 13 | 111133665 | C | T | 13 | 111133669 | A | G |
| 13 | 111133835 | A | T | 13 | 111134015 | A | T | 13 | 111134119 | A | G |
| 13 | 111589152 | G | A | 13 | 111589204 | G | C | 13 | 111589278 | G | A |
| 13 | 111590028 | A | G | 13 | 111590550 | A | G | 13 | 111590930 | C | T |
| 13 | 111591170 | G | A | 13 | 111591265 | G | A | 13 | 111591283 | T | C |
| 13 | 111591853 | G | A | 13 | 111592285 | A | G | 13 | 112802383 | G | A |
| 13 | 112802389 | G | A | 13 | 112803722 | T | C | 13 | 112803822 | G | C |
| 13 | 112803840 | A | T | 13 | 112803842 | A | G | 13 | 112803853 | T | G |
| 13 | 113498860 | C | T | 13 | 114089046 | C | T | 13 | 114089082 | C | T |
| 13 | 114089113 | A | G | 13 | 114089128 | T | C | 13 | 114089164 | C | T |
| 13 | 114089330 | G | A | 13 | 114089440 | G | C | 13 | 114089675 | G | T |
| 13 | 114089876 | A | G | 13 | 114089888 | T | C | 13 | 114089891 | A | G |
| 13 | 114089897 | C | T | 13 | 114090025 | C | T | 13 | 114090109 | C | T |
| 13 | 114090253 | T | C | 13 | 114090689 | G | A | 13 | 114091321 | T | C |
| 13 | 114838339 | C | T | 14 | 19491761 | T | C | 14 | 19629734 | C | G |
| 14 | 19688223 | G | A | 14 | 19688480 | G | C | 14 | 20084306 | C | T |
| 14 | 20084444 | G | C | 14 | 20086576 | G | A | 14 | 20140628 | C | G |
| 14 | 20140803 | G | C | 14 | 20140905 | C | T | 14 | 20414407 | C | A |
| 14 | 20414410 | T | C | 14 | 20414737 | C | T | 14 | 20414884 | G | A |
| 14 | 20811577 | A | C | 14 | 20811577 | A | G | 14 | 20811578 | A | G |
| 14 | 20811588 | T | C | 14 | 20812951 | G | A | 14 | 20815313 | A | G |
| 14 | 20822772 | C | T | 14 | 20826243 | A | C | 14 | 21236526 | T | C |
| 14 | 21237137 | G | A | 14 | 21630781 | C | T | 14 | 21860360 | G | A |
| 14 | 21863716 | C | T | 14 | 21871999 | T | G | 14 | 21875487 | T | C |
| 14 | 21894526 | T | C | 14 | 22320158 | C | A | 14 | 22320728 | T | C |
| 14 | 22371221 | G | T | 14 | 22371246 | A | G | 14 | 22746792 | C | T |
| 14 | 23029407 | T | C | 14 | 23029409 | T | C | 14 | 23029512 | G | C |
| 14 | 23029525 | A | T | 14 | 23652708 | T | A | 14 | 24436511 | T | C |
| 14 | 24436706 | G | C | 14 | 24436798 | G | T | 14 | 24473786 | C | A |
| 14 | 24526287 | G | C | 14 | 24526895 | G | A | 14 | 25515815 | A | G |
| 14 | 25515822 | T | C | 14 | 26605655 | T | C | 14 | 27314804 | C | T |
| 14 | 27855023 | T | C | 14 | 27855025 | C | T | 14 | 31919287 | T | C |
| 14 | 36351085 | G | A | 14 | 36417424 | G | A | 14 | 36417426 | T | A |
| 14 | 36417451 | G | A | 14 | 36417453 | T | A | 14 | 36417478 | A | G |
| 14 | 36417480 | A | T | 14 | 36417498 | T | C | 14 | 36417545 | C | T |
| 14 | 36417546 | A | G | 14 | 36417559 | G | A | 14 | 36417572 | T | C |
| 14 | 36417640 | G | A | 14 | 36417696 | A | T | 14 | 36417716 | T | C |
| 14 | 36417723 | A | T | 14 | 36417741 | T | C | 14 | 36417743 | T | C |
| 14 | 36417761 | C | T | 14 | 36417777 | A | T | 14 | 36417802 | A | G |
| 14 | 36417804 | A | T | 14 | 36417842 | T | C | 14 | 36417883 | A | G |
| 14 | 36417957 | T | C | 14 | 36417959 | T | C | 14 | 36418031 | T | C |
| 14 | 36418355 | T | C | 14 | 36419122 | C | A | 14 | 36419133 | T | G |
| 14 | 36989596 | A | G | 14 | 36989696 | G | T | 14 | 38639257 | C | T |
| 14 | 39354900 | C | G | 14 | 39858941 | A | G | 14 | 41413712 | C | T |
| 14 | 42835190 | G | A | 14 | 45027200 | G | T | 14 | 46316764 | A | G |
| 14 | 47492399 | C | G | 14 | 47562483 | T | A | 14 | 48921178 | T | C |
| 14 | 50520782 | A | G | 14 | 52229607 | C | G | 14 | 52229678 | T | C |
| 14 | 52230158 | T | G | 14 | 52519254 | A | G | 14 | 52519839 | G | A |
| 14 | 55880730 | A | G | 14 | 55880918 | A | G | 14 | 55921280 | A | G |
| 14 | 56232970 | T | C | 14 | 56233561 | G | A | 14 | 56548918 | C | A |
| 14 | 57463704 | G | A | 14 | 57586697 | C | T | 14 | 57586731 | T | G |
| 14 | 59170024 | C | G | 14 | 62003902 | C | T | 14 | 62983672 | A | T |
| 14 | 65473196 | G | A | 14 | 65568563 | C | T | 14 | 65733329 | A | T |
| 14 | 65733701 | A | G | 14 | 65734110 | C | G | 14 | 65734181 | C | G |
| 14 | 65734600 | G | A | 14 | 65934921 | T | C | 14 | 68291907 | G | A |
| 14 | 68352323 | A | G | 14 | 68354152 | C | T | 14 | 68758317 | T | C |
| 14 | 68934690 | A | G | 14 | 68944147 | T | G | 14 | 68944245 | T | C |
| 14 | 70440081 | T | C | 14 | 71632622 | C | T | 14 | 71632640 | C | T |
| 14 | 72266238 | T | A | 14 | 72736899 | G | A | 14 | 73047284 | A | G |
| 14 | 73997686 | G | A | 14 | 74025873 | A | T | 14 | 75506875 | G | A |
| 14 | 77101088 | A | G | 14 | 77704974 | G | C | 14 | 78149486 | T | C |
| 14 | 78149563 | G | C | 14 | 79217473 | C | T | 14 | 80002042 | A | G |
| 14 | 80002096 | T | C | 14 | 80002632 | A | G | 14 | 80874274 | A | T |
| 14 | 81554617 | A | G | 14 | 81558640 | C | G | 14 | 81559221 | G | A |
| 14 | 81562808 | A | G | 14 | 81606422 | G | T | 14 | 81606503 | T | C |
| 14 | 81610869 | C | A | 14 | 81610942 | C | T | 14 | 82053802 | T | C |
| 14 | 82054300 | G | T | 14 | 82602590 | C | T | 14 | 82603182 | C | T |
| 14 | 86166491 | A | G | 14 | 88199938 | T | C | 14 | 91507280 | T | C |
| 14 | 94339436 | C | G | 14 | 94845040 | G | A | 14 | 95389462 | A | G |
| 14 | 95582544 | A | C | 14 | 95600006 | G | C | 14 | 95600072 | T | G |
| 14 | 96092282 | C | T | 14 | 96592735 | C | G | 14 | 97107413 | G | A |
| 14 | 98485583 | T | C | 14 | 98486120 | G | A | 14 | 98868717 | G | A |
| 14 | 100595278 | T | A | 14 | 100595880 | G | A | 14 | 101134984 | G | A |
| 14 | 103050106 | G | A | 14 | 103050397 | C | G | 14 | 103840494 | G | A |
| 14 | 104168701 | C | T | 14 | 104563874 | C | T | 14 | 104563932 | G | T |
| 14 | 104564283 | T | C | 14 | 104564345 | G | A | 14 | 104564410 | T | C |
| 14 | 104564412 | G | A | 14 | 104945518 | C | G | 14 | 104945694 | A | T |
| 14 | 105243435 | G | T | 14 | 105246872 | A | G | 14 | 106049449 | T | C |
| 14 | 106167913 | T | A | 14 | 106725276 | G | A | 14 | 106725315 | G | A |
| 14 | 106725329 | C | T | 14 | 106725339 | A | G | 14 | 106725346 | G | A |
| 14 | 106725515 | A | C | 14 | 106725525 | C | A | 14 | 106725526 | T | A |
| 14 | 106725543 | A | T | 14 | 107046720 | T | A | 14 | 107048702 | G | A |
| 14 | 107048749 | C | G | 14 | 107048929 | T | C | 14 | 107112165 | C | G |
| 14 | 107112665 | A | C | 14 | 107130943 | G | A | 14 | 107130971 | C | T |
| 14 | 107131063 | A | G | 14 | 107131164 | A | C | 14 | 107131246 | C | T |
| 14 | 107131290 | T | C | 14 | 107131300 | T | C | 14 | 107131301 | C | G |
| 14 | 107131349 | A | G | 14 | 107131357 | T | C | 14 | 107131410 | A | G |
| 15 | 20148695 | G | T | 15 | 20148878 | C | A | 15 | 20319405 | C | T |
| 15 | 20319448 | T | C | 15 | 20543982 | T | C | 15 | 20543988 | A | G |
| 15 | 20635860 | C | T | 15 | 20891413 | A | C | 15 | 20891478 | C | T |
| 15 | 20891489 | T | C | 15 | 20891547 | C | T | 15 | 20891566 | C | A |
| 15 | 20891566 | C | G | 15 | 20891651 | A | G | 15 | 20891867 | G | T |
| 15 | 20891893 | C | A | 15 | 20966938 | G | T | 15 | 20967029 | A | G |
| 15 | 21136727 | T | A | 15 | 21136951 | G | A | 15 | 21136968 | C | T |
| 15 | 21137321 | C | A | 15 | 21325716 | C | T | 15 | 21325857 | T | C |
| 15 | 21326120 | C | T | 15 | 21326788 | C | G | 15 | 21966878 | C | T |
| 15 | 21973528 | C | T | 15 | 21973529 | A | G | 15 | 21973572 | T | C |
| 15 | 21973587 | T | C | 15 | 21973588 | A | G | 15 | 21973592 | G | T |
| 15 | 21973797 | T | C | 15 | 21973842 | A | C | 15 | 21973843 | G | A |
| 15 | 22148127 | C | T | 15 | 22148775 | A | G | 15 | 22148845 | A | G |
| 15 | 22151298 | C | T | 15 | 22151704 | A | G | 15 | 22151876 | C | T |
| 15 | 22822510 | G | C | 15 | 22822568 | T | G | 15 | 22822751 | C | G |
| 15 | 22822755 | G | A | 15 | 22822854 | T | A | 15 | 22822955 | G | T |
| 15 | 22823001 | T | A | 15 | 22823078 | A | G | 15 | 22823113 | T | C |
| 15 | 22823116 | C | A | 15 | 22823137 | A | C | 15 | 22823185 | T | G |
| 15 | 22823255 | A | C | 15 | 22823692 | T | C | 15 | 22823783 | G | T |
| 15 | 22823838 | C | A | 15 | 22823855 | A | G | 15 | 22823949 | C | A |
| 15 | 22823963 | T | C | 15 | 22824021 | T | G | 15 | 22824030 | C | A |
| 15 | 22824086 | G | A | 15 | 22824376 | A | G | 15 | 22826375 | T | C |
| 15 | 22826634 | A | C | 15 | 22826679 | C | T | 15 | 22826764 | T | C |
| 15 | 22827002 | G | C | 15 | 22827233 | A | G | 15 | 22827239 | G | C |
| 15 | 22827251 | A | C | 15 | 22827293 | C | A | 15 | 22827319 | G | T |
| 15 | 22827322 | A | T | 15 | 22827437 | A | G | 15 | 22827816 | A | C |
| 15 | 22827928 | T | G | 15 | 22827930 | C | A | 15 | 22827939 | T | C |
| 15 | 22827984 | G | C | 15 | 22827985 | G | A | 15 | 22828037 | G | A |
| 15 | 22828041 | A | G | 15 | 22828139 | A | T | 15 | 22828145 | T | C |
| 15 | 22828147 | T | C | 15 | 22828275 | G | C | 15 | 22828390 | A | T |
| 15 | 22828465 | A | C | 15 | 22829674 | C | G | 15 | 22829687 | G | T |
| 15 | 22829695 | C | T | 15 | 22830185 | T | C | 15 | 22830340 | C | T |
| 15 | 22830453 | G | A | 15 | 22830504 | T | A | 15 | 22830510 | G | T |
| 15 | 22830571 | C | T | 15 | 22830577 | A | G | 15 | 22830592 | G | T |
| 15 | 22830847 | C | G | 15 | 22830891 | T | C | 15 | 22830899 | G | T |
| 15 | 22830903 | G | C | 15 | 22830990 | C | T | 15 | 22830999 | C | T |
| 15 | 22832977 | A | G | 15 | 23782545 | T | C | 15 | 24059444 | T | A |
| 15 | 24059493 | A | T | 15 | 24060123 | T | G | 15 | 24060127 | G | A |
| 15 | 24506371 | C | A | 15 | 24506572 | C | T | 15 | 24672192 | G | T |
| 15 | 24672267 | G | C | 15 | 24672440 | G | C | 15 | 24672460 | A | C |
| 15 | 24672460 | A | T | 15 | 24672504 | G | C | 15 | 24672554 | G | T |
| 15 | 24672804 | T | A | 15 | 24722807 | G | T | 15 | 24722823 | G | C |
| 15 | 24722829 | G | A | 15 | 25414653 | C | T | 15 | 26273690 | G | A |
| 15 | 26274261 | A | G | 15 | 26600681 | A | G | 15 | 26601246 | G | A |
| 15 | 26601264 | G | A | 15 | 29717391 | T | C | 15 | 31490709 | A | C |
| 15 | 31821275 | G | A | 15 | 32535526 | G | A | 15 | 34638030 | C | T |
| 15 | 34639823 | A | G | 15 | 34639826 | A | G | 15 | 34639879 | C | G |
| 15 | 34639904 | G | A | 15 | 35147136 | C | A | 15 | 35147592 | G | A |
| 15 | 35384908 | C | A | 15 | 37292823 | G | A | 15 | 37292836 | G | A |
| 15 | 38190654 | G | T | 15 | 38545725 | A | C | 15 | 38617282 | A | G |
| 15 | 38642971 | G | C | 15 | 38643009 | G | A | 15 | 38643031 | C | T |
| 15 | 39034864 | C | T | 15 | 39034914 | G | A | 15 | 40457042 | A | C |
| 15 | 40457635 | C | T | 15 | 40998305 | T | A | 15 | 41011311 | G | A |
| 15 | 41021280 | C | G | 15 | 42265586 | G | A | 15 | 43853068 | C | A |
| 15 | 43853127 | C | T | 15 | 43853145 | G | A | 15 | 43853167 | T | C |
| 15 | 43853196 | C | G | 15 | 43853216 | T | A | 15 | 45985719 | G | C |
| 15 | 47150907 | T | C | 15 | 49220457 | A | G | 15 | 49615149 | A | G |
| 15 | 51483996 | G | C | 15 | 53022863 | A | C | 15 | 53023345 | C | T |
| 15 | 53178383 | C | G | 15 | 54607264 | T | C | 15 | 55099758 | G | T |
| 15 | 59722664 | C | T | 15 | 59844728 | A | G | 15 | 59845138 | G | A |
| 15 | 62046728 | T | C | 15 | 66736741 | C | T | 15 | 66774559 | A | G |
| 15 | 67458930 | C | T | 15 | 67476735 | G | A | 15 | 67476756 | C | T |
| 15 | 67476768 | C | T | 15 | 67477450 | C | T | 15 | 68123700 | C | T |
| 15 | 69383465 | T | A | 15 | 69676000 | C | A | 15 | 70292710 | C | T |
| 15 | 70573650 | C | A | 15 | 71967893 | G | A | 15 | 74460734 | C | T |
| 15 | 74654146 | T | C | 15 | 76001037 | C | T | 15 | 76039262 | T | C |
| 15 | 78206482 | C | T | 15 | 78206514 | A | T | 15 | 78206928 | G | A |
| 15 | 78206970 | G | T | 15 | 78206973 | C | A | 15 | 78207027 | A | G |
| 15 | 79032355 | T | C | 15 | 79032469 | C | T | 15 | 79155412 | T | C |
| 15 | 79526411 | G | T | 15 | 80012735 | A | G | 15 | 81410106 | G | A |
| 15 | 82636963 | G | A | 15 | 82637357 | T | C | 15 | 83331487 | T | C |
| 15 | 83569832 | C | G | 15 | 84143244 | G | A | 15 | 84857013 | C | G |
| 15 | 84909385 | G | A | 15 | 84951508 | C | A | 15 | 84951578 | G | C |
| 15 | 85052414 | C | T | 15 | 85495400 | C | T | 15 | 85495950 | C | T |
| 15 | 85788238 | G | A | 15 | 85788607 | C | T | 15 | 85788735 | T | C |
| 15 | 86855520 | G | A | 15 | 88428702 | C | A | 15 | 88472170 | A | G |
| 15 | 88483594 | A | G | 15 | 88680100 | G | A | 15 | 88680987 | T | A |
| 15 | 88690951 | C | T | 15 | 88726405 | G | A | 15 | 88727751 | A | T |
| 15 | 89801677 | G | C | 15 | 89811482 | T | C | 15 | 89821732 | G | A |
| 15 | 89824258 | A | C | 15 | 89833165 | T | C | 15 | 89836793 | C | A |
| 15 | 89843950 | A | T | 15 | 89855998 | C | T | 15 | 89859377 | A | G |
| 15 | 90628777 | T | C | 15 | 90631247 | G | A | 15 | 90633416 | A | G |
| 15 | 90645265 | G | A | 15 | 91290366 | G | C | 15 | 91337208 | G | A |
| 15 | 91347135 | C | A | 15 | 91347755 | A | G | 15 | 91347784 | C | T |
| 15 | 91352774 | A | G | 15 | 91969961 | G | C | 15 | 91969970 | T | G |
| 15 | 91969976 | C | G | 15 | 92868324 | A | G | 15 | 94208914 | A | C |
| 15 | 95270619 | C | T | 15 | 95270626 | T | C | 15 | 95271164 | T | A |
| 15 | 95662895 | T | A | 15 | 96479346 | C | T | 15 | 96705724 | T | C |
| 15 | 97586395 | C | A | 15 | 98419797 | C | A | 15 | 98419885 | G | C |
| 15 | 99434357 | G | A | 15 | 99443139 | G | A | 15 | 99443161 | A | G |
| 15 | 99454245 | A | G | 15 | 99454962 | C | T | 15 | 99454994 | G | A |
| 15 | 99458902 | C | T | 15 | 99482903 | C | T | 15 | 99485931 | T | A |
| 15 | 99491540 | G | A | 15 | 99500064 | C | T | 15 | 100285424 | T | G |
| 15 | 100333593 | G | A | 15 | 100339833 | G | C | 15 | 100339897 | T | C |
| 15 | 100339938 | C | T | 15 | 100340316 | A | C | 15 | 100340375 | A | G |
| 15 | 100340413 | C | T | 15 | 100340484 | A | G | 15 | 101110096 | T | C |
| 15 | 102296008 | G | C | 15 | 102296140 | C | T | 15 | 102296231 | C | T |
| 15 | 102300354 | T | C | 15 | 102301640 | T | C | 15 | 102301662 | A | C |
| 15 | 102301669 | G | C | 15 | 102303340 | A | G | 15 | 102303423 | A | G |
| 15 | 102303495 | C | A | 15 | 102303519 | G | T | 15 | 102304191 | G | C |
| 15 | 102305129 | T | C | 15 | 102305169 | G | T | 15 | 102517868 | G | A |
| 16 | 326817 | C | A | 16 | 326826 | C | G | 16 | 900140 | A | G |
| 16 | 1872922 | C | G | 16 | 1873332 | G | A | 16 | 1873372 | C | T |
| 16 | 2107489 | C | T | 16 | 3020860 | C | G | 16 | 3799857 | T | C |
| 16 | 3828348 | A | G | 16 | 4060980 | T | C | 16 | 4985044 | C | T |
| 16 | 5139217 | C | G | 16 | 5143432 | C | G | 16 | 5143976 | C | G |
| 16 | 5145474 | C | T | 16 | 5145847 | G | A | 16 | 5145848 | A | G |
| 16 | 5145883 | A | G | 16 | 5146062 | C | G | 16 | 5166093 | A | G |
| 16 | 5338275 | A | G | 16 | 5338313 | C | G | 16 | 5527601 | T | A |
| 16 | 6553772 | G | A | 16 | 6554204 | A | C | 16 | 6554253 | C | A |
| 16 | 6554268 | C | T | 16 | 7495752 | C | T | 16 | 7496375 | G | T |
| 16 | 8603974 | A | G | 16 | 8603995 | G | C | 16 | 8604458 | A | G |
| 16 | 8604472 | C | T | 16 | 8604479 | A | C | 16 | 8604481 | C | T |
| 16 | 8604503 | A | G | 16 | 8604517 | G | A | 16 | 8604540 | A | G |
| 16 | 9928329 | C | T | 16 | 9943995 | A | G | 16 | 10273536 | G | T |
| 16 | 10632828 | A | G | 16 | 12019830 | G | A | 16 | 12019883 | A | C |
| 16 | 12019895 | A | G | 16 | 12297011 | C | G | 16 | 14781854 | T | A |
| 16 | 14781862 | T | A | 16 | 14782377 | C | A | 16 | 14821494 | C | A |
| 16 | 14864463 | G | A | 16 | 14864910 | G | A | 16 | 15047046 | C | G |
| 16 | 15452301 | C | T | 16 | 15460786 | T | C | 16 | 15867652 | T | G |
| 16 | 16789081 | C | T | 16 | 16789406 | C | T | 16 | 18450847 | C | T |
| 16 | 18450961 | G | A | 16 | 18543753 | C | T | 16 | 19479406 | C | T |
| 16 | 20575093 | G | A | 16 | 20575307 | G | A | 16 | 21844766 | G | A |
| 16 | 21844944 | A | G | 16 | 21844958 | G | C | 16 | 23016538 | G | A |
| 16 | 23632479 | C | T | 16 | 23634026 | A | G | 16 | 23637251 | G | C |
| 16 | 23698547 | G | T | 16 | 24888454 | C | T | 16 | 25668649 | C | T |
| 16 | 25668663 | A | C | 16 | 26131607 | A | G | 16 | 26132278 | G | T |
| 16 | 26798205 | G | T | 16 | 27947757 | G | A | 16 | 28785263 | T | C |
| 16 | 28871657 | C | A | 16 | 29059935 | G | A | 16 | 29060011 | T | A |
| 16 | 29065054 | C | T | 16 | 29314400 | C | T | 16 | 29344359 | C | T |
| 16 | 29344604 | C | T | 16 | 29344690 | C | T | 16 | 29398813 | G | A |
| 16 | 31771741 | T | C | 16 | 31771776 | A | G | 16 | 31771780 | A | G |
| 16 | 31771902 | A | G | 16 | 31771903 | A | C | 16 | 31771904 | C | T |
| 16 | 31771907 | A | G | 16 | 31771911 | C | A | 16 | 31771914 | C | T |
| 16 | 31771943 | G | T | 16 | 31771944 | C | A | 16 | 31771949 | G | A |
| 16 | 31771953 | A | G | 16 | 31772070 | A | G | 16 | 32823016 | C | A |
| 16 | 32823158 | G | A | 16 | 32823526 | C | A | 16 | 33070217 | T | C |
| 16 | 48996185 | C | A | 16 | 48996189 | C | A | 16 | 48996630 | C | G |
| 16 | 50809902 | C | G | 16 | 50816660 | C | T | 16 | 52671770 | C | T |
| 16 | 53391457 | G | C | 16 | 53564401 | G | C | 16 | 55173502 | T | G |
| 16 | 55692499 | G | A | 16 | 56046208 | A | T | 16 | 57045159 | T | C |
| 16 | 57720488 | G | A | 16 | 57720563 | T | C | 16 | 57721127 | G | T |
| 16 | 57721146 | G | A | 16 | 58690676 | G | C | 16 | 58691267 | A | G |
| 16 | 61461212 | A | G | 16 | 62213143 | C | T | 16 | 63217610 | G | C |
| 16 | 65540174 | C | T | 16 | 68771034 | C | A | 16 | 68771122 | A | C |
| 16 | 68771547 | G | C | 16 | 68820718 | A | G | 16 | 68821271 | G | C |
| 16 | 68847044 | C | T | 16 | 68847712 | G | A | 16 | 68849904 | A | G |
| 16 | 68861864 | G | A | 16 | 68863314 | T | C | 16 | 70009172 | G | C |
| 16 | 70224457 | C | G | 16 | 70224541 | T | C | 16 | 70235983 | T | C |
| 16 | 71105150 | A | G | 16 | 72007232 | G | A | 16 | 72763955 | G | T |
| 16 | 72991057 | G | T | 16 | 73141028 | G | A | 16 | 74422136 | T | C |
| 16 | 74422332 | A | G | 16 | 74427116 | G | A | 16 | 76418228 | C | T |
| 16 | 76907109 | C | A | 16 | 77694411 | T | G | 16 | 77694421 | T | A |
| 16 | 78082862 | C | T | 16 | 78082906 | G | A | 16 | 78761364 | A | G |
| 16 | 78761917 | G | C | 16 | 79161532 | A | C | 16 | 79704152 | T | C |
| 16 | 81636255 | A | G | 16 | 81636851 | G | A | 16 | 82150582 | A | G |
| 16 | 82527841 | C | T | 16 | 83459041 | C | T | 16 | 83691187 | G | A |
| 16 | 85437616 | T | C | 16 | 85437625 | T | C | 16 | 85437629 | G | A |
| 16 | 85437709 | G | A | 16 | 85437722 | T | C | 16 | 85437742 | T | C |
| 16 | 85437752 | A | T | 16 | 85437996 | A | T | 16 | 85438220 | G | A |
| 16 | 85438556 | T | A | 16 | 85438649 | A | G | 16 | 85438680 | C | T |
| 16 | 85438700 | C | T | 16 | 85441380 | C | G | 16 | 85441405 | G | C |
| 16 | 85441424 | T | C | 16 | 85441812 | A | C | 16 | 85442570 | T | C |
| 16 | 85442574 | A | G | 16 | 85443843 | T | C | 16 | 85443876 | G | A |
| 16 | 85443989 | T | G | 16 | 85444082 | G | C | 16 | 85444102 | C | A |
| 16 | 85444107 | C | T | 16 | 85444130 | T | C | 16 | 85444204 | C | T |
| 16 | 85444618 | A | G | 16 | 85444655 | A | C | 16 | 85444705 | T | C |
| 16 | 85444849 | A | C | 16 | 85445141 | C | T | 16 | 85445267 | C | G |
| 16 | 85445365 | C | T | 16 | 86005933 | C | G | 16 | 86005964 | T | G |
| 16 | 86005984 | G | A | 16 | 86006286 | C | T | 16 | 87178517 | A | G |
| 16 | 87179026 | T | C | 16 | 87179050 | G | T | 16 | 87179063 | G | A |
| 16 | 87179102 | G | A | 16 | 87179142 | G | A | 16 | 87179180 | C | A |
| 16 | 87648305 | C | T | 16 | 87648315 | T | G | 16 | 87648341 | G | A |
| 16 | 87926558 | G | A | 16 | 87926696 | G | C | 16 | 88073139 | C | T |
| 16 | 88073194 | C | T | 16 | 88073218 | T | C | 16 | 88073296 | A | G |
| 16 | 88310744 | C | T | 16 | 88310829 | T | C | 16 | 88310830 | G | A |
| 16 | 88310910 | A | G | 16 | 88310955 | A | G | 16 | 88310963 | C | G |
| 16 | 88311269 | A | G | 16 | 88311279 | A | T | 16 | 88311285 | A | T |
| 16 | 88311291 | G | C | 16 | 88311355 | T | C | 16 | 88312367 | C | T |
| 16 | 88312750 | C | T | 16 | 88312754 | A | G | 16 | 88312962 | C | T |
| 16 | 88312973 | C | T | 16 | 88313227 | C | T | 16 | 88313229 | C | T |
| 16 | 88313255 | A | G | 16 | 88313383 | A | G | 16 | 88313402 | T | C |
| 16 | 88313449 | T | C | 16 | 88313454 | T | C | 16 | 88313493 | A | G |
| 16 | 88313505 | A | G | 16 | 88313530 | G | T | 16 | 88313585 | A | G |
| 16 | 88313677 | C | T | 16 | 88313845 | C | T | 16 | 88794959 | C | A |
| 16 | 88795086 | T | G | 16 | 88795431 | A | T | 16 | 88795433 | C | T |
| 16 | 88795718 | C | T | 16 | 89299506 | A | T | 16 | 89299519 | G | C |
| 16 | 89299899 | A | G | 16 | 89756171 | G | A | 16 | 89756185 | A | T |
| 16 | 89806732 | G | A | 16 | 89807543 | G | A | 16 | 89808939 | T | C |
| 16 | 89809548 | C | A | 16 | 89809549 | A | G | 16 | 89811151 | A | G |
| 16 | 89811209 | C | T | 16 | 89811663 | T | C | 16 | 89811751 | C | G |
| 16 | 89814818 | T | G | 16 | 89815321 | C | T | 16 | 89815824 | G | A |
| 16 | 89816569 | C | A | 16 | 89818340 | G | C | 16 | 89818908 | C | T |
| 16 | 89828043 | T | A | 16 | 89839976 | T | C | 16 | 89842479 | T | C |
| 16 | 89849735 | G | A | 16 | 89849740 | C | T | 16 | 89851004 | A | G |
| 16 | 89851033 | C | T | 16 | 89998794 | G | A | 16 | 90233312 | C | T |
| 17 | 164435 | C | T | 17 | 164955 | C | A | 17 | 164967 | T | C |
| 17 | 164969 | C | G | 17 | 164980 | C | T | 17 | 164997 | T | G |
| 17 | 164999 | C | G | 17 | 165010 | T | C | 17 | 165025 | T | C |
| 17 | 165071 | C | T | 17 | 165078 | C | A | 17 | 165085 | C | T |
| 17 | 165090 | G | T | 17 | 165147 | T | C | 17 | 165201 | C | G |
| 17 | 165240 | C | G | 17 | 165247 | C | T | 17 | 165256 | C | A |
| 17 | 165295 | T | C | 17 | 165357 | C | A | 17 | 165375 | A | C |
| 17 | 165386 | T | C | 17 | 165409 | C | T | 17 | 165419 | A | C |
| 17 | 165501 | T | C | 17 | 165515 | T | C | 17 | 165562 | C | T |
| 17 | 165565 | G | C | 17 | 165576 | T | C | 17 | 165585 | G | T |
| 17 | 165615 | G | T | 17 | 165643 | C | A | 17 | 165694 | A | G |
| 17 | 165695 | C | T | 17 | 165712 | T | A | 17 | 165732 | T | C |
| 17 | 165736 | C | T | 17 | 165742 | G | C | 17 | 165966 | T | C |
| 17 | 165976 | C | T | 17 | 166081 | T | C | 17 | 166186 | C | A |
| 17 | 166346 | A | G | 17 | 1020183 | G | A | 17 | 1020394 | G | T |
| 17 | 1022697 | C | T | 17 | 1022705 | G | T | 17 | 1022734 | G | A |
| 17 | 1022796 | G | C | 17 | 1022860 | G | C | 17 | 1022923 | C | T |
| 17 | 2257864 | A | G | 17 | 2258637 | A | C | 17 | 3315757 | T | C |
| 17 | 3315904 | G | A | 17 | 3771365 | A | C | 17 | 3771379 | G | A |
| 17 | 3771380 | T | C | 17 | 3771381 | G | A | 17 | 3771384 | C | A |
| 17 | 5033768 | C | A | 17 | 5034469 | G | A | 17 | 5043998 | C | T |
| 17 | 5603793 | C | T | 17 | 6461283 | A | G | 17 | 7569698 | A | C |
| 17 | 7573229 | C | T | 17 | 7576348 | C | T | 17 | 8107979 | G | T |
| 17 | 8109598 | G | A | 17 | 8113186 | A | G | 17 | 9756002 | G | T |
| 17 | 9756057 | T | C | 17 | 9756171 | A | T | 17 | 9756198 | T | C |
| 17 | 10095727 | T | G | 17 | 11355538 | C | T | 17 | 11356161 | A | C |
| 17 | 11356178 | G | A | 17 | 11924471 | C | T | 17 | 12042934 | T | A |
| 17 | 12968947 | T | C | 17 | 12969339 | G | T | 17 | 15486857 | G | A |
| 17 | 15486909 | A | G | 17 | 16012494 | A | T | 17 | 16068096 | C | T |
| 17 | 16528031 | C | T | 17 | 17120668 | G | C | 17 | 17625022 | A | G |
| 17 | 17625107 | T | C | 17 | 17625185 | G | T | 17 | 18297214 | C | G |
| 17 | 18299435 | C | T | 17 | 18299631 | C | G | 17 | 18393867 | C | T |
| 17 | 19486126 | C | T | 17 | 19486133 | T | C | 17 | 19486148 | T | C |
| 17 | 19486537 | A | G | 17 | 19486585 | C | A | 17 | 19486592 | T | C |
| 17 | 20451455 | A | C | 17 | 20453878 | G | T | 17 | 21262861 | G | A |
| 17 | 21408378 | T | C | 17 | 21503001 | G | C | 17 | 21825993 | T | G |
| 17 | 21826038 | T | C | 17 | 21826256 | A | G | 17 | 21859750 | A | G |
| 17 | 21901935 | C | G | 17 | 21901936 | C | T | 17 | 21901945 | G | C |
| 17 | 22213908 | C | T | 17 | 25746737 | C | A | 17 | 25747067 | C | T |
| 17 | 25747244 | C | T | 17 | 25748176 | C | A | 17 | 25748270 | G | T |
| 17 | 25982703 | C | T | 17 | 25983153 | G | A | 17 | 26079849 | T | C |
| 17 | 26087967 | T | A | 17 | 26087978 | A | T | 17 | 26088432 | C | G |
| 17 | 27536646 | T | C | 17 | 28043613 | G | A | 17 | 28883741 | C | T |
| 17 | 29541339 | A | T | 17 | 29558082 | T | C | 17 | 29592550 | T | C |
| 17 | 29675878 | T | G | 17 | 29684553 | C | T | 17 | 30389991 | G | A |
| 17 | 32084335 | G | T | 17 | 32980299 | C | T | 17 | 32980342 | C | A |
| 17 | 33520071 | C | T | 17 | 33520663 | C | T | 17 | 34416275 | T | A |
| 17 | 34416946 | G | A | 17 | 34488320 | G | A | 17 | 34587938 | T | C |
| 17 | 34587942 | C | T | 17 | 34754758 | G | C | 17 | 34754836 | A | C |
| 17 | 36097775 | G | A | 17 | 36102035 | G | C | 17 | 36103872 | C | G |
| 17 | 36221809 | T | A | 17 | 36578907 | C | T | 17 | 36579428 | C | T |
| 17 | 37230242 | T | C | 17 | 37626963 | G | A | 17 | 37646589 | T | C |
| 17 | 37650569 | G | A | 17 | 38561366 | T | C | 17 | 38568249 | C | G |
| 17 | 39383335 | C | G | 17 | 39394674 | A | G | 17 | 39394729 | C | A |
| 17 | 39394755 | C | A | 17 | 39394787 | C | A | 17 | 39394790 | T | C |
| 17 | 39394807 | C | A | 17 | 39394885 | C | T | 17 | 39394911 | A | G |
| 17 | 39406673 | T | G | 17 | 39604697 | A | G | 17 | 40490539 | T | G |
| 17 | 40490559 | G | T | 17 | 40498273 | T | C | 17 | 40498944 | T | C |
| 17 | 41199913 | T | C | 17 | 41205772 | G | A | 17 | 41212169 | C | T |
| 17 | 41213626 | G | T | 17 | 41213660 | T | C | 17 | 41222723 | T | C |
| 17 | 41230228 | G | A | 17 | 41232698 | C | T | 17 | 41248164 | C | T |
| 17 | 41250923 | T | C | 17 | 41257134 | T | C | 17 | 41276348 | T | C |
| 17 | 41310495 | T | C | 17 | 41623212 | G | A | 17 | 43057954 | G | A |
| 17 | 43626858 | C | T | 17 | 45095080 | T | G | 17 | 45095092 | G | A |
| 17 | 45095678 | G | A | 17 | 46319546 | G | A | 17 | 47696070 | T | C |
| 17 | 47696933 | C | T | 17 | 47696939 | G | A | 17 | 47700315 | G | A |
| 17 | 48899222 | A | G | 17 | 49451714 | A | G | 17 | 49451742 | G | C |
| 17 | 49452209 | A | G | 17 | 50762097 | A | G | 17 | 50762653 | G | T |
| 17 | 51901084 | A | G | 17 | 51901643 | C | T | 17 | 51901703 | C | G |
| 17 | 52941883 | G | C | 17 | 53573902 | C | T | 17 | 53574374 | G | A |
| 17 | 55426865 | T | C | 17 | 56447952 | G | C | 17 | 56773834 | T | C |
| 17 | 58082363 | C | G | 17 | 58082421 | G | C | 17 | 58082431 | G | C |
| 17 | 58174022 | G | A | 17 | 59761706 | A | G | 17 | 59878416 | G | C |
| 17 | 59886310 | T | C | 17 | 59934879 | G | C | 17 | 60341389 | G | C |
| 17 | 60341507 | A | G | 17 | 60341507 | A | T | 17 | 60350783 | T | C |
| 17 | 60350874 | C | A | 17 | 61963236 | C | A | 17 | 61963241 | A | G |
| 17 | 61963478 | T | C | 17 | 61963622 | A | C | 17 | 61978159 | T | C |
| 17 | 61978548 | A | G | 17 | 61978622 | G | A | 17 | 62790430 | G | A |
| 17 | 63532175 | A | G | 17 | 63537898 | T | C | 17 | 64914494 | C | T |
| 17 | 65854602 | C | T | 17 | 65855104 | T | A | 17 | 66180139 | C | G |
| 17 | 66180681 | G | C | 17 | 66211771 | G | C | 17 | 66211840 | T | C |
| 17 | 66211969 | T | A | 17 | 66212122 | A | T | 17 | 66511299 | C | T |
| 17 | 66518663 | A | G | 17 | 66520427 | C | T | 17 | 66520838 | A | G |
| 17 | 66523772 | G | A | 17 | 66524755 | C | T | 17 | 67528431 | G | A |
| 17 | 69034000 | A | T | 17 | 71276883 | C | T | 17 | 71978657 | A | T |
| 17 | 71978747 | G | A | 17 | 71979236 | T | G | 17 | 71979237 | T | G |
| 17 | 72005968 | A | G | 17 | 72005970 | A | T | 17 | 72623641 | G | A |
| 17 | 74442413 | T | C | 17 | 74442440 | C | G | 17 | 74442942 | A | G |
| 17 | 75557061 | G | T | 17 | 75558122 | T | C | 17 | 75558750 | A | G |
| 17 | 75558782 | A | T | 17 | 75558791 | A | C | 17 | 75558802 | C | T |
| 17 | 75558814 | C | T | 17 | 75559019 | C | T | 17 | 76168857 | G | T |
| 17 | 76608673 | A | G | 17 | 78299319 | T | C | 17 | 78519169 | G | A |
| 17 | 78704156 | T | C | 17 | 78728314 | G | A | 17 | 78765080 | A | C |
| 17 | 78795868 | A | G | 17 | 78820023 | T | C | 17 | 78820212 | G | A |
| 17 | 78829531 | C | T | 17 | 78831983 | C | T | 17 | 78854056 | A | G |
| 17 | 78854487 | A | G | 17 | 78854496 | C | T | 17 | 78867906 | G | A |
| 17 | 78867951 | G | C | 17 | 78882953 | G | A | 17 | 78882972 | A | C |
| 17 | 78897056 | C | G | 17 | 78897732 | C | T | 17 | 78897733 | T | A |
| 17 | 78919285 | A | G | 17 | 78919324 | G | A | 17 | 78919349 | A | G |
| 17 | 78919387 | G | A | 17 | 78919837 | G | C | 17 | 78919859 | G | C |
| 17 | 78920807 | A | G | 17 | 78922952 | C | G | 17 | 78923007 | T | G |
| 17 | 78935071 | G | A | 17 | 78937165 | G | A | 17 | 78937925 | C | T |
| 17 | 78938011 | C | A | 17 | 80107354 | G | A | 17 | 80108414 | T | C |
| 17 | 80108478 | A | G | 17 | 80108696 | C | T | 17 | 80108822 | C | T |
| 17 | 80108842 | T | C | 17 | 81024201 | C | T | 17 | 81024331 | G | A |
| 17 | 81024640 | C | A | 17 | 81024656 | G | A | 17 | 81024660 | T | C |
| 17 | 81024673 | C | T | 17 | 81024674 | C | G | 17 | 81024677 | A | C |
| 17 | 81024682 | T | G | 17 | 81024691 | A | G | 17 | 81024707 | C | T |
| 17 | 81024731 | A | G | 17 | 81024741 | G | A | 17 | 81024752 | A | C |
| 18 | 13754 | A | T | 18 | 43439 | A | T | 18 | 43535 | A | C |
| 18 | 43538 | T | C | 18 | 86837 | T | C | 18 | 86857 | T | C |
| 18 | 657352 | T | C | 18 | 657443 | A | C | 18 | 657458 | A | G |
| 18 | 657474 | T | C | 18 | 658035 | A | G | 18 | 1963905 | A | G |
| 18 | 2913896 | C | G | 18 | 2913918 | G | A | 18 | 3406591 | A | G |
| 18 | 4106340 | T | G | 18 | 4106917 | G | A | 18 | 4932957 | C | T |
| 18 | 4933515 | C | T | 18 | 4933516 | G | A | 18 | 4933518 | T | G |
| 18 | 6462081 | A | C | 18 | 7382800 | A | G | 18 | 7382881 | C | A |
| 18 | 8408675 | G | A | 18 | 8789233 | G | A | 18 | 11610464 | G | T |
| 18 | 11610468 | A | G | 18 | 11610624 | G | C | 18 | 11610630 | G | A |
| 18 | 11610763 | A | G | 18 | 11618008 | G | A | 18 | 11622851 | T | C |
| 18 | 11623045 | T | C | 18 | 11623100 | C | T | 18 | 11644581 | G | A |
| 18 | 11644667 | G | A | 18 | 11644679 | G | T | 18 | 11644723 | T | C |
| 18 | 11644746 | G | A | 18 | 12101808 | G | A | 18 | 12898367 | A | G |
| 18 | 13369940 | C | T | 18 | 13370544 | G | A | 18 | 13673746 | C | T |
| 18 | 13674057 | A | G | 18 | 13674212 | C | A | 18 | 13674777 | T | C |
| 18 | 13675072 | A | G | 18 | 13675437 | T | C | 18 | 13675449 | A | T |
| 18 | 13675457 | G | C | 18 | 13675494 | C | T | 18 | 13675799 | A | T |
| 18 | 14341320 | T | C | 18 | 14341763 | T | C | 18 | 14436696 | C | A |
| 18 | 14524300 | T | C | 18 | 14594781 | C | A | 18 | 14595105 | A | T |
| 18 | 15165336 | A | G | 18 | 15165362 | G | T | 18 | 15206879 | C | T |
| 18 | 19761756 | C | A | 18 | 19995753 | T | C | 18 | 21252592 | A | G |
| 18 | 21978687 | A | G | 18 | 22598696 | C | T | 18 | 23785015 | C | A |
| 18 | 23785095 | C | T | 18 | 23785115 | C | G | 18 | 23883026 | G | C |
| 18 | 23883058 | T | C | 18 | 24885804 | T | C | 18 | 26757855 | T | C |
| 18 | 27273833 | C | T | 18 | 27587457 | A | G | 18 | 28115656 | T | C |
| 18 | 28115679 | C | G | 18 | 28115710 | C | T | 18 | 28115783 | G | A |
| 18 | 28115955 | G | T | 18 | 28116070 | G | A | 18 | 29573525 | T | C |
| 18 | 31662955 | C | T | 18 | 33187675 | G | C | 18 | 33187676 | T | G |
| 18 | 33187692 | A | G | 18 | 34886895 | T | G | 18 | 34887449 | G | A |
| 18 | 34887505 | A | G | 18 | 35451056 | C | T | 18 | 36001103 | G | C |
| 18 | 38530356 | A | G | 18 | 39614110 | G | A | 18 | 39620376 | T | A |
| 18 | 39620932 | G | A | 18 | 39620950 | C | T | 18 | 40049623 | G | A |
| 18 | 40050171 | T | C | 18 | 43006049 | C | T | 18 | 44223365 | G | T |
| 18 | 44909077 | A | G | 18 | 44909081 | G | A | 18 | 45368587 | G | A |
| 18 | 45371445 | C | T | 18 | 45371509 | C | T | 18 | 45377882 | G | C |
| 18 | 45395111 | G | C | 18 | 45651984 | C | G | 18 | 46453156 | A | T |
| 18 | 46453754 | C | T | 18 | 47701550 | T | C | 18 | 48581601 | C | A |
| 18 | 48603403 | T | C | 18 | 49958403 | G | C | 18 | 49959036 | G | T |
| 18 | 51674135 | A | G | 18 | 54762024 | G | A | 18 | 55396242 | G | A |
| 18 | 55782772 | T | C | 18 | 56757385 | T | C | 18 | 56757399 | A | G |
| 18 | 56757422 | T | C | 18 | 59992577 | T | C | 18 | 59992591 | C | G |
| 18 | 60015207 | G | C | 18 | 60015750 | G | A | 18 | 60026926 | C | T |
| 18 | 60026931 | G | C | 18 | 60027005 | T | A | 18 | 60028762 | G | A |
| 18 | 60029238 | G | C | 18 | 60029281 | A | G | 18 | 60029292 | G | A |
| 18 | 60034276 | C | T | 18 | 60984964 | C | A | 18 | 61654297 | A | G |
| 18 | 63196837 | G | A | 18 | 65928322 | A | T | 18 | 66103401 | C | T |
| 18 | 67182508 | G | T | 18 | 67182512 | G | A | 18 | 67183000 | A | G |
| 18 | 67183043 | T | C | 18 | 68086329 | G | A | 18 | 68086404 | A | G |
| 18 | 68086457 | T | G | 18 | 68086460 | C | T | 18 | 68700027 | G | A |
| 18 | 69291422 | G | A | 18 | 70066366 | G | A | 18 | 70592333 | A | G |
| 18 | 70592333 | A | T | 18 | 70592352 | A | G | 18 | 70592858 | T | C |
| 18 | 71467330 | G | C | 18 | 71640426 | G | A | 18 | 72226809 | C | A |
| 18 | 72226857 | T | C | 18 | 73300822 | T | C | 18 | 74757060 | T | G |
| 18 | 74757768 | A | G | 18 | 76394226 | C | G | 18 | 76798551 | G | A |
| 18 | 76798812 | C | T | 18 | 76798870 | A | T | 18 | 76799194 | T | C |
| 18 | 76799227 | A | G | 18 | 76799231 | C | T | 18 | 76799232 | T | G |
| 18 | 76799233 | G | C | 18 | 76799233 | G | T | 18 | 76799237 | G | A |
| 18 | 76799276 | C | T | 18 | 76799300 | G | A | 18 | 76799500 | C | T |
| 18 | 77378236 | C | A | 18 | 77378238 | C | G | 18 | 77378242 | G | A |
| 18 | 77378897 | C | T | 18 | 77378974 | C | T | 18 | 77379017 | C | T |
| 18 | 77379036 | C | A | 18 | 77379117 | T | C | 18 | 77379177 | T | C |
| 18 | 77379214 | C | T | 18 | 77379461 | T | C | 18 | 77379462 | C | A |
| 18 | 77379462 | C | G | 18 | 77379473 | T | C | 18 | 77379566 | T | C |
| 18 | 77379577 | C | T | 18 | 77379578 | C | G | 18 | 77379589 | T | C |
| 18 | 77379649 | T | C | 18 | 77379742 | T | C | 18 | 77379754 | C | G |
| 18 | 77379765 | C | G | 18 | 77379814 | C | G | 18 | 77379845 | C | T |
| 18 | 77379922 | T | C | 18 | 77379994 | C | G | 18 | 77380025 | T | C |
| 18 | 77380053 | G | C | 18 | 77380053 | G | T | 18 | 77380442 | C | T |
| 18 | 77380579 | C | T | 18 | 77380810 | A | G | 19 | 1218219 | C | T |
| 19 | 1218771 | G | A | 19 | 1219129 | G | A | 19 | 1219702 | G | A |
| 19 | 1222316 | C | T | 19 | 1226901 | G | T | 19 | 2180515 | G | A |
| 19 | 2185651 | C | T | 19 | 2186237 | G | A | 19 | 2190774 | T | A |
| 19 | 2194896 | T | C | 19 | 2199576 | C | T | 19 | 2207041 | A | G |
| 19 | 2207452 | G | A | 19 | 2208859 | T | C | 19 | 2210269 | C | T |
| 19 | 2221792 | T | C | 19 | 2833802 | A | G | 19 | 2833864 | G | A |
| 19 | 3110585 | A | G | 19 | 3113770 | T | C | 19 | 3113780 | C | T |
| 19 | 3113839 | A | G | 19 | 3115402 | C | T | 19 | 3118638 | C | T |
| 19 | 3120739 | T | G | 19 | 3148819 | G | T | 19 | 4094110 | G | T |
| 19 | 4094775 | G | A | 19 | 4098947 | G | A | 19 | 4102132 | A | C |
| 19 | 5524721 | C | T | 19 | 6230241 | G | A | 19 | 6495064 | C | G |
| 19 | 6495079 | G | C | 19 | 6502137 | C | A | 19 | 6990495 | C | T |
| 19 | 6991043 | A | G | 19 | 7032902 | G | A | 19 | 7033122 | T | C |
| 19 | 7037737 | C | A | 19 | 7051588 | G | A | 19 | 7051637 | T | G |
| 19 | 7051643 | T | C | 19 | 7051909 | G | C | 19 | 7051942 | C | A |
| 19 | 7056116 | A | G | 19 | 7514901 | A | G | 19 | 7514901 | A | T |
| 19 | 7515546 | G | T | 19 | 7515550 | A | G | 19 | 7515569 | G | A |
| 19 | 7515658 | A | G | 19 | 7515659 | C | A | 19 | 7515661 | G | A |
| 19 | 7515716 | G | T | 19 | 7515742 | G | A | 19 | 7515750 | G | T |
| 19 | 7515772 | C | T | 19 | 7515788 | C | A | 19 | 7516210 | C | T |
| 19 | 7516243 | A | G | 19 | 7816166 | G | A | 19 | 7816206 | G | T |
| 19 | 7816216 | C | A | 19 | 8202601 | C | T | 19 | 8567500 | G | A |
| 19 | 8567548 | A | G | 19 | 10097667 | C | T | 19 | 10597746 | T | A |
| 19 | 10600753 | A | C | 19 | 11113475 | C | A | 19 | 11129429 | C | G |
| 19 | 11134090 | A | G | 19 | 11136857 | C | T | 19 | 11138226 | T | C |
| 19 | 11143811 | C | G | 19 | 11168798 | G | A | 19 | 11172299 | G | A |
| 19 | 12491498 | C | T | 19 | 12491779 | T | C | 19 | 13481531 | C | T |
| 19 | 13699891 | C | A | 19 | 13699931 | C | T | 19 | 14218491 | A | G |
| 19 | 14404337 | G | C | 19 | 15807884 | A | G | 19 | 15871215 | C | G |
| 19 | 15871285 | T | A | 19 | 15871360 | G | C | 19 | 15871498 | A | G |
| 19 | 16046478 | G | T | 19 | 17700647 | G | A | 19 | 17937300 | T | A |
| 19 | 17942927 | C | T | 19 | 17950674 | C | T | 19 | 18272190 | A | C |
| 19 | 18974059 | G | C | 19 | 19261179 | C | A | 19 | 19261833 | A | G |
| 19 | 19261851 | T | C | 19 | 19664077 | C | T | 19 | 20024912 | T | A |
| 19 | 20024959 | G | A | 19 | 20050037 | G | A | 19 | 20162695 | A | C |
| 19 | 20162824 | G | C | 19 | 20632050 | T | C | 19 | 20632172 | T | G |
| 19 | 20632400 | T | C | 19 | 20832195 | C | T | 19 | 20857905 | T | C |
| 19 | 21057335 | G | A | 19 | 21148018 | G | A | 19 | 21148051 | C | G |
| 19 | 21148086 | G | T | 19 | 21749035 | T | C | 19 | 21749580 | C | T |
| 19 | 22155757 | G | A | 19 | 22642684 | T | C | 19 | 23477341 | C | G |
| 19 | 23477350 | C | G | 19 | 28330492 | T | C | 19 | 28330539 | A | G |
| 19 | 29331628 | C | A | 19 | 29331703 | C | G | 19 | 29518038 | G | C |
| 19 | 30303352 | T | C | 19 | 30311372 | C | T | 19 | 30314371 | G | A |
| 19 | 34080511 | G | C | 19 | 34080579 | C | G | 19 | 34882578 | G | A |
| 19 | 36213072 | A | G | 19 | 36217957 | A | G | 19 | 36219420 | G | A |
| 19 | 36219525 | A | G | 19 | 36222017 | A | G | 19 | 36222023 | T | C |
| 19 | 36224973 | T | C | 19 | 36807142 | A | G | 19 | 36807249 | A | T |
| 19 | 36807297 | T | G | 19 | 36807306 | G | A | 19 | 36807345 | G | A |
| 19 | 36807347 | G | A | 19 | 37147049 | A | G | 19 | 37147639 | C | T |
| 19 | 37752858 | C | T | 19 | 37752861 | T | C | 19 | 37752862 | G | A |
| 19 | 37753071 | T | C | 19 | 37753073 | T | C | 19 | 37753239 | G | A |
| 19 | 38391602 | C | A | 19 | 38932157 | C | T | 19 | 39261999 | C | T |
| 19 | 39990995 | G | A | 19 | 39991023 | G | A | 19 | 39991614 | A | G |
| 19 | 39994023 | A | G | 19 | 39994513 | G | A | 19 | 39997472 | G | A |
| 19 | 40739513 | A | G | 19 | 40743523 | C | T | 19 | 40770860 | G | A |
| 19 | 41349550 | G | C | 19 | 41349640 | G | C | 19 | 41349978 | A | G |
| 19 | 41353107 | T | C | 19 | 41353849 | C | T | 19 | 41381310 | A | G |
| 19 | 41381334 | G | A | 19 | 41382180 | G | C | 19 | 41384067 | T | C |
| 19 | 41384084 | T | C | 19 | 41384102 | G | T | 19 | 41384385 | T | G |
| 19 | 41384386 | C | G | 19 | 41385734 | C | T | 19 | 41386527 | G | A |
| 19 | 41386544 | C | G | 19 | 41386547 | G | C | 19 | 41386584 | G | T |
| 19 | 41386622 | C | T | 19 | 41387241 | G | A | 19 | 41414363 | A | T |
| 19 | 41415087 | G | A | 19 | 41415112 | T | A | 19 | 41415818 | G | A |
| 19 | 41416072 | G | C | 19 | 41416143 | C | T | 19 | 41416260 | T | G |
| 19 | 41416263 | C | A | 19 | 41416264 | A | G | 19 | 41512415 | T | C |
| 19 | 41515018 | G | A | 19 | 41529956 | A | G | 19 | 41600800 | C | T |
| 19 | 41600808 | C | T | 19 | 41736661 | G | A | 19 | 41744831 | C | T |
| 19 | 41745930 | C | T | 19 | 41754217 | G | T | 19 | 41765229 | T | C |
| 19 | 43359392 | G | A | 19 | 43383004 | G | C | 19 | 43421318 | C | T |
| 19 | 43440900 | T | C | 19 | 43586183 | T | C | 19 | 43586267 | C | T |
| 19 | 43586408 | A | G | 19 | 43690192 | T | A | 19 | 43772788 | C | G |
| 19 | 43773287 | G | C | 19 | 43773303 | T | C | 19 | 43860030 | G | A |
| 19 | 43860048 | G | T | 19 | 43860053 | G | A | 19 | 43860076 | C | T |
| 19 | 43860192 | T | G | 19 | 43882832 | A | C | 19 | 43882836 | C | T |
| 19 | 43882895 | A | C | 19 | 43883013 | C | A | 19 | 43883124 | C | T |
| 19 | 43883126 | G | A | 19 | 44055898 | C | T | 19 | 44056052 | A | G |
| 19 | 44057227 | T | C | 19 | 44057276 | G | A | 19 | 44905282 | C | G |
| 19 | 44905683 | C | T | 19 | 44905779 | C | T | 19 | 45862449 | A | G |
| 19 | 45868649 | C | G | 19 | 45871606 | T | C | 19 | 45916587 | T | C |
| 19 | 45924038 | G | A | 19 | 46375779 | C | T | 19 | 46406242 | A | G |
| 19 | 46721467 | G | A | 19 | 48764695 | G | A | 19 | 48764721 | T | C |
| 19 | 48765117 | G | A | 19 | 49457938 | A | G | 19 | 49458570 | T | C |
| 19 | 49459876 | T | A | 19 | 50265230 | C | T | 19 | 50901893 | G | A |
| 19 | 50903004 | A | G | 19 | 50909107 | C | A | 19 | 50920959 | G | T |
| 19 | 51315061 | T | C | 19 | 51315132 | T | C | 19 | 51315175 | T | C |
| 19 | 51315251 | C | T | 19 | 51646187 | C | A | 19 | 52249947 | C | G |
| 19 | 52249959 | G | T | 19 | 52704961 | T | C | 19 | 52709064 | G | T |
| 19 | 52714353 | T | A | 19 | 52715716 | G | A | 19 | 52718799 | C | T |
| 19 | 52718816 | C | T | 19 | 52718884 | T | A | 19 | 52720262 | A | G |
| 19 | 52723930 | C | T | 19 | 52724027 | C | A | 19 | 53927121 | A | C |
| 19 | 53927131 | C | A | 19 | 53927136 | A | G | 19 | 54007749 | G | C |
| 19 | 54208058 | T | C | 19 | 54267981 | C | T | 19 | 54860836 | G | A |
| 19 | 54860845 | A | G | 19 | 54860961 | G | A | 19 | 54860964 | A | C |
| 19 | 54860970 | T | G | 19 | 54861346 | G | A | 19 | 54861759 | G | T |
| 19 | 55271654 | C | G | 19 | 55333096 | T | C | 19 | 55333246 | C | T |
| 19 | 55350666 | C | A | 19 | 55350713 | G | C | 19 | 55350754 | A | G |
| 19 | 55350948 | A | T | 19 | 55350979 | A | G | 19 | 55351022 | A | C |
| 19 | 55543973 | C | T | 19 | 55544430 | A | G | 19 | 55544687 | G | A |
| 19 | 55544750 | A | G | 19 | 55545139 | A | G | 19 | 55545191 | G | T |
| 19 | 56361176 | A | G | 19 | 56361263 | G | A | 19 | 56361279 | A | G |
| 19 | 56361479 | A | G | 19 | 56361733 | T | G | 19 | 56361758 | T | A |
| 19 | 56361933 | G | T | 19 | 57866239 | C | A | 19 | 58464742 | G | C |
| 19 | 59051326 | T | C | 19 | 59051337 | G | C | 20 | 402053 | G | A |
| 20 | 402440 | A | G | 20 | 928228 | T | C | 20 | 1496279 | A | G |
| 20 | 2367161 | C | T | 20 | 2367209 | T | C | 20 | 2545011 | T | C |
| 20 | 4040209 | G | T | 20 | 4040258 | T | C | 20 | 4040763 | T | C |
| 20 | 4040828 | T | C | 20 | 4040923 | T | G | 20 | 4040961 | C | G |
| 20 | 4155648 | C | T | 20 | 4155668 | C | T | 20 | 4776659 | G | A |
| 20 | 5889649 | T | C | 20 | 6922046 | T | A | 20 | 8475010 | A | G |
| 20 | 9318946 | A | G | 20 | 9353445 | C | T | 20 | 9365303 | T | C |
| 20 | 9371499 | G | A | 20 | 9374613 | G | T | 20 | 9440715 | C | G |
| 20 | 9448947 | G | A | 20 | 9449000 | T | G | 20 | 9457170 | C | T |
| 20 | 9459927 | A | G | 20 | 10882821 | C | T | 20 | 11821257 | G | A |
| 20 | 12626712 | G | T | 20 | 12626745 | A | G | 20 | 14706879 | T | A |
| 20 | 15313903 | T | A | 20 | 15314383 | G | A | 20 | 16239362 | C | T |
| 20 | 16239376 | C | T | 20 | 16239426 | C | T | 20 | 18190882 | C | T |
| 20 | 18191172 | T | C | 20 | 18191291 | G | T | 20 | 18466346 | G | A |
| 20 | 20336724 | C | T | 20 | 20336950 | G | T | 20 | 20337707 | A | G |
| 20 | 20337722 | A | G | 20 | 20337869 | G | T | 20 | 20338243 | A | G |
| 20 | 20338247 | T | A | 20 | 20338251 | A | G | 20 | 20338825 | A | G |
| 20 | 20338971 | C | G | 20 | 20817609 | G | C | 20 | 21142523 | C | G |
| 20 | 21885619 | A | C | 20 | 22297047 | A | G | 20 | 22563571 | G | A |
| 20 | 22892171 | G | T | 20 | 23456589 | T | G | 20 | 23456962 | A | T |
| 20 | 23456975 | C | G | 20 | 23457003 | C | G | 20 | 23757966 | G | C |
| 20 | 23758038 | C | A | 20 | 23840240 | T | C | 20 | 23840242 | T | C |
| 20 | 23975455 | C | T | 20 | 24649705 | C | T | 20 | 24650360 | G | A |
| 20 | 25845523 | C | T | 20 | 25845579 | C | A | 20 | 25845591 | C | A |
| 20 | 25845594 | G | T | 20 | 25943298 | G | A | 20 | 26073480 | T | C |
| 20 | 29517367 | G | A | 20 | 29517535 | C | T | 20 | 29520369 | G | A |
| 20 | 29520383 | A | G | 20 | 29551711 | G | T | 20 | 31015643 | T | C |
| 20 | 31019725 | T | C | 20 | 31938046 | C | T | 20 | 33581689 | T | C |
| 20 | 33582279 | C | A | 20 | 34101631 | C | A | 20 | 34854605 | T | C |
| 20 | 36012348 | C | A | 20 | 36022154 | T | C | 20 | 36030592 | G | T |
| 20 | 36030622 | T | C | 20 | 36838422 | T | C | 20 | 37900688 | C | G |
| 20 | 37900691 | C | T | 20 | 39690342 | T | C | 20 | 39710038 | T | G |
| 20 | 41426178 | C | T | 20 | 41677471 | G | C | 20 | 42196825 | T | C |
| 20 | 42733847 | A | G | 20 | 43334843 | G | A | 20 | 43962124 | C | T |
| 20 | 43964620 | C | T | 20 | 45659653 | A | G | 20 | 46456298 | C | T |
| 20 | 46456399 | C | A | 20 | 46749867 | G | A | 20 | 47128176 | G | A |
| 20 | 47201278 | C | G | 20 | 47201306 | C | T | 20 | 47833636 | T | C |
| 20 | 47833697 | T | C | 20 | 48574438 | G | A | 20 | 48574454 | G | A |
| 20 | 50477441 | C | T | 20 | 50677624 | T | A | 20 | 50677714 | G | T |
| 20 | 53503158 | C | T | 20 | 53503198 | C | A | 20 | 54066409 | G | A |
| 20 | 54961073 | A | G | 20 | 55462729 | T | C | 20 | 55462744 | G | A |
| 20 | 55463137 | C | T | 20 | 55991479 | G | A | 20 | 56169863 | C | A |
| 20 | 56994740 | A | G | 20 | 57479133 | A | G | 20 | 57483948 | G | A |
| 20 | 58057058 | C | A | 20 | 58057077 | T | G | 20 | 58057118 | C | A |
| 20 | 58057119 | T | A | 20 | 59495071 | G | A | 20 | 59848508 | G | A |
| 20 | 59848515 | G | T | 20 | 59848519 | A | G | 20 | 59848582 | T | C |
| 20 | 59848585 | A | G | 20 | 60495224 | G | A | 20 | 60495285 | G | A |
| 21 | 9483659 | T | C | 21 | 9910262 | A | C | 21 | 9954440 | C | A |
| 21 | 9954672 | C | A | 21 | 9954692 | C | G | 21 | 9954700 | T | A |
| 21 | 9954700 | T | C | 21 | 9954718 | C | G | 21 | 9954726 | T | C |
| 21 | 9954750 | C | A | 21 | 9954822 | C | T | 21 | 9954874 | G | T |
| 21 | 9954972 | T | G | 21 | 9954978 | G | T | 21 | 9954994 | G | A |
| 21 | 10029331 | A | T | 21 | 14457818 | G | A | 21 | 14513159 | G | A |
| 21 | 14524601 | A | T | 21 | 14900799 | C | T | 21 | 14900897 | A | G |
| 21 | 14900901 | T | C | 21 | 14936098 | T | G | 21 | 15090065 | G | A |
| 21 | 15090071 | T | C | 21 | 15090282 | T | G | 21 | 15090308 | A | G |
| 21 | 15090315 | G | A | 21 | 15090335 | C | T | 21 | 15090336 | G | A |
| 21 | 15090391 | A | G | 21 | 15216110 | T | C | 21 | 15216154 | G | A |
| 21 | 15216190 | T | A | 21 | 15219309 | G | A | 21 | 15219323 | T | C |
| 21 | 15373568 | G | A | 21 | 15475013 | C | T | 21 | 15475149 | C | G |
| 21 | 15475439 | G | A | 21 | 15475515 | T | C | 21 | 16599891 | C | G |
| 21 | 16599895 | T | C | 21 | 16600408 | G | A | 21 | 18236198 | T | A |
| 21 | 19322853 | T | A | 21 | 19322854 | T | C | 21 | 19799542 | C | T |
| 21 | 20144540 | G | C | 21 | 20230102 | C | T | 21 | 20230168 | G | A |
| 21 | 20230169 | T | C | 21 | 20230217 | A | C | 21 | 20230294 | C | A |
| 21 | 20230372 | T | C | 21 | 20230390 | G | A | 21 | 21201593 | G | T |
| 21 | 21201603 | A | G | 21 | 21561669 | C | G | 21 | 21561683 | A | G |
| 21 | 21562354 | A | T | 21 | 22519731 | A | T | 21 | 23687317 | A | G |
| 21 | 24465037 | A | T | 21 | 24563993 | G | T | 21 | 25330218 | A | G |
| 21 | 26832609 | G | T | 21 | 26833183 | C | T | 21 | 26833198 | G | A |
| 21 | 28389497 | A | C | 21 | 30115032 | C | T | 21 | 30115170 | T | G |
| 21 | 30115571 | C | T | 21 | 30115579 | T | A | 21 | 30115629 | C | G |
| 21 | 30115651 | A | G | 21 | 30115655 | G | T | 21 | 30506189 | A | G |
| 21 | 32114077 | A | T | 21 | 32965156 | T | G | 21 | 33471163 | C | T |
| 21 | 33805478 | A | G | 21 | 33805831 | T | C | 21 | 34361496 | G | A |
| 21 | 34776152 | T | G | 21 | 34787638 | C | A | 21 | 34793588 | A | G |
| 21 | 34798982 | G | A | 21 | 35602677 | T | C | 21 | 36232156 | C | A |
| 21 | 36252595 | G | A | 21 | 36252622 | T | C | 21 | 36253194 | T | A |
| 21 | 36261705 | G | A | 21 | 36357463 | C | T | 21 | 36358229 | G | A |
| 21 | 36420874 | C | T | 21 | 36508295 | A | C | 21 | 36972392 | A | G |
| 21 | 41506058 | A | G | 21 | 41506612 | C | T | 21 | 42861181 | G | C |
| 21 | 43528820 | A | G | 21 | 43528822 | C | T | 21 | 43528949 | C | T |
| 21 | 43528970 | C | A | 21 | 43528991 | T | C | 21 | 43529061 | T | G |
| 21 | 43529064 | C | T | 21 | 43529078 | A | G | 21 | 43529162 | C | T |
| 21 | 43529185 | C | A | 21 | 43529216 | G | C | 21 | 43529224 | A | G |
| 21 | 43529228 | C | A | 21 | 45615963 | T | G | 21 | 45616049 | G | A |
| 21 | 46338651 | C | T | 21 | 46339140 | C | T | 21 | 47419697 | C | T |
| 21 | 47419747 | A | G | 21 | 47419770 | G | A | 21 | 47419780 | G | A |
| 21 | 47419794 | T | C | 21 | 47419804 | C | T | 21 | 47419817 | A | G |
| 21 | 47419832 | G | A | 21 | 47419835 | A | G | 21 | 47419856 | G | A |
| 21 | 47419859 | G | A | 21 | 47419859 | G | C | 21 | 47419860 | T | C |
| 21 | 47419872 | G | A | 21 | 47419875 | A | G | 21 | 47419876 | G | A |
| 21 | 47420016 | G | A | 21 | 48021967 | A | T | 22 | 16366289 | C | T |
| 22 | 16366304 | G | A | 22 | 16866125 | G | T | 22 | 16867451 | T | C |
| 22 | 16867494 | T | C | 22 | 16867535 | C | T | 22 | 16867555 | G | A |
| 22 | 16867625 | G | A | 22 | 16867671 | T | G | 22 | 16867807 | G | C |
| 22 | 17000863 | G | A | 22 | 17001006 | G | A | 22 | 17001044 | G | A |
| 22 | 17001074 | T | C | 22 | 17001080 | C | T | 22 | 17001124 | A | G |
| 22 | 17001134 | C | T | 22 | 17001218 | G | C | 22 | 17023726 | T | C |
| 22 | 17349297 | C | G | 22 | 17391914 | A | G | 22 | 17513526 | A | C |
| 22 | 17514026 | G | A | 22 | 17514060 | C | T | 22 | 18465978 | C | T |
| 22 | 18845285 | A | T | 22 | 18845397 | T | C | 22 | 18863659 | C | T |
| 22 | 19486391 | C | T | 22 | 19621254 | A | G | 22 | 20361753 | C | T |
| 22 | 20361768 | A | C | 22 | 21050028 | A | G | 22 | 21050319 | G | A |
| 22 | 21051859 | G | A | 22 | 21051983 | C | A | 22 | 21051987 | T | C |
| 22 | 21051994 | C | A | 22 | 21052014 | A | G | 22 | 21052189 | A | G |
| 22 | 21052193 | T | C | 22 | 21052265 | G | C | 22 | 21052303 | C | T |
| 22 | 21287911 | A | G | 22 | 21303690 | G | T | 22 | 21303711 | G | A |
| 22 | 21304408 | A | G | 22 | 21304463 | T | C | 22 | 21339915 | T | G |
| 22 | 21342169 | G | C | 22 | 21342811 | C | T | 22 | 21343391 | G | C |
| 22 | 21344287 | C | T | 22 | 21344316 | C | T | 22 | 21347410 | G | T |
| 22 | 21347505 | C | T | 22 | 21349548 | A | G | 22 | 21349579 | C | T |
| 22 | 21349676 | T | A | 22 | 21349677 | C | T | 22 | 21351819 | G | A |
| 22 | 21470373 | C | T | 22 | 21644653 | G | A | 22 | 21644684 | T | A |
| 22 | 21644783 | C | T | 22 | 21647786 | T | C | 22 | 21648685 | G | C |
| 22 | 22401267 | G | A | 22 | 22612065 | C | T | 22 | 22625680 | A | C |
| 22 | 22978886 | T | C | 22 | 22978887 | G | A | 22 | 22978887 | G | T |
| 22 | 22980711 | C | G | 22 | 23240831 | T | C | 22 | 23246298 | C | A |
| 22 | 23246323 | C | T | 22 | 23657735 | G | A | 22 | 23659783 | C | G |
| 22 | 23659864 | C | T | 22 | 24129129 | G | T | 22 | 24134394 | T | C |
| 22 | 24136214 | T | C | 22 | 24136252 | C | T | 22 | 24145923 | G | A |
| 22 | 24158697 | G | C | 22 | 24158709 | A | G | 22 | 24159437 | G | T |
| 22 | 24159452 | A | G | 22 | 24167166 | G | A | 22 | 24167185 | C | T |
| 22 | 24167906 | T | C | 22 | 24376322 | G | A | 22 | 24377034 | A | G |
| 22 | 24383954 | C | T | 22 | 24654050 | T | C | 22 | 24655566 | A | G |
| 22 | 24659077 | A | G | 22 | 24659078 | T | C | 22 | 25043518 | C | T |
| 22 | 25068063 | C | T | 22 | 25068076 | C | T | 22 | 25798856 | G | A |
| 22 | 25799013 | T | C | 22 | 25799024 | T | A | 22 | 25832242 | A | G |
| 22 | 26179312 | C | T | 22 | 27863300 | G | A | 22 | 28007568 | G | A |
| 22 | 29090363 | G | A | 22 | 29090890 | G | A | 22 | 29099754 | T | C |
| 22 | 29108112 | A | C | 22 | 29108229 | A | G | 22 | 29129741 | T | C |
| 22 | 29999742 | C | G | 22 | 29999784 | C | A | 22 | 29999878 | G | C |
| 22 | 30032484 | A | C | 22 | 30053963 | A | G | 22 | 30053984 | A | G |
| 22 | 30054024 | G | A | 22 | 30056945 | G | A | 22 | 30064117 | C | T |
| 22 | 30064731 | A | G | 22 | 31071644 | A | C | 22 | 32813600 | T | C |
| 22 | 32813975 | C | G | 22 | 33171838 | G | T | 22 | 34225927 | C | A |
| 22 | 34226068 | G | A | 22 | 34226412 | C | G | 22 | 34226440 | T | C |
| 22 | 34842414 | G | C | 22 | 35843103 | C | T | 22 | 35843109 | C | T |
| 22 | 36678453 | C | G | 22 | 36678476 | C | A | 22 | 36679058 | T | C |
| 22 | 38054038 | A | G | 22 | 41514151 | C | T | 22 | 41514155 | G | A |
| 22 | 41536064 | T | C | 22 | 41543721 | G | A | 22 | 41544167 | C | T |
| 22 | 41552926 | T | G | 22 | 41554690 | C | T | 22 | 41565285 | A | G |
| 22 | 41565827 | A | G | 22 | 41566823 | A | G | 22 | 41570071 | T | C |
| 22 | 42232577 | C | T | 22 | 42232588 | G | A | 22 | 42522312 | T | C |
| 22 | 42522392 | G | A | 22 | 42523003 | A | G | 22 | 42523209 | T | C |
| 22 | 42523211 | T | C | 22 | 42526049 | C | G | 22 | 42536064 | T | C |
| 22 | 42536954 | A | G | 22 | 42537023 | T | C | 22 | 42537029 | C | T |
| 22 | 42537115 | C | T | 22 | 42537119 | C | T | 22 | 42537127 | A | G |
| 22 | 42537196 | T | C | 22 | 42538399 | C | T | 22 | 42539105 | T | C |
| 22 | 42539720 | C | G | 22 | 42540667 | C | T | 22 | 42540668 | A | G |
| 22 | 42547739 | G | A | 22 | 42547755 | C | G | 22 | 42548094 | A | G |
| 22 | 42548880 | G | A | 22 | 43735824 | T | C | 22 | 44510034 | T | A |
| 22 | 44510219 | T | C | 22 | 44510230 | T | C | 22 | 44510240 | A | G |
| 22 | 44510329 | C | T | 22 | 44510554 | G | A | 22 | 44510624 | A | G |
| 22 | 44510631 | G | T | 22 | 44511408 | G | C | 22 | 44511478 | T | C |
| 22 | 44511626 | T | C | 22 | 44511663 | G | C | 22 | 45676445 | C | G |
| 22 | 47002427 | T | C | 22 | 47566744 | G | A | 22 | 48301616 | T | C |
| 22 | 48301642 | C | T | 22 | 48301646 | G | A | 22 | 48301660 | C | T |
| 22 | 49258340 | G | C | 22 | 49258341 | G | C | 22 | 49258485 | A | G |
| 22 | 49258525 | A | G | 22 | 49258700 | G | C | 22 | 49258701 | G | C |
| 22 | 49551354 | A | G | 22 | 49551680 | G | A | 22 | 49551682 | G | A |
| 22 | 49551684 | A | G | 22 | 49551689 | C | A | 22 | 49551690 | C | T |
| 22 | 49551694 | G | A | 22 | 49551724 | G | C | 22 | 49551734 | T | C |
| 22 | 49551749 | G | A | 22 | 49551895 | G | A | 22 | 49551927 | G | A |
| 22 | 49551969 | C | T | 22 | 49551990 | T | C | 22 | 50017556 | G | A |
| 22 | 51115526 | T | C | 22 | 51115575 | C | T | X | 323851 | G | A |
| X | 323960 | C | T | X | 324085 | G | A | X | 324097 | T | C |
| X | 324135 | G | A | X | 324137 | A | C | X | 324137 | A | G |
| X | 324146 | G | A | X | 324224 | C | G | X | 324245 | G | A |
| X | 933046 | T | A | X | 933079 | A | T | X | 933848 | C | T |
| X | 1414936 | C | A | X | 1415582 | A | C | X | 1415726 | G | C |
| X | 1415754 | A | G | X | 1416082 | A | C | X | 1416494 | A | C |
| X | 1416825 | A | G | X | 1416859 | C | G | X | 1417722 | A | G |
| X | 1882476 | C | A | X | 1882644 | G | C | X | 1882671 | T | G |
| X | 1883661 | T | G | X | 2320736 | C | T | X | 2320768 | C | T |
| X | 2550218 | A | G | X | 2550271 | T | C | X | 3354064 | T | C |
| X | 3354073 | C | G | X | 3354304 | T | G | X | 3354320 | C | G |
| X | 3354323 | T | C | X | 3354489 | C | T | X | 3947844 | G | A |
| X | 3947940 | G | A | X | 3948055 | A | G | X | 3948056 | C | A |
| X | 3948072 | C | A | X | 3948325 | A | G | X | 5728321 | G | A |
| X | 5728524 | T | A | X | 6750277 | G | A | X | 6750665 | T | C |
| X | 7811259 | G | C | X | 7811504 | G | A | X | 7811645 | C | G |
| X | 7811747 | T | C | X | 7812017 | C | T | X | 7812081 | C | T |
| X | 7812197 | T | C | X | 8138080 | G | C | X | 8138160 | T | C |
| X | 8138165 | C | G | X | 8138170 | C | G | X | 8138171 | T | C |
| X | 8138182 | A | G | X | 8434256 | A | G | X | 8434367 | C | T |
| X | 8434432 | A | G | X | 8434433 | G | C | X | 8960281 | C | T |
| X | 9372403 | A | G | X | 9374934 | C | A | X | 9375163 | A | C |
| X | 9376287 | T | G | X | 9377057 | G | A | X | 9378063 | T | C |
| X | 9378595 | C | G | X | 9380687 | C | G | X | 9382151 | T | C |
| X | 9909935 | T | C | X | 9910404 | A | T | X | 9910483 | C | A |
| X | 11985475 | C | T | X | 11985898 | A | T | X | 12023301 | C | G |
| X | 12023305 | A | G | X | 12023314 | A | G | X | 12023535 | G | C |
| X | 13222032 | G | A | X | 13668792 | C | T | X | 13669007 | G | A |
| X | 14291097 | G | A | X | 14291174 | G | A | X | 14291313 | T | C |
| X | 14852765 | G | C | X | 15665327 | C | T | X | 16351394 | T | C |
| X | 16351704 | C | T | X | 16351825 | A | G | X | 17387641 | A | G |
| X | 17794293 | A | G | X | 17794390 | G | C | X | 18247605 | T | C |
| X | 21942691 | C | T | X | 22313514 | G | A | X | 22314058 | G | C |
| X | 22553123 | A | G | X | 22553314 | G | A | X | 24007345 | T | C |
| X | 26225293 | C | T | X | 26225571 | C | A | X | 26735568 | A | G |
| X | 26735679 | G | T | X | 27029285 | C | A | X | 28676918 | A | G |
| X | 28871324 | A | G | X | 28871343 | A | G | X | 28871801 | C | T |
| X | 29149454 | G | T | X | 29149490 | A | G | X | 30277764 | C | T |
| X | 30618867 | T | C | X | 31277151 | C | T | X | 32224435 | T | C |
| X | 32889854 | G | A | X | 33050164 | T | C | X | 34444321 | T | G |
| X | 35083559 | C | A | X | 35971670 | A | C | X | 35971684 | A | G |
| X | 36959988 | C | T | X | 39944288 | C | T | X | 40857040 | C | T |
| X | 41546554 | C | T | X | 43440829 | T | C | X | 43440856 | T | C |
| X | 44601065 | T | G | X | 45028722 | T | C | X | 45029140 | A | G |
| X | 45634577 | C | A | X | 47966001 | A | G | X | 48134195 | T | C |
| X | 48134267 | C | A | X | 48134283 | A | G | X | 48134352 | G | A |
| X | 48134363 | T | C | X | 48196483 | C | T | X | 48548091 | C | T |
| X | 49337861 | G | A | X | 49337915 | G | A | X | 50776337 | A | C |
| X | 51361920 | C | T | X | 54846603 | C | T | X | 54976385 | T | C |
| X | 55684550 | G | C | X | 55684552 | A | T | X | 55684553 | T | C |
| X | 57163476 | C | G | X | 62573853 | A | G | X | 67310673 | G | A |
| X | 67965273 | C | T | X | 70338399 | A | G | X | 70354080 | C | A |
| X | 70355942 | G | C | X | 70361608 | T | C | X | 70361619 | T | C |
| X | 70886761 | G | A | X | 70939147 | G | C | X | 72066630 | C | T |
| X | 74375460 | C | T | X | 74803555 | T | A | X | 75449353 | G | C |
| X | 75449473 | A | G | X | 75555810 | C | T | X | 76845060 | C | T |
| X | 76848940 | C | T | X | 76912391 | T | C | X | 76919991 | T | C |
| X | 77671395 | A | G | X | 78526387 | C | G | X | 79376181 | C | G |
| X | 79376359 | G | A | X | 79712450 | C | A | X | 81978259 | A | G |
| X | 82987871 | T | A | X | 82988286 | T | A | X | 83489042 | C | A |
| X | 84535451 | A | G | X | 85996514 | A | G | X | 86125877 | C | T |
| X | 86126034 | A | C | X | 86126184 | C | A | X | 86126214 | C | A |
| X | 88458887 | G | A | X | 88459088 | A | G | X | 88460086 | C | T |
| X | 88460163 | C | A | X | 88460234 | C | T | X | 88460693 | C | G |
| X | 88461023 | A | C | X | 88461098 | G | T | X | 88461193 | G | C |
| X | 88461257 | T | C | X | 88461306 | C | T | X | 88461381 | G | A |
| X | 88461454 | G | T | X | 88461489 | G | A | X | 89179384 | G | A |
| X | 89531825 | G | A | X | 90467751 | C | T | X | 90468268 | A | G |
| X | 90937645 | C | T | X | 90937839 | C | T | X | 91240753 | G | A |
| X | 91241043 | C | T | X | 91651454 | G | A | X | 91651560 | T | C |
| X | 91651589 | T | C | X | 91651601 | C | G | X | 94235658 | G | C |
| X | 97861548 | G | A | X | 97861806 | C | G | X | 100600966 | C | T |
| X | 100604681 | C | T | X | 100611432 | G | A | X | 100614597 | C | T |
| X | 100615025 | G | T | X | 100616989 | C | T | X | 100625293 | C | T |
| X | 100626864 | C | T | X | 100630500 | G | A | X | 101383199 | T | C |
| X | 102633394 | A | T | X | 105034364 | C | G | X | 106516125 | G | C |
| X | 107492960 | G | C | X | 108115548 | C | T | X | 111815818 | T | C |
| X | 114629506 | T | C | X | 114997566 | A | G | X | 115005861 | T | C |
| X | 115005934 | C | T | X | 115959641 | C | A | X | 116167250 | G | A |
| X | 117479815 | C | G | X | 119313694 | G | C | X | 122008986 | A | G |
| X | 122648454 | T | C | X | 122696563 | C | T | X | 122932585 | A | C |
| X | 123159474 | T | C | X | 123181551 | G | A | X | 123194867 | T | C |
| X | 123197557 | T | C | X | 123227604 | T | C | X | 124576157 | G | T |
| X | 125779449 | T | C | X | 125779655 | C | T | X | 126247979 | C | A |
| X | 127724972 | C | T | X | 128495450 | C | G | X | 130974130 | T | C |
| X | 130974165 | A | G | X | 130974206 | G | A | X | 130974276 | A | C |
| X | 130974277 | A | C | X | 130974284 | G | C | X | 130974290 | C | G |
| X | 130974310 | T | C | X | 130974312 | G | A | X | 132426862 | G | C |
| X | 135961796 | C | G | X | 137367771 | T | C | X | 138265862 | A | T |
| X | 138266294 | T | C | X | 139298295 | C | T | X | 139298601 | C | T |
| X | 139298612 | T | C | X | 139960824 | T | C | X | 140147409 | A | G |
| X | 140147500 | C | T | X | 140336615 | T | A | X | 140673061 | C | T |
| X | 140673198 | T | G | X | 140673372 | C | T | X | 140673423 | G | C |
| X | 140786796 | G | A | X | 140993642 | C | T | X | 140993905 | C | T |
| X | 140993912 | C | T | X | 140993913 | C | G | X | 140993945 | T | C |
| X | 140993947 | T | C | X | 140993989 | T | C | X | 140994031 | G | C |
| X | 140994037 | C | A | X | 142605437 | A | C | X | 142605455 | C | T |
| X | 142605483 | A | G | X | 142605491 | G | C | X | 142605583 | C | T |
| X | 142605628 | G | A | X | 142804302 | T | C | X | 142804326 | C | T |
| X | 143901920 | T | C | X | 143902298 | G | T | X | 144328894 | C | T |
| X | 144940393 | G | A | X | 146052064 | T | C | X | 146052294 | G | C |
| X | 146266206 | T | G | X | 146266521 | G | T | X | 146679744 | A | G |
| X | 146679975 | T | A | X | 146680129 | C | T | X | 149382872 | C | G |
| X | 149383074 | A | G | X | 150162380 | C | T | X | 152155921 | C | T |
| X | 152156110 | C | T | X | 152156144 | A | G | X | 152156176 | G | C |
| X | 152538286 | A | T | X | 152538627 | G | A | X | 152582526 | G | A |
| X | 153009797 | G | A | X | 153010224 | C | T | X | 153010267 | G | A |
| X | 154376753 | C | G | X | 155233098 | T | C | | | | |

2. The use according to claim 1, wherein the evaluating homologous recombination deficiency is to calculate scoring HRD according to the following formula:
HRD=LOH+TAI+LST,
wherein
LOH refers to a number of heterozygous loss regions of at least 15 Mb without spanning an entire chromosome;
TAI refers to a number of allelic imbalances in front of telomere ends of chromosomes; and
LST refers to a number of a chromosomal break region not greater than 3 Mb and a fragment of at least 10 Mb.
